(19) United States
(12) Patent Application Publication
(10) Pub. No.: US 2023/0110958 A1
(43) Pub. Date: Apr. 13, 2023

(54) IL27 RECEPTOR AGONISTS AND METHODS OF USE THEREOF

(71) Applicant: Regeneron Pharmaceuticals, Inc., Tarrytown, NY (US)

(72) Inventors: Arielle Glatman Zaretsky, New York, NY (US); Jiaxi Wu, Pleasantville, NY (US); Tong Zhang, New Rochelle, NY (US); Sokol Haxhinasto, Brookfield, CT (US); Nicolin Bloch, Ossining, NY (US)

(21) Appl. No.: 17/887,565

(22) Filed: Aug. 15, 2022

Related U.S. Application Data
(60) Provisional application No. 63/233,651, filed on Aug. 16, 2021.

(51) Int. Cl. C07K 14/54; C07K 14/715

(52) U.S. Cl. CPC C07K 14/54; C07K 14/7155; C07K 2319/30; A61K 38/00

(57) ABSTRACT

The present disclosure relates to IL27 receptor agonists with improved therapeutic profiles.

### 1. CROSS-REFERENCE TO RELATED APPLICATIONS

This application claims the priority benefit of U.S. provisional application No. 63/233,651, filed on Aug. 16, 2021, the contents of which are incorporated herein in its entirety by reference thereto.

### SEQUENCE LISTING

The instant application contains a Sequence Listing which has been submitted electronically and is hereby incorporated by reference in its entirety. Said copy, created on Dec. 1, 2022, is named RGN-007US_SL.xml and is 111,769 bytes in size.

### 2. BACKGROUND

Interleukin-27 (IL27 or IL-27) is a heterodimeric cytokine composed of two subunits: the Epstein-Barr virus-induced gene 3 (EBI3) and IL27 p28 (p28). IL27 is structurally related to both the IL27 and IL6 cytokine families. IL27 binds to and mediates signaling through a receptor complex composed of gp130 and the IL27Ra (WSX1) that activates Janus kinase (JAK)-signal transducer and activator of transcription (STAT) and mitogen activated protein kinase (MAPK) signaling (Kastelein et al., 2007, Annu Rev Immunol. 25:221-242).

IL27 was initially reported as an immune-enhancing cytokine, however subsequent studies demonstrated that IL27 displays complex immune-regulatory functions (reviewed in Fabbi et al., 2017, Mediators of Inflammation 42:1-14). As a result of its pleiotropic activity, IL27 has been linked to a broad range of diseases, disorders and conditions, including inflammatory conditions and immune-related disorders.

One drawback of using IL27 in therapy, and particularly any form of recombinant IL27, is its short serum half-life. The loss of IL27 activity in vivo may be due to several factors, including renal clearance and proteolytic degradation.

It would be an advantage to have an IL27 receptor agonist that is better tolerated during systemic exposure during treatment, by enhancing the circulating life (delayed clearance), solubility, and stability of IL27. It would further be advantageous to have an IL27 receptor agonist with an improved therapeutic index and minimal side effects, while being administrable at therapeutically effective dose. The present disclosure addresses this and other related needs in the art.

### 3. SUMMARY

The present disclosure provides novel IL27 receptor agonists. In certain aspects, IL27 receptor agonists address the drawbacks of IL27 therapy and are characterized by improved therapeutic profiles by virtue of improved half lives and/or improved safety profiles. In further aspects, IL27 receptor agonists address the aggregation problems associated with traditional recombinant IL27 fusion constructs, for example fusion proteins comprising p28, EBI3 and an Fc domain. The IL27 receptor agonists of the disclosure typically comprise or consist of IL27 muteins that vary from native IL27 by primary amino acid sequence of p28 and/or EBI3 and/or by the inclusion of additional domains or moieties not normally present in IL27. The IL27 receptor (used interchangeably with “IL27 agonists”) and muteins typically comprise one or a pair of IL27 monomers that each comprise a p28 and/or EBI3 moiety and an optional multimerization moiety (e.g., an Fc domain), an optional stabilization moiety (e.g., human serum albumin) and/or an optional targeting moiety (e.g., an scFv) antibody) or a component of a targeting moiety (e.g., the VH domain of a Fab targeting moiety), optionally in association with one or more additional polypeptide chains (for example a polypeptide chain comprising another multimerization moiety (e.g., an Fc domain) or a targeting moiety component (e.g., the VL domain of a Fab targeting moiety). Exemplary IL27 monomers are disclosed in Section 5.2. Exemplary IL27 receptor agonists are disclosed in Section 5.2 and numbered embodiments 24 to 24 to 318, and depicted in FIGS. 3 to 6.

The present disclosure further provides variant p28 moieties that incorporate amino acid substitutions that contribute to improved therapeutic profiles. Exemplary variant p28 moieties are disclosed in Section 5.3.2 and numbered embodiments 1 to 23.

The present disclosure further provides p28 proteins and EBI3 proteins. Some IL27 receptor agonists and muteins of the disclosure comprise a p28 protein associated with an EBI3 protein.

In certain aspects, the p28 proteins comprise a p28 moiety and a multimerization (e.g., Fc) domain. The p28 proteins may comprise one, two or more polypeptide chains and are typically configured to associate with an EBI3 moiety, for example the EBI3 moiety of an EBI3 protein. In some embodiments, the p28 proteins do not comprise an EBI3 moiety. Exemplary p28 proteins are disclosed in Section 5.2 and numbered embodiments 319 to 326.

In certain aspects, the EBI3 proteins comprise an EBI3 moiety and a multimerization (e.g., Fc) domain. The EBI3 proteins may comprise one, two or more polypeptide chains and are typically configured to associate with a p28 moiety, for example the p28 moiety of a p28 protein. In some embodiments, the EBI3 proteins do not comprise a p28 moiety. Exemplary EBI3 proteins are disclosed in Section 5.2 and numbered embodiments 327 to 334.

The disclosure further provides nucleic acids encoding the IL27 receptor agonists, the IL27 muteins, the IL27 monomers, the p28 proteins, the EBI3 proteins, the p28 moieties and the EBI3 moieties of the disclosure. The nucleic acids encoding the IL27 receptor agonists, IL27 muteins, p28 proteins and EBI3 proteins that are composed of two or more polypeptide chains can be a single nucleic acid (e.g., a vector encoding all polypeptide chains) or a plurality of nucleic acids (e.g., two or more vectors encoding the different polypeptide chains). The disclosure further provides host cells and cell lines engineered to express the nucleic acids and the IL27 receptor agonists, the IL27 muteins, the IL27 monomers, the p28 proteins, the EBI3 proteins, the p28 moieties and the EBI3 moieties of the disclosure. The disclosure further provides methods of producing an IL27 receptor agonist, an IL27 mutein, an IL27 monomer, the p28 proteins, the EBI3 proteins, a p28 moiety or an EBI3 moiety of the disclosure. Exemplary nucleic acids, host cells, cell lines, and methods of producing the IL27 receptor agonists, the IL27 muteins, the IL27 monomers, the p28 proteins, the EBI3 proteins, the p28 moieties, and the EBI3 moieties are described in Section 5.9 and numbered embodiments 335 to 343, infra.

The disclosure further provides pharmaceutical compositions comprising the IL27 receptor agonists, the IL27 muteins, the IL27 monomers, the p28 proteins, the EBI3 proteins, the p28 moieties and the EBI3 moieties of the disclosure. Exemplary pharmaceutical compositions are described in Section 5.10 and numbered embodiments 344 to 346, infra.

Further provided herein are methods of using the IL27 receptor agonists, the IL27 muteins, the IL27 monomers, the p28 proteins, the EBI3 proteins, the p28 moieties, the EBI3 moieties and the pharmaceutical compositions of the disclosure, e.g., for modulating the immune response, treating autoimmune conditions and/or localized delivery of an IL27 receptor agonist. Exemplary methods are described in Section 5.11 and numbered embodiments 347 to 355, infra.

### 4. BRIEF DESCRIPTION OF THE FIGURES

FIGS. 1A-1B are cartoon schematics of the IL27 heterodimer (FIG. 1A) and the heterodimeric IL27 receptor (FIG. 1B).

FIG. 2 is a cartoon schematic of the wild-type IL27 heterodimer.

FIGS. 3A-3G illustrate various orientations of embodiments of IL27 agonists of the disclosure referred to as IL27M1 through IL27M3 and IL27M12 through IL27M15. The triangle in one of the CH3 domains indicates that the two CH3 are not identical and contain one or more mutations that permit heterodimerization (e.g., knob-in-hole mutations, a star mutation, etc.). The illustrations are intended to depict the configurations, including N- to C-terminal order, of domains included in the IL27 agonists and are not intended to convey specific sequences, scale or three-dimensional structure.

FIGS. 4A-4G illustrate additional orientations of embodiments of IL27 agonists of the disclosure referred to as IL27M4 through IL27M6 and IL27M16 through IL27M19. The triangle in one of the CH3 domains indicates that the two CH3 are not identical and contain one or more mutations that permit heterodimerization (e.g., knob-in-hole mutations, a star mutation, etc.). The illustrations are intended to depict the configurations, including N- to C-terminal order, of domains included in the IL27 agonists and are not intended to convey specific sequences, scale or three-dimensional structure.

FIGS. 5A-5B illustrate additional orientations of embodiments of IL27 agonists of the disclosure referred to as IL27M20 (FIG. 5A) and IL27M21 (FIG. 5B), comprising human serum albumin (HSA). The illustrations are intended to depict the configurations of domains included in the IL27 agonists and are not intended to convey specific sequences, scale or three dimensional structure.

FIGS. 6A-6E illustrate additional orientations of embodiments of IL27 agonists of the disclosure referred to as IL27M7 through IL27M11. The asterisk in one of the CH3 domains indicates that the two CH3 are not identical and contain one or more mutations that permit heterodimerization (e.g., knob-in-hole mutations, a star mutation, etc.). The illustrations are intended to depict the configurations, including N- to C-terminal order, of domains included in the IL27 agonists and are not intended to convey specific sequences, scale or three dimensional structure.

FIGS. 7A-7C illustrate the activity on STAT3-mediated luciferase reporter activity and aggregation profiles of IL27 muteins. Recombinant IL27 and IL27 muteins increase STAT3-response element driven luciferase activity in engineered MC9/STAT3-Luc reporter cells. Black filled circles represent commercially available murine IL27 (purchased from R&D Systems), closed filled triangles represent EBI3×p28-Fc (monovalent) (an example of an IL27 agonist having an orientation of IL27M2), closed filled squares represent EBI3-p28-Fc (bivalent) (an example of an IL27 agonist having an orientation IL27M1) (FIG. 7A). The SE-UPLC profile of EBI3×p28-Fc (monovalent)is shown in FIG. 7B and the aggregation profile of EBI3-p28-Fc (bivalent) is shown in FIG. 7C.

FIG. 8 illustrates the activity of IL27 muteins on STAT1 phosphorylation in CD4+ T cells isolated from naïve spleens. Recombinant IL27 and EBI3×p28-Fc (monovalent), but not EBI3-p28-Fc (bivalent), induce a dose-dependent increase in pSTAT1 in CD4+ T cells isolated from naïve spleens. Circles represent commercially available murine IL27 (purchased from R&D Systems), squares represent EBI3-p28-Fc (bivalent), and triangles represent EBI3×p28-Fc (monovalent).

FIGS. 9A-9L illustrate the SE-UPLC traces for IL27 agonists of the disclosure. FIGS. 9A and 9B: IL27 domain of IL27 agonist was of human origin. FIGS. 9C-9L: IL27 domain of IL27 agonist was of murine origin.

FIGS. 10A-10C illustrate the activity of IL27 agonists of the disclosure on a mouse cell line in an IL27 reporter assay. MC9/STAT3-Luc cells were incubated with a titration of mIL27 monomeric IL2 Fc-fusions (FIG. 10A) or dimeric IL27 Fc-fusions (FIG. 10B) or IL27 HSA fusions (FIG. 10C).

FIG. 11 illustrates the activity of IL27 agonists of the disclosure on a human cell line in an IL27 reporter assay. NCI-H929/GAS-Luc cells were incubated with a titration of human IL27 (closed circle with dashed line) or monovalent (hEBI3×hp28-Fc (e.g., having the configuration of IL27M2); square with solid line) or bivalent (hEBI3-hp28-Fc (e.g., having the configuration of IL27M1); triangle with solid line) IL27-Fc. 5 h and 30 min later, STAT1 activity was assessed by luminescent readout

FIG. 12 illustrates the activity of IL27 muteins on Th0, Th2, and Th17 polarization of naïve CD4+ mouse T cells. Recombinant IL27 and EBI3×p28-Fc (monovalent), but not EBI3-p28-Fc (bivalent), inhibit Th2 polarization of naïve CD4+ mouse T cells as determined by reduced expression of the Th2-associated transcription factor Gata3 under Th2 polarization-inducing conditions (Th0: 10 ug/mL anti-IFNg+10 ug/mL anti-IL-4), Th2: 10 ug/mL anti-IFNg+50 ng/mL rIL-4, Th17: 10 ug/mL anti-IFNg+10 ug/mL anti-IL-4+1 ng/mL rhTGFb+10 ng/mL rIL-6).

FIGS. 13A-13C illustrate the ability of recombinant IL27 muteins of the disclosure to inhibit Gata3 expression during Th2 polarization (10 ug/mL anti-IFNg+50 ng/mL IL-4) as assessed in an in vitro mouse T cell polarization assay. (FIG. 13A) Flow cytometric plots showing Gata3 expression on CD4+ T cells are shown. (FIG. 13B) Gata3 MFI was quantified. (FIG. 13C) The ability of recombinant IL27 muteins of the disclosure to promote PDL1 expression under T cell polarizing conditions (Th0: 10 ug/mL anti-IFNg+10 ug/mL anti-IL-4), Th2: 10 ug/mL anti-IFNg+50 ng/mL rIL-4, Th17: 10 ug/mL anti-IFNg+10 ug/mL anti-IL-4+1 ng/mL rhTGFb+10 ng/mL rIL-6) was assessed in an in vitro mouse T cell polarization assay.

FIG. 14 illustrates the PK of recombinant IL27 muteins of the disclosure that was evaluated in vivo. C57BL/6 mice were treated with 10 ug of the indicated recombinant IL27 mutein intraperitoneally. Serum was collected 2, 6, 24, and 48 hours after treatment. An ELISA (duoset mouse IL27 p28 ELISA; R&D Systems) was performed to quantify the respective IL27 mutein levels in serum at each time point.

FIGS. 15A-15C illustrate a model of the interaction between IL27 and the IL27 receptor complex (FIG. 15A), a three-dimensional model of receptor binding sites occurring on IL27 p28 (FIG. 15B), and the sequence alignment for mouse IL27p28 (SEQ ID NO: 36) and human IL27p28 (SEQ ID NO: 34) (FIG. 15C). Solid arrows indicate mutation sites in binding site 2 involved in receptor binding. Dashed arrows indicate mutation sites in binding site 3 involved in receptor binding.

FIG. 16 illustrates an IL27 reporter assay for mIL27 site 2 and site 3 muteins. MC9/STAT3-Luc were incubated with a titration of mIL27 or monovalent Fc-IL27 muteins, 5 h later, STAT3 activity was assessed by luminescent readout. The right panel is a zoomed-in view of the portion enclosed by the dotted box presented on the left panel.

FIGS. 17A-17D illustrate the activity of mIL27 site 2 and site 3 muteins in primary human T cells.

FIGS. 18A-18B illustrate the flow binding of mIL27 site 2 and site 3 muteins to mouse reporter cells MC9/STAT3-Luc (FIG. 18A) and IL27Ra knock out derivative (FIG. 18B).

FIGS. 19A-19H illustrate the potency of targeted IL27 muteins on target expressing cells (FIGS. B, D, F, and H) versus non-expressing cells (FIGS. A, C, E, and G).

### 5. DETAILED DESCRIPTION

### 5.1. Definitions

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Antigen Binding Domain or ABD: The term “antigen binding domain” or “ABD” as used herein refers to the portion of a targeting moiety that is capable of specific, non-covalent, and reversible binding to a target molecule.

Associated: The term “associated” in the context of an IL27 receptor agonist or a component thereof (e.g., an IL27 EBI3 moiety; an IL27 p28 moiety; a targeting moiety such as an antibody) refers to a functional relationship between two or more polypeptide chains. In particular, the term “associated” means that two or more polypeptides are associated with one another, e.g., non-covalently through molecular interactions or covalently through one or more disulfide bridges or chemical cross-linkages, so as to produce a functional IL27 receptor agonist. Examples of associations that might be present in an IL27 receptor agonist of the disclosure include (but are not limited to) associations between IL27 EBI3 and p28 moieties, associations between homodimeric or heterodimeric Fc domains in an Fc region, associations between VH and VL regions in a Fab or scFv, associations between CH1 and CL in a Fab, and associations between CH3 and CH3 in a domain substituted Fab.

Bivalent: The term “bivalent” as used herein in reference to IL27 and/or a targeting moiety in an IL27 receptor agonist means an IL27 receptor agonist that has two IL27 heterodimers (i.e., two EBI3×p28 heterodimers) and/or targeting moieties, respectively. Typically, IL27 receptor agonists that are bivalent for an IL27 moiety and/or a targeting moiety are dimeric (either homodimeric or heterodimeric).

Cancer: The term “cancer” refers to a disease characterized by the uncontrolled (and often rapid) growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, adrenal gland cancer, autonomic ganglial cancer, biliary tract cancer, bone cancer, endometrial cancer, eye cancer, fallopian tube cancer, genital tract cancers, large intestinal cancer, cancer of the meninges, oesophageal cancer, peritoneal cancer, pituitary cancer, penile cancer, placental cancer, pleura cancer, salivary gland cancer, small intestinal cancer, stomach cancer, testicular cancer, thymus cancer, thyroid cancer, upper aerodigestive cancers, urinary tract cancer, vaginal cancer, vulva cancer, lymphoma, leukemia, lung cancer and the like.

Complementarity Determining Region or CDR: The terms “complementarity determining region” or “CDR,” as used herein, refer to the sequences of amino acids within antibody variable regions which confer antigen specificity and binding affinity. In general, there are three CDRs in each heavy chain variable region (CDR-H1, CDR-H2, HCDR-H3) and three CDRs in each light chain variable region (CDR1-L1, CDR-L2, CDR-L3). Exemplary conventions that can be used to identify the boundaries of CDRs include, e.g., the Kabat definition, the Chothia definition, the ABM definition and the IMGT definition. See, e.g., Kabat, 1991, “Sequences of Proteins of Immunological Interest,” National Institutes of Health, Bethesda, Md. (Kabat numbering scheme); Al-Lazikani et al., 1997, J. Mol. Biol. 273:927-948 (Chothia numbering scheme); Martin et al., 1989, Proc. Natl. Acad. Sci. USA 86:9268-9272 (ABM numbering scheme); and Lefranc et al., 2003, Dev. Comp. Immunol. 27:55-77 (IMGT numbering scheme). Public databases are also available for identifying CDR sequences within an antibody.

EBI3 Moiety or IL27 EBI3 Moiety: The terms “EBI3 moiety” and “IL27 EBI3 moiety” refer to an amino acid sequence comprising at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to a p28 binding portion of a mammalian, e.g., human or murine, EBI3 protein. The sequence of human EBI3 has the Uniprot identifier Q14213 (uniprot.org/uniprot/Q14213). The sequence of murine EBI3 has the Uniprot identifier O35228 (uniprot.org/uniprot/O35228).

In some embodiments, the EBI3 moiety comprises an amino acid sequence comprising at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to a mature mammalian EBI3 protein, e.g., human or murine EBI3 (e.g., amino acids 24-229 of full-length human EBI3).

Further embodiments of EBI3 moieties are described in Section 5.3.1.

EBI3 Polypeptide: The term “EBI3 polypeptide” refers to a polypeptide comprising an EBI3 moiety (e.g., as described in Section 5.2). In some embodiments, the EBI3 polypeptide is a fusion polypeptide, e.g., a polypeptide comprising an Fc domain in addition to the EBI3 moiety.

EBI3 Protein: The term “EBI3 protein” refers to a monomeric or polymeric (e.g., dimeric) protein comprising an EBI3 moiety (e.g., an Fc dimer comprising an EBI3 moiety). The term “EBI3 protein” encompasses an EBI3 polypeptide.

EC50: The term “EC50” refers to the half maximal effective concentration of a molecule (such as an IL27 agonist) which induces a response halfway between the baseline and maximum after a specified exposure time. The EC50 essentially represents the concentration of an antibody or IL27 agonist where 50% of its maximal effect is observed. In certain embodiments, the EC50 value equals the concentration of an IL27 agonist that gives half-maximal STAT3 activation in an assay as described in Section 7.1.2.

Epitope: The term “epitope” is a portion of an antigen (e.g., target molecule) recognized by an antibody or other antigen-binding moiety. An epitope can be linear or conformational.

Fab: The term “Fab” in the context of a targeting moiety of the disclosure refers to a pair of polypeptide chains, the first comprising a variable heavy (VH) domain of an antibody N-terminal to a first constant domain (referred to herein as C1), and the second comprising variable light (VL) domain of an antibody N-terminal to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab.

Fc Domain and Fc Region: The term “Fc domain” refers to a portion of the heavy chain that pairs with the corresponding portion of another heavy chain. The term “Fc region” refers to the region of antibody-based binding molecules formed by association of two heavy chain Fc domains. The two Fc domains within the Fc region may be the same or different from one another. In a native antibody the Fc domains are typically identical, but one or both Fc domains might advantageously be modified to allow for heterodimerization, e.g., via a knob-in-hole interaction. Further, the Fc domains can include chimeric sequences from more than one immunoglobulin isotype.

Host cell: The term “host cell” as used herein refers to cells into which a nucleic acid of the disclosure has been introduced. The terms “host cell” and “recombinant host cell” are used interchangeably herein. It is understood that such terms refer to the particular subject cell and to the progeny or potential progeny of such a cell. Because certain modifications may occur in succeeding generations due to either mutation or environmental influences, such progeny may not, in fact, be identical to the parent cell, but are still included within the scope of the term as used herein. Typical host cells are eukaryotic host cells, such as mammalian host cells. Exemplary eukaryotic host cells include yeast and mammalian cells, for example vertebrate cells such as a mouse, rat, monkey or human cell line, for example HKB11 cells, PER.C6 cells, HEK cells or CHO cells.

IL27 Agonist or IL27 ReceptorAgonist: The terms “IL27 agonist” and “IL27 receptor agonist” are used interchangeably herein to refer to a molecule comprising or consisting of an IL27 mutein and which has IL27 activity. The IL27 activity can be greater than, lower than, or equal to the activity of wild type or recombinant IL27 (e.g., human or murine IL27) in one or more in vitro or in vivo biological assays, for example the STAT3-driven luciferase-based reporter assay described in Section 7.1.2. In various embodiments, the IL27 agonist has activity, relative to recombinant IL27, ranging from 5% to 90%, from 5% to 85%, from 5% to 80%, from 10% to 80%, from 15% to 80%, from 20% to 80%, from 25% to 80%, from 30% to 80%, from 35% to 80%, from 45% to 80%, from 50% to 80%, from 5% to 70%, from 10% to 70%, from 15% to 70%, from 20% to 70%, from 25% to 70%, from 30% to 70%, from 35% to 70%, from 45% to 70%, or from 50% to 70%.

IL27 Moiety: The term “IL27 moiety” as used herein refers to an EBI3 moiety (e.g., as described in Section 5.3.1) or a p28 moiety (e.g., as described in Section 5.3.2). Thus, the related term “intra-IL27 moiety linker” refers to a linker connecting two IL27 moieties, e.g., an EBI3 moiety and a p28 moiety.

IL27 Monomer or Monomer: The terms monomer and IL27 monomer as used herein refer to a molecule comprising a first polypeptide chain which (a) comprises an EBI3 moiety and a p28 moiety and is capable of associating with a second polypeptide chain; (b) comprises a EBI3 moiety and is capable of associating with a p28 moiety on a second polypeptide chain; (c) comprises a p28 moiety and is capable of associating with a EBI3 moiety on a second polypeptide chain; (d) comprises a multimerization moiety (e.g., an Fc domain) and is capable of associating with a corresponding multimerization moiety (e.g., another Fc domain) on a second polypeptide chain; (e) comprises a stabilization moiety (e.g., human serum albumin) and a p28 moiety and/or an EBI3 moiety; or (f) any combination of (a), (b), (c), (d), and (e) above. In some embodiments, monomers are capable of associating with other monomers through a EBI3/p28 moiety pairing and/or a multimerization moiety (e.g., Fc domain) pairing. In some embodiments, the monomers form associations through hinge sequences or other portions of Fc domains. Thus, a monomer of the disclosure is capable of associating with another monomer to form a dimer. The dimers can be homodimeric, in which each constituent monomer is identical, or heterodimeric, in which case each constituent monomer is different. As used herein, the reference to a “monomer” does not preclude the presence of a second polypeptide chain that does not comprise an EBI3, p28, or multimerization moiety, for example a light chain of a Fab domain. Thus, a “dimer” of two monomers may include more than two polypeptide chains, e.g., may include three or four polypeptide chains.

In some embodiments, two or more IL27 monomers (e.g., two, three, or four IL27 monomers) associate with one another to form an IL27 receptor agonist of the disclosure. In other embodiments, a single IL27 monomer forms the IL27 receptor agonist of the disclosure.

IL27 Mutein: An “IL27 mutein” is a variant IL27 molecule composed or one or more polypeptide chains (e.g., one, two, three or four polypeptide chains) comprising an IL27 EBI3 (referred to as “EBI3”) moiety and an IL27 p28 (“p28”) moiety in association with one another and which varies from native IL27 by (a) primary amino acid sequence and/or (b) association with additional domains not naturally associated with IL27, for example (i) a multimerization moiety (e.g., a dimerization domain such as an Fc domain) and/or (ii) a targeting moiety and/or (iii) a stabilization moiety.

In some embodiments, the term mutein refers to a structure (a) with or without a targeting moiety and/or (b) with or without a stabilization moiety and/or (c) with or without a multimerization moiety. In the context of the IL27 agonists of the disclosure, the term “IL27 mutein” sometimes refers to the core components of a variant IL27 molecule, namely the EBI3 and p28 moieties and sometimes also the multimerization moieties, such as Fc domains and any/or associated linker moieties, and/or the stabilization moieties, such as human serum albumin (HSA) and it is to be understood that the term “IL27 mutein” extends also to IL27 molecules comprising additional features, e.g., one or more targeting moieties, one or more stabilization moieties, one or more multimerization moieties, one or more linker moieties, and any combination of the foregoing, unless the context dictates otherwise.

The IL27 mutein can thus comprise an EBI3 and/or p28 moiety with one or more amino acid substitutions, deletions and/or insertions compared to wild type EBI3 and/or p28.

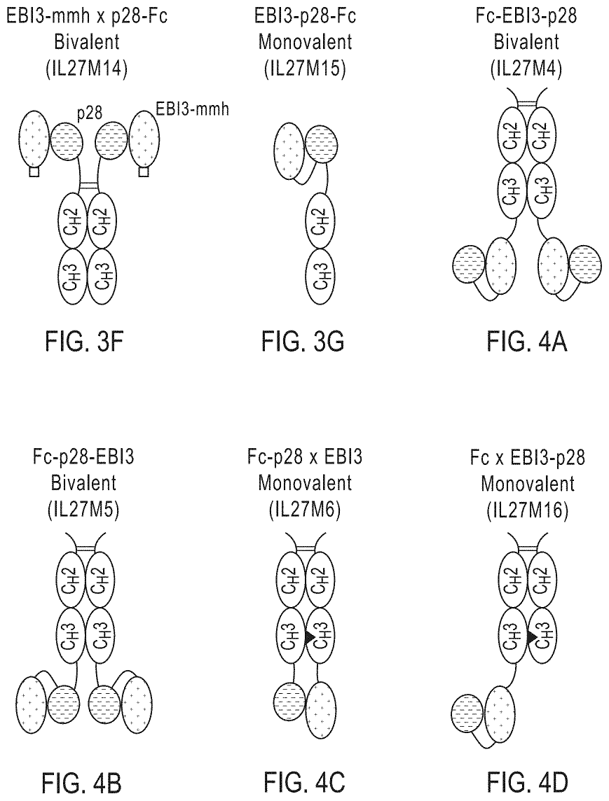

In some embodiments, the IL27 mutein has one or more mutations in its p28 moiety. Exemplary mutations, e.g., substitutions, are disclosed, inter alia, in Section 5.3.2 and subsections thereof, in Table 1, as well as in numbered embodiments 1 to 23. The EBI3 and p28 subunits of an IL27 mutein can be included in the same polypeptide chain, or can be included in different polypeptide chains. Exemplary configurations of the IL27 muteins and agonists of the disclosure are disclosed, inter alia, in FIGS. 3 to 6, Section 5.2 and as well as in numbered embodiments 24 to 318.

The IL27 mutein can be monovalent for EBI3 and p28 (i.e., has a single EBI3 moiety and a single p28 moiety) or multivalent for EBI3 and p28 (i.e., has multiple EBI3 moieties and p28 moieties). In some embodiments, the IL27 mutein is bivalent for EBI3 and p28 (i.e., has two EBI3 moieties and two p28 moieties). When an IL27 mutein is multivalent for EBI3 and p28, the multiple EBI3 moieties can be the same or different from one another and/or the multiple p28 moieties can be the same or different from one another.

An IL27 mutein can have altered function (e.g., receptor binding, affinity, cytokine activity) and/or altered pharmacokinetics as compared to wild type IL27.

Major histocompatibility complex and MHC: These terms refer to naturally occurring MHC molecules, individual chains of MHC molecules (e.g., MHC class I α (heavy) chain, β2 microglobulin, MHC class II α chain, and MHC class II β chain), individual subunits of such chains of MHC molecules (e.g., α1, α2, and/or α3 subunits of MHC class I α chain, α1-α2 subunits of MHC class II α chain, β1-β2 subunits of MHC class II β chain) as well as portions (e.g., the peptide-binding portions, e.g., the peptide-binding grooves), mutants and various derivatives thereof (including fusions proteins), wherein such portion, mutants and derivatives retain the ability to display an antigenic peptide for recognition by a T-cell receptor (TCR), e.g., an antigen-specific TCR. An MHC class I molecule comprises a peptide binding groove formed by the α1 and α2 domains of the heavy a chain that can stow a peptide of around 8-10 amino acids. Despite the fact that both classes of MHC bind a core of about 9 amino acids (e.g., 5 to 17 amino acids) within peptides, the open-ended nature of MHC class II peptide binding groove (the α1 domain of a class II MHC a polypeptide in association with the p1 domain of a class II MHC β polypeptide) allows for a wider range of peptide lengths. Peptides binding MHC class II usually vary between 13 and 17 amino acids in length, though shorter or longer lengths are not uncommon. As a result, peptides may shift within the MHC class II peptide binding groove, changing which 9-mer sits directly within the groove at any given time. Conventional identifications of particular MHC variants are used herein. The terms encompass “human leukocyte antigen” or “HLA”.

Monovalent: The term “monovalent” as used herein in reference to IL27 and/or a targeting moiety in an IL27 receptor agonist means an IL27 receptor agonist that has only a single IL27 heterodimer (i.e., one EBI3×p28 heterodimer) and/or targeting moiety, respectively.

Operably linked: The term “operably linked” as used herein refers to a functional relationship between two or more regions of a polypeptide chain in which the two or more regions are linked so as to produce a functional polypeptide, or two or more nucleic acid sequences, e.g., to produce an in-frame fusion of two polypeptide components or to link a regulatory sequence to a coding sequence.

p28 Moiety or IL27 p28 Moiety: The terms “p28 moiety” and “IL27 p28 moiety” refer to an amino acid sequence comprising at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to an IL27Ra (IL27Rα) binding portion and/or a gp130 binding portion of a mammalian, e.g., human or murine, p28 protein. The sequence of full-length human p28 has the Uniprot identifier Q8NEV9 (uniprot.org/uniprot/Q8NEV9). The sequence of full-length murine p28 has the Uniprot identifier Q8K3I6 (uniprot.org/uniprot/Q8K3I6).

In some embodiments, the p28 moiety comprises an amino acid sequence comprising at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to a mature mammalian p28 protein, e.g., human or murine EBI3 (e.g., amino acids 29-243 of full-length human EBI3).

Further embodiments of EBI3 moieties are described in Section 5.3.1.

p28 Polypeptide: The term “p28 polypeptide” refers to a polypeptide comprising a p28 moiety (e.g., as described in Section 5.3.2). In some embodiments, the p28 polypeptide is a fusion polypeptide, e.g., a polypeptide comprising an Fc domain in addition to the p28 moiety.

p28 Protein: The term “p28 protein” refers to a monomeric or polymeric (e.g., dimeric) protein comprising a p28 moiety (e.g., an Fc dimer comprising a p28 moiety). The term “p28 protein” encompasses a p28 polypeptide.

Peptide-MHC complex, pMHC complex, peptide-in-groove: A “peptide-MHC complex,” “pMHC complex,” and “peptide-in-groove” refer to (i) an MHC domain (e.g., a human MHC molecule or portion thereof (e.g., the peptide-binding groove thereof and e.g., the extracellular portion thereof), (ii) an antigenic peptide, and, optionally, (iii) a β2 microglobulin domain (e.g., a human β2 microglobulin or portion thereof), where the MHC domain, the antigenic peptide and optional β2 microglobulin domain are complexed in such a manner that permits specific binding to a T-cell receptor. In some embodiments, a pMHC complex comprises at least the extracellular domains of a human HLA class I/human β2 microglobulin molecule and/or a human HLA class II molecule.

Single Chain Fv or scFv: The term “single chain Fv” or “scFv” as used herein refers to a polypeptide chain comprising the VH and VL domains of antibody, where these domains are present in a single polypeptide chain.

Specifically (or selectively) binds: The term “specifically (or selectively) binds” as used herein means that a targeting moiety, e.g., an antibody, or antigen binding domain (“ABD”) thereof, forms a complex with a target molecule that is relatively stable under physiologic conditions. Specific binding can be characterized by a K_{D} of about 5×10⁻²M or less (e.g., less than 5×10⁻²M, less than 10⁻²M, less than 5×10⁻³M, less than 10⁻³M, less than 5×10⁻⁴M, less than 10⁻⁴M, less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, less than 10⁻⁹M, or less than 10⁻¹⁰M). Methods for determining the binding affinity of an antibody or an antibody fragment, e.g., an IL27 agonist or a component targeting moiety, to a target molecule are well known in the art and include, for example, equilibrium dialysis, surface plasmon resonance (e.g., Biacore assays), fluorescent-activated cell sorting (FACS) binding assays and the like. An IL27 agonist of the disclosure comprising a targeting moiety or an ABD thereof that specifically binds a target molecule from one species can, however, have cross-reactivity to the target molecule from one or more other species.

Subject: The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

Target Molecule: The term “target molecule” as used herein refers to any biological molecule (e.g., protein, carbohydrate, lipid or combination thereof) expressed on a cell surface or in the extracellular matrix that can be specifically bound by a targeting moiety in an IL27 agonist of the disclosure.

Targeting Moiety: The term “targeting moiety” as used herein refers to any molecule or binding portion (e.g., an immunoglobulin or an antigen binding fragment) thereof that can bind to a cell surface or extracellular matrix molecule at a site to which an IL27 agonist of the disclosure is to be localized, for example on lymphocytes implicated in an autoimmune condition. The targeting moiety can also have a functional activity in addition to localizing an IL27 agonist to a particular site. For example, a targeting moiety that is an anti-PD1 antibody or an antigen binding portion thereof can also enhance the activity of an IL27 mutein, and a targeting moiety that is a component of the IL27 receptor can sequester and inhibit the activity of an IL27 mutein until it reaches its target cell or tissue.

Treat, Treatment, Treating: As used herein, the terms “treat”, “treatment” and “treating” refer to the reduction or amelioration of the progression, severity and/or duration of a disorder as described herein, or the amelioration of one or more symptoms (preferably, one or more discernible symptoms) of a condition or disorder as described herein, e.g., an inflammatory or immune disorder, resulting from the administration of one or more IL27 agonists of the disclosure. In specific embodiments, the terms “treat”, “treatment” and “treating” refer to the amelioration of at least one measurable physical parameter of a disorder, e.g., an autoimmune disorder, not necessarily discernible by the patient. In other embodiments the terms “treat”, “treatment” and “treating” refer to the inhibition of the progression of a disorder, either physically by, e.g., stabilization of a discernible symptom, physiologically by, e.g., stabilization of a physical parameter, or both.

Tumor: The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

Tumor-Associated Antigen: The term “tumor-associated antigen” or “TAA” refers to a molecule (typically a protein, carbohydrate, lipid or some combination thereof) that is expressed on the surface of a cancer cell, either entirely or as a fragment (e.g., MHC/peptide), and which is useful for the preferential targeting of a pharmacological agent to the cancer cell. In some embodiments, a TAA is a marker expressed by both normal cells and cancer cells, e.g., a lineage marker, e.g., CD19 on B cells. In some embodiments, a TAA is a cell surface molecule that is overexpressed in a cancer cell in comparison to a normal cell, for instance, 1-fold over expression, 2-fold overexpression, 3-fold overexpression or more in comparison to a normal cell. In some embodiments, a TAA is a cell surface molecule that is inappropriately synthesized in the cancer cell, for instance, a molecule that contains deletions, additions or mutations in comparison to the molecule expressed on a normal cell. In some embodiments, a TAA will be expressed exclusively on the cell surface of a cancer cell, entirely or as a fragment (e.g., MHC/peptide), and not synthesized or expressed on the surface of a normal cell. Accordingly, the term “TAA” encompasses antigens that are specific to cancer cells, sometimes known in the art as tumor-specific antigens (“TSAs”).

Universal Light Chain: The term “universal light chain” as used herein in the context of a targeting moiety refers to a light chain polypeptide capable of pairing with the heavy chain region of the targeting moiety and also capable of pairing with other heavy chain regions. Universal light chains are also known as “common light chains.”

VH: The term “VH” refers to the variable region of an immunoglobulin heavy chain of an antibody, including the heavy chain of an scFv or a Fab.

VL: The term “VL” refers to the variable region of an immunoglobulin light chain, including the light chain of an scFv or a Fab.

### 5.2. IL27 Receptor Agonists

The present disclosure provides IL27 agonists comprising or consisting of an IL27 monomer and/or an IL27 mutein. The IL27 agonists comprise an EBI3 moiety and a p28 moiety and differ from wild type IL27 by (a) primary amino acid sequence (e.g., an amino acid insertion, deletion, or substitution as compared to EBI3 and/or p28 or any combination of the foregoing) and/or (b) association with additional domains not naturally associated with IL27, for example (i) a multimerization moiety (e.g., dimerization domain such as an Fc domain) domain and/or (ii) a targeting moiety and/or (iii) a stabilization moiety (e.g., such as human serum albumin (HSA)).

In some embodiments, the IL27 receptor agonists, IL27 muteins, and IL27 monomers of the disclosure may comprise IL27 receptor sequences, for example IL27Ra (IL27Rα) and/or gp130 sequences, as described in Section 5.6 and subsections thereof, which may attenuate off-site effects of IL27 receptor agonist treatment.

An IL27 receptor agonist or IL27 mutein can be composed of one or more polypeptides, e.g., one or more IL27 monomers. In some embodiments, the IL27 receptor agonist is composed of a plurality (e.g. two) of IL27 monomers comprising EBI3 and/or p28 moieties and in some embodiments also comprising multimerization and/or stabilization moieties.

An IL27 receptor agonist, IL27 mutein, or IL27 monomer may further include one or more targeting moieties and/or one or more stabilization moieties and/or one or more IL27R1 moieties and/or one or more gp130 moieties. Exemplary multimerization moieties are described in Section 5.4 and include Fc domains that confer homodimerization or heterodimerization capability to the IL27 receptor agonist. Exemplary stabilization moieties are described in Section 5.5 and include human serum albumin (HSA). Free IL27 has poor pharmacokinetics (a serum half-life of less than about 2 h) and, without being bound by theory, it is believed that the inclusion of a multimerization domain, such as an Fc domain, and/or a stabilization moiety, such as HSA, improves serum stability and the pharmacokinetic profile of an IL27 receptor agonist. Thus, the Fc domain can be a dual-purpose domain, conferring stabilization properties of a stabilization moiety as described in Section 5.5.

Exemplary targeting moieties are described in Section 5.7 and include an antigen binding domain (e.g., a scFv or Fab) that binds to a cell surface molecule, binds to an immune cell associated cell surface molecule, binds to a tumor associated antigen, binds to a tumor microenvironment antigen, or binds to tumor lymphocytes, as well as a peptide-MHC complex that recognizes tumor lymphocytes.

In some embodiments, the IL27 receptor agonist includes an IL27Ra (IL27Rα) moiety, a gp130 moiety, or both an IL27Ra (IL27Rα) moiety and a gp130 moiety. Exemplary IL27Ra (IL27Rα) moieties are described in Section 5.6.1. Exemplary gp130 moieties are described in Section 5.6.2.

In some embodiments, the IL27 agonist of the disclosure is composed of two IL27 monomers, optionally in association with one or more additional polypeptide chains (e.g., a polypeptide chain comprising the light chain of a Fab targeting moiety). The monomers can be identical, thereby forming a homodimer, or different, thereby forming a heterodimer. The multimerization moieties of each monomer of an IL27 receptor agonist can be configured to dimerize together. Exemplary multimerization moieties are described in Section 5.4.

In some embodiments, an IL27 mutein or IL27 receptor agonist can include one or more linker sequences connecting the various components of its one or more polypeptide chains, for example (1) the EBI3 moiety and the p28 moiety of IL27 via an intra-IL27 moiety linker when present on the same polypeptide chain, (2) an EBI3 moiety and a multimerization moiety (e.g., an Fc domain) via a multimerization moiety linker, (3) a p28 moiety and a multimerization domain (e.g., an Fc domain) via a multimerization moiety linker, (4) a multimerization domain (e.g. an Fc domain) and a targeting moiety or component thereof (e.g., an scFv or a heavy chain of a Fab), (5) an EBI3 moiety, a p28 moiety, a multimerization domain or a targeting moiety or component thereof and an IL27Ra (IL27Rα) moiety, (6) an EBI3 moiety, a p28 moiety, a multimerization domain or a targeting moiety or component thereof and a gp130 moiety, or (8) any combination of the foregoing. Exemplary linkers are described in Section 5.8.

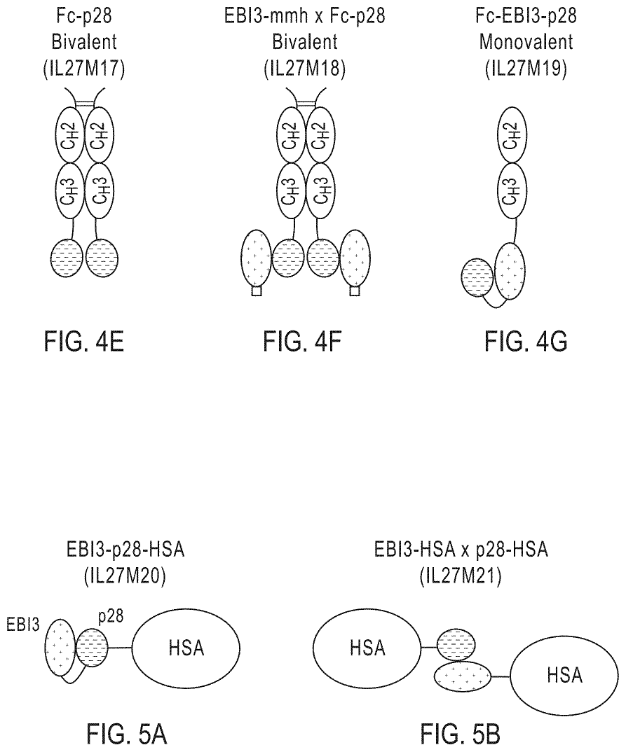

In some embodiments, the IL27 agonists comprise an EBI3 moiety, a p28 moiety, and a multimerization moiety (e.g., an Fc domain capable of homodimerizing or heterodimerizing with another Fc domain to form an Fc region) and/or a stabilization moiety (e.g., HSA), where the EBI3 moiety and the p28 moiety are configured such that they are capable of associating to form a functional IL27 receptor agonist. Exemplary configurations of IL27 receptor agonists, referred to as IL27M2, IL27M3, IL27M4, IL27M5, IL27M6, IL27M7, IL27M8, IL27M9, IL27M10, IL27M11, IL27M12, IL27M13, IL27M14, IL27M15, IL27M16, IL27M17, IL27M18, IL27M19, IL27M20 and IL27M21 are depicted in FIGS. 3-6.

Where the IL27 agonist comprises a multimerization moiety such as an Fc domain, the EBI3 and/or p28 moieties may be fused to the N-terminus or the C-terminus of the Fc domains of the Fc region. As illustrated in FIGS. 3A-3G, the IL27 agonists designated IL27M2, IL27M3, IL27M9, IL27M10, IL27M12, IL27M13, IL27M14 and IL27M15 contain EBI3 and p28 moieties at the N-termini of Fc domains. As illustrated in FIGS. 4A-4G, the IL27 agonists designated IL27M4, IL27M5, IL27M6, IL27M7, IL27M8, IL27M11, IL27M16, IL27M17, IL27M18 and IL27M19 contain EBI3 and p28 moieties at the C-termini of Fc domains

Most IL27 muteins and IL27 agonists are multimeric, e.g., dimeric, by virtue of association of an EBI3 moiety and a p28 moiety present on different polypeptide chains and/or by virtue of association of multimerization moieties configured to associate with one another (e.g., Fc domains). In some embodiments, the associated EBI3 moiety and p28 moiety are on the same polypeptide chain (e.g., in IL27M1, IL27M3, IL27M4, IL27M5, IL27M8, IL27M9, IL27M12, IL27M15, IL27M16, and IL27M19 as shown in FIGS. 3A, 3C, 4A, 4B, 6B, 6C, 3D, 3G, 4D, and 4G, respectively). Further, when the EBI3 and p27 domain are present on the same polypeptide chain, the EBI3 moiety can be N-terminal to the p28 moiety (e.g., in IL27M4, IL27M8, IL27M12, and IL27M15 as shown in FIGS. 4A, 6B, 3D, and 3G, respectively) or C-terminal to the p28 moiety (e.g., in IL27M3, IL27M5, and IL27M9, shown in FIGS. 3C, 4B, and 6C, respectively).

In other embodiments, the associated EBI3 moiety and p28 moiety are on different polypeptide chains associated through a multimerization moiety (e.g., Fc domains forming an Fc region) (e.g., in IL27M2, IL27M6, and IL27M7, as shown in FIGS. 3B, 4C and 6A, respectively).

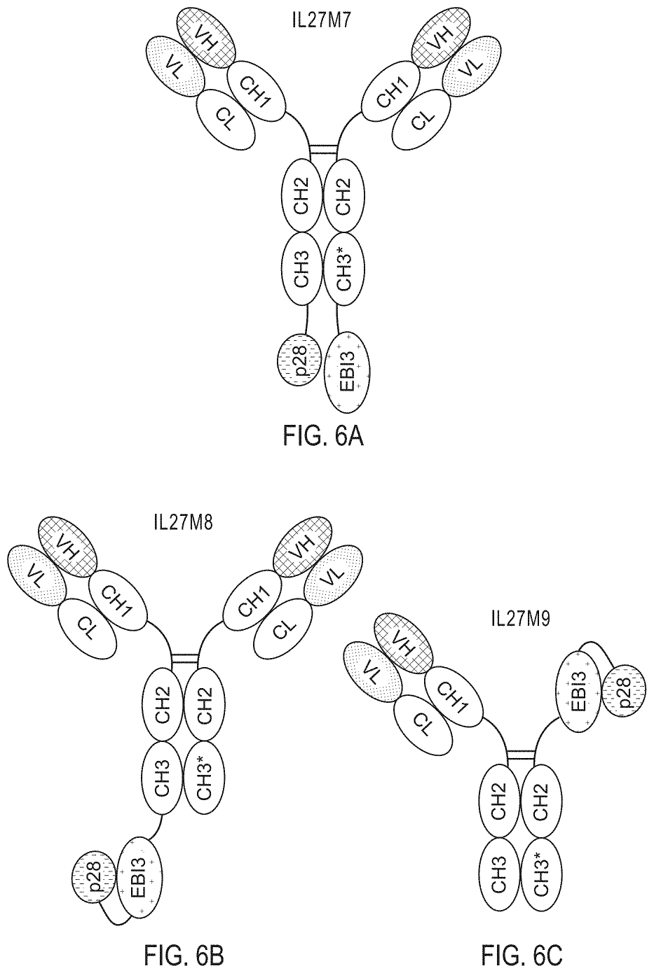

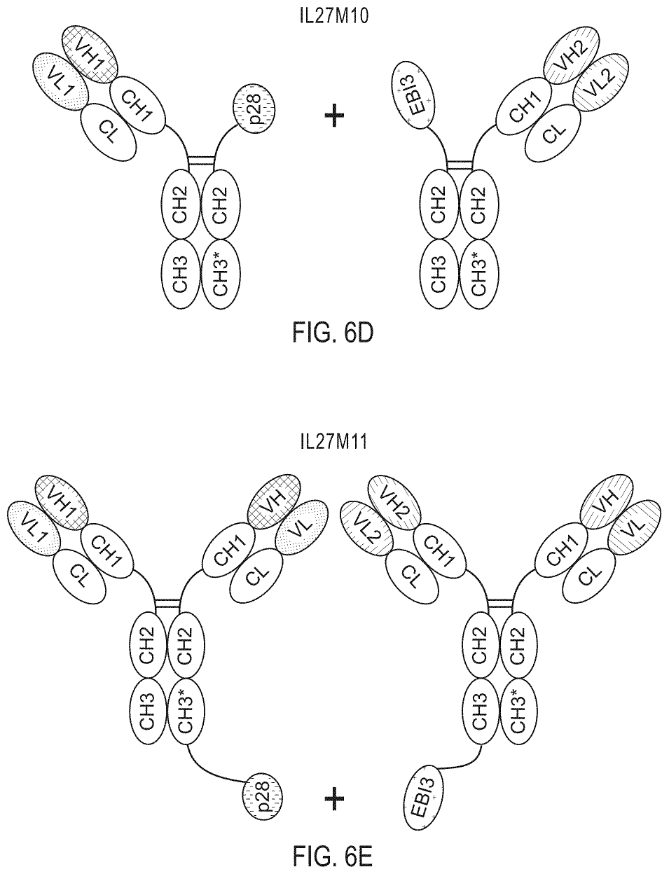

In yet other embodiments, the associated EBI3 moiety and p28 moiety are present in a bi-molecular structure in which the EBI3 moiety and the p28 moiety are present on different polypeptides, for example by association of an EBI3 moiety in an EBI polypeptide or EBI protein with a p28 moiety in a p28 polypeptide or p28 protein (e.g., IL27M9, IL27M10, IL27M11, IL27M21, shown in FIGS. 6C, 6D, 6E, and 5B, respectively).

The present disclosure generally refers to polypeptide chains containing an EBI3 moiety and/or a p28 moiety, and/or a multimerization moiety (e.g., a first Fc domain) that is capable of associating with another polypeptide chain containing an EBI3 moiety and/or a p28 moiety and/or a corresponding multimerization moiety (e.g., a second Fc domain), respectively, as “monomers” or “IL27 monomers”. The term “monomer” also encompasses polypeptide chains containing an EBI3 moiety and/or a p28 moiety, and/or a stabilization moiety (e.g., a first HSA domain). In some embodiments, a monomer comprising a stabilization moiety is capable of associating with another monomer comprising a stabilization moiety (e.g., a second HSA domain) via the EBI3 and p28 moieties of the two monomers. Below are some illustrative examples of IL27 monomers of the disclosure, described in an N- to -C terminal orientation.

Individual elements of each monomer are described in detail herein, for example in the subsections that follow and the numbered embodiments.

(1) Exemplary Monomer 1: IL27 p28 moiety-optional linker-multimerization moiety (see, e.g., FIGS. 3B (left monomer), 3E).

(2) Exemplary Monomer 2: IL27 EBI3 moiety-optional linker-multimerization moiety (see, e.g., FIG. 3B, right monomer).

(3) Exemplary Monomer 3: Optional targeting moiety (e.g., scFV) or targeting moiety component (e.g., VH or VL of a Fab)-optional linker-multimerization moiety-IL27 p28 moiety (see, e.g., FIGS. 4C (left monomer), 4E, 6A (left monomer), 6D (right monomer of left construct)).

(4) Exemplary Monomer 4: Optional targeting moiety (e.g., scFV) or targeting moiety component (e.g., VH or VL of a Fab)-multimerization moiety-optional linker-IL27 EBI3 moiety (see, e.g., FIGS. 4C (right monomer), 6A (right monomer), 6D (left monomer of right construct)).

(5) Exemplary Monomer 5: IL27 EBI3 moiety-optional linker-IL27 p28 moiety-optional linker-multimerization moiety (see, e.g., FIGS. 3A (both monomers), 3D (left monomer), and 3G).

(6) Exemplary Monomer 6: IL27 p28moiety-optional linker-IL27 EBI3 moiety-optional linker-multimerization moiety (see, e.g., FIG. 4C, both monomers).

(7) Exemplary Monomer 7: Optional targeting moiety (e.g., scFV) or targeting moiety component (e.g., VH or VL of a Fab)-optional linker-multimerization moiety-optional linker-IL27 EBI3 moiety-optional linker-IL27 p28 moiety (see, e.g., FIGS. 4A (both monomers), 4D, 6B (left monomer)).

(8) Exemplary Monomer 8: Optional targeting moiety (e.g., scFV) or targeting moiety component (e.g., VH or VL of a Fab)-optional linker-multimerization moiety-IL27 p28 moiety-optional linker-IL27 EBI3 moiety (see, e.g., FIG. 4B (both monomers).

(9) Exemplary Monomer 9: IL27 EBI3 moiety-optional linker-stabilization moiety (see, e.g., FIG. 5B, right polypeptide).

(10) Exemplary Monomer 10: IL27 p28 moiety-optional linker-stabilization moiety (see, e.g., FIG. 5B, right polypeptide).

(11) Exemplary Monomer 11: Stabilization moiety-optional linker-IL27 EBI3 moiety.

(12) Exemplary Monomer 12: Stabilization moiety-optional linker-IL27 p28 moiety.

(13) Exemplary Monomer 13: IL27 EBI3 moiety-optional linker-IL27 p28 moiety-optional linker-stabilization moiety (see, e.g., FIG. 5A).

(14) Exemplary Monomer 14: IL27 p28 moiety-optional linker-IL27 EBI3 moiety-optional linker-stabilization moiety.

(15) Exemplary Monomer 15: Stabilization moiety-optional linker-IL27 EBI3 moiety-optional linker-IL27 p28 moiety.

(16) Exemplary Monomer 16: Stabilization moiety-optional linker-IL27 p28 moiety-optional linker-IL27 EBI3 moiety.

Where the present disclosure refers to a monomer that includes a targeting moiety component, and unless the context dictates otherwise, such a reference to the monomer encompasses monomers associated with another polypeptide chain comprising a counterpart targeting moiety component, e.g., the counterpart VL or VH of a Fab.

Exemplary combinations of the Exemplary Monomers are provided in numbered embodiments 31 to 79.

In certain aspects, the IL27 agonist comprises an IL27 mutein having the configuration of IL27M2, IL27M3, IL27M4, IL27M5, IL27M6, IL27M12, IL27M13, IL27M14, IL27M15, IL27M16, IL27M17, IL27M18, IL27M19, IL27M20, or IL27M21, with or without an optional targeting moiety. In other aspects, the IL27 agonist comprises an IL27 mutein having the configuration of IL27M7, IL27M8, IL27M10, or IL27M11 and includes a targeting moiety.

Reference to a particular IL27 mutein or IL27 receptor agonist architecture (e.g., IL27M1, IL27M2, etc.) is not intended to be limiting but rather to serve as an indication of the generic architecture of a subgenus of IL27 receptor agonists. Accordingly, reference to a particular IL27 mutein or IL27 receptor agonist architecture is not intended to limit, for example, the particular amino acid sequence or pair of amino acid sequences of the polypeptide(s) that form the IL27 mutein or the IL27 receptor agonist. For example, IL27M1 comprises two polypeptides (i.e., IL27 monomers) where the first polypeptide and the second polypeptide each have the configuration of Exemplary Monomer 5 (IL27 EBI3 moiety-optional linker-IL27 p28 moiety-optional linker-multimerization moiety). In this example, the EBI3 moiety of each monomer can be any EBI3 moiety described herein. The EBI3 moieties on the two monomers can be the same, or can be different. Likewise, the p28 moiety of each monomer can be any p28 moiety described herein. The p28 moieties on the two monomers can be the same, or can be different. The multimerization moiety of each monomer can be any multimerization moiety described herein. The multimerization moieties (e.g., Fc domains) on the two monomers can be the same (e.g., allowing for homodimerization), or can be different (e.g., allowing for heterodimerization). Further, the linkers can be present or absent, and when present can be identical or different. In view of the present disclosure, it will be apparent to those skilled in the art that the IL27M__nomenclature thus serves to represent a generic architecture of a subgenus of IL27 agonists in which individual species share the overall architecture, but can differ in amino acid sequence(s), or presence or absence of optional moieties as provided above in the description of the Exemplary Monomers (e.g., an optional targeting moiety). In some instances an IL27 agonist comprising one or more optional moieties (e.g., a targeting moiety) is given a distinct reference (e.g., IL27M7) to define a further subgenus of the IL27 mutein or IL27 receptor agonist configuration. With reference to the Exemplary Monomers, exemplary IL27 architectures are described below.

(1) IL27M1: A first Exemplary Monomer 5 associated with a second Exemplary Monomer 5 (e.g., as represented in FIG. 3A).

(2) IL27M2: An Exemplary Monomer 1 associated with an Exemplary Monomer 2 (e.g., as represented in FIG. 3B).

(3) IL27M3: A first Exemplary Monomer 6 associated with a second Exemplary Monomer 6 (e.g., as represented in FIG. 3C).

(4) IL27M4: A first Exemplary Monomer 7 associated with a second Exemplary Monomer 7 (e.g., as represented in FIG. 4A).

(5) IL27M5: A first Exemplary Monomer 8 associated with a second Exemplary Monomer 8 (e.g., as represented in FIG. 4B).

(6) IL27M6: An Exemplary Monomer 3 associated with an Exemplary Monomer 4 (e.g., as represented in FIG. 4C).

(7) IL27M7: An Exemplary Monomer 3 comprising a first targeting moiety or a first targeting moiety component, associated with an Exemplary Monomer 4 comprising a second targeting moiety or a second targeting moiety component (e.g., as represented in FIG. 6A), optionally wherein when comprising first and/or second targeting moiety components, IL27M7 further comprises a third targeting moiety component capable of associating with the first targeting moiety component and/or a fourth targeting moiety component capable of associating with the second targeting moiety component.

(8) IL27M8: An Exemplary Monomer 7 comprising a first targeting moiety or a first targeting moiety component associated with a polypeptide comprising a second targeting moiety or second targeting moiety component and a multimerization moiety (e.g., as represented in FIG. 6B), optionally wherein when comprising first and/or second targeting moiety components, IL27M8 further comprises a third targeting moiety component capable of associating with the first targeting moiety component and/or a fourth targeting moiety component capable of associating with the second targeting moiety component.

(9) IL27M9: An Exemplary Monomer 6 associated with a polypeptide comprising a first targeting moiety or a first targeting moiety component and a multimerization moiety (e.g., as represented in FIG. 6C), optionally wherein when comprising a first targeting moiety component, IL27M9 further comprises a second targeting moiety component capable of associating with the first targeting moiety component.

(10) IL27M10: A first protein comprising (i) an Exemplary Monomer 1 associated with (ii) a polypeptide comprising a first targeting moiety or a first targeting moiety component and a multimerization moiety; associated with a second protein comprising (i) an Exemplary Monomer 2 associated with (ii) a polypeptide comprising a second targeting moiety or a second targeting moiety component and a multimerization moiety (e.g., as represented in FIG. 6D), optionally wherein when comprising first and/or second targeting moiety components, IL27M10 further comprises a third targeting moiety component capable of associating with the first targeting moiety component and/or a fourth targeting moiety component capable of associating with the second targeting moiety component.

(11) IL27M11: A first protein comprising (i) an Exemplary Monomer 3 comprising a first targeting moiety or a first targeting moiety component associated with (ii) a polypeptide comprising a second targeting moiety or a second targeting moiety component and a multimerization moiety; associated with a second protein comprising (i) an Exemplary Monomer 4 comprising a third targeting moiety or a third targeting moiety component associated with (ii) a polypeptide comprising a fourth targeting moiety a fourth targeting moiety component and a multimerization moiety (e.g., as represented in FIG. 6E), optionally wherein when comprising first, second, third and/or fourth targeting moiety components, IL27M11 further comprises a fifth targeting moiety component capable of associating with the first targeting moiety component and/or a sixth targeting moiety component capable of associating with the second targeting moiety component and/or a seventh targeting moiety component capable of associating with the third targeting moiety component, and/or an eighth targeting moiety component capable of associating with the fourth targeting moiety component.

(12) IL27M12: An Exemplary Monomer 5 associated with a polypeptide chain comprising a multimerization moiety and optionally a first targeting moiety or targeting moiety component (e.g., as represented in FIG. 3D), optionally wherein when comprising a first targeting moiety component, IL27M12 further comprises a second targeting moiety component capable of associating with the first targeting moiety component.

(13) IL27M13: A first Exemplary Monomer 1 associated with a second Exemplary Monomer 1 (e.g., as represented in FIG. 3E).

(14) IL27M14: A first Exemplary Monomer 1 having an EBI3 moiety capable of associating with the p28 moiety of the first Exemplary Monomer 1, associated with a second Exemplary Monomer 1 having an EBI3 moiety capable of associating with the p28 moiety of the second Exemplary Monomer 1 (e.g., as represented in FIG. 3E).

(15) IL27M15: An Exemplary Monomer 5 (e.g., as represented in FIG. 3G).

(16) IL27M16: An Exemplary Monomer 7 associated with a polypeptide chain comprising a multimerization moiety and optionally a targeting moiety or a first targeting moiety component (FIG. 4D), optionally wherein when comprising a first targeting moiety component, IL27M16 further comprises a second targeting moiety component capable of associating with the first targeting moiety component.

(17) IL27M17: A first Exemplary Monomer 3 associated with a second Exemplary Monomer 3 (e.g., as represented in FIG. 4E).

(18) IL27M18: A first Exemplary Monomer 3 having an EBI3 moiety capable of associating with the p28 moiety of the first Exemplary Monomer 3, associated with a second Exemplary Monomer 3 having an EBI3 moiety capable of associating with the p28 moiety of the second Exemplary Monomer 3 (e.g., as represented in FIG. 4F).

(19) IL27M19: An Exemplary Monomer 7 (e.g., as represented in FIG. 4G).

(20) IL27M20: An Exemplary Monomer 18 or an Exemplary Monomer 21 (e.g., as represented in FIG. 5A).

(21) IL27M21: IL27M21 generally has the configuration represented in FIG. 5B. Particular embodiments of IL27M21 include (1) An Exemplary Monomer 14 associated with an Exemplary Monomer 15; (2) an Exemplary Monomer 14 associated with an Exemplary Monomer 17; (3) an Exemplary Monomer 16 associated with an Exemplary Monomer 15; and (4) an Exemplary Monomer 16 associated with an Exemplary Monomer 17.

In the IL27 receptor agonists of the disclosure, when the targeting moiety is an antigen binding domain (“ABD”) of an antibody, each monomer can comprise a targeting moiety component (e.g., a heavy chain variable region (VH) or a light chain variable region (VL)) and a counterpart targeting moiety component (e.g., a VL wherein the targeting moiety is VH, or VH wherein the targeting moiety is VL). The targeting moiety component can associate with the counterpart targeting moiety component to form the targeting moiety. Thus, a single monomer can be composed of two polypeptide chains, one polypeptide chain bearing one targeting moiety component (e.g., VH) and the other polypeptide chain bearing counterpart targeting moiety component (e.g., VL). Thus, the targeting moiety itself can comprise heavy and light chain variable domains on separate polypeptide chains. For example, with respect to an IL27 receptor agonist monomer including a targeting moiety, the monomer can be composed of a Polypeptide A and a Polypeptide B. Polypeptide A can include, for example, from N-terminus to C-terminus: the heavy chain variable domain of a targeting moiety (e.g., the targeting moiety component)-optional linker-multimerization moiety-optional linker-IL27 EBI3 moiety-IL27 p28 moiety; and Polypeptide B can comprise the light chain variable domain of a targeting moiety (i.e., the counterpart targeting moiety component). Targeting moieties are further described and defined in Section 5.7 and numbered embodiments 259 to 315.

Alternatively, an scFv can be used as a targeting moiety, in which the heavy and light chain variable regions of the targeting moiety are fused to one another in a single polypeptide.

In various embodiments, the IL27 receptor agonist does not comprise (a) a cytokine other than IL27; (b) an anti-IL27 antibody or antibody fragment; (c) an anti-DNA antibody or antibody fragment; (b) a non-binding antibody variable domain; or any combination of two, three or all four of these

The present disclosure further provides EBI3 protein and p28 protein components of a bi-molecular IL27 agonist as shown in FIGS. 3D and 3E. Such polypeptides are useful, inter alia, in conjunction with one another for combination therapy and are also referred to herein as IL27 agonists.

The IL27 receptor agonists of the disclosure and/or the IL27 muteins in the IL27 receptor agonists of the disclosure and/or the IL27 monomers in the IL27 receptor agonists of the disclosure can have amino acid modifications that result in a reduction of binding affinity of to an IL27 receptor complex (e.g., a receptor complex comprising gp130 and IL27Ra (IL27Rα)) as compared to wild type IL27. Overall, the IL27 receptor agonists of the disclosure and/or the IL27 muteins in the IL27 receptor agonists of the disclosure and/or the IL27 monomers in the IL27 receptor agonists of the disclosure can have normal or attenuated binding (i.e., reduced affinity) to the IL27 receptor complex (e.g., by up to 10-fold, by up to 50-fold, by up to 100-fold, by up to-200 fold, by up to 500-fold, by up to 1,000-fold or by up to 5,000-fold). Binding can be attenuated through one or more amino acid substitutions in the EBI3 and/or p28 sequences and/or the inclusion of one or more IL27Ra (IL27Rα) moieties in the IL27receptor agonist.

In certain embodiments, the IL27 receptor agonists, IL27 muteins, and/or IL27 monomers of the disclosure have one or more amino acid substitutions in an IL27 EBI3 moiety, an IL27 p28 moiety, or both IL27 EBI3 and p28 moieties that reduce binding to the IL27 receptor complex, for example as disclosed in Section 5.6 and subsections thereof. For example, in some embodiments, an IL27 mutein can have up to 10-fold to 1,000-fold attenuated binding to human IL27 receptor complex as compared to wild-type human IL27.

Binding affinity of IL27 to its receptor complex be assayed by, for example, surface plasmon resonance (SPR) techniques (analyzed on a Biacore instrument) (Liljeblad et al., 2000, Glyco J, 17:323-329).

The present disclosure further provides p28 proteins and EBI3 proteins. Some IL27 receptor agonists and muteins of the disclosure comprise a p28 protein associated with an EBI3 protein.

In some embodiments, an EBI3 protein is composed of two polypeptides. Is some embodiments, an EBI3 protein comprises a first polypeptide comprising (i) a first targeting moiety, (ii) an optional first linker, and (iii) a first multimerization moiety; and a second peptide comprising (i) an EBI3 moiety, (ii) an optional second linker, and (iii) a second multimerization moiety associated with the first multimerization moiety. The heterodimer on the right of FIG. 6D depicts a first exemplary EBI3 protein.

In other embodiments, an EBI3 protein comprises a first polypeptide comprising (i) a first targeting moiety, an optional first linker, and a first multimerization moiety, and a second polypeptide comprising (i) a second targeting moiety, (ii) an optional first linker, and (iii) a first multimerization moiety; and a second polypeptide comprising (i) a second targeting moiety, (ii) an optional second linker, (iii) a second multimerization moiety associated with the first multimerization moiety, (iv) an optional third linker, and (v) an EBI3 moiety. The heterodimer on the right of FIG. 6E depicts a second exemplary EBI3 protein.

In certain aspects, an EBI3 protein of the disclosure lacks a p28 moiety (but is capable of associating with a p28 moiety, e.g., a p28 moiety in a p28 protein).

In some embodiments, a p28 protein is composed of two polypeptides. Is some embodiments, a p28 protein comprises a first polypeptide comprising (i) a first targeting moiety, (ii) an optional first linker, and (iii) a first multimerization moiety; and a second peptide comprising (i) a p28 moiety, (ii) an optional second linker, and (iii) a second multimerization moiety associated with the first multimerization moiety. The heterodimer on the left of FIG. 6D depicts a first exemplary p28 protein.

In other embodiments, a p28 protein comprises a first polypeptide comprising (i) a first targeting moiety, an optional first linker, and a first multimerization moiety, and a second polypeptide comprising (i) a second targeting moiety, (ii) an optional first linker, and (iii) a first multimerization moiety; and a second polypeptide comprising (i) a second targeting moiety, (ii) an optional second linker, (iii) a second multimerization moiety associated with the first multimerization moiety, (iv) an optional third linker, and (v) a p28 moiety. The heterodimer on the left of FIG. 6E depicts a second exemplary EBI3 protein.

In certain aspects, a p28 protein of the disclosure lacks an EBI3 moiety (but is capable of associating with an EBI3 moiety, e.g., an EBI3 moiety in an EBI3 protein).

The p28 proteins are typically configured to associate with an EBI3 moiety, for example the EBI3 moiety of an EBI3 protein (see, e.g., FIGS. 6D and 6E). The EBI3 proteins are typically configured to associate with a p28 moiety, for example the p28 moiety of a p28 protein (see, e.g., FIGS. 6D and 6E). An EBI3 protein of the disclosure can associate with a p28 protein of the disclosure to form an IL27 agonist.

Further details of the components of the IL27 receptor agonists, EBI3 proteins, and p28 proteins of the disclosure are presented below.

### 5.3. The IL27 EBI3 and p28 Moieties

The present disclosure provides IL27 receptor agonists with EBI3 and p28 moieties with wild type or variant EBI3 and p28 sequences. The present disclosure further provides p28 moieties with variant p28 sequences. Exemplary EBI3 moieties are disclosed in Section 5.3.1 and exemplary p28 moieties are disclosed in Section 5.3.2.

IL27 is a heterodimer consisting of Epstein-Barr virus-induced gene 3 (EBI3) and p28 subunits. Thus, as used herein, the term “IL27 domain” refers to an EBI3 moiety and/or p28 moiety.

The IL27 domain encompasses mature human and non-human (e.g., murine, rat, porcine, non-human primate) EBI3 and p28 polypeptides, including homologs, variants, and fragments thereof, as well as EBI3 and p28 polypeptides having, for example, a leader sequence (e.g., the signal peptide), and modified versions of the foregoing. In certain embodiments, the IL27 agonists of the disclosure have one or more amino acid modifications, e.g., substitutions, deletions or insertions, in the EBI3 moiety or a p28 binding domain thereof, and/or in the p28 moiety or an IL27Ra (IL27Rα) binding domain and/or gp130 binding domain thereof, as compared to those moieties or domains in a wild type or naturally occurring IL27 variant. Hence, the terms an “EBI3 moiety” and a “p28 moiety” encompass proteins of substantially similar sequence as mature wild type human, murine, porcine, or rat EBI3 and p28, respectively, more preferably a protein of substantially similar sequence as mature wild type human EBI3 and p28 respectively.

In various embodiments, the EBI3 moiety and/or p28 moiety comprises an amino acid sequence with at least about 90%, at least about 95%, at least about 96%, at least about 97%, at least about 98%, at least about 99% or 100% sequence identity to human, murine, porcine, or rat EBI3 and/or p28 moiety sequences, such as those exemplified in Sections 5.3.1 and 5.3.2, respectively.

5.3.1. EBI3 Moieties

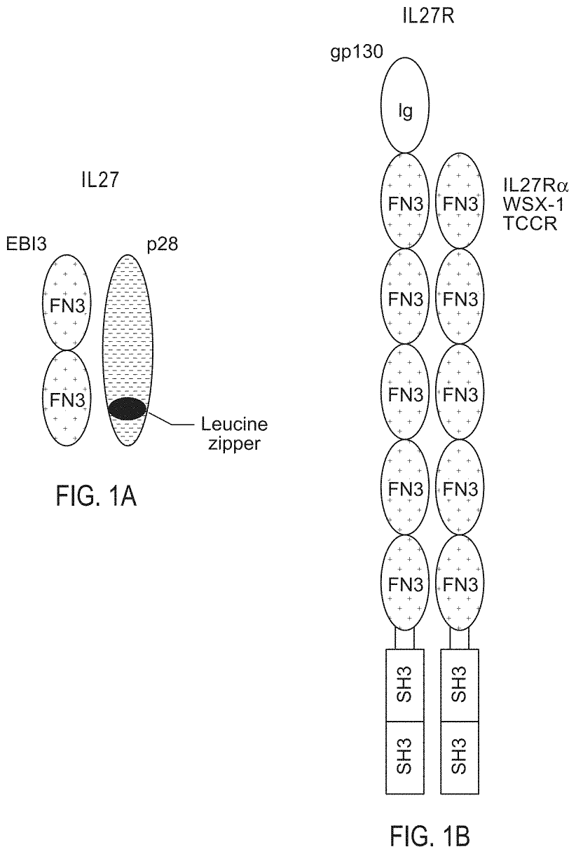

EBI3 was initially described as being expressed in B lymphocytes infected by Epstein-Barr virus. The EBI3 structure consists of a tandem pair of modified fibronectin type III (FnIII) domains named the cytokine-binding domain (CBD) (see FIG. 1A). This domain typically contains two pairs of cysteine residues involved in disulfide bridge formation and a characteristic WSXWS signature motif. EBI3 is known to exist in three forms, one of which is a heterodimer with p28.

Each IL27 EBI3 moiety of the IL27 receptor agonists of the disclosure comprises a p28-binding domain of a wild type or variant IL27 EBI3. In some embodiments, an IL27 receptor agonist of the disclosure comprises a single IL27 EBI3 moiety (e.g., an IL27 EBI3 moiety on a first monomer or on a second monomer in embodiments where the IL27 receptor agonist is monovalent for IL27). In some embodiments, an IL27 receptor agonist of the disclosure comprises two IL27 EBI3 moieties (e.g., a first IL27 EBI3 moiety on a first monomer and a second IL27 EBI3 moiety on a second monomer in embodiments where the IL27 agonist is bivalent for IL27). In such embodiments, the two IL27 EBI3 moieties can be identical, or they can be different.

In some embodiments, an IL27 EBI3 moiety is or comprises an amino acid sequence comprising at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to a p28 binding domain of a mammalian, e.g., human or murine, EBI3.

In some embodiments, the p28 binding domain of EBI3 comprises amino acids corresponding to F97, E124, E159, and D210 of full-length human EBI3. F97, E124, E159, and D210 are predicted to be involved in the binding of EBI3 to p28 (Rousseau et al., 2010, Proc Natl Acad Sci USA. 107(45):19420-19425). Thus, in some embodiments, the p28 binding domain of EBI3 comprises amino acids 97 to 210 of full-length human EBI3, or equivalent amino acids of another mammalian, e.g., murine, EBI3.

In some embodiments, the mammalian EBI3 is full-length human EBI3. In other embodiments, the mammalian EBI3 is mature human EBI3. The sequence of human EBI3 has the Uniprot identifier Q14213 (uniprot.org/uniprot/Q14213). In some embodiments, the mammalian EBI3 moiety is full-length murine EBI3. In some embodiments, the mammalian EBI3 is mature murine EBI3. The sequence of murine EBI3 has the Uniprot identifier 035228 (uniprot.org/uniprot/035228).

Human EBI3 is synthesized as a precursor polypeptide of 229 amino acids, from which 20 amino acids are removed to generate mature secreted EBI3. The first fibronectin III domain spans amino acids 24-130 of EBI3 and the second fibronectin III domain spans amino acids 131-227 of EBI3. Accordingly, in some embodiments, the EBI3 moiety of the disclosure comprises full length human EBI3. In other embodiments, the EBI3 moiety of the disclosure comprises mature human EBI3, corresponding to positions 21-229 of the 229-amino acid precursor sequence shown below, or the two fibronectin domains, corresponding to positions 24-228 of the 229-amino acid precursor sequence shown below:

| (SEQ ID NO: 1) | |
|---|---|
| | 10 20 30 40 |
| | MTPQLLLALV LWASCPPCSG RKGPPAALTL PRVQCRASRY |
| | 50 60 70 80 |
| | PIAVDCSWTL PPAPNSTSPV SFIATYRLGM AARGHSWPCL |
| | 90 100 110 120 |
| | QQTPTSTSCT ITDVQLFSMA PYVLNVTAVH PWGSSSSFVP |
| | 130 140 150 160 |
| | FITEHIIKPD PPEGVRLSPL AERQLQVQWE PPGSWPFPEI |
| | 170 180 190 200 |
| | FSLKYWIRYK RQGAARFHRV GPIEATSFIL RAVRPRARYY |
| | 210 220 |
| | VQVAAQDLTD YGELSDWSLP ATATMSLGK. |

Amino acid 24 of full-length human EBI3 is amino acid 1 of mature human EBI3.

Naturally existing sequence variants of EBI3 have been reported. The EBI3 sequence having European Nucleotide Archive accession no. AAA93193.1 has an QL→HV substitution at positions 144-145 of the amino acid sequence shown above. SNP variant rs1803524 has a A→V substitution at position 174. SNP variant rs4740 has a V→I substitution at position 201. Accordingly, the EBI3 moiety of the disclosure may contain any combination of the foregoing variants, e.g., one, two or all three of (1) QL→HV substitution at positions 144-145; (2) A→V substitution at position 174; and V→I substitution at position 201.

Human EBI3 contains potential N-linked glycosylation sites at amino acids 55 and 105. The present disclosure encompasses EBI3 moiety molecules with or without N-linked glycans at N55 and/or N105 or the equivalent position in EBI3 of other species.

The EBI3 moiety may comprise a peptide tag, e.g., a peptide tag that facilitates purification, at its N-terminus or C-terminus. In some embodiments the peptide tag is a myc-myc-his (mmh) tag.

In some embodiments, the EBI3 moiety comprises an EBI3 amino acid sequence shown in Section 5.1.1.

5.3.2. p28 Moieties

p28 is a “long-chain” cytokine with a four-helix bundle fold; these four helices are named A-D from the N terminus to the C terminus. p28 contains a leucine zipper motif indicative of homo- or heterodimerization (see FIG. 1A). p28 is normally found to be co-expressed with EBI3 in activated macrophages and dendritic cells, forming a non-covalently linked heterodimer.

Each IL27 p28 moiety of the IL27 receptor agonists of the disclosure comprises a wild type or variant IL27 p28 moiety. In some embodiments, an IL27 receptor agonist of the disclosure comprises a single IL27 p28 moiety (e.g., an IL27 p28 moiety on a first monomer or on a second monomer in embodiments where the IL27 receptor agonist is monovalent for IL27). In some embodiments, an IL27 receptor agonist of the disclosure comprises two IL27 p28 moieties (e.g., a first IL27 p28 moiety on a first monomer and a second IL27 p28 moiety on a second monomer in embodiments where the IL27 agonist is bivalent for IL27). In such embodiments, the two IL27 p28 moieties can be identical, or they can be different.

In some embodiments, an IL27 p28 moiety is or comprises an amino acid sequence comprising at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to an IL27Ra (IL27Rα) binding domain of a mammalian, e.g., human or murine, p28.

In some embodiments, the IL27Ra (IL27Rα) binding domain comprises p28 contact site 2 (see, e.g., Rousseau et al., 2010, Proc Natl Acad Sci USA. 107(45):19420-19425). Contact site 2 comprises solvent-exposed residues of the αA and αC helices of p28 (Rousseau et al., 2010, Proc Natl Acad Sci USA. 107(45):19420-19425). In some embodiments, the IL27Ra (IL27Rα) binding domain of p28 comprises amino acids corresponding to H52, K56, S59, E60, W138, L142, R145, D146, R149, and H150 of full-length human p28. In some embodiments, the IL27Ra (IL27Rα) binding domain of p28 comprises amino acids 52 to 150 of full-length human p28, or equivalent amino acids of another mammalian, e.g., murine, p28.

In some embodiments, an IL27 p28 moiety is or comprises an amino acid sequence comprising at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to a gp130 binding domain of a mammalian, e.g., human or murine, p28.

In some embodiments, the gp130 binding domain comprises p28 contact site 3 (see, e.g., Rousseau et al., 2010, Proc Natl Acad Sci USA. 107(45):19420-19425). Contact site 3 is located at the N terminus of the αD helix and engages an Ig domain of gp130 (Rousseau et al., 2010, Proc Natl Acad Sci USA. 107(45):19420-19425). In some embodiments, the gp130 binding domain of p28 comprises amino acids corresponding to W197, L200, L201, Y204, and R205 of full-length human p28. In some embodiments, the gp130 binding domain of p28 comprises amino acids 197 to 205 of full-length human p28, or equivalent amino acids of another mammalian, e.g., murine, p28. In certain embodiments, the gp210 binding domain, in addition to W197, L200, L201, Y204, and R205 of full-length human p28, further comprises L73 and V76. Thus, in certain embodiments, the gp130 binding domain of p28 comprises amino acids 73 to 205 of full-length human p28, or equivalent amino acids of another mammalian, e.g., murine, p28.

In some embodiments, the mammalian p28 is full-length human p28. In other embodiments, the mammalian p28 is mature human p28. The sequence of human p28 has the Uniprot identifier Q8NEV9 (uniprot.org/uniprot/Q8NEV9). In some embodiments, the mammalian p28 moiety is full-length murine p28. In some embodiments, the mammalian p28 is mature murine p28. The sequence of murine p28 has the Uniprot identifier Q8K316 (uniprot.org/uniprot/Q8K316).

Human p28 is synthesized as a precursor polypeptide of 243 amino acids, from which 28 amino acids are removed to generate mature secreted p28. Accordingly, in some embodiments, the p28 moiety of the disclosure comprises mature human p28, corresponding to positions 29-243 of the 243-amino acid precursor sequence shown below:

(SEQ ID NO: 2)

```
                  10 20 30 40
MGQTAGDLGW RLSLLLLPLL LVQAGVWGFP RPPGRPQLSL
                  50 60 70 80
QELRREFTVS LHLARKLLSE VRGQAHRFAE SHLPGVNLYL
                  90 100 110 120
LPLGEQLPDV SLTFQAWRRL SDPERLCFIS TTLQPFHALL
                  130 140 150 160
GGLGTQGRWT NMERMQLWAM RLDLRDLQRH LRFQVLAAGF
                  170 180 190 200
NLPEEEEEEE EEEEEERKGL LPGALGSALQ GPAQVSWPQL
                  210 220 230 240
LSTYRLLHSL ELVLSRAVRE LLLLSKAGHS VWPLGFPTLS PQP.
```

Amino acid 29 of full-length human p28 is amino acid 1 of mature human p28.

The four helices (A, B, C and D) of p28 are shown in bold font above. In some embodiments, the p28 moiety of the disclosure comprises the region spanning the four helices of p28, corresponding to positions 39-224 of the 243-amino acid precursor sequence shown above.

Naturally existing sequence variants of p28 have been reported. SNP variant rs17855740 has a S→A substitution at position 59. SNP variant rs181206 has a L→P substitution at position 119. Accordingly, the p28 moiety of the disclosure may contain one or both of (1) the S→A substitution at position 59 and/or (2) the L→P substitution at position 119.

In certain embodiments, the IL27 p28 moiety comprises one or more amino acid substitutions that reduce binding to IL27Ra (IL27Rα) and/or gp130. For example, in some embodiments, the IL27 p28 moiety can have up to 1,000-fold attenuated binding to human IL27Ra (IL27Rα) and/or gp130 as compared to wild type human IL27 p28. In some embodiments, the IL27 p28 moiety can have up to 100-fold, up to 50-fold, up to 25-fold, up to 20-fold, up to 15-fold, up to 10-fold, or up to 5-fold attenuated binding to human IL27Ra (IL27Rα) and/or gp130 as compared to wild type human IL27 p28.

Exemplary amino acid substitutions include, but are not limited to substitutions at amino acids H52, K56, S59, E60, L73, V76, W138, L142, R145, D146, R149, H150, W197, L200, L201, Y204, and R205, wherein amino acid positions are relative to the full length human IL27 p28 amino acid sequence. Corresponding amino acid positions in the mature human sequence, full-length murine sequence, and mature murine sequence are provided in Table 1. In certain embodiments, the amino acid at each identified residue is substituted by an alanine.

TABLE 1
IL27 p28 Moiety Amino Acid Substitutions

| Amino Acid | Amino Acid | Amino Acid | Amino Acid |
|---|---|---|---|
| (Human - | (Human - | (Murine - | (Murine - |
| Full Length) | Mature) | Full Length) | Mature) |
| H52 | H24 | Y48 | Y20 |
| K56 | K28 | K52 | K24 |
| S59 | S31 | S55 | S27 |
| E60 | E32 | E56 | E28 |
| L73 | L45 | L69 | L41 |
| V76 | V48 | V72 | V44 |
| W138 | W110 | W134 | W106 |
| L142 | L114 | L138 | L110 |
| R145 | R117 | R141 | R113 |
| D146 | D118 | D142 | D114 |
| R149 | R121 | R145 | R117 |
| H150 | H122 | H146 | H118 |
| W197 | W169 | W195 | W167 |
| L200 | L172 | L198 | L170 |
| L201 | L173 | L199 | L171 |
| Y204 | Y176 | Y202 | Y174 |
| R205 | R177 | Q203 | Q175 |

An exemplary amino acid substitution at full-length human p28 H52 is H52A.

An exemplary amino acid substitution at full-length human p28 K56 is K56A.

An exemplary amino acid substitution at full-length human p28 S59 is S59A.

An exemplary amino acid substitution at full-length human p28 E60 is E60A.

An exemplary amino acid substitution at full-length human p28 L73 is L73A.

An exemplary amino acid substitution at full-length human p28 V76 is V76A.

An exemplary amino acid substitution at full-length human p28 W138 is W138A.

An exemplary amino acid substitution at full-length human p28 L142 is L142A.

An exemplary amino acid substitution at full-length human p28 R145 is R145A.

An exemplary amino acid substitution at full-length human p28 D146 is D146A.

An exemplary amino acid substitution at full-length human p28 R149 is R149A.

An exemplary amino acid substitution at full-length human p28 H150 is H150A.

An exemplary amino acid substitution at full-length human p28 HW197 is HW197A.

An exemplary amino acid substitution at full-length human p28 L200 is L200A.

An exemplary amino acid substitution at full-length human p28 L201 is L201A.

An exemplary amino acid substitution at full-length human p28 Y204 is Y204A.

An exemplary amino acid substitution at full-length human p28 R205 is R205A.

In some embodiments, the p28 moiety is fused, either directly or indirectly, to an IL27 p28 binding domain of IL27Ra (IL27Rα) (i.e., the IL27Ra (IL27Rα) moiety), optionally via a linker (e.g., as described in Section 5.8). When present, the IL27 p28 binding domain of IL27Ra (IL27Rα) can be N-terminal or C-terminal to the IL27 p28 moiety. When the p28 moiety is “directly” fused to the IL27 p28 binding domain of IL27Ra (IL27Rα), the p28 moiety and the IL27 p28 binding domain of IL27Ra (IL27Rα) are positioned adjacently on the same monomer, separated only by a linker, if present. When the p28 moiety is “indirectly” fused to the IL27 p28 binding domain of IL27Ra (IL27Rα), the p28 moiety and the IL27 p28 binding domain of IL27Ra (IL27Rα) are separated by one or more other domains (e.g., an IL27 EBI3 moiety) on the same monomer, or are located on separate monomers.

In some embodiments, the p28 moiety is fused, either directly or indirectly, to an IL27 p28 binding domain of gp130 (i.e., the gp130 moiety), optionally via a linker (e.g., as described in Section 5.8). When present, the IL27 p28 binding domain of gp130 can be N-terminal or C-terminal to the IL27 p28 moiety. When the p28 moiety is “directly” fused to the IL27 p28 binding domain of gp130, the p28 moiety and the IL27 p28 binding domain of gp130 are positioned adjacently on the same monomer, separated only by a linker, if present. When the p28 moiety is “indirectly” fused to the IL27 p28 binding domain of gp130, the p28 moiety and the IL27 p28 binding domain of gp130 are separated by one or more other domains (e.g., an IL27 EBI3 moiety) on the same monomer, or are located on separate monomers.

Human p28 contains several potential O-linked glycosylation sites but no N-linked glycosylation sites. Murine p28 contains a potential N-linked glycosylation site at amino acid 85. The present disclosure encompasses p28 moieties with or without N-linked glycans and/or 0-linked glycans.

The p28 moiety may comprise a peptide tag, e.g., a peptide tag that facilitates purification, at its N-terminus or C-terminus. In some embodiments the peptide tag is a myc-myc-his (mmh) tag.

In some embodiments, the p28 moiety comprises a p28 amino acid sequence shown in Section 5.1.1.

### 5.4. The Multimerization Moiety

5.4.1. Fc Domains

In some embodiments, the IL27 agonists and IL27 monomers of the disclosure include one or more multimerization moieties. In certain embodiments, an IL27 monomer of the disclosure comprises a single multimerization moiety (e.g., a single Fc domain) and/or an IL27 agonist of the disclosure comprises two multimerization moieties (e.g., two Fc domains that can associate to form an Fc region).

The IL27 agonists and IL27 monomers of the disclosure can include an Fc domain, or a pair of Fc domains that associate to form an Fc region, derived from any suitable species. In one embodiment the Fc domain is derived from a human Fc domain. In preferred embodiments, the EBI3 and/or p28 moieties of an IL27 agonist or an IL27 monomer of the disclosure are fused to an IgG Fc molecule (e.g., and IgG1 or an IgG4 Fc domain).

The EBI3 and/or p28 moieties may be fused to the N-terminus or the C-terminus of the Fc molecule, e.g., an IgG Fc domain (e.g., as represented in FIGS. 3 and 4).

One embodiment of the present disclosure is directed to a dimer comprising two Fc-fusion polypeptide monomers created by fusing an IL27 domain (e.g., an EBI3 and/or a p28 moiety) to the Fc region of an antibody, e.g., by fusing an EBI3 moiety and/or a p28 moiety to an Fc domain that can upon expression form an IL27 monomer capable of dimerization or by fusing an EBI3 moiety to a first Fc domain and a p28 moiety to a second Fc domain that upon expression form two different IL27 monomers that are capable of dimerizing. The dimer can be made by, for example, inserting a gene fusion encoding the fusion protein(s) into an appropriate expression vector, expressing the gene fusion(s) in host cells transformed with the recombinant expression vector, and allowing the expressed fusion protein(s) to assemble much like antibody molecules, whereupon interchain bonds form between the Fc domains to yield the dimer. In some embodiments, an Fc dimer polypeptide contains an EBI3 moiety or a p28 moiety, and the IL27 agonist is formed by association of two Fc domains. In other embodiments, an Fc dimer polypeptide comprises both an EBI3 moiety and a p28 moiety, on different Fc polypeptide monomers or on the same Fc polypeptide monomer. Accordingly, in various embodiments, the IL27 agonists of the disclosure have an Fc domain: EBI3 moiety or Fc domain: p28 moiety stoichiometry of 1:1, 2:1 or 4:1.

The Fc domains that can be incorporated into IL27 monomer can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment the Fc domain is derived from IgG1. In one embodiment the Fc domain is derived from IgG4.

The two Fc domains within the Fc region can be the same or different from one another. In a native antibody the Fc domains are typically identical, but for the purpose of producing multispecific binding molecules, e.g., the IL27 agonists of the disclosure, the Fc domains might advantageously be different to allow for heterodimerization, as described in Section 5.4.2 below.

In native antibodies, the heavy chain Fc domain of IgA, IgD and IgG is composed of two heavy chain constant domains (CH2 and CH3) and that of IgE and IgM is composed of three heavy chain constant domains (CH2, CH3 and CH4). These dimerize to create an Fc region.

In IL27 agonists of the present disclosure, the Fc region, and/or the Fc domains within it, may be chimeric, combining sequences derived from one or more different classes of antibody. Thus, the Fc region, and/or the Fc domains within it, can comprise heavy chain constant domains from one or more different classes of antibody, for example one, two or three different classes.

In one embodiment the Fc region comprises CH2 and CH3 domains derived from IgG1.

In one embodiment the Fc region comprises CH2 and CH3 domains derived from IgG2.

In one embodiment the Fc region comprises CH2 and CH3 domains derived from IgG3.

In one embodiment the Fc region comprises CH2 and CH3 domains derived from IgG4.

In one embodiment the Fc region comprises a CH4 domain from IgM. The IgM CH4 domain is typically located at the C-terminus of the CH3 domain.

In one embodiment the Fc region comprises CH2 and CH3 domains derived from IgG and a CH4 domain derived from IgM.

In a further embodiment, a chimeric Fc domain can comprise part or all of a CH2 sequence derived from a human IgG1, human IgG2 or human IgG4 CH2 region, and part or all of a CH3 sequence derived from a human IgG1, human IgG2 or human IgG4. A chimeric Fc domain can also contain a chimeric hinge region, as described in Section 5.8.2.1. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the IL27 muteins set forth herein comprises, from N- to C-terminus: [IgG4 CH1]-[IgG4 upper hinge]-[IgG2 lower hinge]-[IgG4 CH2]-[IgG4 CH3]. Another example of a chimeric Fc domain that can be included in any of the antigen-binding molecules set forth herein comprises, from N- to C-terminus: [IgG1 CH1]-[IgG1 upper hinge]-[IgG2 lower hinge]-[IgG4 CH2]-[IgG1 CH3]. These and other examples of chimeric Fc domains that can be included in any of the antigen-binding molecules of the present invention are described in WO 2014/121087. Chimeric Fc regions having these general structural arrangements, and variants thereof, can have altered Fc receptor binding, which in turn affects Fc effector function.

It will be appreciated that the heavy chain constant domains for use in producing an Fc region for the IL27 agonists of the present disclosure may include variants of naturally occurring constant domains. Such variants may comprise one or more amino acid variations compared to wild type constant domains. In one example the Fc region of the present disclosure comprises at least one constant domain that varies in sequence from the wild type constant domain. It will be appreciated that the variant constant domains may be longer or shorter than the wild type constant domain. Preferably the variant constant domains are at least 60% identical or similar to a wild type constant domain. In another example the variant constant domains are at least 70% identical or similar. In another example the variant constant domains are at least 80% identical or similar. In another example the variant constant domains are at least 90% identical or similar. In another example the variant constant domains are at least 95% identical or similar.

IgM and IgA occur naturally in humans as covalent multimers of the common H2L2 antibody unit. IgM occurs as a pentamer when it has incorporated a J-chain, or as a hexamer when it lacks a J-chain. IgA occurs as monomer and dimer forms. The heavy chains of IgM and IgA possess an 18 amino acid extension to the C-terminal constant domain, known as a tailpiece. The tailpiece includes a cysteine residue that forms a disulfide bond between heavy chains in the polymer, and is believed to have an important role in polymerization. The tailpiece also contains a glycosylation site. In certain embodiments, the IL27 agonists of the present disclosure do not comprise a tailpiece.

The Fc domains that are incorporated into the IL27 agonists of the present disclosure may comprise one or more modifications that alter the functional properties of the proteins, for example, binding to Fc-receptors such as FcRn or leukocyte receptors, binding to complement, modified disulfide bond architecture, or altered glycosylation patterns. Exemplary Fc modifications that alter effector function are described in Section 5.4.2.

The Fc domains can also be altered to include modifications that improve manufacturability of asymmetric IL27 agonists, for example by allowing heterodimerization, which is the preferential pairing of non-identical Fc domains over identical Fc domains. Heterodimerization permits the production of IL27 agonists in which different polypeptide components are connected to one another by an Fc region containing Fc domains that differ in sequence. Examples of heterodimerization strategies are exemplified in Section 5.4.2.1.

Alternatively, the Fc domain can be a soluble monomeric Fc domain that has a reduced ability to self-associate. See, e.g., Helm et al., 1996, J. Biol. Chem. 271: 7494-7500 and Ying et al., 2012, J Biol Chem. 287(23):19399-19408. The IL27 agonist can still dimerize through the association of an EBI3 moiety and a p28 moiety. An example of a soluble monomeric Fc domain comprises amino acid substitutions in the positions corresponding to T366 and/or Y407 in CH3, as described in U.S. Patent Publication No. 2019/0367611. The monomeric Fc domains can be of any Ig subtype and can include additional substitutions that reduce effector function, as described in Section 5.4.2.

As used herein, the term “Fc region” can include Fc domains with or without hinge sequences. In various embodiments in which the Fc region comprises a heavy chain constant region including a hinge domain, positions 233-236 within the hinge domain may be G, G, G and unoccupied; G, G, unoccupied, and unoccupied; G, unoccupied, unoccupied, and unoccupied; or all unoccupied, with positions numbered by EU numbering. Optionally, the heavy chain constant region comprises from N-terminal to C-terminal the hinge domain, a CH2 domain and a CH3 domain. Optionally, the heavy chain constant region comprises from N-terminal to C-terminal a CH1 domain, the hinge domain, a CH2 domain and a CH3 domain. Optionally, the CH1 region, if present, remainder of the hinge region, if any, CH2 region and CH3 region are the same human isotype. Optionally, the CH1 region, if present, remainder of the hinge region, if any, CH2 region and CH3 region are human IgG1. Optionally, the CH1 region, if present, remainder of the hinge region, if any, CH2 region and CH3 region are human IgG2. Optionally, the CH1 region if present, remainder of the hinge region, if any, CH2 region and CH3 region are human IgG4.

Optionally, the constant region has a CH3 domain modified to reduce binding to protein A.

These and other examples of Fc regions that can be included in any of the IL27 muteins of the present disclosure are described in WO 2016/161010. Exemplary hinge sequences are set forth in Section 5.8.2 and subsections thereof.

It will be appreciated that any of the modifications mentioned above can be combined in any suitable manner to achieve the desired functional properties and/or combined with other modifications to alter the properties of the IL27 agonists.

5.4.2. Fc Domains with Altered Effector Function

In some embodiments, the Fc domain comprises one or more amino acid substitutions that reduces binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain (e.g., an Fc domain of an IL27 monomer) or the Fc region (e.g., one or both Fc domains of an IL27 receptor agonist that can associate to form an Fc region) comprises an amino acid substitution at a position selected from the group of E233, L234, L235, N297, P331 and P329 (numberings according to Kabat EU index). In a more specific embodiment, the Fc domain or the Fc region comprises an amino acid substitution at a position selected from the group of L234, L235 and P329 (numberings according to Kabat EU index). In some embodiments, the Fc domain or the Fc region comprises the amino acid substitutions L234A and L235A (numberings according to Kabat EU index). In one such embodiment, the Fc domain or Fc region is an Igd Fc domain or Fc region, particularly a human Igd Fc domain or Fc region. In one embodiment, the Fc domain or the Fc region comprises an amino acid substitution at position P329. In a more specific embodiment, the amino acid substitution is P329A or P329G, particularly P329G (numberings according to Kabat EU index). In one embodiment, the Fc domain or Fc region comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain or Fc region comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one embodiment, the Fc domain is an IgG1 Fc domain, particularly a human IgG1 Fc. In some embodiments, the IgG Fc domain is a variant IgG comprising D265A, N297A mutations (EU numbering) to reduce effector function.

In another embodiment, the Fc domain is an IgG4 Fc domain with reduced binding to Fc receptors. Exemplary IgG4 Fc domains with reduced binding to Fc receptors may comprise an amino acid sequence selected from Table 2 below. In some embodiments, the Fc domain includes only the bolded portion of the sequences shown below:

TABLE 2

| Fc Domain | Sequence |
|---|---|
| SEQ ID NO: 3 | Asp Lys Arg Val Glu Ser Lys Tyr Gly Pro Pro Cys Pro Pro Cys Pro |
| (SEQ ID NO: 1 of | Ala Pro Pro Val Ala Gly Pro Ser Val Phe Leu Phe Pro Pro Lys Pro |
| WO2014/121087) | Lys Asp Thr Leu Met Ile Ser Arg Thr Pro Glu Val Thr Cys Val Val |
| | Val Asp Val Ser Gln Glu Asp Pro Glu Val Gln Phe Asn Trp Tyr Val |
| | Asp Gly Val Glu Val His Asn Ala Lys Thr Lys Pro Arg Glu Glu Gln |
| | Phe Asn Ser Thr Tyr Arg Val Val Ser Val Leu Thr Val Leu His Gln |
| | Asp Trp Leu Asn Gly Lys Glu Tyr Lys Cys Lys Val Ser Asn Lys Gly |
| | Leu Pro Ser Ser Ile Glu Lys Thr Ile Ser Lys Ala Lys Gly Gln Pro |
| | Arg Glu Pro Gln Val Tyr Thr Leu Pro Pro Ser Gln Glu Glu Met Thr |
| | Lys Asn Gln Val Ser Leu Thr Cys Leu Val Lys Gly Phe Tyr Pro Ser |
| | Asp Ile Ala Val Glu Trp Glu Ser Asn Gly Gln Pro Glu Asn Asn Tyr |
| | Lys Thr Thr Pro Pro Val Leu Asp Ser Asp Gly Ser Phe Phe Leu Tyr |
| | Ser Arg Leu Thr Val Asp Lys Ser Arg Trp Gln Glu Gly Asn Val Phe |
| | Ser Cys Ser Val Met His Glu Ala Leu His Asn His Tyr Thr Gln Lys |
| | Ser Leu Ser Leu Ser Leu Gly Lys |
| SEQ ID NO: 4 | Asp Lys Lys Val Glu Pro Lys Ser Cys Asp Lys Thr His Thr Cys Pro |
| (SEQ ID NO: 2 of | Pro Cys Pro Ala Pro Pro Val Ala Gly Pro Ser Val Phe Leu Phe Pro |
| WO2014/121087) | Pro Lys Pro Lys Asp Thr Leu Met Ile Ser Arg Thr Pro Glu Val Thr |
| | Cys Val Val Val Asp Val Ser Gln Glu Asp Pro Glu Val Gln Phe Asn |
| | Trp Tyr Val Asp Gly Val Glu Val His Asn Ala Lys Thr Lys Pro Arg |
| | Glu Glu Gln Phe Asn Ser Thr Tyr Arg Val Val Ser Val Leu Thr Val |
| | Leu His Gln Asp Trp Leu Asn Gly Lys Glu Tyr Lys Cys Lys Val Ser |
| | Asn Lys Gly Leu Pro Ser Ser Ile Glu Lys Thr Ile Ser Lys Ala Lys |
| | Gly Gln Pro Arg Glu Pro Gln Val Tyr Thr Leu Pro Pro Ser Arg Asp |
| | Glu Leu Thr Lys Asn Gln Val Ser Leu Thr Cys Leu Val Lys Gly Phe |
| | Tyr Pro Ser Asp Ile Ala Val Glu Trp Glu Ser Asn Gly Gln Pro Glu |
| | Asn Asn Tyr Lys Thr Thr Pro Pro Val Leu Asp Ser Asp Gly Ser Phe |
| | Phe Leu Tyr Ser Lys Leu Thr Val Asp Lys Ser Arg Trp Gln Gln Gly |
| | Asn Val Phe Ser Cys Ser Val Met His Glu Ala Leu His Asn His Tyr |
| | Thr Gln Lys Ser Leu Ser Leu Ser Pro Gly Lys |
| SEQ ID NO: 5 | Ala Ser Thr Lys Gly Pro Ser Val Phe Pro Leu Ala Pro Ser Ser Lys |
| (SEQ ID NO: 30 of | Ser Thr Ser Gly Gly Thr Ala Ala Leu Gly Cys Leu Val Lys Asp Tyr |
| WO2014/121087) | Phe Pro Glu Pro Val Thr Val Ser Trp Asn Ser Gly Ala Leu Thr Ser |
| | Gly Val His Thr Phe Pro Ala Val Leu Gln Ser Ser Gly Leu Tyr Ser |
| | Leu Ser Ser Val Val Thr Val Pro Ser Ser Ser Leu Gly Thr Gln Thr |
| | Tyr Ile Cys Asn Val Asn His Lys Pro Ser Asn Thr Lys Val Asp Lys |
| | Lys Val Glu Pro Lys Ser Cys Asp Lys Thr His Thr Cys Pro Pro Cys |
| | Pro Ala Pro Pro Val Ala Gly Pro Ser Val Phe Leu Phe Pro Pro Lys |
| | Pro Lys Asp Thr Leu Met Ile Ser Arg Thr Pro Glu Val Thr Cys Val |
| | Val Val Asp Val Ser Gln Glu Asp Pro Glu Val Gln Phe Asn Trp Tyr |
| | Val Asp Gly Val Glu Val His Asn Ala Lys Thr Lys Pro Arg Glu Glu |
| | Gln Phe Asn Ser Thr Tyr Arg Val Val Ser Val Leu Thr Val Leu His |
| | Gln Asp Trp Leu Asn Gly Lys Glu Tyr Lys Cys Lys Val Ser Asn Lys |
| | Gly Leu Pro Ser Ser Ile Glu Lys Thr Ile Ser Lys Ala Lys Gly Gln |
| | Pro Arg Glu Pro Gln Val Tyr Thr Leu Pro Pro Ser Arg Asp Glu Leu |
| | Thr Lys Asn Gln Val Ser Leu Thr Cys Leu Val Lys Gly Phe Tyr Pro |
| | Ser Asp Ile Ala Val Glu Trp Glu Ser Asn Gly Gln Pro Glu Asn Asn |
| | Tyr Lys Thr Thr Pro Pro Val Leu Asp Ser Asp Gly Ser Phe Phe Leu |
| | Tyr Ser Lys Leu Thr Val Asp Lys Ser Arg Trp Gln Gln Gly Asn Val |
| | Phe Ser Cys Ser Val Met His Glu Ala Leu His Asn His Tyr Thr Gln |
| | Lys Ser Leu Ser Leu Ser Pro Gly Lys |
| SEQ ID NO: 6 | Ala Ser Thr Lys Gly Pro Ser Val Phe Pro Leu Ala Pro Cys Ser Arg |
| (SEQ ID NO: 31 of | Ser Thr Ser Glu Ser Thr Ala Ala Leu Gly Cys Leu Val Lys Asp Tyr |
| WO2014/121087) | Phe Pro Glu Pro Val Thr Val Ser Trp Asn Ser Gly Ala Leu Thr Ser |
| | Gly Val His Thr Phe Pro Ala Val Leu Gln Ser Ser Gly Leu Tyr Ser |
| | Leu Ser Ser Val Val Thr Val Pro Ser Ser Ser Leu Gly Thr Lys Thr |
| | Tyr Thr Cys Asn Val Asp His Lys Pro Ser Asn Thr Lys Val Asp Lys |
| | Arg Val Glu Ser Lys Tyr Gly Pro Pro Cys Pro Pro Cys Pro Ala Pro |
| | Pro Val Ala Gly Pro Ser Val Phe Leu Phe Pro Pro Lys Pro Lys Asp |
| | Thr Leu Met Ile Ser Arg Thr Pro Glu Val Thr Cys Val Val Val Asp |
| | Val Ser Gln Glu Asp Pro Glu Val Gln Phe Asn Trp Tyr Val Asp Gly |
| | Val Glu Val His Asn Ala Lys Thr Lys Pro Arg Glu Glu Gln Phe Asn |
| | Ser Thr Tyr Arg Val Val Ser Val Leu Thr Val Leu His Gln Asp Trp |
| | Leu Asn Gly Lys Glu Tyr Lys Cys Lys Val Ser Asn Lys Gly Leu Pro |
| | Ser Ser Ile Glu Lys Thr Ile Ser Lys Ala Lys Gly Gln Pro Arg Glu |
| | Pro Gln Val Tyr Thr Leu Pro Pro Ser Gln Glu Glu Met Thr Lys Asn |
| | Gln Val Ser Leu Thr Cys Leu Val Lys Gly Phe Tyr Pro Ser Asp Ile |
| | Ala Val Glu Trp Glu Ser Asn Gly Gln Pro Glu Asn Asn Tyr Lys Thr |
| | Thr Pro Pro Val Leu Asp Ser Asp Gly Ser Phe Phe Leu Tyr Ser Arg |
| | Leu Thr Val Asp Lys Ser Arg Trp Gln Glu Gly Asn Val Phe Ser Cys |
| | Ser Val Met His Glu Ala Leu His Asn His Tyr Thr Gln Lys Ser Leu |
| | Ser Leu Ser Leu Gly Lys |
| SEQ ID NO: 7 | Ala Ser Thr Lys Gly Pro Ser Val Phe Pro Leu Ala Pro Ser Ser Lys |
| (SEQ ID NO: 37 of | Ser Thr Ser Gly Gly Thr Ala Ala Leu Gly Cys Leu Val Lys Asp Tyr |
| WO2014/121087) | Phe Pro Glu Pro Val Thr Val Ser Trp Asn Ser Gly Ala Leu Thr Ser |
| | Gly Val His Thr Phe Pro Ala Val Leu Gln Ser Ser Gly Leu Tyr Ser |
| | Leu Ser Ser Val Val Thr Val Pro Ser Ser Ser Leu Gly Thr Gln Thr |
| | Tyr Ile Cys Asn Val Asn His Lys Pro Ser Asn Thr Lys Val Asp Lys |
| | Lys Val Glu Pro Lys Ser Cys Asp Lys Thr His Thr Cys Pro Pro Cys |
| | Pro Ala Pro Pro Val Ala Gly Pro Ser Val Phe Leu Phe Pro Pro Lys |
| | Pro Lys Asp Thr Leu Met Ile Ser Arg Thr Pro Glu Val Thr Cys Val |
| | Val Val Asp Val Ser Gln Glu Asp Pro Glu Val Gln Phe Asn Trp Tyr |
| | Val Asp Gly Val Glu Val His Asn Ala Lys Thr Lys Pro Arg Glu Glu |
| | Gln Phe Asn Ser Thr Tyr Arg Val Val Ser Val Leu Thr Val Leu His |
| | Gln Asp Trp Leu Asn Gly Lys Glu Tyr Lys Cys Lys Val Ser Asn Lys |
| | Gly Leu Pro Ser Ser Ile Glu Lys Thr Ile Ser Lys Ala Lys Gly Gln |
| | Pro Arg Glu Pro Gln Val Tyr Thr Leu Pro Pro Ser Arg Asp Glu Leu |
| | Thr Lys Asn Gln Val Ser Leu Thr Cys Leu Val Lys Gly Phe Tyr Pro |
| | Ser Asp Ile Ala Val Glu Trp Glu Ser Asn Gly Gln Pro Glu Asn Asn |
| | Tyr Lys Thr Thr Pro Pro Val Leu Asp Ser Asp Gly Ser Phe Phe Leu |
| | Tyr Ser Lys Leu Thr Val Asp Lys Ser Arg Trp Gln Gln Gly Asn Val |
| | Phe Ser Cys Ser Val Met His Glu Ala Leu His Asn Arg Phe Thr Gln |
| | Lys Ser Leu Ser Leu Ser Pro Gly Lys |
| SEQ ID NO: 8 | Ala Ser Thr Lys Gly Pro Ser Val Phe Pro Leu Ala Pro Cys Ser Arg |
| (SEQ ID NO: 38 of | Ser Thr Ser Glu Ser Thr Ala Ala Leu Gly Cys Leu Val Lys Asp Tyr |
| WO2014/121087) | Phe Pro Glu Pro Val Thr Val Ser Trp Asn Ser Gly Ala Leu Thr Ser |
| | Gly Val His Thr Phe Pro Ala Val Leu Gln Ser Ser Gly Leu Tyr Ser |
| | Leu Ser Ser Val Val Thr Val Pro Ser Ser Ser Leu Gly Thr Lys Thr |
| | Tyr Thr Cys Asn Val Asp His Lys Pro Ser Asn Thr Lys Val Asp Lys |
| | Arg Val Glu Ser Lys Tyr Gly Pro Pro Cys Pro Pro Cys Pro Ala Pro |
| | Pro Val Ala Gly Pro Ser Val Phe Leu Phe Pro Pro Lys Pro Lys Asp |
| | Thr Leu Met Ile Ser Arg Thr Pro Glu Val Thr Cys Val Val Val Asp |
| | Val Ser Gln Glu Asp Pro Glu Val Gln Phe Asn Trp Tyr Val Asp Gly |
| | Val Glu Val His Asn Ala Lys Thr Lys Pro Arg Glu Glu Gln Phe Asn |
| | Ser Thr Tyr Arg Val Val Ser Val Leu Thr Val Leu His Gln Asp Trp |
| | Leu Asn Gly Lys Glu Tyr Lys Cys Lys Val Ser Asn Lys Gly Leu Pro |
| | Ser Ser Ile Glu Lys Thr Ile Ser Lys Ala Lys Gly Gln Pro Arg Glu |
| | Pro Gln Val Tyr Thr Leu Pro Pro Ser Gln Glu Glu Met Thr Lys Asn |
| | Gln Val Ser Leu Thr Cys Leu Val Lys Gly Phe Tyr Pro Ser Asp Ile |
| | Ala Val Glu Trp Glu Ser Asn Gly Gln Pro Glu Asn Asn Tyr Lys Thr |
| | Thr Pro Pro Val Leu Asp Ser Asp Gly Ser Phe Phe Leu Tyr Ser Arg |
| | Leu Thr Val Asp Lys Ser Arg Trp Gln Glu Gly Asn Val Phe Ser Cys |
| | Ser Val Met His Glu Ala Leu His Asn Arg Phe Thr Gln Lys Ser Leu |
| | Ser Leu Ser Leu Gly Lys |

In a particular embodiment, the IgG4 with reduced effector function comprises the bolded portion of the amino acid sequence of SEQ ID NO:6 (SEQ ID NO:31 of WO2014/121087), sometimes referred to herein as IgG4s or hIgG4s.

For IL27 agonists of the disclosure that are heterodimers, it is possible to incorporate a combination of the variant IgG4 Fc sequences set forth above, for example an Fc region comprising an Fc domain comprising the amino acid sequence of SEQ ID NO:5 (or the bolded portion thereof) and an Fc domain comprising the amino acid sequence of SEQ ID NO:7 (or the bolded portion thereof) or an Fc region comprising an Fc domain comprising the amino acid sequence of SEQ ID NO:6 (or the bolded portion thereof) and the Fc domain comprising the amino acid sequence of SEQ ID NO:8 (or the bolded portion thereof) (corresponding to SEQ ID NOS: 30, 37, 31 and 38, respectively).

In a particular embodiment, the Fc domain comprises the amino acid sequence designated in Section 7.1.1 as hIgG4s.

In another particular embodiment, the Fc domain comprises the amino acid sequence designated in Section 7.1.1 as hIgG1, which is a variant IgG1-based Fc sequence comprising D265A, N297A mutations (EU numbering) to reduce effector function.

5.4.2.1. Fc Heterodimerization Variants

Certain IL27 agonists entail dimerization between two Fc domains that, unlike a native immunoglobulin, are operably linked to non-identical N-terminal regions, e.g., one Fc domain connected to a Fab and the other Fc domain connected to an IL27 domain. Inadequate heterodimerization of two Fc domain to form an Fc region can be an obstacle for increasing the yield of desired heterodimeric molecules and represents challenges for purification. A variety of approaches available in the art can be used in for enhancing dimerization of Fc domains that might be present in the IL27 agonists of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO 2009/089004A1.

The present disclosure provides IL27 agonists comprising Fc heterodimers, i.e., Fc regions comprising heterologous, non-identical Fc domains. Typically, each Fc domain in the Fc heterodimer comprises a CH3 domain of an antibody. The CH3 domains are derived from the constant region of an antibody of any isotype, class or subclass, and preferably of IgG (IgG1, IgG2, IgG3 and IgG4) class, as described in the preceding section.

Heterodimerization of the two different heavy chains at CH3 domains give rise to the desired IL27 agonist, while homodimerization of identical heavy chains will reduce yield of the desired IL27 agonist. Thus, in a preferred embodiment, the polypeptides that associate to form an IL27 agonist of the disclosure will contain CH3 domains with modifications that favor heterodimeric association relative to unmodified Fc domains.

In a specific embodiment said modification promoting the formation of Fc heterodimers is a so-called “knob-into-hole” or “knob-in-hole” modification, comprising a “knob” modification in one of the Fc domains and a “hole” modification in the other Fc domain. The knob-into-hole technology is described e.g., in U.S. Pat. Nos. 5,731,168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g., by site-specific mutagenesis, or by peptide synthesis. An exemplary substitution is Y470T.

In a specific such embodiment, in the first Fc domain the threonine residue at position 366 is replaced with a tryptophan residue (T366W), and in the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second Fc domain additionally the tyrosine residue at position 349 is replaced by a cysteine residue (Y349C) (numbering according to Kabat EU index). In a particular embodiment, the first Fc domain comprises the amino acid substitutions S354C and T366W, and the second Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285(25): 19637-46) can be used to promote the association of the first and the second Fc domains of the Fc region.

As an alternative, or in addition, to the use of Fc domains that are modified to promote heterodimerization, an Fc domain can be modified to allow a purification strategy that enables selections of Fc heterodimers. In one such embodiment, one polypeptide comprises a modified Fc domain that abrogates its binding to Protein A, thus enabling a purification method that yields a heterodimeric protein. See, for example, U.S. Pat. No. 8,586,713. As such, the IL27 agonists comprise a first CH3 domain and a second Ig CH3 domain, wherein the first and second Ig CH3 domains differ from one another by at least one amino acid, and wherein at least one amino acid difference reduces binding of the IL27 agonist to Protein A as compared to a corresponding IL27 agonist lacking the amino acid difference. In one embodiment, the first CH3 domain binds Protein A and the second CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU). This class of modifications is referred to herein as “star” mutations.

### 5.5. Stabilization Moieties

The IL27 agonists of the disclosure can comprise a stabilization moiety that can further extend the IL27 agonist's half-life in vivo. Serum half-life is often divided into an alpha phase and a beta phase. Either or both phases may be improved significantly by addition of an appropriate stabilization moiety. For example, the stabilization moiety can increase the serum half-life of the IL27 agonist by more than 5, 10, 20, 30, 40, 50, 60, 70, 80, 90, 100, 120, 150, 200, 400, 600, 800, 1000% or more relative to a corresponding IL27 agonist not containing the stabilization moiety. For the purpose of this disclosure, serum half-life can refer to the half-life in humans or other mammals (e.g., mice or non-human primates). Further, it is recognized the inclusion of an Fc domain in an IL27 agonist extends the half-life of the IL27 domain; in the context of this disclosure the term “stabilization moiety” refers to a moiety other than an Fc domain/Fc region.

Wild type IL27 has a serum half-life of less than 2 hours. The IL27 agonists of the disclosure have preferably a serum half-life in humans and/or mice of at least about 3 hours, at least about 4 hours, at least about 6 hours, or at least about 8 hours. In some embodiments, the IL27 agonists of the disclosure have a serum half-life of at least 10 hours, at least 12 hours, at least 15 hours, at least 18 hours, at least 24 hours, at least 36 hours, at least 48 hours, at least 60 hours, or at least 72 hours.

Stabilization moieties, include polyoxyalkylene moieties (e.g., polyethylene glycol), sugars (e.g., sialic acid), and well-tolerated protein moieties (e.g., transferrin and serum albumin).

Other stabilization moieties that can be used in the IL27 agonists of the disclosure include those described in Kontermann et al., 2011, Current Opinion in Biotechnology 22:868-76. Such Stabilization moieties include, but are not limited to, human serum albumin fusions, human serum albumin conjugates, human serum albumin binders (e.g., Adnectin PKE, AlbudAb, the albumin-binding domain from protein G), XTEN fusions, PAS fusions (i.e., recombinant PEG mimetics based on the three amino acids proline, alanine, and serine), carbohydrate conjugates (e.g., hydroxyethyl starch (HES)), glycosylation, polysialic acid conjugates, and fatty acid conjugates.

Accordingly, in some embodiments the disclosure provides an IL27 agonist comprising a stabilization moiety that is a polymeric sugar.

Serum albumin can also be engaged in half-life extension through modules with the capacity to non-covalently interact with albumin. Accordingly, the IL27 agonists of the disclosure can include as a stabilization moiety an albumin-binding protein. The albumin-binding protein can be either conjugated or genetically fused to one or more other components of the IL27 agonist of the disclosure. Proteins with albumin-binding activity are known from certain bacteria. For example, streptococcal protein G contains several small albumin-binding domains composed of roughly 50 amino acid residues (6 kDa). Additional examples of serum albumin binding proteins such as those described in U.S. Publication Nos. 2007/0178082 and 2007/0269422. Fusion of an albumin binding domain to a protein results in a strongly extended half-life (see Kontermann et al., 2011, Current Opinion in Biotechnology 22:868-76).

In other embodiments, the stabilization moiety is human serum albumin, as described in Section 5.5.1 below. See, for example, IL27M20 and IL27M21 (FIGS. 5A and 5B). In other embodiments, the stabilization moiety is transferrin.

In yet other embodiments, the stabilization moiety is a polyethylene glycol moiety or another polymer, as described in Section 5.5.1 below.

The stabilization moiety may comprise a peptide tag, e.g., a peptide tag that facilitates purification, at its N-terminus or C-terminus. In some embodiments the peptide tag is a myc-myc-his (mmh) tag.

The stabilization moiety can be connected to one or more other components of the IL27 agonists of the disclosure via a linker, for example as described in Section 5.8 below.

5.5.1. Human Serum Albumin

In some embodiments, an IL27 agonist of the disclosure comprises human serum albumin (HSA), a natural variant thereof, an engineered variant thereof, or a fragment of any one thereof.

The EBI3 and/or p28 moieties may be fused to the N-terminus or the C-terminus of the HSA (e.g., as represented in FIGS. 5A and 5B). In certain embodiments, the EBI3 and/or p28 moieties are fused to the N-terminus of the HSA.

One embodiment of the present disclosure is directed to a dimer comprising two HSA polypeptides created through the association of an EBI3 moiety with a p28 moiety, where each of the EBI3 moiety and the p28 moiety are fused to a separate HSA polypeptide.

Another embodiment of the present disclosure is directed to a monomer comprising a single HSA polypeptide to which both an EBI3 moiety and a p28 moiety have been fused (e.g., as represented in FIG. 5A). In some embodiments, the monomer is arranged, from N- to C-terminus, in the order EBI3 moiety-optional linker-p28 moiety-optional linker-HSA. In other embodiments, the monomer is arranged, from N- to C-terminus, in the order p28 moiety-optional linker-EBI3 moiety-optional linker-HSA.

The HSA polypeptide, or each HSA polypeptide when the IL27 agonist comprises two or more HSA polypeptides, comprises a wild type or variant HSA polypeptide, or a fragment thereof. The variant may be a natural variant or an engineered variant.

In some embodiments, an HSA polypeptide is or comprises an amino acid sequence comprising at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to a HSA.

In some embodiments, the HSA polypeptide is full-length HSA. In other embodiments, the HSA polypeptide is mature HSA. The sequence of HSA has the Uniprot identifier P02768 (uniprot.org/uniprot/P02768). HSA is synthesized as a precursor polypeptide of 609 amino acids, including a signal peptide (amino acids 1 to 18) and a propeptide (amino acids 19 to 24). The mature HSA includes amino acids 25 to 609. In some embodiments, the HSA of the disclosure comprises full length HSA. In other embodiments, the HSA of the disclosure comprises mature HSA. The amino acid sequence of HSA is provided below:

| (SEQ ID NO: 9) | |
|---|---|
| | 10 20 30 40 |
| | MKWVTFISLL FLFSSAYSRG VFRRDAHKSE VAHRFKDLGE |
| | 50 60 70 80 |
| | ENFKALVLIA FAQYLQQCPF EDHVKLVNEV TEFAKTCVAD |
| | 90 100 110 120 |
| | ESAENCDKSL HTLFGDKLCT VATLRETYGE MADCCAKQEP |
| | 130 140 150 160 |
| | ERNECFLQHK DDNPNLPRLV RPEVDVMCTA FHDNEETFLK |
| | 170 180 190 200 |
| | KYLYEIARRH PYFYAPELLE FAKRYKAAFT ECCQAADKAA |
| | 210 220 230 240 |
| | CLLPKLDELR DEGKASSAKQ RLKCASLQKF GERAFKAWAV |
| | 250 260 270 280 |
| | ARLSQRFPKA EFAEVSKLVT DLTKVHTECC HGDLLECADD |
| | 290 300 310 320 |
| | RADLAKYICE NQDSISSKLK ECCEKPLLEK SHCIAEVEND |
| | 330 340 350 360 |
| | EMPADLPSLA ADFVESKDVC KNYAEAKDVF LGMFLYEYAR |
| | 370 380 390 400 |
| | RHPDYSVVLL LRLAKTYETT LEKCCAAADP HECYAKVFDE |
| | 410 420 430 440 |
| | FKPLVEEPQN LIKQNCELFE QLGEYKFQNA LLVRYTKKVP |
| | 450 460 470 480 |
| | QVSTPTLVEV SRNLGKVGSK CCKHPEAKRM PCAEDYLSVV |
| | 490 500 510 520 |
| | LNQLCVLHEK TPVSDRVTKC CTESLVNRRP CFSALEVDET |
| | 530 540 550 560 |
| | YVPKEFNAET FTFHADICTL SEKERQIKKQ TALVELVKHK |
| | 570 580 590 600 |
| | PKATKEQLKA VMDDFAAFVE KCCKADDKET CFAEEGKKLV |
| | AASQAALGL. |

Amino acid 25 of full-length HSA is amino acid 1 of mature HSA.

A significant number of naturally occurring HSA variants have been reported and are summarized at uniprot.org/uniprot/P02768, any of which may be used in the stabilization moiety of the disclosure.

5.5.2. Polyethylene Glycol

In some embodiments, the IL27 agonist comprises polyethylene glycol (PEG) or another hydrophilic polymer as a stabilization moiety, for example a copolymer of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), dextran or poly(n-vinyl pyrrolidone)polyethylene glycol, a propropylene glycol homopolymer, a prolypropylene oxide/ethylene oxide co-polymer, a polyoxyethylated polyol (e.g., glycerol), polyvinyl alcohol, and mixtures thereof. The polymer may be of any molecular weight, and may be branched or unbranched.

PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented by the formula: X—O(CH₂CH₂O)ₙ-1CH₂CH₂OH, where n is 20 to 2300 and X is H or a terminal modification, e.g., a C₁₋₄ alkyl. PEG can contain further chemical groups which are necessary for binding reactions, which result from the chemical synthesis of the molecule; or which act as a spacer for optimal distance of parts of the molecule. In addition, such a PEG can consist of one or more PEG side-chains which are linked together. PEGs with more than one PEG chain are called multiarmed or branched PEGs. Branched PEGs are described in, for example, European Application No. 473084A and U.S. Pat. No. 5,932,462.

One or more PEG molecules can be attached at different positions on the IL27 agonist, and such attachment may be achieved by reaction with amines, thiols or other suitable reactive groups. The amine moiety may be, for example, a primary amine found at the N-terminus of the IL27 agonist (or a component thereof) or an amine group present in an amino acid, such as lysine or arginine.

PEGylation can be achieved by site-directed PEGylation, wherein a suitable reactive group is introduced into the protein to create a site where PEGylation preferentially occurs. In some embodiments, the IL27 agonist is modified to introduce a cysteine residue at a desired position, permitting site-directed PEGylation on the cysteine. Mutations can be introduced into the coding sequence of an IL27 agonist of the disclosure to generate cysteine residues. This might be achieved, for example, by mutating one or more amino acid residues to cysteine. Preferred amino acids for mutating to a cysteine residue include serine, threonine, alanine and other hydrophilic residues. Preferably, the residue to be mutated to cysteine is a surface-exposed residue. Algorithms are well-known in the art for predicting surface accessibility of residues based on primary sequence or three dimensional structure. The three dimensional structure of IL27 is described in, e.g., Wang et al., 2005, Science 310(5751):1159-63, and can be used to identify surface-exposed residues that can be mutated to cysteine. The mutations can be chosen to avoid disrupting the interaction between IL27 and one or more of its receptors. PEGylation of cysteine residues may be carried out using, for example, PEG-maleimide, PEG-vinylsulfone, PEG-iodoacetamide, or PEG-orthopyridyl disulfide.

The PEG is typically activated with a suitable activating group appropriate for coupling to a desired site on the polypeptide. PEGylation methods are well-known in the art and further described in Zalipsky et al., “Use of Functionalized Poly(Ethylene Glycols) for Modification of Polypeptides” in Polyethylene Glycol Chemistry: Biotechnical and Biomedical Applications, J. M. Harris, Plenus Press, New York (1992), and in Zalipsky, 1995, Advanced Drug Reviews 16: 157-182.

PEG moieties may vary widely in molecular weight and may be branched or linear. Typically, the weight-average molecular weight of PEG is from about 100 Daltons to about 150,000 Daltons. Exemplary weight-average molecular weights for PEG include about 20,000 Daltons, about 40,000 Daltons, about 60,000 Daltons and about 80,000 Daltons. In certain embodiments, the molecular weight of PEG is 40,000 Daltons. Branched versions of PEG having a total molecular weight of any of the foregoing can also be used. In some embodiments, the PEG has two branches. In other embodiments, the PEG has four branches. In another embodiment, the PEG is a bis-PEG (NOF Corporation, DE-200MA), in which two IL27-containing polypeptide chains are conjugated.

Conventional separation and purification techniques known in the art can be used to purify PEGylated IL27 agonists, such as size exclusion (e.g., gel filtration) and ion exchange chromatography. Products can also be separated using SDS-PAGE. Products that can be separated include mono-, di-, tri-, poly- and un-PEGylated IL27 agonists, as well as free PEG. The percentage of mono-PEG conjugates can be controlled by pooling broader fractions around the elution peak to increase the percentage of mono-PEG in the composition. About 90% mono-PEG conjugates represent a good balance of yield and activity.

In some embodiments, the PEGylated IL27 agonists will preferably retain at least about 25%, 50%, 60%, 70%, 80%, 85%, 90%, 95% or 100% of the biological activity associated with the unmodified IL27 agonist. In some embodiments, biological activity refers to its ability to bind to IL27Ra (IL27Rα), gp130 or an IL27 dimer comprising IL27Ra (IL27Rα) and gp130. Binding to the IL27 receptor or its constituent subunits can be assessed by K_{D}, kₒₙ, or k_{off}.

### 5.6. The IL27Ra (IL27Rα) and gp130 Moieties

The present disclosure provides IL27 receptor agonists with IL27Ra (IL27Rα) and/or gp130 moieties capable of binding p28 moieties of the disclosure. Exemplary IL27Ra (IL27Rα) moieties are disclosed in Section 5.6.1 and exemplary gp130 moieties are disclosed in Section 5.6.2.

The IL27 receptor is a heterodimer consisting of interleukin-27 receptor subunit alpha (IL27Ra or IL27Rα) and gp130 subunits. Thus, as used herein, the term “IL27 receptor moiety” refers to an IL27Ra (IL27Rα) moiety and/or gp130 moiety.

The IL27 receptor moiety encompass mature human and non-human (e.g., murine, rat, porcine, non-human primate) IL27Ra (IL27Rα) and gp130 polypeptides, including homologs, variants, and fragments thereof, as well as IL27Ra (IL27Rα) and gp130 polypeptides having, for example, a leader sequence (e.g., the signal peptide), and modified versions of the foregoing. In certain embodiments, the IL27 receptor moieties of the disclosure have one or more amino acid modifications, e.g., substitutions, deletions or insertions, in a p28 binding domain of a IL27Ra (IL27Rα) moiety and/or a gp130 moiety as compared to a wild type or naturally occurring IL27 variant. Hence, the terms an “IL27Ra moiety” (also referred to as the “IL27Ra moiety”) and a “gp130 moiety” encompass proteins of substantially similar sequence as mature wild type human, murine, porcine, or rat IL27Ra and gp130, respectively, more preferably a protein of substantially similar sequence as mature wild type human IL27Ra and gp130 respectively. In various embodiments, the IL27Ra domain and/or gp130 domain comprises an amino acid sequence with at least about 90%, at least about 95%, at least about 96%, at least about 97%, at least about 98%, at least about 99% or 100% sequence identity to human, murine, porcine, or rat IL27Ra and/or gp130 domain sequences, such as those exemplified in Sections 5.6.1 and 5.6.2, respectively.

5.6.1. IL27Ra (IL27Rα) Moiety

IL27Ra or IL27Ra (formerly called T cell cytokine receptor (TCCR) or WSX-1) was initially identified in lymphocytes, including naïve T cells, and shown in vitro to bind IL27. IL27Ra is a single-pass type I cytokine receptor membrane protein and its structure consists of (i) an extracellular domain comprising three modified fibronectin type III (FnIII) domains named the cytokine-binding domain (CBD) (see FIG. 1B); (ii) a single, helical transmembrane domain; and (iii) a cytoplasmic domain containing a Box 1 motif required for JAK interaction and/or activation. The CBD typically contains two pairs of cysteine residues involved in disulfide bridge formation and a characteristic WSXWS signature motif. IL27Ra is also known to exist in soluble forms, at least in humans, where it is spontaneously released from cells as N-glycosylated proteins of 70/90 kDa, through proteolytic cleavage by metalloproteases. The soluble form can bind IL27 in vitro, is complexed with IL27 in vivo, and inhibits IL27 signaling.

IL27 receptor agonists of the disclosure optionally include one or more IL27Ra (IL27Rα) moieties. Each of the one or more IL27Ra (IL27Rα) moieties is capable of binding an IL27 p28 moiety of the disclosure. An IL27Ra (IL27Rα) moiety is or comprises an amino acid sequence comprising at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to an IL27 p28 binding portion of a mammalian, e.g., human or murine, IL27 receptor subunit alpha (IL27Ra or IL27Rα). The IL27 p28 binding portion of IL27Ra (IL27Rα) comprises or consists of the extracellular domain of the receptor subunit, or a p28 binding fragment thereof. The sequence of human IL27Ra (IL27Rα) has the Uniprot identifier Q6UWB1 (uniprot.org/uniprot/Q6UWB1), with amino acids 33 to 516 making up the extracellular domain. The sequence of murine IL27Ra (IL27Rα) has the Uniprot identifier 070394 (uniprot.org/uniprot/070394), with amino acids 25 to 510 making up the extracellular domain.

Human IL27Ra (IL27Rα) is synthesized as a precursor polypeptide of 636 amino acids, from which 32 amino acids are removed to generate mature IL27Ra (IL27Rα). The extracellular domain of IL27Ra (IL27Rα) spans amino acids 33-516 and includes the first fibronectin III domain spanning amino acids 131-231 of IL27Ra (IL27Rα), the second fibronectin III domain spanning amino acids 322-417 of IL27Ra (IL27Rα), and the third fibronectin III domain spanning amino acids 419-511 of IL27Ra (IL27Rα). Accordingly, in some embodiments, the IL27Ra (IL27Rα) domain of the disclosure comprises the extracellular domain of human IL27Ra (IL27Rα), corresponding to positions 33-516 of the 636-amino acid precursor sequence shown below, or the three fibronectin domains, corresponding to positions 131-511 of the 636-amino acid precursor sequence shown below:

| (SEQ ID NO: 10) | |
|---|---|
| | 10 20 30 40 |
| | MRGGRGAPFW LWPLPKLALL PLLWVLFQRT RPQGSAGPLQ |
| | 50 60 70 80 |
| | CYGVGPLGDI NCSWEPLGDL GAPSELHLQS QKYRSNKTQT |
| | 90 100 110 120 |
| | VAVAAGRSWV AIPREQLTMS DKLLVWGTKA GQPLWPPVFV |
| | 130 140 150 160 |
| | NLETQMKPNA PRLGPDVDFS EDDPLEATVH WAPPTWPSHK |
| | 170 180 190 200 |
| | VLICQFHYRR CQEAAWTLLE PELKTIPLTP VEIQDLELAT |
| | 210 220 230 240 |
| | GYKVYGRCRM EKEEDLWGEW SPILSFQTPP SAPKDVWVSG |
| | 250 260 270 280 |
| | NLCGTPGGEE PLLLWKAPGP CVQVSYKVWF WVGGRELSPE |
| | 290 300 310 320 |
| | GITCCCSLIP SGAEWARVSA VNATSWEPLT NLSLVCLDSA |
| | 330 340 350 360 |
| | SAPRSVAVSS IAGSTELLVT WQPGPGEPLE HVVDWARDGD |
| | 370 380 390 400 |
| | PLEKLNWVRL PPGNLSALLP GNFTVGVPYR ITVTAVSASG |
| | 410 420 430 440 |
| | LASASSVWGF REELAPLVGP TLWRLQDAPP GTPAIAWGEV |
| | 450 460 470 480 |
| | PRHQLRGHLT HYTLCAQSGT SPSVCMNVSG NTQSVTLPDL |
| | 490 500 510 520 |
| | PWGPCELWVT ASTIAGQGPP GPILRLHLPD NTLRWKVLPG |
| | 530 540 550 560 |
| | ILFLWGLFLL GCGLSLATSG RCYHLRHKVI PRWVWEKVPD |
| | 570 580 590 600 |
| | PANSSSGQPH MEQVPEAQPL GDLPILEVEE MEPPPVMESS |
| | 610 620 630 |
| | QPAQATAPLD SGYEKHFLPT PHELGLLGPP RPQVLA. |

In some embodiments, the IL27Ra (IL27Rα) moiety includes an extracellular domain (or an amino acid sequence comprising at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the extracellular domain) of a mammalian, e.g., human or murine, IL27Ra (IL27Rα).

In certain aspects, the IL27Ra (IL27Rα) moiety can comprise or consist of an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to amino acids 33 to 516 of full-length human IL27Ra (IL27Rα) (i.e., Uniprot identifier Q6UWB1), optionally wherein the binding portion has an amino acid sequence of (a) at least 160 amino acids, at least 161 amino acids, at least 162 amino acids, at least 164 amino acids or at least 165 amino acids and/or (b) up to 251, up to 240, up to 230, up to 220, up to 210, up to 200, up to 190, up to 180 or up to 170 amino acids from amino acids 33 to 516 of full-length human IL27Ra (IL27Rα). In particular embodiments, the portion of human IL27Ra (IL27Rα) is bounded by any one of (a) and (b) in the preceding sentence, e.g., at least 160 and up to 180 amino acids from human IL27Ra (IL27Rα), at least 162 and up to 200 amino acids from human IL27Ra (IL27Rα), at least 160 and up to 220 amino acids from human IL27Ra (IL27Rα), at least 164 and up to 190 amino acids from human IL27Ra (IL27Rα), and so on and so forth.

In some embodiments, the IL27Ra (IL27Rα) moiety comprises or consists of an amino acid sequence having at least about 90%, at least about 95%, at least about 96%, at least about 97%, at least about 98%, at least about 99% or 100% sequence identity to amino acids 33 to 516 of full-length human IL27Ra (IL27Rα), with or without an additional up to 5 amino acids, up to 10 amino acids, up to 15 amino acids, up to 20 amino acids, up to 30 amino acids, or up to 40 amino acids C-terminal to amino acid residue 516, of IL27Ra (IL27Rα).

The IL27Ra (IL27Rα) moiety-containing IL27receptor agonists of the disclosure can have the IL27Ra (IL27Rα) extracellular domain at the N- or C-terminus of the IL27 p28 moiety when located on the same monomer. In some embodiments, the IL27Ra (IL27Rα) moiety-containing IL27 receptor agonists of the disclosure preferably have the IL27Ra (IL27Rα) extracellular domain at the N-terminus of the IL27 p28 moiety.

Human IL27Ra (IL27Rα) contains potential N-linked glycosylation sites at amino acids 51, 76, 302, 311, 373, 382, and 467. The present disclosure encompasses IL27Ra (IL27Rα) domain molecules with or without N-linked glycans at N51 and/or N76 and/or N302 and/or N311 and/or N373 and/or N382 and/or N467 or the equivalent position in IL27Ra (IL27Rα) of other species.

5.6.2. gp130 Moiety

gp130 was identified as a Type I cytokine receptor required to mediate intracellular signaling by IL27Ra (IL27Rα) in response to IL27. Class I cytokine receptors are characterized by the presence of at least one cytokine binding domain (CBM) that consists of two fibronectin-type III-like (FNIII) domains. The N-terminal domain contains a set of four conserved cysteine residues, and the C-terminal domain contains a WSXWS motif or a closely related sequence. Receptors belonging to this family are engaged by helical cytokines consisting of four tightly packed alpha-helices. gp130 is a promiscuous cytokine receptor, involved in the transduction of at least eight cytokines including IL27. gp130 is the founding member of the IL-6/IL-12 family of “tall” receptors.

The extracellular part of gp130 contains an Ig-like domain (D1) followed by a single CBD (D2 and D3) and three FNIII domains (D4, D5, and D6). The conserved two-domain CBD the Ig-domain as well as the three FNIII domains are required for activation.

IL27 receptor agonists of the disclosure optionally include one or more gp130 moieties. Each of the one or more gp130 moieties is capable of binding an IL27 p28 moiety of the disclosure. An gp130 moiety is or comprises an amino acid sequence comprising at least 70% sequence identity, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, to an IL27 p28 binding portion of a mammalian, e.g., human or murine, membrane glycoprotein 130 (gp130). The IL27 p28 binding portion of gp130 comprises or consists of the extracellular domain of the receptor subunit, or a p28 binding fragment thereof. The sequence of human gp130 has the Uniprot identifier P40189 (uniprot.org/uniprot/P40189), with amino acids 23 to 619 making up the extracellular domain. The sequence of murine gp130 has the Uniprot identifier Q00560 (uniprot.org/uniprot/Q00560), with amino acids 23 to 617 making up the extracellular domain.

Human gp130 is synthesized as a precursor polypeptide of 918 amino acids, from which 22 amino acids are removed to generate mature gp130. The extracellular domain of gp130 spans amino acids 23-619 of gp130, including the IgG-like domain spanning amino acids 26-120, the first fibronectin III domain spanning amino acids 125-216 of gp130, the second fibronectin III domain spanning amino acids 224-324 of gp130, the third fibronectin III domain spanning amino acids 329-424 of gp130, the fourth fibronectin domain spanning amino acids 426-517 of gp130 and the fifth fibronectin domain spanning amino acids 518-613 of gp130. Accordingly, in some embodiments, the gp130 domain of the disclosure comprises the extracellular domain of human gp130, corresponding to positions 23-619 of the 918-amino acid precursor sequence shown below, or the IgG-like and five fibronectin domains, corresponding to positions 26-613 of the 918-amino acid precursor sequence shown below:

| (SEQ ID NO: 11) | |
|---|---|
| | 10 20 30 40 |
| | MLTLQTWLVQ ALFTFLTTES TGELLDPCGY ISPESPVVQL |
| | 50 60 70 80 |
| | HSNFTAVCVI KEKCMDYFHV NANYIVWKTN HFTIPKEQYT |
| | 90 100 110 120 |
| | IINRTASSVT FTDIASLNIQ LTCNILTFGQ LEQNVYGITI |
| | 130 140 150 160 |
| | ISGLPPEKPK NLSCIVNEGK KMRCEWDGGR ETHLETNFTL |
| | 170 180 190 200 |
| | KSEWATHKFA DCKAKPDTPT SCTVDYSTVY FVNIEVWVEA |
| | 210 220 230 240 |
| | ENALGKVTSD HINEDPVYKV KPNPPHNLSV INSEELSSIL |
| | 250 260 270 280 |
| | KLTWTNPSIK SVIILKYNIQ YRTKDASTWS QIPPEDTAST |
| | 290 300 310 320 |
| | RSSFTVQDLK PFTEYVFRIR CMKEDGKGYW SDKSEEASGI |
| | 330 340 350 360 |
| | TYEDRPSNAP SFWYKIDPSH TQGYRTVQLV WKTLPPFEAN |
| | 370 380 390 400 |
| | GKILDYEVTL TRWKSHLQNY TVNATKLTVN LTNDRYLATL |
| | 410 420 430 440 |
| | TVRNLVGKSD AAVLTIPACD FQATHPVMDL KAFPKDNMLW |
| | 450 460 470 480 |
| | VEWTTPRESV KKYILEWCVL SDKAPCITDW QQEDGTVHRT |
| | 490 500 510 520 |
| | YLRGNLAESK CYLITVTPVY ADGPGSPESI KAYLKQAPPS |
| | 530 540 550 560 |
| | KGPTVRTKKV GKNEAVLEWD QLPVDVQNGF IRNYTIFYRT |
| | 570 580 590 600 |
| | IIGNETAVNV DSSHTEYTLS SLTSDTLYMV RMAAYTDEGG |
| | 610 620 630 640 |
| | KDGPEFTFTT PKFAQGEIEA IVVPVCLAFL LTTLLGVLFC |
| | 650 660 670 680 |
| | FNKRDLIKKH IWPNVPDPSK SHIAQWSPHT PPRHNFNSKD |
| | 690 700 710 720 |
| | QMYSDGNFTD VSVVEIEAND KKPEPEDLKS LDEEKKEKIN |
| | 730 740 750 760 |
| | TEGHSSGIGG SSCMSSSRPS LSSSDENESS QNTSSTVQYS |
| | 770 780 790 800 |
| | TVVHSGYRHQ VPSVQVFSRS ESTQPLLDSE ERPEDLQLVD |
| | 810 820 830 840 |
| | HVDGGDGILP RQQYFKQNCS QHESSPDISH FERSKQVSSV |
| | 850 860 870 880 |
| | NEEDFVRLKQ QISDHISQSC GSGQMKMFQE VSAADAFGPG |
| | 890 900 910 |
| | TEGQVERFET VGMEAATDEG MPRSYLPQTV RQGGYMPQ. |

In some embodiments, the gp130 moiety includes an extracellular domain (or an amino acid sequence comprising at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the extracellular domain) of a mammalian, e.g., human or murine, gp130.

In certain aspects, the gp130 moiety can comprise or consist of an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to amino acids 23 to 619 of full-length human gp130 (i.e., Uniprot identifier P40189), optionally wherein the binding portion has an amino acid sequence of (a) at least 160 amino acids, at least 161 amino acids, at least 162 amino acids, at least 164 amino acids or at least 165 amino acids and/or (b) up to 251, up to 240, up to 230, up to 220, up to 210, up to 200, up to 190, up to 180 or up to 170 amino acids from amino acids 33 to 516 of full-length human gp130. In particular embodiments, the portion of human gp130 is bounded by any one of (a) and (b) in the preceding sentence, e.g., at least 160 and up to 180 amino acids from human gp130, at least 162 and up to 200 amino acids from human gp130, at least 160 and up to 220 amino acids from human gp130, at least 164 and up to 190 amino acids from human gp130, and so on and so forth.

In some embodiments, the gp130 moiety comprises or consists of an amino acid sequence having at least about 90%, at least about 95%, at least about 96%, at least about 97%, at least about 98%, at least about 99% or 100% sequence identity to amino acids 23 to 619 of full-length human gp130, with or without an additional up to 5 amino acids, up to 10 amino acids, up to 15 amino acids, up to 20 amino acids, up to 30 amino acids, or up to 40 amino acids C-terminal to amino acid residue 619, of gp130.

The gp130 moiety-containing IL27 receptor agonists of the disclosure can have the gp130 extracellular domain at the N- or C-terminus of the IL27 p28 moiety when located on the same monomer. In some embodiments, the gp130 moiety-containing IL27 receptor agonists of the disclosure preferably have the IL27Ra (IL27Rα) extracellular domain at the N-terminus of the IL27 p28 moiety.

Multiple naturally existing sequence variants of gp130 have been reported in the extracellular domain of the protein (see www.uniprot.org/uniprot/EBI3189). Accordingly, the gp130 domain of the disclosure may contain amino acid substitutions present in one or more natural variants. Exemplary sequence variants include SNP variant rs2228044, which has a G→R substitution at position 148; SNP variant rs199905033, which has a A→G substitution at position 200; and SNP variant rs34417936, which has a V→I substitution at position 499.

Human gp130 contains multiple N-linked glycosylation sites. The present disclosure encompasses gp130 domains with or without N-linked glycans.

### 5.7. The Targeting Moiety

The incorporation of one or more targeting moieties in the IL27 agonists of the disclosure permits the delivery of high concentrations of IL27 into the desired microenvironment or to disease-reactive lymphocytes, for example CD4+CD8+ T lymphocytes, where they can exert a localized effect.

Suitable targeting moiety formats are described in Sections 5.7.2 and 5.7.3. The targeting moiety is preferably an antigen binding moiety, for example an antibody or an antigen-binding fragment of an antibody, e.g., an scFv, as described in Section 5.7.2.1, or a Fab, as described in Section 5.7.2.2.

In other embodiments, the targeting moiety is a peptide-MHC complex, as described in Section 5.7.3, e.g., a peptide-MHC complex that is recognized by tumor lymphocytes.

Some IL27 agonist formats comprise more than one targeting moiety. When an IL27 agonist of the disclosure comprises two or more different targeting moieties (for example, IL27 agonists having the formats schematized in FIG. 3D or FIG. 3E), the different targeting moieties can suitably bind to the same cell (whether to different polypeptides or to different epitopes of the same polypeptide) or tissue type. Alternatively, the different targeting moieties can bind to different cells or tissues and in doing so bring them into proximity of one another.

Some exemplary targeting moiety target and formats are described below.

5.7.1. Target Molecules

The antibodies and antigen-binding fragments generally bind to specific antigenic determinants and are able to direct the IL27 agonist to a target site, for example to a specific type of disease cell that bears the antigenic determinant.

The target molecules recognized by the targeting moieties of the IL27 agonists of the disclosure are generally found, for example, on the cell surface of immune cells or on the cell surface on a tissue.

Non-limiting examples of target molecules found on the cell surface of immune cells include CD2, CD3, CD4, CD7, CD8, XCR1, Clec9a, and CD20.

Non-limiting examples of target molecules found on the cell surface of a tissue include MADCAM, a4b7 integrin, TSHR and EpCam.

In some embodiments, the targeting moiety binds to a cytokine, for example IL27 or a subunit thereof. In some embodiments, the targeting moiety is an IL27-binding domain of the IL27 receptor.

In some embodiments, the targeting moiety is a peptide-MHC complex, for example a peptide-MHC complex targeting autoreactive T cells.

Other target molecules recognized by the targeting moieties of the IL27 receptor agonists of the disclosure are can be found, for example, on the surfaces of activated T cells, on the surfaces of tumor cells, on the surfaces of virus-infected cells, on the surfaces of other diseased cells, free in blood serum, in the extracellular matrix (ECM), or immune cells present in the target site, e.g., tumor reactive lymphocytes. Where the immune cells are exogenously administered (e.g., chimeric antigen receptor (“CAR”) expressing T cells), the targeting moiety can recognize the chimeric antigen receptor (CAR) or another molecule found on the surface of the CAR T cells. In various embodiments, the CAR comprises CDRs or VH and VL sequences (e.g., in the format of an scFv) that specifically recognize a TAA or a pMHC complex.

Exemplary target molecules are Fibroblast Activation Protein (FAP), the A1 domain of Tenascin-C (TNC A1), the A2 domain of Tenascin-C (TNC A2), the Extra Domain B of Fibronectin (EDB), the Melanoma-associated Chondroitin Sulfate Proteoglycan (MCSP), MART-1/Melan-A, gp100, Dipeptidyl peptidase IV (DPPIV), adenosine deaminase-binding protein (ADAbp), cyclophilin b, colorectal associated antigen (CRC)-C017-1A/GA733, Carcinoembryonic Antigen (CEA) and its immunogenic epitopes CAP-1 and CAP-2, etv6, amli, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, c-erbB-2, Her2, EGFR, IGF-1R, CD2 (T-cell surface antigen), CD3 (heteromultimer associated with the TCR), CD22 (B-cell receptor), CD23 (low affinity IgE receptor), CD30 (cytokine receptor), CD33 (myeloid cell surface antigen), CD40 (tumor necrosis factor receptor), IL-6R-(IL6 receptor), CD20, MCSP, PDGFpR (p-platelet-derived growth factor receptor), ErbB2 epithelial cell adhesion molecule (EpCAM), EGFR variant III (EGFRvIII), CD19, disialoganglioside GD2, ductal-epithelial mucine, gp36, TAG-72, glioma-associated antigen, p-human chorionic gonadotropin, alphafetoprotein (AFP), lectin-reactive AFP, thyroglobulin, MN-CA IX, human telomerase reverse transcriptase, RU1, RU2 (AS), intestinal carboxyl esterase, mut hsp70-2, M-CSF, prostase, prostase specific antigen (PSA), PAP, LAGA-1a, p53, prostein, PSMA, surviving and telomerase, prostate-carcinoma tumor antigen-1 (PCTA-1), ELF2M, neutrophil elastase, ephrin B2, insulin growth factor (IGF1)-I, IGF-II, IGFI receptor, 5T4, ROR1, Nkp30, NKG2D, tumor stromal antigens, CA166-9, the extra domain A (EDA) and extra domain B (EDB) of fibronectin and the A1 domain of tenascin-C (TnC A1).

Non-limiting examples of viral antigens include an EBV antigen (e.g., Epstein-Barr virus LMP-1), a hepatitis C virus antigen (e.g., hepatitis C virus E2 glycoprotein), an HIV antigen (e.g., HIV gp160, and HIV gp120); a CMV antigen; a HPV-specific antigen, or an influenza virus antigen (e.g., influenza virus hemagglutinin).

Non-limiting examples of ECM antigens include syndecan, heparanase, integrins, osteopontin, link, cadherins, laminin, laminin type EGF, lectin, fibronectin, extra domain B (ED-B) of fibronectin, notch, tenascin, collagen and matrixin.

Other target molecules are cell surface molecules of tumor or viral lymphocytes, for example T-cell co-stimulatory proteins such as CD27, CD28, 4-1BB (CD137), OX40, CD30, CD40, ICOS, lymphocyte function-associated antigen-1 (LFA-1), CD2, CD7, LIGHT, NKG2C, and B7-H3.

In particular embodiments, the target molecules are checkpoint inhibitors, for example CTLA-4, PD1, PDL1, PDL2, B7-H3, B7-H4, BTLA, HVEM, TIM3, GAL9, LAG3, VISTA, KIR, 2B4, CD160, CGEN-15049, CHK1, CHK2. In particular embodiments, the target molecule is PD1. In other embodiments, the target molecule is LAG3.

In some embodiments, the targeting moieties target the exemplary target molecules set forth in Table 3 below, together with references to exemplary antibodies or antibody sequences upon which the targeting moiety can be based.

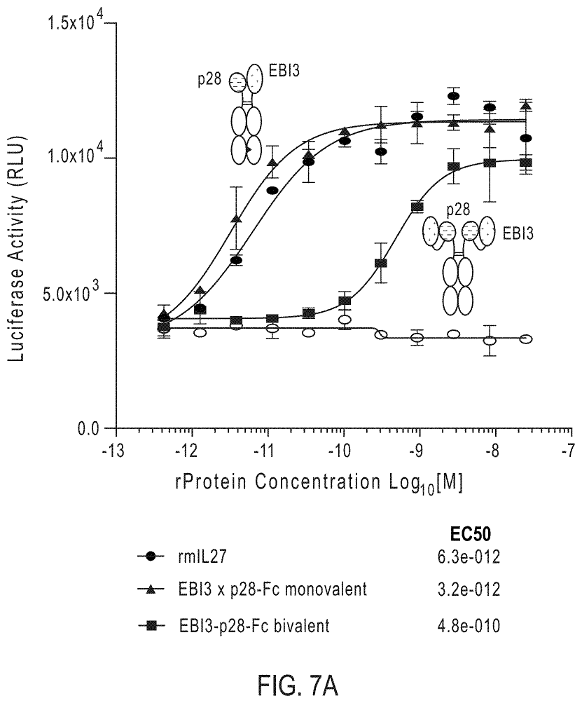

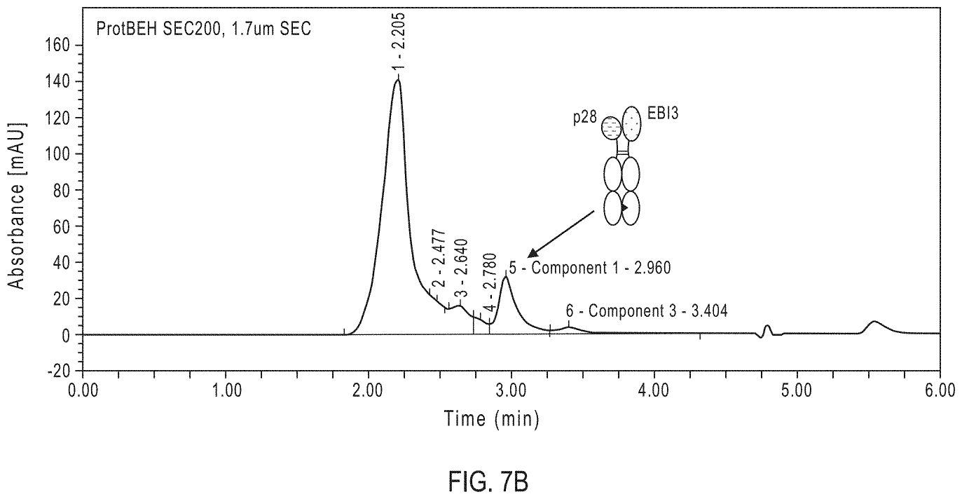

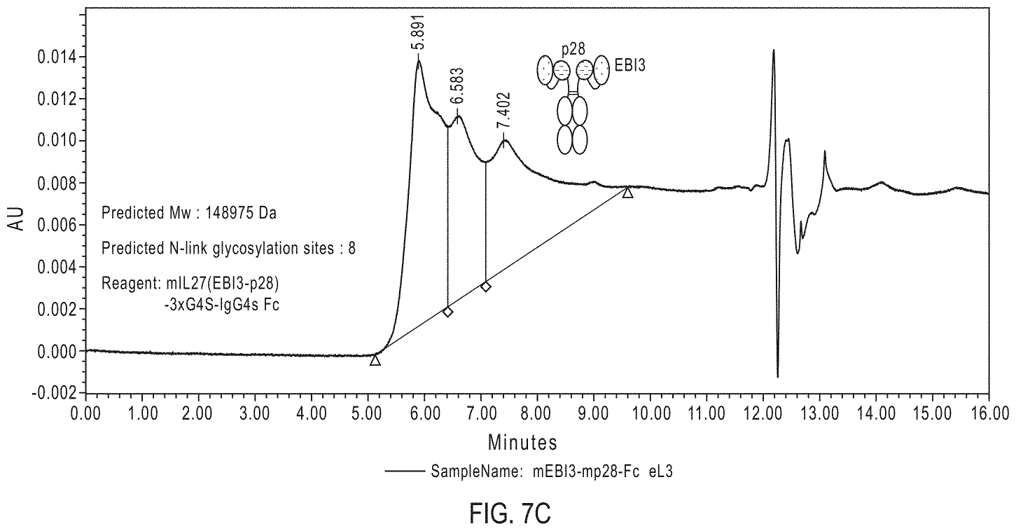

TABLE 3
Exemplary Target Molecules

| Target | Antibody Name and/or Binding Sequences |
|---|---|
| 1-92-LFA-3 | Amevive ™ (alefacept) |
| 5T4 | GEN1044 |
| Activin Receptor | Bimagrumab |
| Type II | VH: SEQ ID NOs: 107, 109 of U.S. Pat. No. 8,388,968 B2 |
| | VL: SEQ ID NOs: 93, 95 of U.S. Pat. No. 8,388,968 B2 |
| B7-H3 | Obrindatamab (MGD009) |
| B7-H3 (CD276) | Enoblituzumab (MGA271) |
| B7-H3 (CD276) | MGC018 |
| B7-H3 (CD276) | MGA012 |
| B7-H3 (CD276) | 8H9 |
| B7-H3 (CD276) | VH: the VH sequence of the heavy chain of SEQ ID NO: 21, |
| | 26 or 31 of US 2021/0171641 A1. |
| | VL: the VL sequence of the light chain of SEQ ID NO: 20, |
| | 22 or 30 of US 2021/0171641 A1. |
| B7-H3 (CD276) | VH: the VH sequence of the heavy chain of SEQ ID NO: 21, |
| | 29 or 37 of US 2019/0002563 A1. |
| | VL: the VL sequence of the light chain of SEQ ID NO: 17, |
| | 25 or 33 of US 2019/0002563 A1. |
| B7-H3 (CD276) | VH: the VH sequence of the heavy chain of SEQ ID NO: 146, |
| | 147 or 148 of U.S. Pat. No. 10,640,563. |
| | VL: the VL sequence of the light chain of SEQ ID NO: 143, |
| | 144 or 145 of U.S. Pat. No. 10,640,563. |
| BAFF/B Lymphocyte | Benlysta ™ (velimumab) |
| Stimulator | |
| BAFF/B Lymphocyte | VH: amino acids 1-123 of SEQ ID NO: 327 of U.S. Pat. No. |
| Stimulator | 7,138,501 |
| | VL: amino acids 139-249 of SEQ ID NO: 327 of U.S. Pat. No. |
| | 7,138,501. |
| BAFF/B Lymphocyte | VH: amino acids 1-126 of SEQ ID NO: 1321 of U.S. Pat. No. |
| Stimulator | 7,605,236; |
| | VL: amino acids 143-251 of SEQ ID NO: 1049 of U.S. Pat. No. |
| | 7,605,236. |
| BAFF/B Lymphocyte | Belimumab |
| Stimulator | |
| BCMA | VH: the VH sequence of the heavy chain of SEQ ID NO. 126 |
| | of US 2021/0206865 A1 |
| | VL: the VL sequence of the light chain of SEQ ID NO. 129 |
| | or SEQ ID NO. 132 of US 2021/0206865 A1 |
| CA125 | Igobumab |
| CA125 | OvaRex ™ (oregobumab) |
| Cadherin | The antibodies described in US Pub. No. US 2006/0039915. |
| N-cadherin | An antibody that binds to the amino acid sequence of |
| | SEQ ID NO: 10, 17 or 18 of US Pub. No. US 2010/0278821. |
| CD3 | Catumaxomab |
| CD3 | Ertumaxomab |
| CD3 | The CD3 portion of anti-PSMA/anti-CD3 antibodies |
| | described in WO2011121110A1 |
| CD3 | Anti-CD3 antibody sequences in U.S. Pat. No. 10,266,593B2 |
| CD3 | Anti-CD3 antibody sequences in U.S. Pat. No. 8,846,042B2 |
| CD3 | Anti-CD3 antibody sequences US 2016/0355600 |
| CD3 | Anti-CD3 antibody sequences in WO 2014/110601 |
| CD3 | Anti-CD3 antibody sequences in WO 2014/145806 |
| CD3 | Anti-CD3 antibody sequences in U.S. Pat. No. 10,066,015 |
| CD3 | Anti-CD3 antibody sequences in WO 2019/034580 |
| CD3 | Anti-CD3 antibody sequences in WO 2014/056783 |
| CD3 | Anti-CD3 antibody sequences in WO 2013/055809 A1 |
| CD3 | Anti-CD3 antibody sequences in U.S. Pat. No. 10,066,016 |
| CD3 | Anti-CD3 antibody sequences in US 2010/0150918 |
| CD3 | MT110 |
| CD3 | Acapatamab (AMG160) |
| CD3 | AMG199 |
| CD3 | AMG330 |
| CD3 | AMG427 |
| CD3 | AMG562 |
| CD3 | AMG596 |
| CD3 | AMG673 |
| CD3 | AMG701 |
| CD3 | Tarlatamab (AMG757) |
| CD3 | AMG910 |
| CD3 | BAY2010112 |
| CD3 | AMG420 |
| CD3 | AMG424 |
| CD3 | AMG509 |
| CD3 | AMV564 |
| CD3 | APVO436 |
| CD3 | Alnuctamab (CC-93269) |
| CD3 | ERY974 |
| CD3 | A-319 |
| CD3 | GEM333 |
| CD3 | GEM3PSCA |
| CD3 | Cevostamab |
| CD3 | Runimotamab |
| CD3 | GEN1044 |
| CD3 | Epcoritamab |
| CD3 | HPN424 |
| CD3 | ISB1302 |
| CD3 | ISB1342 |
| CD3 | IGM-2323 |
| CD3 | IMC-F106C |
| CD3 | IMC-C103C |
| CD3 | IMCnyeso |
| CD3 | JNJ-63709178 |
| CD3 | JNJ-63898081 |
| CD3 | Teclistamab |
| CD3 | Talquetamab |
| CD3 | JNJ-67571244 |
| CD3 | MGD007 |
| CD3 | Orlotamab |
| CD3 | Duvortuxizumab |
| CD3 | Flotetuzumab |
| CD3 | MCLA-117 |
| CD3 | PF-06671008 |
| CD3 | Elranatamab |
| CD3 | Odronextamab |
| CD3 | REGN5458 |
| CD3 | REGN5459 |
| CD3 | REGN4018 |
| CD3 | Glofitamab |
| CD3 | RO6958688 |
| CD3 | SAR440234 |
| CD3 | TNB-383B |
| CD3 | M802 |
| CD3 | Xmab13676 |
| CD3 | Xmab18087 |
| CD3 | Vibecotamab |
| CD3 | Nivatrotamab |
| CD3 | Anti-CD3 antibody sequences in US20190211100 |
| CD3 | Anti-CD3 antibody sequences in EP1629011B |
| CD3 | VH of SEQ ID NOS. 90 and 98 disclosed in US 2021/0206865 A1 |
| | CDR-H1 of SEQ ID NO. 92 and 100 disclosed in US 2021/0206865 A1 |
| | CDR-H2 of SEQ ID NO. 94 and 102 disclosed in US 2021/0206865 A1 |
| | CDR-H3 of SEQ ID NO. 96 and 104 disclosed in US 2021/0206865 A1 |
| | HC of SEQ ID NO. 127 or SEQ ID NO. 128 disclosed in US 2021/0206865 A1 |
| | LC of SEQ ID NO. 129 or SEQ ID NO. 132 disclosed in US 2021/0206865 A1 |
| CD3 | Anti-CD3 Heavy chain of SEQ ID NO: 2 disclosed in US 2021/0206870 A1 |
| | Anti-CD3 VH SEQ ID NO. 5 disclosed in US 2021/0206870 A1 |
| | Anti-CD3 VL of SEQ ID NO. 6 disclosed in US 2021/0206870 A1 |
| | Anti-CD3 CDR-H1 of SEQ ID NO. 10 disclosed in US 2021/0206870 A1 |
| | Anti-CD3 CDR-H2 of SEQ ID NO. 11 disclosed in US 2021/0206870 A1 |
| | Anti-CD3 CDR-H3 of SEQ ID NO. 12 disclosed in US 2021/0206870 A1 |
| CD3 | Anti-CD3 VH of SEQ ID NO: 92, 102, 112, 122, 132, 142, 156, |
| | 166, 176, 186, 196 or 206 disclosed in US 2022/0119525 A1 |
| | Anti-CD3 CDR-H1 of SEQ ID NO. 93, 103, 113, 123, 133, 143, |
| | 157, 167, 177, 187, 197 or 207 disclosed in US 2022/0119525 A1 |
| | Anti-CD3 CDR-H2 of SEQ ID NO. 94, 104, 114, 124, 134, 144, |
| | 158, 168, 178, 188, 198 or 208 disclosed in US 2022/0119525 A1 |
| | Anti-CD3 CDR-H3 of SEQ ID NO. 95, 105, 115, 125, 135, 145, |
| | 159, 169, 179, 189, 199 or 209 disclosed in US 2022/0119525 A1 |
| | Anti-CD3 VL of SEQ ID NO. 96, 106, 116, 126, 136, 146, 152, 162, |
| | 172, 182, 192 or 202 disclosed in US 2022/0119525 A1 |
| | Anti-CD3 CDR-L1 of SEQ ID NO. 97, 107, 117, 127, 137, 147, |
| | 153, 163, 173, 183, 193 or 203 disclosed in US 2022/0119525 A1 |
| | Anti-CD3 CDR-L2 of SEQ ID NO. 98, 108, 118, 128, 138, 148, |
| | 154, 164, 174, 184, 194 or 204 disclosed in US 2022/0119525 A1 |
| | Anti-CD3 CDR-L3 of SEQ ID NO. 99, 109, 119, 129, 139, 149, |
| | 155, 165, 175, 185, 195 or 205 disclosed in US 2022/0119525 A1 |
| CD11a | Raptiva ™ (efalizumab) |
| | Sequence in Werther et al., 1996, The Journal of Immunology |
| | 157(11): 4986-4995. |
| CD19 | Blincyto ™ (blinatumomab) |
| CD19 | SGN-CD19A |
| CD20 | Bexxar ™ (tositumomab) |
| | VH: the VH sequence of the heavy chain of SEQ ID NO: 124 of US |
| | Patent Pub. US 2017/0002060 A1 |
| | VL: the VL sequence of the light chain of SEQ ID NO: 125 of US |
| | Patent Pub. US 2017/0002060 A1 |
| CD20 | Zevalin ™ (ibritumomab tiuxetan) |
| | VH: SEQ ID NO: 9 of U.S. Pat. No. 5,736,137 |
| | VL: SEQ ID NO: 6 of U.S. Pat. No. 5,736,137 |
| CD20 | Rituxan ™ (rituximab) |
| | VH: SEQ ID NO: 9 of U.S. Pat. No. 5,736,137 |
| | VL: SEQ ID NO: 6 of U.S. Pat. No. 5,736,137 |
| CD20 | Ocrevus ™ (ocrelizumab) |
| CD20 | Okaratuzumab |
| CD20 | Arzerra ™ (ofatumumab) |
| | VH: SEQ ID NO: 2 of U.S. Pat. No. 8,529,902 |
| | VL: SEQ ID NO: 4 of U.S. Pat. No. 8,529,902 |
| CD20 | Gazyva ™ (obinutuzumab) |
| CD20 | VH: SEQ ID NO: 4 of US 2021/0206870 A1 |
| | VL of SEQ ID NO: 6 of US 2021/0206870 A1 |
| CD20 | Epcoritamab |
| CD22 | Belimumab |
| CD22 | Epratuzumab |
| CD22 | Besponsa ™ (inotuzumab ozogamicin) |
| CD22 | Lumoxiti ™ (moxetumumab pasudox) |
| CD22 | pinatuzumab vedotin |
| CD25 | Zenapax ™ (daclizumab) |
| | VH: SEQ ID NO: 9 of U.S. Pat. No. 7,060,269 |
| | VL: SEQ ID NO: 10 of U.S. Pat. No. 7,060,269 |
| CD30 | Adcetris ™ (brentuximab vedotin) |
| | VH: SEQ ID NO: 2 of U.S. Pat. No. 7,090,843 |
| | VL: SEQ ID NO: 10 of U.S. Pat. No. 7,090,843 |
| CD33 | Myelotarg ™ (gemtuzumab) |
| | Sequence in Man Sung, et al., 1993, Molecular |
| | immunology 30: 1361-1367 |
| CD33 | Lintuzumab |
| CD38 | Darzalex ™ (daratumumab) |
| CD40 | Lukatumumab |
| CD40 | Dacetuzumab |
| CD40L | Hu5c8 (ruplizumab) |
| CD44v6 | vibatuzumab mertansine |
| CD52 | Campath ™ (alemtuzumab) |
| | VH: SEQ ID NO: 1 of US Patent Pub. US 2017/0002060 A1 |
| | VL: SEQ ID NO: 2 of US Patent Pub. US 2017/0002060 A1 |
| CD70 | Blenrep ™ (borsetuzumab mafodotin) |
| CD123 | Flotetuzumab |
| CD221 | Tepezza ™ (teprotumumab) |
| CEA | Hybri-Ceaker ® (altumomab pentetate) |
| CEA | Scintimun ™ (besilesomab) |
| CEA | CEA-CIDE ™ (labetuzumab)) |
| CEA | CEA-Scan ™ (arcitumomab) |
| CEA | hMN-15 |
| | CDR-H1, CDR-H2 and CDR-H3 sequences of SEQ ID NOs: 4-6 of U.S. |
| | Pat. No. 8,771,690 B2 |
| | CDR-L1, CDR-L2 and CDR-L3 sequences of SEQ ID NOs: 1-3 of U.S. |
| | Pat. No. 8,771,690 B2 |
| CEA | CEA binding portion of RO6958688/RG7802 from clinical trial |
| | NCT02324257 |
| CEA | Cibisatamab |
| CEA | CEA binding portion of MEDI-565/MT110/AMG211 from clinical trials |
| | NCT01284231 and NCT02291614 |
| | VH: SEQ ID NO: 49 or 51 of PCT Publication No. WO 2013/012414 A1 |
| | VL: SEQ ID NO: 48 of PCT Publication No. WO 2013/012414 A1. |
| CEA | Rabetuzumab |
| CEA | Atezolizumab |
| CEA | Cibisatamab |
| CEA | MEDI-565 (AMG211, MT111) |
| CEA | RO6958688! |
| CEA | VH: SEQ ID No. 9 described in WO2022/048883A1 |
| | VL: SEQ ID No. 10 described in WO2022/048883A1 |
| CLDN18.2 | AMG910 |
| Collagen | TRC093 (MT293) |
| alpha-4 chain | |
| Collagen | The collagen binding antibody fragment described in Liang, H. et al. A |
| | collagen-binding EGFR antibody fragment targeting tumors with a |
| | collagen-rich extracellular matrix. Sci. Rep. 5, 18205; doi: |
| | 10.1038/srep18205 (2016). |
| Collagen type I | Cetuximab (Erbitux) |
| Collagen type X | The amino acid sequences of SEQ ID NO: 1 or 2 of PCT Pub No. WO |
| | 2019/020797. |
| Collagen type X | The amino acid sequences of SEQ ID NO: 1 of PCT Pub No. WO |
| | 2014/180992. |
| Collagen type X | Antibody X34 as described in I. Girkontaite et al., “Immunolocalization |
| | of type X collagen in normal fetal and adult osteoarthritic cartilage with |
| | monoclonal antibodies,” Matrix Biol 15, 231-238 (1996). |
| Collagen type X | Antibodies X53 or 1H8 or ARC0659 or JF0961 collagen X polyclonal |
| | antibody sold under catalog number PA5-115039 or PA5-116871 or |
| | PA5-97603 or PA5-49198 from ThermoFisher Scientific. |
| Collagen type X | Antibody sold under catalog number RDI-COLL10abr from RDI. |
| Complement C5 | Soliris ™ (eculizumab) |
| | VH: amino acids 1-122 of SEQ ID NO: 10 of U.S. Pat. No. 6,355,245 |
| | VL: amino acids 3-110 of SEQ ID NO: 9 of U.S. Pat. No. 6,355,245 |
| CTLA-4 | Yervoy ™ (ipilimumab) |
| | VH: SEQ ID NO: 17 of WO 2001/014424 A2 |
| | VL: SEQ ID NO: 7 of WO 2001/014424 A2 |
| CTLA-4 | (tremelimumab) |
| CTLA-4 | Orencia ™ (abatacept) |
| DLL3 | AMG757 |
| EGFR | Erbitux ™ (cetuximab) |
| | VH: SEQ ID NO: 11 of U.S. Pat. No. 6,217,866 |
| | VL: SEQ ID NO: 13 of U.S. Pat. No. 6,217,866 |
| EGFR | Vectibix ™ (panitumumab) |
| | VH: SEQ ID NO: 37 of U.S. Pat. No. 6,235,883 |
| | VL: SEQ ID NO: 38 of U.S. Pat. No. 6,235,883 |
| EGFR | Zalutumumab |
| | VH: SEQ ID NO: 64 of WO 2018/140831 A2 |
| | VL: SEQ ID NO: 69 of WO 2018/140831 A2 |
| EGFR | mapatumumab |
| EGFR | Matuzumab |
| EGFR | Nimotuzumab |
| | VH: SEQ ID NO: 51 of WO 2018/140831 A2 |
| | VL: SEQ ID NO: 56 of WO 2018/140831 A2 |
| EGFR | ICR62 |
| EGFR | mAb 528 |
| EGFR | CH806 |
| EGFRv3 | AMG596 |
| EGFRv3 | AMG404 |
| EGFR/CD64 | MDX-447 |
| EpCAM | Panorex ™ (edrecolomab) |
| | VH: SEQ ID NO: 129 of WO 2018/140831 A2 |
| | VL: SEQ ID NO: 134 of WO 2018/140831 A2 |
| EpCAM | Adecatumumab |
| | VH: SEQ ID NO: 142 of WO 2018/140831 A2 |
| | VL: SEQ ID NO: 147 of WO 2018/140831 A2 |
| EpCAM | tucotuzumab celmoleukin |
| EpCAM | citatuzumab bogatox |
| EpCAM | EP1629013 B1 |
| | VH: SEQ ID NOs: 80, 84, 88, 92 or 96 |
| | VL: SEQ ID NOs: 82, 86, 90, 94 or 98 |
| EpCAM | G8.8 |
| | HC: SEQ ID NO: 4 of US Patent Pub. No. US 2020/0317806 A1 |
| | HL: SEQ ID NO: 3 of US Patent Pub. No. US 2020/0317806 A1 |
| EpCAM | VH: SEQ ID NOs: 17-22 of WO 2021/211510 A2. |
| | VL: SEQ ID NO: 15-16 of WO 2021/211510 A2. |
| EpCAM | Removab ™(catumaxomab) |
| EpCAM | Vicineum ™ (oportuzumab monatox) |
| EpCAM | M701 |
| F protein of RSV | Synagic ™ (palivizumab) |
| GD2 | 3F8 |
| Glycoprotein | ReoPro ™ (abiciximab) |
| receptor IIb/IIIa | |
| gpA33 | MGD007 |
| GPC3 | ERY974 |
| GUCY2C | PF-07062119 |
| Heparanase | An antibody selected from HP130, HP 239, HP 108.264, HP 115.140, |
| | HP 152.197, HP 110.662, HP 144.141, HP 108.371, HP 135.108, HP |
| | 151.316, HP 117.372, HP 37/33, HP3/17, HP 201 or HP 102 or an |
| | amino acid sequence of SEQ ID NO: 1-11 described in US Patent |
| | Pub. US 2004/0170631. |
| Her2 | Herceptin ™ (trastuzumab) |
| Her2 | Aldesleukin (proleukine) |
| Her2 | Sargramustim (leukine) |
| Her2 | M802 |
| Her2 | Runimotamab (BTRC4017A, R07227780) |
| Her2 | ISB1302 |
| Her2-neu | Perjeta ™ (pertuzumab) |
| | VH: SEQ ID NO: 16 of WO 2013/096812 A1. |
| | VL: SEQ ID NO: 15 of WO 2013/096812 A1. |
| Her2-neu | Rexomun ™ (ertumaxomab) |
| IgE | Xolair ™ (omalizumab) |
| IGFIR | (figitumumab) |
| IL1β | Ilaris ™ (canakinumab) |
| | VH: SEQ ID NO: 1 of U.S. Pat. No. 7,446,175. |
| | VL: SEQ ID NO: 2 of U.S. Pat. No. 7,446,175 |
| IL1Ra | Antril ™, Kineret ™ (ankinra) |
| IL2R | Simulect ™ (basiliximab) |
| | VH: SEQ ID NO: 3 of U.S. Pat. No. 6,383,487 |
| | VL: SEQ ID NO: 6 of U.S. Pat. No. 6,383,487 |
| IL6 | Clazakizumab |
| IL6 receptor | Actemra ™ (tocilizumab) |
| | VH: SEQ ID NO: 31 of U.S. Pat. No. 7,479,543 |
| | VL: SEQ ID NO: 29 of U.S. Pat. No. 7,479,543 |
| Integrinα4 | Tysabri ™ (natalizumab) |
| | VH: SEQ ID NOs: 11-13 of U.S. Pat. No. 5,840,299 |
| | VL: SEQ ID NOs: 7-8 of U.S. Pat. No. 5,840,299 |
| Integrinα4 β7 | Entyvio ™ (vedolizumab) |
| | HC: SEQ ID NO: 2 of US Patent Pub. US 2012/0282249. |
| | LC: SEQ ID NO: 4 of US Patent Pub. US 2012/0282249. |
| Integrinα5 β1 | VH: SEQ ID NO: 2 of European Patent No. 1 755 659. |
| | VL: SEQ ID NO: 4 of European Patent No. 1 755 659. |
| Integrin β1 | VH: SEQ ID NO: 2, 6, 8, 10, 12, 14, 29-43 or 91-100 of US Patent |
| | Pub. US 2022/0089744. |
| | VL:, SEQ ID NO: 4, 16, 18, 20, 22, 44-57 or 107-116 of US Patent |
| | Pub. US 2022/0089744. |
| LAG3 | Relatlimab (BMS-98016) |
| LAG3 | Sym022 |
| LAG3 | HLX26 |
| LAG3 | TSR-033 |
| LAG3 | ABL501 |
| LAG3 | INCAGN02385 |
| LAG3 | Fianlimab (REGN3767) |
| LAG3 | RO7247669 |
| LAG3 | EMB-02 |
| LAG3 | FS118 |
| LAG3 | GSK2831781 |
| LAG3 | IBI323 |
| LAG3 | IBI110 |
| LAG3 | LAG525 |
| LAG3 | XmAb ®22841 |
| LAG3 | LBL-007 |
| LAG3 | VH: SEQ ID NO: 1, 8, 10 or 12 of U.S. Pat. No. 9,902,772. |
| | VL: SEQ ID NO: 2, 3, 4, 5, 6, 7, 9, 11, 13 or 14 of U.S. Pat. No. |
| | 9,902,772. |
| LAG3 | VH: SEQ ID NO: 182 of US Patent Pub. US 2021/0095026. |
| | VL: SEQ ID NO: 88 of US Patent Pub. US 2021/0095026. |
| LAG3 | Antibodies having VH/VL amino acid sequences of SEQ ID NOs |
| | 23/24, 3/4 and 11/12 of US Pub. US2022/0056126A1. |
| Laminin | Lam-89 from Sigma Aldrich |
| Mesothelin | Amatuximab |
| Mesothelin | HPN536 |
| MUC1 | civatuzumab tetraxetane |
| MUC1 | Pankomab ™ (gatipotuzumab) |
| MUC1 | Femtumumab |
| MUC1 | Cantuzumab ravtansine |
| MUC16 (CA125) | Anti-MUC16 antibodies having VH and VL sequences having the |
| | amino acid sequences of any one of the following SEQ ID NO: pairs |
| | from US 2018/0118848A1: 18/26; 82/858; 98/170 |
| MUC17 | AMG199 |
| Nectin-4 | Enfortumab (ASP7465, ASG-22CE, ASG-22ME) |
| | VH: SEQ ID NO: 3 of PCT Pub. WO 2021/151984. |
| | VL: SEQ ID NO: 4 of PCT Pub. WO 2021/151984. |
| Nectin-4 | SBT290 |
| Nectin-4 | VH: SEQ ID NO: 1 of U.S. Pat. No. 11,274,160. |
| | VL: SEQ ID NO: 2 of U.S. Pat. No. 11,274,160. |
| NGF | (tanezumab) |
| Osteopontin | HC: SEQ ID NO: 22 of PCT Pub. WO 2021/030209. |
| | LC: SEQ ID NO: 24 of PCT Pub. WO 2021/030209. |
| PD1 | MDX-1106/BMS-936558 (nivolumab), a human IgG4 mAb with the |
| | structure described in WHO Drug Information, Vol. 27, No. 1, pages |
| | 68-69 (2013) and whose heavy and light chain sequences are |
| | disclosed in FIG. 7 of US Pub. No. US20190270812A1 |
| PD1 | MK-3475 (pembrolizumab), a humanized IgG4 mAb with the structure |
| | described in WHO Drug Information, Vol. 27, No. 2, pages 161-162 |
| | (2013) and whose heavy and light chain sequences are disclosed in |
| | FIG. 6 of US Pub. No. US20190270812A1 |
| PD1 | REGN2810 (disclosed as H4H7798N in US Pub No. 20150203579) |
| PD1 | Anti-PD1 antibodies having CDR H1-H3 and CDR L1-L3 sequences |
| | corresponding to the following SEQ ID NOs. of U.S. Pat. No. |
| | 11,034,765 B2: |
| | a) SEQ ID NOs: 18, 19, 20, 21, 22, and 23, respectively; |
| | b) SEQ ID NOs: 24, 25, 26, 27, 28, and 29, respectively; |
| | c) SEQ ID NOs: 30, 31, 32, 33, 34, and 35, respectively; |
| | d) SEQ ID NOs: 36, 37, 38, 39, 40, and 41, respectively; |
| | e) SEQ ID NOs: 42, 43, 44, 45, 46, and 47, respectively; |
| | f) SEQ ID NOs: 48, 49, 50, 51, 52, and 53, respectively; |
| | g) SEQ ID NOs: 54, 55, 56, 57, 58, and 59, respectively; and |
| | h) SEQ ID NOs: 60, 61, 62, 63, 64, and 65, respectively. |
| PD1 | Anti-PD1 antibodies disclosed in Tables 1-3 of PCT Pub. |
| | WO2015112800A1, including but not limited to anti-PD1 antibodies |
| | having VH/VL pairs having SEQ ID NOs: 2/10, 18/26, 34/42, 50/58, |
| | 66/74, 82/90, 98/106, 1 14/122, 130/138, 146/154, 162/170, 178/186, |
| | 194/202, 210/202, 218/202, 226/202, 234/202, 242/202, 250/202, |
| | 258/202, 266/202, 274/202, 282/202, 290/202, 298/186, 306/186 and |
| | 314/186 of PCT Pub. WO2015112800A1. |
| PD1 | Anti-PD1 antibodies disclosed in U.S. Pat. No. 10,294,299 B2 as |
| | having the following SEQ ID NO. pair for heavy and light chain |
| | variable domains: |
| | SEQ ID NOs. 164/178 |
| | SEQ ID NOs. 165/179 |
| | SEQ ID NOs. 166/180 |
| | SEQ ID NOs. 167/181 |
| | SEQ ID NOs. 168/182 |
| | SEQ ID NOs. 169/183 |
| | SEQ ID NOs. 170/184 |
| | SEQ ID NOs. 171/185 |
| | SEQ ID NOs. 172/186 |
| | SEQ ID NOs. 173/187 |
| | SEQ ID NOs. 174/188 |
| | SEQ ID NOs. 175/189 |
| | SEQ ID NOs. 176/190 |
| | SEQ ID NOs. 177/190 |
| PD1 | MEDI-0680 (AMP-514) |
| PD1 | PDR001 |
| PD1 | BGB-108 |
| PD1 | h409A11, described in WO2008/156712 |
| PD1 | h409A16, described in WO2008/156712 |
| PD1 | h409A17, described in WO2008/156712 |
| PD1 | Anti-PD1 antibodies described in U.S. Pat. No. 7,488,802 |
| PD1 | Anti-PD1 antibodies described in U.S. Pat. No. 7,521,051 |
| PD1 | Anti-PD1 antibodies described in U.S. Pat. No. 8,008,449 |
| PD1 | Anti-PD1 antibodies described in U.S. Pat. No. 8,354,509 |
| PD1 | Anti-PD1 antibodies described in U.S. Pat. No. 8,168,757 |
| PD1 | Anti-PD1 antibodies described in PCT Pub. No. W02004/004771 |
| PD1 | Anti-PD1 antibodies described in PCT Pub. No. W02004/056875 |
| PD1 | Anti-PD1 antibodies described in PCT Pub. No. W02004/072286 |
| PD1 | Anti-PD1 antibodies described in US Pub. No. US2011/0271358 |
| PDL1 | Durvalumab (MEDI4736) |
| PDL1 | Atezolizumab (Tecentriq, MPDL3280A)) |
| PDL1 | MDX 1105 (BMS-936559) |
| PDL1 | Avelumab |
| PDL1 | ZKAB001 (Socazolimab) |
| PDL1 | TQB2450 |
| PDL1 | MEDI4736 |
| PDL1 | HLX20 |
| PDL1 | KN035 |
| PDL1 | LY3434172 |
| PDL1 | LY3300054 |
| PDL1 | LDP |
| PDL1 | EMB-09 |
| PDL1 | ABL501 |
| PDL1 | INBRX-105 |
| PDL1 | SHR-1210 |
| PDL1 | STI-3031 (IMC-001) |
| PDL1 | MPDL3280A (RG7446) |
| PDL1 | KN035 |
| PDL1 | BGB-A333 |
| PDL1 | HLX301 |
| PDL1 | Y101D |
| PDL1 | ES101 |
| PDL1 | IBI322 |
| PDL1 | Envafolimab |
| PDL1 | VH: SEQ ID NO: 46, 48, 50 or 52 of U.S. Pat. No. 11,168,144. |
| | VL: SEQ ID NO: 58, 137 or 12 of U.S. Pat. No. 11,168,144. |
| PDL1 | VH: SEQ ID NO: 23, 124, 126, 127, 128, 130, 140 or 145 of |
| | U.S. Pat. No. 11,208,486. |
| | VL: SEQ ID NO: 24 or 125 of U.S. Pat. No. 11,208,486. |
| Phosphatidylserine | (bavituximab) |
| PSCA | GEM3PSCA |
| PSMA | huJ591 |
| PSMA | Anti-PSMA antibodies having VH and VL sequences having the amino |
| | acid sequences of any one of the following SEQ ID NO: pairs from |
| | WO 2017/023761A1: 2/1642; 10/1642; 18/1642; 26/1642; 34/1642; |
| | 42/1642; 50/1642; 58/1642; 66/1642; 74/1642; 82/1642; 90/1642; |
| | 98/1642; 106/1642; 1 14/1642; 122/130; and 138/146. |
| PSMA | An antibody such as: PSMA 3.7, PSMA 3.8, PSMA 3.9, PSMA 3.11, |
| | PSMA 5.4, PSMA 7.1, PSMA 7.3, PSMA 10.3, PSMA 1.8.3, PSMA |
| | A3.1.3, PSMA A3.3.1, Abgenix 4.248.2, Abgenix 4.360.3, Abgenix |
| | 4.7.1, Abgenix 4.4.1, Abgenix 4.177.3, Abgenix 4.16.1, Abgenix |
| | 4.22.3, Abgenix 4.28.3, Abgenix 4.40.2, Abgenix 4.48.3, Abgenix |
| | 4.49.1, Abgenix 4.209.3, Abgemx 4.219.3, Abgenix 4.288.1, Abgenix |
| | 4.333.1, Abgemx 4.54.1, Abgenix 4.153.1, Abgenix 4.232.3, Abgenix |
| | 4.292.3, Abgenix 4.304.1, Abgenix 4.78.1 and Abgenix 4.152.1 |
| | described in WO2003034903A2 |
| | A hybridoma cell line such as: PSMA 3.7 (PTA-3257), PSMA 3.8, |
| | PSMA 3.9 (PTA- 3258), PSMA 3.11 (PTA-3269), PSMA 5.4 (PTA- |
| | 3268), PSMA 7.1 (PTA-3292), PSMA 7.3 (PTA-3293), PSMA 10.3 |
| | (PTA-3247), PSMA 1.8.3 (PTA-3906), PSMA A3.1.3 (PTA- 3904), |
| | PSMA A3.3.1 (PTA-3905), Abgenix 4.248.2 (PTA-4427), Abgenix |
| | 4.360.3 (PTA- 4428), Abgenix 4.7.1 (PTA-4429), Abgenix 4.4.1 (PTA- |
| | 4556), Abgenix 4.177.3 (PTA-4557), Abgenix 4.16.1 (PTA-4357), |
| | Abgenix 4.22.3 (PTA-4358), Abgenix 4.28.3 (PTA-4359), Abgenix |
| | 4.40.2 (PTA-4360), Abgenix 4.48.3 (PTA-4361), Abgenix 4.49.1 (PTA- |
| | 4362), Abgenix 4.209.3 (PTA-4365), Abgenix 4.219.3 (PTA-4366), |
| | Abgenix 4.288.1 (PTA-4367), Abgenix 4.333.1 (PTA-4368), Abgenix |
| | 4.54.1 (PTA-4363), Abgenix 4.153.1 (PTA-4388), Abgenix 4.232.3 |
| | (PTA-4389), Abgenix 4.292.3 (PTA-4390), Abgenix 4.304.1 (PTA- |
| | 4391), Abgenix 4.78.1 (PTA-4652), and Abgemx 4.152.1(PTA-4653) |
| | described in WO 2003/034903A2. |
| | VH of SEQ ID NOs: 2-7 described in WO 2003/034903A2 |
| | VL of SEQ ID NOs: 8-13 described in WO 2003/034903A2 |
| PMSA | VH: SEQ ID NOs: 225, 239, 253, 267, 281, 295, 309, 323, 337, 351, |
| | 365, 379, 393, 407, 421, 435, 449, 463, 477, 491, 505, 519, 533, 547, |
| | 561, 575, 589, 603 or 617 described in WO 2011/121110A1. |
| | VL SEQ ID NOs: 230, 244, 258, 272, 286, 300, 314, 328, 342, 356, |
| | 370, 384, 398, 412, 426, 440, 454, 468, 482, 496, 510, 524, 538, 552, |
| | 566, 580, 594, 608 or 622 described in WO 2011/121110A1. |
| | VH and VL SEQ ID Nos: 235, 249, 263, 277, 291, 305, 319, 333, 347, |
| | 361, 375, 389, 403, 417, 431, 445, 459, 473, 487, 501, 515, 529, 543, |
| | 557, 571, 585, 599, 613 or 627 described in WO 2011/121110A1. |
| PMSA | An anti-PMSA antibody having a VL amino acid sequence of any one |
| | of SEQ ID NOs: 229-312 of US 2022/0119525 A1 and a VH of SEQ |
| | ID NO: 217 of US 2022/0119525 A1. |
| PMSA | ES414 |
| PMSA | BAY2010112 (pasotuxizumab) |
| PMSA | CCW702 |
| PMSA | JNJ-63898081 |
| PMSA | CC-1 |
| PMSA | Acapatamab |
| PSMA | HPN424 |
| RAAG12 | RAV12 |
| RANKL | Prolia ™ (denosumab) |
| | VH: SEQ ID NO: 51 of US Patent Pub. 2017/0002060 |
| | VL: SEQ ID NO: 52 of US Patent Pub. 2017/0002060 |
| SLAMF7 | Empliciti ™ (elotuzumab) |
| SSTR2 | XmAb ®18087 |
| STEAP1 | VHCDR1 SEQ ID NOs: 14, 33, 182, 184 or 185 described in |
| | US20210179731A1. |
| | VHCDR2 SEQ ID NOs: 15, 21, 34, 182, 184 or 185 described in |
| | US20210179731A1. |
| | VHCDR3 SEQ ID NOs: 16 and 35 described in US20210179731A1. |
| | VH SEQ ID NOs: 182 or 184 described in US20210179731A1. |
| | VLCDR1 SEQ ID NOs: 11 or 30 described in US20210179731A1. |
| | VLCDR2 SEQ ID NOs: 12 or 31 described in US20210179731A1. |
| | VLCDR3 SEQ ID NOs: 13 or 32 described in US20210179731A1. |
| | VL SEQ ID NOs: 183 or 186 described in US20210179731A1. |
| STEAP1 | AMG509 |
| STEAP2 | Anti-STEAP 2 antibodies having CDR-H1, CDR-H2, CDR-H3, CDR- |
| | L1, CDR-L2 and CDR-L3 sequences selected from SEQ ID NOS: (1) |
| | 4-6-8-12-14-16; (2) 20-22-24-28-30-32; (3) 36-38-40-44-46-48; (4) 52- |
| | 54-56-60-62-64; (5) 68-70-72-60-62-64; (6) 76-78-80-60-62-64; (7) |
| | 84-86-88-60-62-64; (8) 92-94-96-60-62-64; (9) 100-102-104-60-62-64; |
| | (10) 108-110-112-116-118-120; (11) 124-126-128-132-134-136; (12) |
| | 140-142-144-148-150-152; (13) 156-158-160-164-166-168; (14) 172- |
| | 174-176-180-182-184; (15) 188-190-192-196-198-200; (16) 204-206- |
| | 208-212-214-216; (17) 220-222-224-228-230-232; (18) 236-238-240- |
| | 244-246-248; (19) 252-254-256-260-262-264; (20) 268-270-272-276- |
| | 278-280; (21) 284-286-288-292-294-296; (22) 300-302-304-308-310- |
| | 312; (23) 316-318-320-324-326-328; (24) 332-334-336-340-342-344; |
| | (25) 348-350-352-356-358-360; (26) 364-366-368-372-374-376; and |
| | (27) 380-382-384-388-390-392 of U.S. Pat. No. 10,772,972 B2. |
| | Anti-STEAP 2 antibodies having (a) a VH comprising the amino acid |
| | of any one of SEQ ID NOs: 2, 18, 34, 50, 66, 74, 82, 90, 98, 106, 122, |
| | 138, 154, 170, 186, 202, 218, 234, 250, 266, 282, 298, 314, 330, 346, |
| | 362, and 378 of U.S. Pat. No. 10,772,972 B2; and (b) a VL |
| | comprising the amino acid sequence of any one of SEQ ID NOs: 10; |
| | 26; 42; 58; 114; 130; 146; 162; 178; 194; 210; 226, 242; 258; 274; |
| | 290; 306; 322; 338; 354; 370; and 386 of U.S. Pat. No. 10,772,972 B2. |
| | Anti-STEAP 2 antibodies having a VH/VL pair comprising the amino |
| | acid sequences of any of the following pairs of SEQ ID NOs of U.S. |
| | Pat. No. 10,772,972 B2: 2/10; 18/26; 34/42; 50/58; 66/58; 74/58; |
| | 82/58; 90/58; 98/58; 106/114; 122/130; 138/146; 154/162; 170/178; |
| | 186/194; 202/210; 218/226; 234/242; 250/258; 266/274; 282/290; |
| | 298/306; 314/322; 330/338; 346/354; 362/370; and 378/386. |
| Syndecan-1 | The B-B4 antibody described in Wijdenes et al., 1996, Br. J. |
| (CD 138) | Haematol., 94: 318-323 |
| Syndecan-4 | The amino acid sequence of amino acids 93 and 121 of SEQ ID NO: |
| | 1 or the amino acid sequence of amino acids 92 and 122 of SEQ ID |
| | NO: 2 described in European Patent Pub. EP 2 603 236. |
| TCRαβ | BMA031 sequences disclosed in US 2012/0034221 |
| TCRγδ | 6TCS1 antibody disclosed in U.S. Pat. No. 5,980,892 |
| TGFβ | GC1008 |
| TNFR | Enbrel ™ (etanercept) |
| TNFα | Remicade ™ (infliximab) |
| | VH: SEQ ID NO: 2 of Int. Patent Publication WO201/3087911 A1 |
| | VH: SEQ ID NO: 3 of Int. Patent Publication WO2013/A1087911 |
| TNFα | Humira ™ (adalimumab) |
| | VH: SEQ ID NO: 4 of U.S. Pat. No. 6,258,562 |
| | VL: SEQ ID NO: 3 of U.S. Pat. No. 6,258,562 |
| TNFα | Cimzia ™ (certolizumab pegol) |
| | VH: SEQ ID NO: 14 of U.S. Pat. No. 7,012,135 |
| | VL: SEQ ID NO: 9 of U.S. Pat. No. 7,012,135 |
| TNFα | Simponi ™ (golimumab) |
| | VH: SEQ ID NO: 7 of U.S. Pat. No. 7,250,165 |
| | VL: SEQ ID NO: 8 of U.S. Pat. No. 7,250,165 |
| VEGF | Avastin ™ (bevacizumab) |
| | VH: SEQ ID NO: 9 of U.S. Pat. No. 7,060,269 |
| | VL: SEQ ID NO: 10 of U.S. Pat. No. 7,060,269 |
| VEGF | Lucentis ™ (ranibizumab) |
| | VH: SEQ ID NO: 4 of U.S. Pat. No. 9,914,770 |
| | VL: SEQ ID NO: 2 of U.S. Pat. No. 9,914,770 |

In some aspects, the targeting moiety competes with an antibody set forth above, including in Table 3, for binding to the target molecule. In further aspects, the targeting moiety comprises CDRs having CDR sequences of an antibody set forth above, including in Table 3. In some embodiments, the targeting moiety comprises all 6 CDR sequences of the antibody set forth above, including the antibody set forth in Table 3. In other embodiments, the targeting moiety comprises at least the heavy chain CDR sequences (CDR-H1, CDR-H2, CDR-H3) of such antibody and the light chain CDR sequences of a universal light chain. In further aspects, a targeting moiety comprises a VH comprising the amino acid sequence of the VH of an antibody set forth above, e.g., in Table 3. In some embodiments, the targeting moiety further comprises a VL comprising the amino acid sequence of the VL of the antibody set forth above, e.g., in Table 3. In other embodiments, the targeting moiety further comprises a universal light chain VL sequence.

In some embodiments, the checkpoint inhibitor targeting moiety is non-blocking or poorly-blocking of ligand-receptor binding. Examples of non-blocking or poorly-blocking anti-PD1 antibodies includes antibodies having VH/VL amino acid sequences of SEQ ID NOs: 2/10 of PCT Pub. No. WO2015/112800A1; SEQ ID NOs: 16/17 of U.S. Pat. No. 11,034,765 B2; SEQ ID NOs. 164/178, 165/179, 166/180, 167/181, 168/182, 169/183, 170/184, 171/185, 172/186, 173/187, 174/188, 175/189, 176/190 and 177/190 of U.S. Pat. No. 10,294,299 B2. Examples of non-blocking or poorly-blocking anti-LAG3 antibodies includes antibodies having VH/VL amino acid sequences of SEQ ID NOs 23/24, 3/4 and 11/12 of US Pub. US2022/0056126A1.

5.7.2. Antibodies and Antigen Binding Domains

In certain aspects, the targeting moiety can be any type of antibody or fragment thereof that retains specific binding to an antigenic determinant. In one embodiment the antigen binding moiety is a full-length antibody. In one embodiment the antigen binding moiety is an immunoglobulin molecule, particularly an IgG class immunoglobulin molecule, more particularly an IgG₁ or IgG₄ immunoglobulin molecule. Antibody fragments include, but are not limited to, VH (or V_{H}) fragments, VL (or V_{L}) fragments, Fab fragments, F(ab′)₂ fragments, scFv fragments, Fv fragments, minibodies, diabodies, triabodies, and tetrabodies. In particular embodiments, the antigen binding fragment of an antibody is an scFv or Fab, such as an scFv or Fab that binds to CD2, CD3, CD4, CD7, CD8, XCR1, Clec9a, CD20, MADCAM, a4b7 integrin, TSHR and EpCam.

5.7.2.1. scFv

Single chain Fv or “scFv” antibody fragments comprise the VH and VL domains of an antibody in a single polypeptide chain, are capable of being expressed as a single chain polypeptide, and retain the specificity of the intact antibodies from which they are derived. Generally, the scFv polypeptide further comprises a polypeptide linker between the VH and VL domain that enables the scFv to form the desired structure for target binding. Examples of linkers suitable for connecting the VH and VL chains of an scFv are the linkers identified in Section 5.8.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The scFv can comprise VH and VL sequences from any suitable species, such as murine, human or humanized VH and VL sequences.

To create an scFv-encoding nucleic acid, the VH and VL-encoding DNA fragments are operably linked to another fragment encoding a linker, e.g., encoding any of the linkers described in Section 5.8 (typically a repeat of a sequence containing the amino acids glycine and serine, such as the amino acid sequence (Gly4-Ser)₃, such that the VH and VL sequences can be expressed as a contiguous single-chain protein, with the VL and VH regions joined by the flexible linker (see, e.g., Bird et al., 1988, Science 242:423-426; Huston et al., 1988, Proc. Natl. Acad. Sci. USA 85:5879-5883; McCafferty et al., 1990, Nature 348:552-554).

5.7.2.2. Fab

Fab domains were traditionally produced from by proteolytic cleavage of immunoglobulin molecules using enzymes such as papain. In the IL27 agonists of the disclosure, the Fab domains are typically recombinantly expressed as part of the IL27 agonist.

The Fab domains can comprise constant domain and variable region sequences from any suitable species, and thus can be murine, chimeric, human or humanized.

Fab domains typically comprise a CH domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding module. A disulfide bond between the two constant domains can further stabilize the Fab domain.

For the L27 agonists of the disclosure, particularly when the IL27 contains two different Fab domains and the light chain is not a common or universal light chain, it is advantageous to use Fab heterodimerization strategies to permit the correct association of Fab domains belonging to the same Fab and minimize aberrant pairing of Fab domains belonging to different Fabs. For example, the Fab heterodimerization strategies shown in Table 4 below can be used:

TABLE 4
Fab Heterodimerization Strategies

| STRATEGY | VH | CH1 | VL | CL | REFERENCE |
|---|---|---|---|---|---|
| CrossMabCH1-CL | WT | CL domain | WT | CH1 domain | Schaefer et al., 2011, |
| | | | | | Cancer Cell 2011; |
| | | | | | 20: 472-86; |
| | | | | | PMID: 22014573. |
| orthogonal Fab | 39K, 62E | H172A, F174G | 1R, 38D, (36F) | L135Y, S176W | Lewis et al., 2014, Nat |
| VHVRD1CH1CRD2 - | | | | | Biotechnol 32: 191-8 |
| VLVRD1CλCRD2 | | | | | |
| orthogonal Fab | 39Y | WT | 38R | WT | Lewis et al., 2014, Nat |
| VHVRD2CH1wt - | | | | | Biotechnol 32: 191-8 |
| VLVRD2Cλwt | | | | | |
| TCR CαCβ | 39K | TCR Cα | 38D | TCR Cβ | Wu et al., 2015, MAbs |
| | | | | | 7: 364-76 |
| CR3 | WT | T192E | WT | N137K, S114A | Golay at al., 2016, J |
| | | | | | Immunol 196: 3199-211. |
| MUT4 | WT | L143Q, S188V | WT | V133T, S176V | Golay at al., 2016, J |
| | | | | | Immunol 196: 3199-211. |
| DuetMab | WT | F126C | WT | S121C | Mazor et al., 2015, MAbs |
| | | | | | 7: 377-89; Mazor et al., |
| | | | | | 2015, MAbs 7: 461-669. |
| Domain | WT | CH3 + knob or | WT | CH3 + hole or | Wozniak-Knopp et al., |
| exchanged | | hole mutation | | knob mutation | 2018, |
| | | | | | PLoSONE13(4): e0195442 |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or more amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, e.g., Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, e.g., Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1 R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121 C in the CL domain (see, e.g., Mazor et al., 2015, MAbs 7:377-89).

Fab domains can also be modified by replacing the CH1 domain and CL domain with alternative domains that promote correct assembly. For example, Wu et al., 2015, MAbs 7:364-76, describes substituting the CH1 domain with the constant domain of the a T cell receptor and substituting the CL domain with the b domain of the T cell receptor, and pairing these domain replacements with an additional charge-charge interaction between the VL and VH domains by introducing a 38D modification in the VL domain and a 39K modification in the VH domain.

In lieu of, or in addition to, the use of Fab heterodimerization strategies to promote correct VH-VL pairings, the VL of common light chain (also referred to as a universal light chain) can be used for each Fab VL region of an IL27 agonist of the disclosure. In various embodiments, employing a common light chain as described herein reduces the number of inappropriate species of IL27 agonists as compared to employing original cognate VLs. In various embodiments, the VL domains of the IL27 agonists are identified from monospecific antibodies comprising a common light chain. In various embodiments, the VH regions of the IL27 agonists comprise human heavy chain variable gene segments that are rearranged in vivo within mouse B cells that have been previously engineered to express a limited human light chain repertoire, or a single human light chain, cognate with human heavy chains and, in response to exposure with an antigen of interest, generate an antibody repertoire containing a plurality of human VHs that are cognate with one or one of two possible human VLs, wherein the antibody repertoire specific for the antigen of interest. Common light chains are those derived from a rearranged human VK1-39JK5 sequence or a rearranged human VK3-20JK1 sequence, and include somatically mutated (e.g., affinity matured) versions. See, for example, U.S. Pat. No. 10,412,940.

5.7.3. Peptide-MHC Fusions

The targeting moiety of an IL27 agonist of the disclosure can be a peptide-MHC complex (a “pMHC complex”), e.g., a peptide complexed with an MHC class I domain or a peptide complexed with an MHC class II domain, optionally with a β2 microglobulin domain.

Naturally-occurring MHCs are encoded by a cluster of genes on human chromosome 6. MHCs include, but are not limited to, HLA specificities such as A (e.g., A1-A74), B (e.g., B1-B77), C (e.g., C1-C11), D (e.g., D1-D26), DR (e.g., DR1-DR8), DQ (e.g., DQ1-DQ9) and DP (e.g., DP1-DP6). HLA specificities include A1, A2, A3, All, A23, A24, A28, A30, A33, B7, B8, B35, B44, B53, B60, B62, DR1, DR2, DR3, DR4, DR7, DR8, and DR11.

Naturally occurring MHC class I molecules bind peptides derived from proteolytically degraded proteins. Small peptides obtained accordingly are transported into the endoplasmic reticulum where they associate with nascent MHC class I molecules before being routed through the Golgi apparatus and displayed on the cell surface for recognition by cytotoxic T lymphocytes.

Naturally occurring MHC class I molecules consist of an a (heavy) chain associated with β2 microglobulin. The heavy chain consists of subunits a1-a3. The β2 microglobulin protein and a3 subunit of the heavy chain are associated. In certain embodiments, β2 microglobulin and a3 subunit are associated by covalent binding. In certain embodiments, β2 microglobulin and a3 subunit are associated non-covalently. The α1 and α2 subunits of the heavy chain fold to form a groove for a peptide, e.g., antigenic determinant, to be displayed and recognized by TCR.

Class I molecules generally associate with, e.g., bind, peptides of about 8-9 amino acids (e.g., 7-11 amino acids) in length. All humans have between three and six different class I molecules, which can each bind many different types of peptides. In one specific embodiment, the class I MHC polypeptide is a human class I MHC polypeptide selected from the group consisting of HLA-A, HLA-B, HLA-C, HLA-E, HLA-F, and HLA-G.

In some embodiments, the targeting moiety comprises an MHC class I α heavy chain extracellular domain (human a1, a2, and/or a3 domains) without a transmembrane domain. In some embodiments, the class I α heavy chain polypeptide is HLA-A, HLA-B, HLA-C, HLA-E, HLA-F, HLA-G, HLA-K, or HLA-L. In some embodiments, the HLA-A sequence can be an HLA-A*0201 sequence.

The peptide in the pMHC complex can have the amino acid sequence of a peptide which can be associated with, e.g., presented by, an MHC class I molecule. In certain embodiments, the sequence can comprise from 6 to 20 contiguous amino acids. In certain embodiments, a peptide sequence can be that of a protein fragment, wherein the protein is a derived from, e.g., a portion of a cell surface protein, such as, for example, a cell surface protein associated with immune cells or with a tissue, and wherein the peptide can be bound to the MHC class I heavy chain.

In some embodiments, a pMHC complex targeting moiety comprises (i) an antigenic peptide; (ii) a class I MHC polypeptide or a fragment, mutant or derivative thereof (e.g., the extracellular domain), and optionally, (iii) a β2 microglobulin polypeptide or a fragment, mutant or derivative thereof. For example, the pMHC complex can comprise, from the N- to C-terminus, (i) an antigenic peptide, (ii) a p2M sequence, and (iii) a class I α (heavy) chain sequence. Alternatively, the pMHC complex can comprise, from the N- to C-terminus, (i) an antigenic peptide, (ii) a class I α (heavy) chain sequence, and (iii) a p2M sequence.

In one specific embodiment, the antigenic peptide and the MHC sequence and/or the MHC sequence and the p2M domain are linked to one another via a peptide linker, e.g., as described in Section 5.8. In some embodiments, a single-chain pMHC complex can comprise a first flexible linker between the peptide segment and the β2 microglobulin segment. For example, linkers can extend from and connect the carboxy terminal of the peptide to the amino terminal of the β2 microglobulin segment. In some embodiments, the linkers are structured to allow the peptide to fold into the binding groove resulting in a functional pMHC complex. In some embodiments, this linker can comprise at least 3 amino acids, up to about 15 amino acids (e.g., 20 amino acids). The pMHC linker can comprise a second flexible linker inserted between the β2 microglobulin and MHC I heavy chain segment. For example, linkers can extend from and connect the carboxy terminal of the β2 microglobulin segment to the amino terminal of the MHC I heavy chain segment. In certain embodiments, the β2 microglobulin and the MHC I heavy chain can fold into the binding groove resulting in a molecule which can function in promoting T cell expansion.

When β2M is present, the pMHC complex can include mutations in β2M and in the MHC class I α heavy chain domain such that a disulfide bond may form between them. Exemplary amino acid pairs that can be substituted with cysteines to allow for disulfide bonding between the two domains are identified in Table 5 below or as described in PCT Pub. WO/2015195531, incorporated herein b reference in its entirety:

TABLE 5

| β2M Domain | MHC Domain |
|---|---|
| 12 | 236 |
| 12 | 237 |
| 8 | 234 |
| 10 | 235 |
| 24 | 236 |
| 28 | 232 |
| 98 | 192 |
| 99 | 234 |
| 3 | 120 |
| 31 | 96 |
| 53 | 35 |
| 60 | 96 |
| 60 | 122 |
| 63 | 27 |
| R3 | G120 |
| H31 | Q96 |
| D53 | R35 |
| W60 | Q96 |
| W60 | D122 |
| Y63 | Y27 |
| K6 | E232 |
| Q8 | R234 |
| Y10 | P235 |
| S11 | Q242 |
| N24 | A236 |
| S28 | E232 |
| D98 | H192 |
| M99 | R234 |
| R12 | G237 |

In further embodiments, the single-chain pMHC complex can comprise a peptide covalently attached to an MHC class I α (heavy) chain via a disulfide bridge (i.e., a disulfide bond between two cysteines). See, e.g., U.S. Pat. Nos. 8,992,937 and 8,895,020, each of which is incorporated in its entirety by reference. In certain embodiments, the disulfide bond comprises a first cysteine, that is positioned within a linker extending from the carboxy terminal of the peptide, and a second cysteine that is positioned within an MHC class I heavy (e.g., an MHC class I α (heavy) chain which has a non-covalent binding site for the antigen peptide). In certain embodiments, the second cysteine can be a mutation (addition or substitution) in the MHC class I α (heavy) chain. Preferably, the pMHC complex can comprise one contiguous polypeptide chain as well as a disulfide bridge. Alternatively, the pMHC complex can comprise two contiguous polypeptide chains which are attached via the disulfide bridge as the only covalent linkage. In some embodiments, the linking sequences can comprise at least one amino acid in addition to the cysteine, including one or more glycines, one or more, alanines, and/or one or more serines. In some embodiments, the single-chain molecule comprises from N-terminus to C-terminus an MHC class I peptide (e.g., an antigenic peptide), a first linker that comprises a first cysteine, a p2-microglobulin sequence, a second linker, and a MHC class I heavy chain sequence comprising a second cysteine, wherein the first cysteine and the second cysteine comprise a disulfide bridge. In some embodiments, the second cysteine is a substitution of an amino acid of the MHC class I heavy chain selected from the group consisting of T80C, Y84C and N86C (Y84C refers to a mutation at position 108 in a mature protein, where the mature protein lacks a signal sequence. Alternatively, when the protein still includes a 24 mer signal sequence, the position is instead referred to as Y108C).

In certain embodiments, the disulfide bridge can link a peptide in the class I groove of the pMHC complex if the pMHC complex comprises a first cysteine in a Gly-Ser linker extending between the C-terminus of the peptide and the β2 microglobulin, and a second cysteine in a proximal heavy chain position.

When present, the β2 microglobulin sequence can comprise a full-length (human or non-human) β2 microglobulin sequence. In certain embodiments, the β2 microglobulin sequence lacks the leader peptide sequence. As such, the β2 microglobulin sequence can comprise about 99 amino acids. An exemplary human β2 microglobulin sequence is Genbank accession no. AF072097.1.

As an alternative to type I MHC-based pMHC complexes, the IL27 agonists of the disclosure can include a class II MHC-based pMHC complexes as targeting moieties. A class II MHC-based pMHC complex generally includes a class I MHC polypeptide or a fragment, mutant or derivative thereof. In one specific embodiment, the MHC comprises α and β polypeptides of a class II MHC molecule or a fragment, mutant or derivative thereof. In one specific embodiment, the α and β polypeptides are linked by a peptide linker. In one specific embodiment, the MHC comprises α and β polypeptides of a human class II MHC molecule selected from the group consisting of HLA-DP, HLA-DR, HLA-DQ, HLA-DM and HLA-DO.

MHC class II molecules generally consist of two polypeptide chains, α and β. The chains may come from the DP, DQ, or DR gene groups. There are about 40 known different human MHC class II molecules. All have the same basic structure but vary subtly in their molecular structure. MHC class II molecules bind peptides of 13-18 amino acids in length.

In some embodiments, the pMHC complex comprises one or more MHC class II a chains or an extracellular portion thereof. In some embodiments, the class II α chain is HLA-DMA, HLA-DOA, HLA-DPA, HLA-DQA or HLA-DRA.

In other embodiments, the pMHC complex comprises one or more MHC class II β chains or an extracellular portion thereof. In some embodiments, the class II β chain is HLA-DMB, HLA-DOB, HLA-DPB, HLA-DQB or HLA-DRB.

The peptide in a pMHC complex can be any peptide that is capable of binding to an MHC protein in a manner such that the pMHC complex can bind to a TCR, e.g., in a specific manner.

Examples include peptides produced by hydrolysis and most typically, synthetically produced peptides, including randomly generated peptides, specifically designed peptides, and peptides where at least some of the amino acid positions are conserved among several peptides and the remaining positions are random.

In nature, peptides that are produced by hydrolysis undergo hydrolysis prior to binding of the antigen to an MHC protein. Class I MHC typically present peptides derived from proteins actively synthesized in the cytoplasm of the cell. In contrast, class II MHC typically present peptides derived either from exogenous proteins that enter a cell's endocytic pathway or from proteins synthesized in the ER. Intracellular trafficking permits a peptide to become associated with an MHC protein.

The binding of a peptide to an MHC peptide binding groove can control the spatial arrangement of MHC and/or peptide amino acid residues recognized by a TCR, or pMHC-binding protein produced by an animal genetically modified as disclosed herein. Such spatial control is due in part to hydrogen bonds formed between a peptide and an MHC protein. Based on the knowledge of how peptides bind to various MHCs, the major MHC anchor amino acids and the surface exposed amino acids that are varied among different peptides can be determined. In some embodiments, the length of an MHC-binding peptide is from 5 to 40 amino acid residues, e.g., from 6 to 30 amino acid residues, e.g., from 8 to 20 amino acid residues, e.g., between 9 and 11 amino acid residues, including any size peptide between 5 and 40 amino acids in length, in whole integer increments (i.e., 5, 6, 7, 8, 9 . . . 40). While naturally MHC Class II-bound peptides vary from about 9-40 amino acids, in nearly all cases the peptide can be truncated to a 9-11 amino acid core without loss of MHC binding activity or T-cell recognition.

For therapy of autoimmune diseases, the peptide bound to the MHC can be associated with an autoimmune antigen. Examples of such peptides include human cartilage glycoprotein-39 peptides associated with rheumatoid arthritis (see, e.g., Steenbakkers et al., 2003, J. Immunol. 170:5719-5727 insulin peptides associated with type I diabetes (see, e.g., Zhang et al., 2014, Proc. Nat'l Acad. Sci. USA 111(7) 2656-2661; myelin basic protein peptides associated with multiple sclerosis (Krogsgaard et al., 2000, J Exp Med. 191(8):1395-1412), and gluten peptide associated with celiac disease (see, e.g., Hoydahl et al., 2019, Gastroenterology. 156(5):1428-1439.e10).

### 5.8. Linkers

In certain aspects, the present disclosure provides IL27 agonist in which two or more components of an IL27 agonist are connected to one another by a peptide linker. By way of example and not limitation, linkers can be used to connect (a) an IL27 domain and an Fc domain; (b) an IL27 domain and an HSA polypeptide; (c) an IL27 domain and a targeting moiety; (d) an Fc domain and a targeting moiety (e.g., a Fab domain or an scFv); (e) different domains within a targeting moiety (e.g., the VH and VL domains in a scFv); and/or (f) two IL27 domains (e.g., a p28 moiety and EBI3 moiety).

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a peptide linker is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

Charged (e.g., charged hydrophilic linkers) and/or flexible linkers are particularly preferred.

Examples of flexible linkers that can be used in the IL27 receptor agonists of the disclosure include those disclosed by Chen et al., 2013, Adv Drug Deliv Rev. 65(10): 1357-1369 and Klein et al., 2014, Protein Engineering, Design & Selection 27(10): 325-330. Particularly useful flexible linkers are or comprise repeats of glycines and serines, e.g., a monomer or multimer of GnS or SGn, where n is an integer from 1 to 10, e.g., 1 2, 3, 4, 5, 6, 7, 8, 9 or 10. In one embodiment, the linker is or comprises a monomer or multimer of repeat of G4S (SEQ ID NO: 38) e.g., (GGGGS)n (SEQ ID NO: 81).

Polyglycine linkers can suitably be used in the IL27 receptor agonists of the disclosure. In some embodiments, a peptide linker comprises two consecutive glycines (2Gly), three consecutive glycines (3Gly), four consecutive glycines (4Gly) (SEQ ID NO: 82), five consecutive glycines (5Gly) (SEQ ID NO: 83), six consecutive glycines (6Gly) (SEQ ID NO: 84), seven consecutive glycines (7Gly) (SEQ ID NO: 85), eight consecutive glycines (8Gly) (SEQ ID NO: 86) or nine consecutive glycines (9Gly) (SEQ ID NO: 87).

5.8.1. pMHC Linkers

For pMHC complexes, suitable linkers can range from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. In addition to the linkers above, pMHC linkers include glycine polymers (G)n, glycine-serine polymers (including, for example, (GS)n, (GSGGS)n (SEQ ID NO: 88) and (GGGS)n (SEQ ID NO: 89), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, 1992, Rev. Computational Chem. 1 1173-142, incorporated herein in its entirety by reference). Exemplary linkers can comprise amino acid sequences including, but not limited to, GGSG (SEQ ID NO: 12), GGSGG (SEQ ID NO: 13), GSGSG (SEQ ID NO: 14), GSGGG (SEQ ID NO: 15), GGGSG (SEQ ID NO: 16), GSSSG (SEQ ID NO: 17), GCGASGGGGSGGGGS (SEQ ID NO: 18), GGGGSGGGGS (SEQ ID NO: 19), GGGASGGGGSGGGGS (SEQ ID NO: 20), GGGGSGGGGSGGGGS (SEQ ID NO: 21), GGGASGGGGS (SEQ ID NO: 22), GGGGSGGGGSGGGGSGGGGS (SEQ ID NO: 23), GCGGS (SEQ ID NO: 24) and the like. In some embodiments, a linker polypeptide includes a cysteine residue that can form a disulfide bond with a cysteine residue present in another portion of the pMHC complex. In certain embodiments, the linker comprises the amino acid sequence GCGGS (SEQ ID NO: 24). The substitution of a glycine in the G₄S linker with cysteine can result in the formation of a disulfide bond, for example an MHC targeting moiety with a corresponding cysteine substitution in HLA.A2 that stabilizes the MHC peptide within the MHC complex.

5.8.2. Hinge Sequences

In other embodiments, the IL27 agonist of the disclosure comprise a linker that is a hinge region. In particular, where an IL27 agonist contains an immunoglobulin-based targeting moiety, the hinge can be used to connect the targeting moiety, e.g., a Fab domain, to a multimerization domain, e.g., an Fc domain. Even in the absence of a targeting moiety, a hinge sequence can be utilized to stabilize the IL27 agonist dimer

The hinge region can be a native or a modified hinge region. Hinge regions are typically found at the N-termini of Fc regions.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc domain or Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO 99/15549, WO 2005/003170, WO 2005/003169, WO 2005/003170, WO 98/25971 and WO 2005/003171 and these are incorporated herein by reference.

In various embodiments, positions 233-236 within a hinge domain may be G, G, G and unoccupied; G, G, unoccupied, and unoccupied; G, unoccupied, unoccupied, and unoccupied; or all unoccupied, with positions numbered by EU numbering.

In some embodiments, the IL27 muteins of the disclosure comprise a modified hinge domain that reduces binding affinity for an Fcγ receptor relative to a wild-type hinge domain of the same isotype (e.g., human IgG1 or human IgG4).

In one embodiment, the Fc domain of one or both chains of a dimeric IL27 agonist of the disclosure possesses an intact hinge region at its N-terminus.

In one embodiment the Fc domain of one or both chains of a dimeric IL27 agonist of the disclosure and the hinge region are derived from IgG4 and the hinge region comprises the modified sequence CPPC (SEQ ID NO: 25). The core hinge region of human IgG4 contains the sequence CPSC (SEQ ID NO: 26) compared to IgG1 which contains the sequence CPPC (SEQ ID NO: 25). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30(1):105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

5.8.2.1. Chimeric Hinge Sequences

The hinge region can be a chimeric hinge region.

For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region.

In particular embodiments, a chimeric hinge region comprises the amino acid sequence EPKSCDKTHTCPPCPAPPVA (SEQ ID NO: 27; SEQ ID NO:8 of WO2014/121087, which is incorporated by reference in its entirety herein) or ESKYGPPCPPCPAPPVA (SEQ ID NO: 28; SEQ ID NO:9 of WO2014/121087). Such chimeric hinge sequences can be suitably linked to an IgG4 CH2 region (for example by incorporation into an IgG4 Fc domain, for example a human or murine Fc domain, which can be further modified in the CH2 and/or CH3 domain to reduce effector function, for example as described in Section 5.4.2).

5.8.2.2. Hinge Sequences with Reduced Effector Function

In further embodiments, the hinge region can be modified to reduce effector function, for example as described in WO2016161010A2, which is incorporated by reference in its entirety herein. In various embodiments, the positions 233-236 of the modified hinge region are G, G, G and unoccupied; G, G, unoccupied, and unoccupied; G, unoccupied, unoccupied, and unoccupied; or all unoccupied, with positions numbered by EU numbering (as shown in FIG. 1 of WO2016161010A2). These segments can be represented as GGG-, GG--, G--- or ---- with “-” representing an unoccupied position.

Position 236 is unoccupied in canonical human IgG2, but is occupied by in other canonical human IgG isotypes. Positions 233-235 are occupied by residues other than G in all four human isotypes (as shown in FIG. 1 of WO2016161010A2).

The hinge modification within positions 233-236 can be combined with position 228 being occupied by P. Position 228 is naturally occupied by P in human IgG1 and IgG2 but is occupied by S in human IgG4 and R in human IgG3. An S228P mutation in an IgG4 antibody is advantageous in stabilizing an IgG4 antibody and reducing exchange of heavy chain light chain pairs between exogenous and endogenous antibodies. Preferably positions 226-229 are occupied by C, P, P and C respectively.

Exemplary hinge regions have residues 226-236, sometimes referred to as middle (or core) and lower hinge, occupied by the modified hinge sequences designated GGG-(233-236), GG--(233-236), G---(233-236) and no G(233-236). Optionally, the hinge domain amino acid sequence comprises CPPCPAPGGG-GPSVF (SEQ NO: 29; SEQ ID NO:1 of WO2016161010A2), CPPCPAPGG--GPSVF (SEQ ID NO: 30; SEQ ID NO:2 of WO2016161010A2), CPPCPAPG---GPSVF (SEQ ID NO: 31; SEQ ID NO:3 of WO2016161010A2), or CPPCPAP----GPSVF (SEQ ID NO: 32; SEQ ID NO:4 of WO2016161010A2).

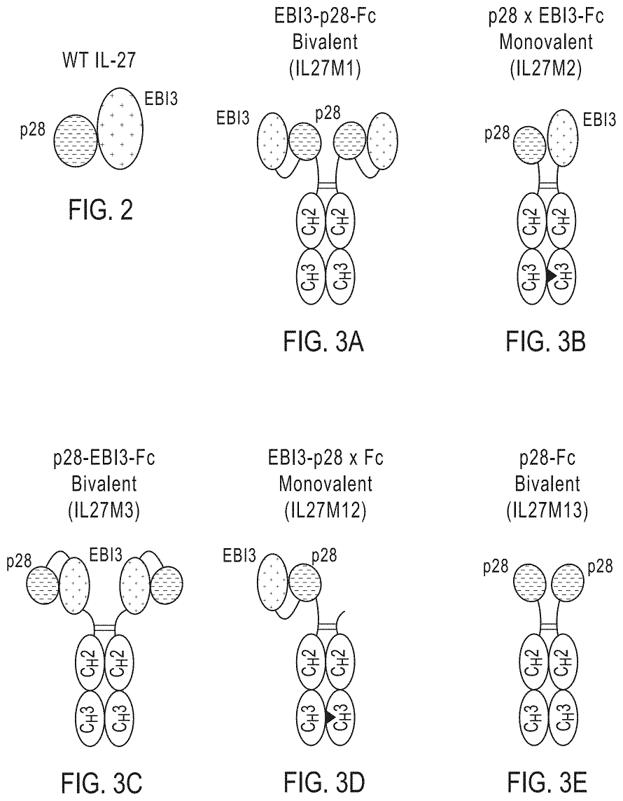

The modified hinge regions described above can be incorporated into a heavy chain constant region, which typically include CH2 and CH3 domains, and which may have an additional hinge segment (e.g., an upper hinge) flanking the designated region. Such additional constant region segments present are typically of the same isotype, preferably a human isotype, although can be hybrids of different isotypes. The isotype of such additional human constant regions segments is preferably human IgG4, but can also be human IgG1, IgG2, or IgG3 or hybrids thereof in which domains are of different isotypes. Exemplary sequences of human IgG1, IgG2 and IgG4 are shown in FIGS. 2-4 of WO2016161010A2.

In specific embodiments, the modified hinge sequences can be linked to an IgG4 CH2 region (for example by incorporation into an IgG4 Fc domain, for example a human or murine Fc domain, which can be further modified in the CH2 and/or CH3 domain to reduce effector function, for example as described in Section 5.4.2).

### 5.9. Nucleic Acids and Host Cells

In another aspect, the disclosure provides nucleic acids encoding the IL27 agonists of the disclosure. In some embodiments, the IL27 agonists are encoded by a single nucleic acid. In other embodiments, for example in the case of a heterodimeric molecule or a molecule comprising a targeting moiety composed of more than one polypeptide chain, the IL27 agonists can be encoded by a plurality (e.g., two, three, four or more) nucleic acids.

A single nucleic acid can encode an IL27 agonist that comprises a single polypeptide chain, an IL27 agonist that comprises two or more polypeptide chains, or a portion of an IL27 agonist that comprises more than two polypeptide chains (for example, a single nucleic acid can encode two polypeptide chains of an IL27 agonist comprising three, four or more polypeptide chains, or three polypeptide chains of an IL27 agonist comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

In some embodiments, an IL27 agonist comprising two or more polypeptide chains is encoded by two or more nucleic acids. The number of nucleic acids encoding an IL27 agonist can be equal to or less than the number of polypeptide chains in the IL27 agonist (for example, when more than one polypeptide chains are encoded by a single nucleic acid).

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

5.9.1. Vectors

The disclosure provides vectors comprising nucleotide sequences encoding an IL27 agonist or an IL27 agonist component described herein, for example one or two of the polypeptide chains of a dimeric IL27 agonist. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. Methods and conditions for culturing the resulting transfected cells and for recovering the expressed polypeptides are known to those skilled in the art, and may be varied or optimized depending upon the specific expression vector and mammalian host cell employed, based upon the present description.

5.9.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### 5.10. Pharmaceutical Compositions

5.10.1. Pharmaceutical Compositions Comprising IL27 Agonist Polypeptide

The IL27 agonists of the disclosure may be in the form of compositions comprising the IL27 agonist and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the IL27 agonist and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intrathecally, topically or locally. The most suitable route for administration in any given case will depend on the particular antibody, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of an IL27 agonist of the disclosure per dose. The quantity of IL27 agonist included in a unit dose will depend on the disease being treated, as well as other factors as are well known in the art. Such unit dosages may be in the form of a lyophilized dry powder containing an amount of IL27 agonist suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of IL27 agonist suitable for a single administration.

The pharmaceutical compositions may also be supplied in bulk from containing quantities of IL27 agonist suitable for multiple administrations.

Pharmaceutical compositions may be prepared for storage as lyophilized formulations or aqueous solutions by mixing an IL27 agonist having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of IL27 agonist.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

5.10.2. Pharmaceutical Compositions For Delivery of IL27 Agonist Encoding Nucleic Acids

An IL27 agonist of the disclosure can be delivered by any method useful for gene therapy, for example as mRNA or through viral vectors encoding the IL27 agonist under the control of a suitable promoter.

Exemplary viral vectors include recombinant adenovirus and adeno-associated virus vectors (rAAV). rAAV vectors are based on the defective and nonpathogenic parvovirus adeno-associated type 2 virus. Most such vectors are derived from a plasmid that retains only the AAV inverted terminal repeats flanking the transgene expression cassette. Efficient gene transfer and stable transgene delivery due to integration into the genomes of the transduced cell are key features for this vector system. AAV serotypes useful for delivering IL27 transgenes AAV1, AAV2, AAV3, AAV4, AAV5, AAV6, AAV8, AAV 8.2, AAV9, and AAV rh10 and pseudotyped AAV such as AAV2/8, AAV2/5 and AAV2/6.

AAV may be manufactured at a clinical scale by a number of different processes. Examples of systems that can be used include (1) plasmid DNA transfection in mammalian cells, (2) Ad infection of stable mammalian cell lines, (3) infection of mammalian cells with recombinant herpes simplex viruses (rHSVs), and (4) infection of insect cells (Sf9 cells) with recombinant baculoviruses (reviewed by Penaud-Budloo et al., 2018, Mol Ther Methods Clin Dev. 8: 166-180).

Replication-deficient recombinant adenoviral vectors (Ad) can be produced at high titer and readily infect a number of different cell types. Most adenovirus vectors are engineered such that a transgene replaces the Ad Ela, Elb, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Packaging cells are used to form virus particles that are capable of infecting a host cell. Such cells include 293 cells, which package adenovirus, and w2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

The nucleic acid molecule (e.g., mRNA) or virus can be formulated as the sole pharmaceutically active ingredient in a pharmaceutical composition or can be combined with other active agents for the particular disorder treated. Optionally, other medicinal agents, pharmaceutical agents, carriers, adjuvants, diluents can be included in the compositions provided herein. For example, any one or more of a wetting agents, emulsifiers and lubricants, such as sodium lauryl sulfate and magnesium stearate, as well as coloring agents, release agents, coating agents, sweetening, flavoring and perfuming agents, preservatives, antioxidants, chelating agents and inert gases also can be present in the compositions. Exemplary other agents and excipients that can be included in the compositions include, for example, water soluble antioxidants, such as ascorbic acid, cysteine hydrochloride, sodium bisulfate, sodium metabisulfite, sodium sulfite; oil-soluble antioxidants, such as ascorbyl palmitate, butylated hydroxyanisole (BHA), butylated hydroxytoluene (BHT), lecithin, propyl gallate, α-tocopherol; and metal chelating agents, such as citric acid, ethylenediamine tetraacetic acid (EDTA), sorbitol, tartaric acid and phosphoric acid.

### 5.11. Therapeutic Indications and Methods of Treatment

IL27 agonists of the disclosure are useful in treating conditions which are treatable with IL27, e.g., inflammatory- and immune-related conditions or disorders, e.g., autoimmune disorders.

In some embodiments, the disclosure provides a method of treating an inflammatory or immune (e.g., autoimmune) condition with an IL27 receptor agonist that is targeted to the desired microenvironment or to disease-reactive lymphocytes, comprising administering to a subject in need thereof an IL27 receptor agonist or pharmaceutical composition as described herein, where the IL27 receptor agonist comprises a targeting moiety that recognizes a target molecule that is expressed in the disease microenvironment or to disease-reactive lymphocytes.

The present disclosure further provides a method of localized delivery of an IL27 protein, comprising administering to a subject an IL27 receptor agonist or pharmaceutical composition as described herein, where the IL27 receptor agonist comprises a targeting moiety that recognizes a target molecule that is expressed by a tissue to which the IL27 receptor agonist is to be locally delivered. As used herein, the term “locally delivered” does not require local administration but rather indicates that the IL27 receptor agonist be selectively localized to a tissue of interest following administration.

The present disclosure further provides a method of administering to the subject IL27 therapy with reduced systemic exposure and/or reduced systemic toxicity, comprising administering to a subject the IL27 therapy in the form of an IL27 receptor agonist or pharmaceutical composition as described herein. Accordingly, the foregoing methods permit IL27 therapy with reduced off-target side effects by virtue of preferential targeting of an IL27 receptor agonist to a particular target cell or tissue and/or attenuation and/or masking of the IL27 moiety until at the site of intended activity.

The present disclosure further provides method of locally modulating an immune response in a target cell or tissue, comprising administering to a subject IL27 receptor agonist or pharmaceutical composition as described herein which has one or more targeting moieties capable of binding a target molecule expressed in the disease microenvironment or by disease-reactive lymphocytes. The IL27 receptor agonist can then modulate the immune response against at least one cell type in the target tissue.

In some embodiments, the administration is not local to the tissue. For example, the administration can be systemic or subcutaneous.

In particular embodiments, the condition treated by the IL27 agonists of the disclosure is an autoimmune condition, transplantation rejection (e.g., organ or bone marrow transplant rejection), post-traumatic immune response, infectious disease (e.g., a parasitic infection), or graft-versus-host disease. Particular examples of autoimmune conditions include arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, scleroderma, systemic sclerosis, or allergic asthma.

The IL27 agonists of the disclosure will generally be used in an amount effective to achieve the intended purpose. For use to treat or prevent a disease condition, the IL27 agonists of the disclosure, or pharmaceutical compositions thereof, are administered or applied in a therapeutically effective amount. Determination of a therapeutically effective amount is well within the capabilities of those skilled in the art, especially in light of the detailed disclosure provided herein. A skilled artisan readily recognizes that in many cases, treatment with an IL27 agonist may not provide a cure, but may only provide partial benefit. In some embodiments, a physiological change having some benefit is also considered therapeutically beneficial. Thus, the terms “effective amount” and “therapeutically effective amount” encompass dosages and dosing regimens that confer a partial benefit.

The subject, patient, or individual in need of treatment is typically a mammal, more specifically a human. For the prevention or treatment of disease, the appropriate dosage of an IL27 agonist of the disclosure (when used alone or in combination with one or more other additional therapeutic agents) will depend on the type of disease to be treated, the route of administration, the body weight of the patient, the particular IL27 agonist, the severity and course of the disease, whether the antibody is administered for preventive or therapeutic purposes, previous or concurrent therapeutic interventions, the patient's clinical history and response to the IL27 agonist, and the discretion of the attending physician. The practitioner responsible for administration will, in any event, determine the concentration of active ingredient(s) in a composition and appropriate dose(s) for the individual subject. Various dosing schedules including, but not limited to, single or multiple administrations over various time-points, bolus administration, and pulse infusion are contemplated herein.

A single administration of unconjugated IL27 agonist can range from about 50,000 IU/kg to about 1,000,000 IU/kg or more, more typically about 600,000 IU/kg of IL27 agonist. This may be repeated several times a day (e.g., 2-3 times), for several days (e.g., about 3-5 consecutive days) and then may be repeated one or more times following a period of rest (e.g., about 7-14 days). Thus, a therapeutically effective amount may comprise only a single administration or many administrations over a period of time (e.g., about 20-30 individual administrations of about 600,000 IU/kg of IL27 agonist, each given over about a 10-20 day period).

Similarly, the IL27 agonist is suitably administered to the patient at one time or over a series of treatments. Depending on the type and severity of the disease, about 1 μg/kg to 15 mg/kg (e.g., 0.1 mg/kg-10 mg/kg) of IL27 agonist can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the IL27 agonist would be in the range from about 0.005 mg/kg to about 10 mg/kg. In other non-limiting examples, a dose may also comprise from about 1 μg/kg/body weight, about 5 μg/kg/body weight, about 10 μg/kg/body weight, about 50 μg/kg/body weight, about 100 μg/kg/body weight, about 200 μg/kg/body weight, about 350 μg/kg/body weight, about 500 μg/kg/body weight, about 1 mg/kg/body weight, about 5 mg/kg/body weight, about 10 mg/kg/body weight, about 50 mg/kg/body weight, about 100 mg/kg/body weight, about 200 mg/kg/body weight, about 350 mg/kg/body weight, about 500 mg/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 μg/kg/body weight to about 500 mg/kg/body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g., every week or every three weeks (e.g., such that the patient receives from about two to about twenty, or e.g., about six doses of the IL27 agonist). An initial higher loading dose, followed by one or more lower doses may be administered. However, other dosage regimens may be useful. The progress of this therapy is easily monitored by conventional techniques and assays.

For systemic administration, a therapeutically effective dose can be estimated initially from in vitro assays, such as cell culture assays. A dose can then be formulated in animal models to achieve a circulating concentration range that includes the EC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the IL27 agonists which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by ELISA HPLC.

In cases of local administration or selective uptake, the effective local concentration of the IL27 agonists may not be related to plasma concentration. One having skill in the art will be able to optimize therapeutically effective local dosages without undue experimentation.

A therapeutically effective dose of the IL27 agonists described herein will generally provide therapeutic benefit without causing substantial toxicity. Toxicity and therapeutic efficacy of an IL27 agonist can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. IL27 agonists that exhibit large therapeutic indices are preferred. In one embodiment, the IL27 agonist according to the present disclosure exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition. (See, e.g., Fingl et al., 1975, In: The Pharmacological Basis of Therapeutics, Ch. 1, p. 1, incorporated herein by reference in its entirety).

The attending physician for patients treated with IL27 agonists of the disclosure would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

### 5.12. Combination Therapy

The IL27 agonists according to the disclosure may be administered in combination with one or more other additional agents in therapy. For instance, an IL27 agonist of the disclosure may be co-administered with at least one additional therapeutic agent. The term “therapeutic agent” encompasses any agent administered to treat a symptom or disease in a subject in need of such treatment. Such additional therapeutic agent may comprise any active ingredients suitable for the particular indication being treated, preferably those with complementary activities that do not adversely affect each other.

The IL27 agonists are generally used in the same dosages and with administration routes as described herein, or about from 1 to 99% of the dosages described herein, or in any dosage and by any route that is empirically/clinically determined to be appropriate.

For treatment of immune and inflammatory conditions, the IL27 agonists of the disclosure can be used in combination with immunosuppressive or immunomodulatory therapies. Non-limiting examples of immunosuppressive therapies include immunosuppressive compounds such as cyclosporin A, cyclophosphamide, FK506, tacrolimus, corticosteroids, azathioprine, mycophenolate mofetil, sirolimus, rapamycin, rapamycin analogs, deoxyspagarin, prednisone, and the like.

Such combination therapies noted above encompass combined administration (where two or more therapeutic agents are included in the same or separate compositions), and separate administration, in which case, administration of the IL27 agonist of the disclosure can occur prior to, simultaneously, and/or following, administration of the additional therapeutic agent and/or adjuvant.

### 6. NUMBERED EMBODIMENTS

While various specific embodiments have been illustrated and described, it will be appreciated that various changes can be made without departing from the spirit and scope of the disclosure(s). The present disclosure is exemplified by the numbered embodiments set forth below. Unless otherwise specified, features of any of the concepts, aspects and/or embodiments described in the detailed description above are applicable mutatis mutandis to any of the following numbered embodiments.

In preferred aspects of the numbered embodiments below and the claims which follow, the EBI3 moieties, p28 moieties, Fc domains, and the variants thereof preferably comprise the amino acid sequences of human EBI3, human p28, human Fc domains, and variants thereof, for example variants with at least about 90%, at least about 95%, at least about 96%, at least about 97%, at least about 98%, at least about 99% or 100% sequence identity to such human sequence.

1. A p28 moiety comprising a variant p28 domain, wherein the variant p28 domain:
(a) has an amino acid sequence with at least 90%, at least 95% or at least 97% sequence identity to an IL27Ra binding domain of mature human or mature murine p28 and comprises one or more amino acid substitutions at the position corresponding to:
   (i) amino acid H52 of full length human p28 or amino acid Y48 of full length murine p28, wherein the substitution is optionally alanine;
   (ii) amino acid K56 of full length human p28 or amino acid K52 of full length murine p28, wherein the substitution is optionally alanine,
   (iii) amino acid S59 of full length human p28 or amino acid S55 of full length murine p28, wherein the substitution is optionally alanine;
   (iv) amino acid E60 of full length human p28 or amino acid E56 of full length murine p28, wherein the substitution is optionally alanine;
   (v) amino acid W138 of full length human p28 or amino acid W134 of full length murine p28, wherein the substitution is optionally alanine;
   (vi) amino acid L142 of full length human p28 or amino acid L138 of full length murine p28, wherein the substitution is optionally alanine;
   (vii) amino acid R145 of full length human p28 or amino acid R141 of full length murine p28, wherein the substitution is optionally alanine;
   (viii) amino acid D146 of full length human p28 or amino acid D142 of full length murine p28, wherein the substitution is optionally alanine;
   (ix) amino acid R149 of full length human p28 or amino acid R145 of full length murine p28, wherein the substitution is optionally alanine;
   (x) amino acid H150 of full length human p28 or amino acid H146 of full length murine p28, wherein the substitution is optionally alanine; or
   (xi) any combination of (a)(i) to (a)(x); and/or
(b) has an amino acid sequence with at least 90%, at least 95% or at least 97% sequence identity to a gp130 binding domain of mature human or mature murine p28 and comprises one or more amino acid substitutions at the position corresponding to:
   (i) amino acid L73 of full length human p28 or amino acid L69 of full length murine p28, wherein the substitution is optionally alanine;
   (ii) amino acid V76 of full length human p28 or amino acid V72 of full length murine p28, wherein the substitution is optionally alanine;
   (iii) amino acid W197 of full length human p28 or amino acid W195 of full length murine p28, wherein the substitution is optionally alanine;
   (iv) amino acid L200 of full length human p28 or amino acid L198 of full length murine p28, wherein the substitution is optionally alanine;
   (v) amino acid L201 of full length human p28 or amino acid L199 of full length murine p28, wherein the substitution is optionally alanine;
   (vi) amino acid Y204 of full length human p28 or amino acid Y202 of full length murine p28, wherein the substitution is optionally alanine;
   (vii) amino acid R205 of full length human p28 or amino acid Q203 of full length murine p28, wherein the substitution is optionally alanine; or
   (viii) any combination of (b)(i) to (b)(vii).

2. The p28 moiety of embodiment 1, wherein the p28 moiety has a single variant p28 domain.

3. The p28 moiety of embodiment 1 or embodiment 2, wherein the p28 moiety lacks an EBI3 domain.

4. The p28 moiety of any one of embodiments 1 to 3, which comprises an amino acid substitution at the position corresponding to amino acid H52 of full length human p28 or amino acid Y48 of full length murine p28, wherein the substitution is optionally alanine.

5. The p28 moiety of any one of embodiments 1 to 4, which comprises an amino acid substitution at the position corresponding to amino acid K56 of full length human p28 or amino acid K52 of full length murine p28, wherein the substitution is optionally alanine.

6. The p28 moiety of any one of embodiments 1 to 5, which comprises an amino acid substitution at the position corresponding to amino acid S59 of full length human p28 or amino acid S55 of full length murine p28, wherein the substitution is optionally alanine.

7. The p28 moiety of any one of embodiments 1 to 6, which comprises an amino acid substitution at the position corresponding to amino acid E60 of full length human p28 or amino acid E56 of full length murine p28, wherein the substitution is optionally alanine.

8. The p28 moiety of any one of embodiments 1 to 7, which comprises an amino acid substitution at the position corresponding to amino acid L73 of full length human p28 or amino acid L69 of full length murine p28, wherein the substitution is optionally alanine.

9. The p28 moiety of any one of embodiments 1 to 8, which comprises an amino acid substitution at the position corresponding to amino acid V76 of full length human p28 or amino acid V72 of full length murine p28, wherein the substitution is optionally alanine.

10. The p28 moiety of any one of embodiments 1 to 9, which comprises an amino acid substitution at the position corresponding to amino acid W138 of full length human p28 or amino acid W134 of full length murine p28, wherein the substitution is optionally alanine.

11. The p28 moiety of any one of embodiments 1 to 10, which comprises an amino acid substitution at the position corresponding to amino acid L142 of full length human p28 or amino acid L138 of full length murine p28, wherein the substitution is optionally alanine.

12. The p28 moiety of any one of embodiments 1 to 11, which comprises an amino acid substitution at the position corresponding to amino acid R145 of full length human p28 or amino acid R141 of full length murine p28, wherein the substitution is optionally alanine.

13. The p28 moiety of any one of embodiments 1 to 12, which comprises an amino acid substitution at the position corresponding to amino acid D146 of full length human p28 or amino acid D142 of full length murine p28, wherein the substitution is optionally alanine.

14. The p28 moiety of any one of embodiments 1 to 13, which comprises an amino acid substitution at the position corresponding to amino acid R149 of full length human p28 or amino acid R145 of full length murine p28, wherein the substitution is optionally alanine.

15. The p28 moiety of any one of embodiments 1 to 14, which comprises an amino acid substitution at the position corresponding to amino acid H150 of full length human p28 or amino acid H146 of full length murine p28, wherein the substitution is optionally alanine.

16. The p28 moiety of any one of embodiments 1 to 15, which comprises an amino acid substitution at the position corresponding to amino acid W197 of full length human p28 or amino acid W195 of full length murine p28, wherein the substitution is optionally alanine.

17. The p28 moiety of any one of embodiments 1 to 16, which comprises an amino acid substitution at the position corresponding to amino acid L200 of full length human p28 or amino acid L198 of full length murine p28, wherein the substitution is optionally alanine.

18. The p28 moiety of any one of embodiments 1 to 17, which comprises an amino acid substitution at the position corresponding to amino acid L201 of full length human p28 or amino acid L199 of full length murine p28, wherein the substitution is optionally alanine.

19. The p28 moiety of any one of embodiments 1 to 18, which comprises an amino acid substitution at the position corresponding to amino acid Y204 of full length human p28 or amino acid Y202 of full length murine p28, wherein the substitution is optionally alanine

20. The p28 moiety of any one of embodiments 1 to 19, which comprises an amino acid substitution at the position corresponding to amino acid R205 of full length human p28 or amino acid Q203 of full length murine p28, wherein the substitution is optionally alanine.

21. The p28 moiety of any one of embodiments 1 to 3, which comprises amino acid substitutions at the positions corresponding to amino acid L200 of full length human p28 or amino acid L198 of full length murine p28; and amino acid L201 of full length human p28 or amino acid L199 of full length murine p28, wherein each substitution is optionally alanine.

22. The p28 moiety of any one of embodiments 1 to 3, which comprises amino acid substitutions at the positions corresponding to amino acid Y204 of full length human p28 or amino acid Y202 of full length murine p28; and amino acid R205 of full length human p28 or amino acid Q203 of full length murine p28, wherein the substitution is optionally alanine, wherein each substitution is optionally alanine.

23. The p28 moiety of any one of embodiments 1 to 3, which comprises amino acid substitutions at the positions corresponding to amino acid amino acid L142 of full length human p28 or amino acid L138 of full length murine p28; amino acid R149 of full length human p28 or amino acid R145 of full length murine p28; and amino acid H150 of full length human p28 or amino acid H146 of full length murine p28, wherein the substitution is optionally alanine, wherein each substitution is optionally alanine.

24. An IL27 receptor agonist comprising
(a) a p28 moiety comprising an IL27Ra binding domain and/or a gp130 binding domain of p28, optionally wherein the p28 moiety is a p28 moiety according to any one of embodiments 1 to 23; and
(b) an EBI3 moiety comprising a p28 binding domain of EBI3; or
(c) both a p28 moiety as defined in (a) and an EBI3 moiety as defined in (b).

25. An IL27 receptor agonist, which is optionally an IL27 receptor agonist according to embodiment 24, which comprises one, two, or more IL27 monomers.

26. An IL27 receptor agonist, which is optionally an IL27 agonist according to embodiment 25, comprising two IL27 monomers having the configuration of Exemplary Monomer 1.

27. The IL27 receptor agonist of embodiment 26, in which each p28 moiety is associated with an EBI3 moiety.

28. The IL27 receptor agonist of embodiment 27, in which each EBI3 moiety comprises a myc-myc-his (mmh) tag.

29. The IL27 receptor agonist of embodiment 28, the mmh tag is N-terminal to each EBI3.

30. The IL27 receptor agonist of embodiment 28, the mmh tag is C-terminal to each EBI3.

31. An IL27 receptor agonist, which is optionally an IL27 agonist according to embodiment 25, comprising a first IL27 monomer having the configuration of Exemplary Monomer 1 and a second IL27 monomer having the configuration of Exemplary Monomer 2.

32. An IL27 receptor agonist, which is optionally an IL27 agonist according to embodiment 25, comprising a first IL27 monomer having the configuration of Exemplary Monomer 1 and a separate polypeptide chain comprising a multimerization moiety capable of associating with the Exemplary Monomer 1.

33. The IL27 receptor agonist of embodiment 32, wherein the separate polypeptide chain further comprises
(a) a targeting moiety or a targeting moiety component, optionally wherein (i) the targeting moiety or targeting moiety component is N-terminal to the multimerization moiety and/or (ii) the targeting moiety or targeting moiety component and the multimerization moiety are separated by a linker; or
(b) a means for binding to a target molecule or component thereof, optionally wherein (i) the means for binding to a target molecule or component thereof is N-terminal to the multimerization moiety and/or (ii) the means for binding to a target molecule or component thereof and the multimerization moiety are separated by a linker.

34. The IL27 receptor agonist of embodiment 33, wherein:
(a) the targeting moiety component is associated with a counterpart targeting moiety component (e.g., a VH with a VL); or
(b) the component of the means for binding to a target molecule is associated with a counterpart component to form the means for binding to the target molecule.

35. An IL27 receptor agonist, which is optionally an IL27 agonist according to embodiment 25, comprising a first IL27 monomer having the configuration of Exemplary Monomer 2 and a separate polypeptide chain comprising a multimerization moiety capable of associating with the Exemplary Monomer 2.

36. The IL27 receptor agonist of embodiment 35, wherein the separate polypeptide chain further comprises:
(a) a targeting moiety or a targeting moiety component, optionally wherein (i) the targeting moiety or targeting moiety component is N-terminal to the multimerization moiety and/or (ii) the targeting moiety or targeting moiety component and the multimerization moiety are separated by a linker; or
(b) a means for binding to a target molecule or component thereof, optionally wherein (i) the means for binding to a target molecule or component thereof is N-terminal to the multimerization moiety and/or (ii) the means for binding to a target molecule or component thereof and the multimerization moiety are separated by a linker.

37. The IL27 receptor agonist of embodiment 36, wherein:
(a) the targeting moiety component is associated with a counterpart targeting moiety component (e.g., a VH with a VL); or
(b) the component of the means for binding to a target molecule is associated with a counterpart component to form the means for binding to the target molecule.

38. An IL27 receptor agonist, which is optionally an IL27 agonist according to embodiment 25, comprising two IL27 monomers having the configuration of Exemplary Monomer 3.

39. The IL27 receptor agonist of embodiment 38, wherein each p28 moiety is associated with an EBI3 moiety.

40. The IL27 receptor agonist of embodiment 39, wherein each EBI3 moiety comprises a myc-myc-his (mmh) tag.

41. The IL27 receptor agonist of embodiment 40, the mmh tag is N-terminal to each EBI3.

42. The IL27 receptor agonist of embodiment 40, the mmh tag is C-terminal to each EBI3.

43. An IL27 receptor agonist, which is optionally an IL27 agonist according to embodiment 25, comprising a first IL27 monomer having the configuration of Exemplary Monomer 3 and a second IL27 monomer having the configuration of Exemplary Monomer 4.

44. An IL27 receptor agonist, which is optionally an IL27 agonist according to embodiment 25, comprising a first IL27 monomer having the configuration of Exemplary Monomer 3 and a separate polypeptide chain comprising a multimerization moiety capable of associating with the Exemplary Monomer 3.

45. The IL27 receptor agonist of embodiment 44, wherein the separate polypeptide chain further comprises:
(a) a targeting moiety or a targeting moiety component, optionally wherein (i) the targeting moiety or targeting moiety component is N-terminal to the multimerization moiety and/or (ii) the targeting moiety or targeting moiety component and the multimerization moiety are separated by a linker; or
(b) a means for binding to a target molecule or component thereof, optionally wherein (i) the means for binding to a target molecule or component thereof is N-terminal to the multimerization moiety and/or (ii) the means for binding to a target molecule or component thereof and the multimerization moiety are separated by a linker.

46. The IL27 receptor agonist of embodiment 45, wherein:
(a) the targeting moiety component is associated with a counterpart targeting moiety component (e.g., a VH with a VL); or
(b) the component of the means for binding to a target molecule is associated with a counterpart component to form the means for binding to the target molecule.

47. An IL27 receptor agonist, which is optionally an IL27 agonist according to embodiment 25, comprising a first IL27 monomer having the configuration of Exemplary Monomer 4 and a separate polypeptide chain comprising a multimerization moiety capable of associating with the Exemplary Monomer 4.

48. The IL27 receptor agonist of embodiment 53, wherein the separate polypeptide chain further comprises:
(a) a targeting moiety or a targeting moiety component, optionally wherein (i) the targeting moiety or targeting moiety component is N-terminal to the multimerization moiety and/or (ii) the targeting moiety or targeting moiety component and the multimerization moiety are separated by a linker; or
(b) a means for binding to a target molecule or component thereof, optionally wherein (i) the means for binding to a target molecule or component thereof is N-terminal to the multimerization moiety and/or (ii) the means for binding to a target molecule or component thereof and the multimerization moiety are separated by a linker.

49. The IL27 receptor agonist of embodiment 48, wherein:
(a) the targeting moiety component is associated with a counterpart targeting moiety component (e.g., a VH with a VL); or
(b) the component of the means for binding to a target molecule is associated with a counterpart component to form the means for binding to the target molecule.

50. An IL27 receptor agonist, which is optionally an IL27 agonist according to embodiment 25, comprising an IL27 monomer having the configuration of Exemplary Monomer 5.

51. An IL27 receptor agonist, which is optionally an IL27 agonist according to embodiment 25, comprising two IL27 monomers having the configuration of Exemplary Monomer 5.

52. An IL27 receptor agonist, which is optionally an IL27 agonist according to embodiment 25, comprising a first IL27 monomer having the configuration of Exemplary Monomer 5 and a second IL27 monomer having the configuration of Exemplary Monomer 6.

53. An IL27 receptor agonist, which is optionally an IL27 agonist according to embodiment 25, comprising an IL27 monomer having the configuration of Exemplary Monomer 5 and a separate polypeptide chain comprising a multimerization moiety capable of associating with the Exemplary monomer 5.

54. The IL27 receptor agonist of embodiment 53, wherein the separate polypeptide chain further comprises:
(a) a targeting moiety or a targeting moiety component, optionally wherein (i) the targeting moiety or targeting moiety component is N-terminal to the multimerization moiety and/or (ii) the targeting moiety or targeting moiety component and the multimerization moiety are separated by a linker; or
(b) a means for binding to a target molecule or component thereof, optionally wherein (i) the means for binding to a target molecule or component thereof is N-terminal to the multimerization moiety and/or (ii) the means for binding to a target molecule or component thereof and the multimerization moiety are separated by a linker.

55. The IL27 receptor agonist of embodiment 33, wherein:
(a) the targeting moiety component is associated with a counterpart targeting moiety component (e.g., a VH with a VL); or
(b) the component of the means for binding to a target molecule is associated with a counterpart component to form the means for binding to the target molecule.

56. An IL27 receptor agonist, which is optionally an IL27 agonist according to embodiment 25, comprising an IL27 monomer having the configuration of Exemplary Monomer 6.

57. An IL27 receptor agonist, which is optionally an IL27 agonist according to embodiment 25, comprising two IL27 monomers having the configuration of Exemplary Monomer 6.

58. An IL27 receptor agonist, which is optionally an IL27 agonist according to embodiment 25, comprising an IL27 monomer having the configuration of Exemplary Monomer 6 and a separate polypeptide chain comprising a multimerization moiety capable of associating with the Exemplary monomer 6.

59. The IL27 receptor agonist of embodiment 58, wherein the separate polypeptide chain further comprises:
(a) a targeting moiety or a targeting moiety component, optionally wherein (i) the targeting moiety or targeting moiety component is N-terminal to the multimerization moiety and/or (ii) the targeting moiety or targeting moiety component and the multimerization moiety are separated by a linker;
(b) a means for binding to a target molecule or component thereof, optionally wherein (i) the means for binding to a target molecule or component thereof is N-terminal to the multimerization moiety and/or (ii) the means for binding to a target molecule or component thereof and the multimerization moiety are separated by a linker.

60. The IL27 receptor agonist of embodiment 59, wherein:
(a) the targeting moiety component is associated with a counterpart targeting moiety component (e.g., a VH with a VL); or
(b) the component of the means for binding to a target molecule is associated with a counterpart component to form the means for binding to the target molecule.

61. An IL27 receptor agonist, which is optionally an IL27 agonist according to embodiment 25, comprising an IL27 monomer having the configuration of Exemplary Monomer 7.

62. An IL27 receptor agonist, which is optionally an IL27 agonist according to embodiment 25, comprising two IL27 monomers having the configuration of Exemplary Monomer 7.

63. An IL27 receptor agonist, which is optionally an IL27 agonist according to embodiment 25, comprising a first IL27 monomer having the configuration of Exemplary Monomer 7 and a second IL27 monomer having the configuration of Exemplary Monomer 8.

64. An IL27 receptor agonist, which is optionally an IL27 agonist according to embodiment 25, comprising an IL27 monomer having the configuration of Exemplary Monomer 7 and a separate polypeptide chain comprising a multimerization moiety capable of associating with the Exemplary monomer 7.

65. The IL27 receptor agonist of embodiment 64, wherein the separate polypeptide chain further comprises:
(a) a targeting moiety or a targeting moiety component, optionally wherein (i) the targeting moiety or targeting moiety component is N-terminal to the multimerization moiety and/or (ii) the targeting moiety or targeting moiety component and the multimerization moiety are separated by a linker; or
(b) a means for binding to a target molecule or component thereof, optionally wherein (i) the means for binding to a target molecule or component thereof is N-terminal to the multimerization moiety and/or (ii) the means for binding to a target molecule or component thereof and the multimerization moiety are separated by a linker.

66. The IL27 receptor agonist of embodiment 33, wherein:
(a) the targeting moiety component is associated with a counterpart targeting moiety component (e.g., a VH with a VL); or
(b) the component of the means for binding to a target molecule is associated with a counterpart component to form the means for binding to the target molecule.

67. An IL27 receptor agonist, which is optionally an IL27 agonist according to embodiment 25, comprising an IL27 monomer having the configuration of Exemplary Monomer 8.

68. An IL27 receptor agonist, which is optionally an IL27 agonist according to embodiment 25, comprising two IL27 monomers having the configuration of Exemplary Monomer 8.

69. An IL27 receptor agonist, which is optionally an IL27 agonist according to embodiment 25, comprising a first IL27 monomer having the configuration of Exemplary Monomer 8 and a separate polypeptide chain comprising a multimerization moiety capable of associating with the Exemplary monomer 8.

70. The IL27 receptor agonist of embodiment 69, wherein the separate polypeptide chain further comprises:
(a) a targeting moiety or a targeting moiety component, optionally wherein (i) the targeting moiety or targeting moiety component is N-terminal to the multimerization moiety and/or (ii) the targeting moiety or targeting moiety component and the multimerization moiety are separated by a linker; or
(b) a means for binding to a target molecule or component thereof, optionally wherein (i) the means for binding to a target molecule or component thereof is N-terminal to the multimerization moiety and/or (ii) the means for binding to a target molecule or component thereof and the multimerization moiety are separated by a linker.

71. The IL27 receptor agonist of embodiment 70, wherein:
(a) the targeting moiety component is associated with a counterpart targeting moiety component (e.g., a VH with a VL); or
(b) the component of the means for binding to a target molecule is associated with a counterpart component to form the means for binding to the target molecule.

72. An IL27 receptor agonist, which is optionally an IL27 agonist according to embodiment 25, comprising a first IL27 monomer having the configuration of Exemplary Monomer 9 and a second IL27 monomer having the configuration of Exemplary Monomer 10.

73. An IL27 receptor agonist, which is optionally an IL27 agonist according to embodiment 25, comprising a first IL27 monomer having the configuration of Exemplary Monomer 9 and a second IL27 monomer having the configuration of Exemplary Monomer 12.

74. An IL27 receptor agonist, which is optionally an IL27 agonist according to embodiment 25, comprising a first IL27 monomer having the configuration of Exemplary Monomer 11 and a second IL27 monomer having the configuration of Exemplary Monomer 10.

75. An IL27 receptor agonist, which is optionally an IL27 agonist according to embodiment 25, comprising a first IL27 monomer having the configuration of Exemplary Monomer 11 and a second IL27 monomer having the configuration of Exemplary Monomer 12.

76. An IL27 receptor agonist, which is optionally an IL27 agonist according to embodiment 25, comprising an IL27 monomer having the configuration of Exemplary Monomer 13.

77. An IL27 receptor agonist, which is optionally an IL27 agonist according to embodiment 25, comprising an IL27 monomer having the configuration of Exemplary Monomer 14.

78. An IL27 receptor agonist, which is optionally an IL27 agonist according to embodiment 25, comprising an IL27 monomer having the configuration of Exemplary Monomer 15.

79. An IL27 receptor agonist, which is optionally an IL27 agonist according to embodiment 25, comprising an IL27 monomer having the configuration of Exemplary Monomer 16.

80. An IL27 receptor agonist, which is optionally an IL27 agonist according to embodiment 25, comprising the IL27 receptor agonist of embodiment 33 and the IL27 receptor agonist of embodiment 36, wherein the p28 moiety of the IL27 receptor agonist of embodiment 33 is associated with the EBI3 moiety of the IL27 receptor agonist of embodiment 36.

81. An IL27 receptor agonist, which is optionally an IL27 agonist according to embodiment 25, comprising the IL27 receptor agonist of embodiment 45 and the IL27 receptor agonist of embodiment 48, wherein the p28 moiety of the IL27 receptor agonist of embodiment 45 is associated with the EBI3 moiety of the IL27 receptor agonist of embodiment 48.

82. An IL27 receptor agonist, which is optionally an IL27 agonist according to embodiment 25, comprising:
(a) a first polypeptide chain comprising:
   (i) optionally, a first multimerization moiety;
   (ii) optionally, (A) a first targeting moiety or a first targeting moiety component or (B) a first means for binding to a target molecule or component thereof; and
   (iii) optionally, a stabilization moiety; and
(b) a second polypeptide comprising:
   (i) optionally, a second multimerization moiety;
   (ii) optionally, (A) a second targeting moiety or a second targeting moiety component or (B) a second means for binding to a target molecule or component thereof; and
   (iii) optionally, a second stabilization moiety;
(c) a first p28 moiety which is optionally a p28 moiety as defined in any one of embodiments 1 to 23; and
(d) a first EBI3 moiety.

83. The IL27 receptor agonist of embodiment 82, which is bivalent for IL27.

84. The IL27 receptor agonist of embodiment 82 or embodiment 83, wherein:
(a) the first polypeptide comprises the first p28 moiety and the first EBI3 moiety; and
(b) the second polypeptide comprises a second p28 moiety and a second p28 moiety which is optionally a p28 moiety as defined in any one of embodiments 1 to 23.

85. The IL27 receptor agonist of any one of embodiments 82 to 84, which comprises a first IL27 monomer and a second IL27 monomer.

86. The IL27 receptor agonist of embodiment 85, wherein the first IL27 monomer and the second IL27 monomer are not identical.

87. The IL27 receptor agonist of embodiment 85, wherein the first IL27 monomer and the second IL27 monomer are identical.

88. The IL27 receptor agonist of any one of embodiments 84 to 87, which comprises a first multimerization moiety and a second multimerization moiety and wherein the first p28 moiety and the first EBI3 moiety are N-terminal to the first multimerization moiety, and the second p28 moiety and the second EBI3 moiety are N-terminal to the second multimerization moiety.

89. The IL27 receptor agonist of any one of embodiments 84 to 87, which comprises a first multimerization moiety and a second multimerization moiety and wherein the first p28 moiety and the first EBI3 moiety are C-terminal to the first multimerization moiety, and the second p28 moiety and the second EBI3 moiety are C-terminal to the second multimerization moiety.

90. The IL agonist of any one of embodiments 84 to 89, wherein the first EBI3 moiety is N-terminal to the first p28 moiety and the second EBI3 moiety is N-terminal to the p28 moiety of the second IL27 moiety.

91. The IL agonist of any one of embodiments 84 to 89, wherein the first EBI3 moiety is C-terminal to the first p28 moiety and the second EBI3 moiety is C-terminal to the p28 moiety of the second IL27 moiety.

92. The IL27 receptor agonist of any one of embodiments 84 to 91, which comprises a first multimerization moiety and a second multimerization moiety and wherein:
(a) the first multimerization moiety and either the first EBI3 moiety or the first p28 moiety are connected via a first multimerization moiety linker; and
(b) the second multimerization moiety and either the second EBI3 moiety or the second p28 moiety are connected via a second multimerization moiety linker.

93. The IL27 receptor agonist of embodiment 92, wherein each of the first multimerization moiety linker and the second multimerization moiety linker is at least 5 or at least 10 amino acids in length.

94. The IL27 receptor agonist of embodiment 92 or embodiment 93, wherein each of the first multimerization moiety linker and the second multimerization moiety linker is or comprises a glycine-serine linker.

95. The IL27 receptor agonist of any one of embodiments 92 to 94, wherein each of the first multimerization moiety linker and the second multimerization moiety linker comprises the amino acid sequence G₄S (SEQ ID NO: 38).

96. The IL27 receptor agonist of any one of embodiments 92 to 95, wherein each of the first multimerization moiety linker and the second multimerization moiety linker is or comprises a repeat of the amino acid sequence G₄S (SEQ ID NO: 38).

97. The IL27 receptor agonist of embodiment 96, wherein the repeat comprises, 2, 3, 4, 5, 6, or more repeats of the amino acid sequence G₄S (SEQ ID NO: 38).

98. The IL27 receptor agonist of any one of embodiments 84 to 97, wherein the first EBI3 moiety and the first p28 moiety are connected via a first intra-IL27 moiety linker, and the second EBI3 moiety and the second p28 moiety are connected via a second intra-IL27 moiety linker.

99. The IL27 receptor agonist of embodiment 98, wherein each of the first intra-IL27 moiety linker and the second intra-IL27 moiety linker is at least 5 or at least 10 amino acids in length.

100. The IL27 receptor agonist of embodiment 98 or 99, wherein each of the first intra-IL27 moiety linker and the second intra-IL27 moiety linker is or comprises a glycine-serine linker.

101. The IL27 receptor agonist of any one of embodiments 98 to 100, wherein each of the first intra-IL27 moiety linker and the second intra-IL27 moiety linker comprises the amino acid sequence G₄S (SEQ ID NO: 38).

102. The IL27 receptor agonist of any one of embodiments 98 to 101, wherein each of the first intra-IL27 moiety linker and the second linker is or comprises a repeat of the amino acid sequence G₄S (SEQ ID NO: 38).

103. The IL27 receptor agonist of embodiment 102, wherein the repeat comprises, 2, 3, 4, 5, 6, or more repeats of the amino acid sequence G₄S (SEQ ID NO: 38).

104. The IL27 receptor agonist of embodiment 82, which is monovalent for IL27.

105. The IL27 receptor agonist of embodiment 104, wherein:
(a) the first polypeptide comprises the first p28 moiety which is optionally a p28 moiety as defined in any one of embodiments 1 to 23; and
(b) the second polypeptide comprises the first EBI3 moiety.

106. The IL27 receptor agonist of embodiment 105, which comprises a first multimerization moiety and a second multimerization moiety and wherein the first EBI3 moiety is N-terminal to the first multimerization moiety and the first p28 moiety is N-terminal to the second multimerization moiety.

107. The IL27 receptor agonist of embodiment 105, which comprises a first multimerization moiety and a second multimerization moiety and wherein the first EBI3 moiety is C-terminal to the first multimerization moiety and the first p28 moiety is C-terminal to the second multimerization moiety.

108. The IL27 receptor agonist of any one of embodiments 105 to 107, which comprises a first multimerization moiety and a second multimerization moiety and wherein:
(a) the first multimerization moiety and the first EBI3 moiety are connected via a first multimerization moiety linker; and
(b) the second multimerization moiety and the first p28 moiety are connected via a second multimerization moiety linker.

109. The IL27 receptor agonist of embodiment 108, wherein each of the first multimerization moiety linker and the second multimerization moiety linker is at least 5 or at least 10 amino acids in length.

110. The IL27 receptor agonist of embodiment 108 or embodiment 109, wherein each of the first multimerization moiety linker and the second multimerization moiety linker is or comprises a glycine-serine linker.

111. The IL27 receptor agonist of any one of embodiments 108 to 110, wherein each of the first multimerization moiety linker and the second multimerization moiety linker comprises the amino acid sequence G₄S (SEQ ID NO: 38).

112. The IL27 receptor agonist of any one of embodiments 108 to 111, wherein each of the first multimerization moiety linker and the second multimerization moiety linker is or comprises a repeat of the amino acid sequence G₄S (SEQ ID NO: 38).

113. The IL27 receptor agonist of embodiment 112, wherein the repeat comprises 2, 3, 4, 5, 6, or more repeats of the amino acid sequence G₄S (SEQ ID NO: 38).

114. The IL27 receptor agonist of embodiment 104, wherein:
(a) the first polypeptide comprises the first EBI3 moiety and the first p28 moiety; and
(b) the second polypeptide lacks both an EBI3 moiety and a p28 moiety.

115. The IL27 receptor agonist of embodiment 114, which comprises a first multimerization moiety and a second multimerization moiety and wherein the first EBI3 moiety and the first p28 moiety are N-terminal to the first multimerization moiety.

116. The IL27 receptor agonist of embodiment 114, which comprises a first multimerization moiety and a second multimerization moiety and wherein the first EBI3 moiety and the first p28 moiety are C-terminal to the first multimerization moiety.

117. The IL27 agonist of any one of embodiments 114 to 116, wherein the first EBI3 moiety is N-terminal to the first p28 moiety.

118. The IL27 agonist of any one of embodiments 114 to 116, wherein the first EBI3 moiety is C-terminal to the first p28 moiety.

119. The IL27 receptor agonist of any one of embodiments 114 to 118, which comprises a first multimerization moiety and a second multimerization moiety and wherein the first multimerization moiety and either the first EBI3 moiety or the first p28 moiety are connected via a first multimerization moiety linker.

120. The IL27 receptor agonist of embodiment 119, wherein the first multimerization moiety linker is at least 5 or at least 10 amino acids in length.

121. The IL27 receptor agonist of embodiment 119 or embodiment 120, wherein the first multimerization moiety linker is or comprises a glycine-serine linker.

122. The IL27 receptor agonist of any one of embodiments 119 to 121, wherein the first multimerization moiety linker comprises the amino acid sequence G₄S (SEQ ID NO: 38).

123. The IL27 receptor agonist of any one of embodiments 119 to 122, wherein each of the first multimerization moiety linker and the second multimerization moiety linker is or comprises a repeat of the amino acid sequence G₄S (SEQ ID NO: 38).

124. The IL27 receptor agonist of embodiment 123, wherein the repeat comprises, 2, 3, 4, 5, 6, or more repeats of the amino acid sequence G₄S (SEQ ID NO: 38).

125. The IL27 receptor agonist of any one of embodiments 114 to 124, wherein the first EBI3 moiety and the first p28 moiety are connected via a first intra-IL27 moiety linker.

126. The IL27 receptor agonist of embodiment 125, wherein the intra-IL27 moiety linker is at least 5 or at least 10 amino acids in length.

127. The IL27 agonist of embodiment 125 or embodiment 126, wherein the first intra-IL27 moiety linker is or comprises a glycine-serine linker.

128. The IL27 receptor agonist of any one of embodiments 125 to 127, wherein the first intra-IL27 moiety linker comprises the amino acid sequence G₄S.

129. The IL27 receptor agonist of any one of embodiments 125 to 128, wherein first intra-IL27 moiety linker is or comprises a repeat of the amino acid sequence G₄S.

130. The IL27 receptor agonist of embodiment 129, wherein the repeat comprises, 2, 3, 4, 5, 6, or more repeats of the amino acid sequence G₄S.

131. The IL27 agonist of any one of embodiments 82 to 130, which has a multimerization moiety:EBI3 moiety stoichiometry of 1:1.

132. The IL27 agonist of any one of embodiments 82 to 130, which has a multimerization moiety:p28 moiety stoichiometry of 2:1.

133. The IL27 agonist of any one of embodiments 82 to 130, which has a multimerization moiety:p28 moiety stoichiometry of 4:1.

134. The IL27 agonist of any one of embodiments 82 to 133, which has an Fc domain:p28 moiety stoichiometry of 1:1.

135. The IL27 agonist of any one of embodiments 82 to 133, which has an Fc domain:EBI3 moiety stoichiometry of 2:1.

136. The IL27 agonist of any one of embodiments 82 to 133, which has an Fc domain:EBI3 moiety stoichiometry of 4:1.

137. An IL27 receptor agonist, which is optionally an IL27 agonist according to embodiment 25, comprising a polypeptide chain comprising:
(a) a stabilization moiety; and
(b) an EBI3 moiety, a p28 moiety, or both an EBI3 moiety and a p28 moiety, optionally wherein the p28 moiety as defined in any one of embodiments 1 to 23.

138. The IL27 receptor agonist of embodiment 137, which is monovalent for IL27.

139. The IL27 receptor agonist of embodiment 137 or embodiment 138, wherein the EBI3 moiety and the p28 moiety are N-terminal to the stabilization moiety.

140. The IL27 receptor agonist of any one of embodiments 137 to 139, wherein the EBI3 moiety and the p28 moiety are C-terminal to the stabilization moiety.

141. The IL27 receptor agonist of any one of embodiments 137 to 140, wherein the EBI3 moiety is N-terminal to the p28 moiety.

142. The IL27 receptor agonist of any one of embodiments 137 to 140, wherein the EBI3 moiety is C-terminal to the p28 moiety.

143. The IL27 receptor agonist of any one of embodiments 137 to 142, wherein the stabilization moiety and either the EBI3 moiety or the p28 moiety are connected via a stabilization moiety linker.

144. The IL27 receptor agonist of embodiment 143, wherein the stabilization moiety linker is at least 5 or at least 10 amino acids in length.

145. The IL27 receptor agonist of embodiment 143 or embodiment 144, wherein the stabilization moiety linker is or comprises a glycine-serine linker.

146. The IL27 receptor agonist of any one of embodiments 143 to 145, wherein the stabilization moiety linker comprises the amino acid sequence G₄S (SEQ ID NO: 38).

147. The IL27 receptor agonist of any one of embodiments 143 to 146, wherein the stabilization moiety linker is or comprises a repeat of the amino acid sequence G₄S (SEQ ID NO: 38).

148. The IL27 receptor agonist of embodiment 147, wherein the repeat comprises, 2, 3, 4, 5, 6, or more repeats of the amino acid sequence G₄S (SEQ ID NO: 38).

149. The IL27 receptor agonist of any one of embodiments 137 to 148, wherein the EBI3 moiety and the p28 moiety are connected via an intra-IL27 moiety linker.

150. The IL27 receptor agonist of embodiment 149, wherein the intra-IL27 moiety linker is at least 5 or at least 10 amino acids in length.

151. The IL27 receptor agonist of embodiment 149 or embodiment 150, wherein the intra-IL27 moiety linker is or comprises a glycine-serine linker.

152. The IL27 receptor agonist of any one of embodiments 149 to 151, wherein the intra-IL27 moiety linker comprises the amino acid sequence G₄S (SEQ ID NO: 38).

153. The IL27 receptor agonist of any one of embodiments 143 to 146, wherein the intra-IL27 moiety linker is or comprises a repeat of the amino acid sequence G₄S (SEQ ID NO: 38).

154. The IL27 receptor agonist of embodiment 153, wherein the repeat comprises, 2, 3, 4, 5, 6, or more repeats of the amino acid sequence G₄S (SEQ ID NO: 38).

155. The IL27 receptor agonist of any one of embodiments 82 to 130, wherein the first EBI3 moiety has an amino acid sequence with at least about 90%, at least about 95%, at least about 97%, at least about 98%, or at least about 99% sequence identity to a p28 binding domain of mature human or mature murine EBI3 or a variant thereof.

156. The IL27 receptor agonist of any one of embodiments 82 to 155, wherein the first EBI3 moiety has an amino acid sequence with at least about 90% sequence identity to a p28 binding domain of mature human or mature murine EBI3.

157. The IL27 receptor agonist of any one of embodiments 82 to 156, wherein the first EBI3 moiety has an amino acid sequence with at least about 95% sequence identity to a p28 binding domain of mature human or mature murine EBI3.

158. The IL27 receptor agonist of any one of embodiments 82 to 157, wherein the first EBI3 moiety has an amino acid sequence with at least about 97% sequence identity to a p28 binding domain of mature human or mature murine EBI3.

159. The IL27 receptor agonist of any one of embodiments 82 to 158, wherein the first EBI3 moiety has an amino acid sequence with at least about 98% sequence identity to a p28 binding domain of mature human or mature murine EBI3.

160. The IL27 receptor agonist of any one of embodiments 82 to 159, wherein the first EBI3 moiety has an amino acid sequence with at least about 99% sequence identity to a p28 binding domain of mature human or mature murine EBI3.

161. The IL27 receptor agonist of any one of embodiments 82 to 160, wherein the first EBI3 moiety has an amino acid sequence with an amino acid sequence with at least about 90%, at least about 95%, at least about 97%, at least about 98%, or at least about 99% sequence identity to mature human or mature murine EBI3 or a variant thereof.

162. The IL27 receptor agonist of any one of embodiments 82 to 161, wherein the first EBI3 moiety has an amino acid sequence with at least about 90% sequence identity to mature human or mature murine EBI3.

163. The IL27 receptor agonist of any one of embodiments 82 to 162, wherein the first EBI3 moiety has an amino acid sequence with at least about 95% sequence identity to mature human or mature murine EBI3.

164. The IL27 receptor agonist of any one of embodiments 82 to 163, wherein the first EBI3 moiety has an amino acid sequence with at least about 97% sequence identity to mature human or mature murine EBI3.

165. The IL27 receptor agonist of any one of embodiments 82 to 164, wherein the first EBI3 moiety has an amino acid sequence with at least about 98% sequence identity to mature human or mature murine EBI3.

166. The IL27 receptor agonist of any one of embodiments 82 to 165, wherein the first EBI3 moiety has an amino acid sequence with at least about 99% sequence identity to mature human or mature murine EBI3.

167. The IL27 receptor agonist of any one of embodiments 82 to 166, wherein the first p28 moiety has an amino acid sequence with at least about 90%, at least about 95%, at least about 97%, at least about 98%, or at least about 99% sequence identity to an IL27Ra binding domain of mature human or mature murine p28 and/or a gp130 binding domain of mature human or mature murine p28, optionally wherein the p28 moiety is as defined in any one of embodiments 1 and 23.

168. The IL27 receptor agonist of any one of embodiments 82 to 167, wherein the first p28 moiety has an amino acid sequence with at least about 90% sequence identity to an IL27Ra and/or a gp130 binding domain of mature human or mature murine p28.

169. The IL27 receptor agonist of any one of embodiments 82 to 168, wherein the first p28 moiety has an amino acid sequence with at least about 95% sequence identity to an IL27Ra and/or a gp130 binding domain of mature human or mature murine p28.

170. The IL27 receptor agonist of any one of embodiments 82 to 169, wherein the first p28 moiety has an amino acid sequence with at least about 97% sequence identity to an IL27Ra and/or a gp130 binding domain of mature human or mature murine p28

171. The IL27 receptor agonist of any one of embodiments 82 to 170, wherein the first p28 moiety has an amino acid sequence with at least about 98% sequence identity to an IL27Ra and/or a gp130 binding domain of mature human or mature murine p28.

172. The IL27 receptor agonist of any one of embodiments 82 to 171, wherein the first p28 moiety has an amino acid sequence with at least about 99% sequence identity to an IL27Ra and/or a gp130 binding domain of mature human or mature murine p28.

173. The IL27 receptor agonist of any one of embodiments 82 to 172, wherein the first p28 moiety has an amino acid sequence with an amino acid sequence with at least about 90%, at least about 95%, at least about 97%, at least about 98%, or at least about 99% sequence identity to mature human or mature murine p28.

174. The IL27 receptor agonist of any one of embodiments 82 to 173, wherein the first p28 moiety has an amino acid sequence with at least about 90% sequence identity to mature human or mature murine p28.

175. The IL27 receptor agonist of any one of embodiments 82 to 174, wherein the first p28 moiety has an amino acid sequence with at least about 95% sequence identity to mature human or mature murine p28.

176. The IL27 receptor agonist of any one of embodiments 82 to 175, wherein the first p28 moiety has an amino acid sequence with at least about 97% sequence identity to mature human or mature murine p28

177. The IL27 receptor agonist of any one of embodiments 82 to 176, wherein the first p28 moiety has an amino acid sequence with at least about 98% sequence identity to mature human or mature murine p28.

178. The IL27 receptor agonist of any one of embodiments 82 to 177, wherein the first p28 moiety has an amino acid sequence with at least about 99% sequence identity to mature human or mature murine p28.

179. The IL27 receptor agonist of any one of embodiments 84 to 178, wherein the second EBI3 moiety has an amino acid sequence with at least about 90%, at least about 95%, at least about 97%, at least about 98%, or at least about 99% sequence identity to a p28 binding domain of mature human or mature murine EBI3 or a variant thereof.

180. The IL27 receptor agonist of any one of embodiments 84 to 179, wherein the second EBI3 moiety has an amino acid sequence with at least about 90% sequence identity to a p28 binding domain of mature human or mature murine EBI3.

181. The IL27 receptor agonist of any one of embodiments 84 to 180, wherein the second EBI3 moiety has an amino acid sequence with at least about 95% sequence identity to a p28 binding domain of mature human or mature murine EBI3.

182. The IL27 receptor agonist of any one of embodiments 84 to 181, wherein the second EBI3 moiety has an amino acid sequence with at least about 97% sequence identity to a p28 binding domain of mature human or mature murine EBI3.

183. The IL27 receptor agonist of any one of embodiments 84 to 182, wherein the second EBI3 moiety has an amino acid sequence with at least about 98% sequence identity to a p28 binding domain of mature human or mature murine EBI3.

184. The IL27 receptor agonist of any one of embodiments 84 to 183, wherein the first EBI3 moiety has an amino acid sequence with at least about 99% sequence identity to a p28 binding domain of mature human or mature murine EBI3.

185. The IL27 receptor agonist of any one of embodiments 84 to 184, wherein the second EBI3 moiety has an amino acid sequence with an amino acid sequence with at least about 90%, at least about 95%, at least about 97%, at least about 98%, or at least about 99% sequence identity to mature human or mature murine EBI3 or a variant thereof.

186. The IL27 receptor agonist of any one of embodiments 84 to 185, wherein the second EBI3 moiety has an amino acid sequence with at least about 90% sequence identity to mature human or mature murine EBI3.

187. The IL27 receptor agonist of any one of embodiments 84 to 186, wherein the second EBI3 moiety has an amino acid sequence with at least about 95% sequence identity to mature human or mature murine EBI3.

188. The IL27 receptor agonist of any one of embodiments 84 to 187, wherein the second EBI3 moiety has an amino acid sequence with at least about 97% sequence identity to mature human or mature murine EBI3.

189. The IL27 receptor agonist of any one of embodiments 84 to 188, wherein the second EBI3 moiety has an amino acid sequence with at least about 98% sequence identity to mature human or mature murine EBI3.

190. The IL27 receptor agonist of any one of embodiments 84 to 189, wherein the second EBI3 moiety has an amino acid sequence with at least about 99% sequence identity to mature human or mature murine EBI3.

191. The IL27 receptor agonist of any one of embodiments 84 to 190, wherein the second p28 moiety has an amino acid sequence with at least about 90%, at least about 95%, at least about 97%, at least about 98%, or at least about 99% sequence identity to an IL27Ra binding domain of mature human or mature murine p28 and/or a gp130 binding domain of mature human or mature murine p28, optionally wherein the p28 moiety is as defined in any one of embodiments 1 and 23.

192. The IL27 receptor agonist of any one of embodiments 84 to 191, wherein the second p28 moiety has an amino acid sequence with at least about 90% sequence identity to an IL27Ra and/or a gp130 binding domain of mature human or mature murine p28.

193. The IL27 receptor agonist of any one of embodiments 84 to 192, wherein the second p28 moiety has an amino acid sequence with at least about 95% sequence identity to an IL27Ra and/or a gp130 binding domain of mature human or mature murine p28.

194. The IL27 receptor agonist of any one of embodiments 84 to 193, wherein the second p28 moiety has an amino acid sequence with at least about 97% sequence identity to an IL27Ra and/or a gp130 binding domain of mature human or mature murine p28

195. The IL27 receptor agonist of any one of embodiments 84 to 194, wherein the second p28 moiety has an amino acid sequence with at least about 98% sequence identity to an IL27Ra and/or a gp130 binding domain of mature human or mature murine p28.

196. The IL27 receptor agonist of any one of embodiments 84 to 195, wherein the second p28 moiety has an amino acid sequence with at least about 99% sequence identity to an IL27Ra and/or a gp130 binding domain of mature human or mature murine p28.

197. The IL27 receptor agonist of any one of embodiments 84 to 196, wherein the second p28 moiety has an amino acid sequence with an amino acid sequence with at least about 90%, at least about 95%, at least about 97%, at least about 98%, or at least about 99% sequence identity to mature human or mature murine p28.

198. The IL27 receptor agonist of any one of embodiments 84 to 197, wherein the second p28 moiety has an amino acid sequence with at least about 90% sequence identity to mature human or mature murine p28.

199. The IL27 receptor agonist of any one of embodiments 84 to 198, wherein the second p28 moiety has an amino acid sequence with at least about 95% sequence identity to mature human or mature murine p28.

200. The IL27 receptor agonist of any one of embodiments 84 to 199, wherein the second p28 moiety has an amino acid sequence with at least about 97% sequence identity to mature human or mature murine p28

201. The IL27 receptor agonist of any one of embodiments 84 to 200, wherein the second p28 moiety has an amino acid sequence with at least about 98% sequence identity to mature human or mature murine p28.

202. The IL27 receptor agonist of any one of embodiments 84 to 201, wherein the second p28 moiety has an amino acid sequence with at least about 99% sequence identity to mature human or mature murine p28

203. The IL27 receptor agonist of any one of embodiments 82 to 202, wherein neither the first polypeptide nor the second polypeptide comprise a cytokine moiety other than an IL27 (e.g., p28 or EBI3) moiety.

204. The IL27 receptor agonist of any one of embodiments 82 to 203, wherein the first p28 moiety, and if present, the second p28 moiety comprise(s) at least one amino acid substitution.

205. The IL27 receptor agonist of any one of embodiments 82 to 204, wherein the first p28 moiety, and if present, the second p28 moiety comprise(s) a p28 domain having an amino acid substitution at a position corresponding to residue 52 of full length human p28 (e.g., full length murine p28 residue 48).

206. An IL27 receptor agonist, which is optionally an IL27 receptor agonist according to embodiment 205, which comprises a first p28 moiety comprising a p28 domain having an alanine substitution at a position corresponding to residue 52 of full length human p28 (e.g., full length murine p28 residue 48), and which optionally further comprises a second p28 moiety comprising a p28 domain having an alanine substitution at a position corresponding to residue 52 of full length human p28 (e.g., full length murine p28 residue 48).

207. The IL27 receptor agonist of any one of embodiments 82 to 206, wherein the first p28 moiety, and if present, the second p28 moiety comprise(s) a p28 domain having an amino acid substitution at a position corresponding to residue 56 of full length human p28 (e.g., full length murine p28 residue 52).

208. An IL27 receptor agonist, which is optionally an IL27 receptor agonist according to embodiment 207, which comprises a first p28 moiety comprising a p28 domain having an alanine substitution at a position corresponding to residue 56 of full length human p28 (e.g., full length murine p28 residue 52), and which optionally further comprises a second p28 moiety comprising a p28 domain having an alanine substitution at a position corresponding to residue 56 of full length human p28 (e.g., full length murine p28 residue 52).

209. The IL27 receptor agonist of any one of embodiments 82 to 208, wherein the first p28 moiety, and if present, the second p28 moiety comprise(s) a p28 domain having an amino acid substitution at a position corresponding to residue 59 of full length human p28 (e.g., full length murine p28 residue 55).

210. An IL27 receptor agonist, which is optionally an IL27 receptor agonist according to embodiment 209, which comprises a first p28 moiety comprising a p28 domain having an alanine substitution at a position corresponding to residue 59 of full length human p28 (e.g., full length murine p28 residue 55), and which optionally further comprises a second p28 moiety comprising a p28 domain having an alanine substitution at a position corresponding to residue 59 of full length human p28 (e.g., full length murine p28 residue 55).

211. The IL27 receptor agonist of any one of embodiments 82 to 210, wherein the first p28 moiety, and if present, the second p28 moiety comprise(s) a p28 domain having an amino acid substitution at a position corresponding to residue 60 of full length human p28 (e.g., full length murine p28 residue 56).

212. An IL27 receptor agonist, which is optionally an IL27 receptor agonist according to embodiment 211, which comprises a first p28 moiety comprising a p28 domain having an alanine substitution at a position corresponding to residue 60 of full length human p28 (e.g., full length murine p28 residue 56), and which optionally further comprises a second p28 moiety comprising a p28 domain having an alanine substitution at a position corresponding to residue 60 of full length human p28 (e.g., full length murine p28 residue 56).

213. The IL27 receptor agonist of any one of embodiments 82 to 212, wherein the first p28 moiety, and if present, the second p28 moiety comprise(s) a p28 domain having an amino acid substitution at a position corresponding to residue 73 of full length human p28 (e.g., full length murine p28 residue 69).

214. An IL27 receptor agonist, which is optionally an IL27 receptor agonist according to embodiment 213, which comprises a first p28 moiety comprising a p28 domain having an alanine substitution at a position corresponding to residue 73 of full length human p28 (e.g., full length murine p28 residue 69), and which optionally further comprises a second p28 moiety comprising a p28 domain having an alanine substitution at a position corresponding to residue 73 of full length human p28 (e.g., full length murine p28 residue 69).

215. The IL27 receptor agonist of any one of embodiments 82 to 214, wherein the first p28 moiety, and if present, the second p28 moiety comprise(s) a p28 domain having an amino acid substitution at a position corresponding to residue 76 of full length human p28 (e.g., full length murine p28 residue 72).

216. An IL27 receptor agonist, which is optionally an IL27 receptor agonist according to embodiment 215, which comprises a first p28 moiety comprising a p28 domain having an alanine substitution at a position corresponding to residue 76 of full length human p28 (e.g., full length murine p28 residue 72), and which optionally further comprises a second p28 moiety comprising a p28 domain having an alanine substitution at a position corresponding to residue 76 of full length human p28 (e.g., full length murine p28 residue 72).

217. The IL27 receptor agonist of any one of embodiments 82 to 216, wherein the first p28 moiety, and if present, the second p28 moiety comprise(s) a p28 domain having an amino acid substitution at a position corresponding to residue 138 of full length human p28 (e.g., full length murine p28 residue 134).

218. An IL27 receptor agonist, which is optionally an IL27 receptor agonist according to embodiment 217, which comprises a first p28 moiety comprising a p28 domain having an alanine substitution at a position corresponding to residue 138 of full length human p28 (e.g., full length murine p28 residue 134), and which optionally further comprises a second p28 moiety comprising a p28 domain having an alanine substitution at a position corresponding to residue 138 of full length human p28 (e.g., full length murine p28 residue 134).

219. The IL27 receptor agonist of any one of embodiments 82 to 218, wherein the first p28 moiety, and if present, the second p28 moiety comprise(s) a p28 domain having an amino acid substitution at a position corresponding to residue 142 of full length human p28 (e.g., full length murine p28 residue 138).

220. An IL27 receptor agonist, which is optionally an IL27 receptor agonist according to embodiment 219, which comprises a first p28 moiety comprising a p28 domain having an alanine substitution at a position corresponding to residue 142 of full length human p28 (e.g., full length murine p28 residue 138), and which optionally further comprises a second p28 moiety comprising a p28 domain having an alanine substitution at a position corresponding to residue 142 of full length human p28 (e.g., full length murine p28 residue 138).

221. The IL27 receptor agonist of any one of embodiments 82 to 220, wherein the first p28 moiety, and if present, the second p28 moiety comprise(s) a p28 domain having an amino acid substitution at a position corresponding to residue 145 of full length human p28 (e.g., full length murine p28 residue 141).

222. An IL27 receptor agonist, which is optionally an IL27 receptor agonist according to embodiment 221, which comprises a first p28 moiety comprising a p28 domain having an alanine substitution at a position corresponding to residue 145 of full length human p28 (e.g., full length murine p28 residue 141), and which optionally further comprises a second p28 moiety comprising a p28 domain having an alanine substitution at a position corresponding to residue 145 of full length human p28 (e.g., full length murine p28 residue 141).

223. The IL27 receptor agonist of any one of embodiments 82 to 222, wherein the first p28 moiety, and if present, the second p28 moiety comprise(s) a p28 domain having an amino acid substitution at a position corresponding to residue 146 of full length human p28 (e.g., full length murine p28 residue 142).

224. An IL27 receptor agonist, which is optionally an IL27 receptor agonist according to embodiment 223, which comprises a first p28 moiety comprising a p28 domain having an alanine substitution at a position corresponding to residue 146 of full length human p28 (e.g., full length murine p28 residue 142), and which optionally further comprises a second p28 moiety comprising a p28 domain having an alanine substitution at a position corresponding to residue 146 of full length human p28 (e.g., full length murine p28 residue 142).

225. The IL27 receptor agonist of any one of embodiments 82 to 224, wherein the first p28 moiety, and if present, the second p28 moiety comprise(s) a p28 domain having an amino acid substitution at a position corresponding to residue 149 of full length human p28 (e.g., full length murine p28 residue 145).

226. An IL27 receptor agonist, which is optionally an IL27 receptor agonist according to embodiment 225, which comprises a first p28 moiety comprising a p28 domain having an alanine substitution at a position corresponding to residue 149 of full length human p28 (e.g., full length murine p28 residue 145), and which optionally further comprises a second p28 moiety comprising a p28 domain having an alanine substitution at a position corresponding to residue 149 of full length human p28 (e.g., full length murine p28 residue 145).

227. The IL27 receptor agonist of any one of embodiments 82 to 226, wherein the first p28 moiety, and if present, the second p28 moiety comprise(s) a p28 domain having an amino acid substitution at a position corresponding to residue 150 of full length human p28 (e.g., full length murine p28 residue 146).

228. An IL27 receptor agonist, which is optionally an IL27 receptor agonist according to embodiment 227, which comprises a first p28 moiety comprising a p28 domain having an alanine substitution at a position corresponding to residue 150 of full length human p28 (e.g., full length murine p28 residue 146), and which optionally further comprises a second p28 moiety comprising a p28 domain having an alanine substitution at a position corresponding to residue 150 of full length human p28 (e.g., full length murine p28 residue 146).

229. The IL27 receptor agonist of any one of embodiments 82 to 228, wherein the first p28 moiety, and if present, the second p28 moiety comprise(s) a p28 domain having an amino acid substitution at a position corresponding to residue 197 of full length human p28 (e.g., full length murine p28 residue 195).

230. An IL27 receptor agonist, which is optionally an IL27 receptor agonist according to embodiment 229, which comprises a first p28 moiety comprising a p28 domain having an alanine substitution at a position corresponding to residue 197 of full length human p28 (e.g., full length murine p28 residue 195), and which optionally further comprises a second p28 moiety comprising a p28 domain having an alanine substitution at a position corresponding to residue 197 of full length human p28 (e.g., full length murine p28 residue 195).

231. The IL27 receptor agonist of any one of embodiments 82 to 230, wherein the first p28 moiety, and if present, the second p28 moiety comprise(s) a p28 domain having an amino acid substitution at a position corresponding to residue 200 of full length human p28 (e.g., full length murine p28 residue 198).

232. An IL27 receptor agonist, which is optionally an IL27 receptor agonist according to embodiment 231, which comprises a first p28 moiety comprising a p28 domain having an alanine substitution at a position corresponding to residue 200 of full length human p28 (e.g., full length murine p28 residue 198), and which optionally further comprises a second p28 moiety comprising a p28 domain having an alanine substitution at a position corresponding to residue 200 of full length human p28 (e.g., full length murine p28 residue 198).

233. The IL27 receptor agonist of any one of embodiments 82 to 232, wherein the first p28 moiety, and if present, the second p28 moiety comprise(s) a p28 domain having an amino acid substitution at a position corresponding to residue 201 of full length human p28 (e.g., full length murine p28 residue 199).

234. An IL27 receptor agonist, which is optionally an IL27 receptor agonist according to embodiment 233, which comprises a first p28 moiety comprising a p28 domain having an alanine substitution at a position corresponding to residue 201 of full length human p28 (e.g., full length murine p28 residue 199), and which optionally further comprises a second p28 moiety comprising a p28 domain having an alanine substitution at a position corresponding to residue 200 of full length human p28 (e.g., full length murine p28 residue 199).

235. The IL27 receptor agonist of any one of embodiments 82 to 234, wherein the first p28 moiety, and if present, the second p28 moiety comprise(s) a p28 domain having an amino acid substitution at a position corresponding to residue 204 of full length human p28 (e.g., full length murine p28 residue 202).

236. An IL27 receptor agonist, which is optionally an IL27 receptor agonist according to embodiment 235, which comprises a first p28 moiety comprising a p28 domain having an alanine substitution at a position corresponding to residue 204 of full length human p28 (e.g., full length murine p28 residue 202), and which optionally further comprises a second p28 moiety comprising a p28 domain having an alanine substitution at a position corresponding to residue 204 of full length human p28 (e.g., full length murine p28 residue 202).

237. The IL27 receptor agonist of any one of embodiments 82 to 236, wherein the first p28 moiety, and if present, the second p28 moiety comprise(s) a p28 domain having an amino acid substitution at a position corresponding to residue 205 of full length human p28 (e.g., full length murine p28 residue 203).

238. An IL27 receptor agonist, which is optionally an IL27 receptor agonist according to embodiment 237, which comprises a first p28 moiety comprising a p28 domain having an alanine substitution at a position corresponding to residue 205 of full length human p28 (e.g., full length murine p28 residue 203), and which optionally further comprises a second p28 moiety comprising a p28 domain having an alanine substitution at a position corresponding to residue 205 of full length human p28 (e.g., full length murine p28 residue 203).

239. The IL27 receptor agonist of any one of embodiments 24 to 238, which comprises a first multimerization moiety and a second multimerization moiety, wherein the first multimerization moiety and the second multimerization moiety are configured to dimerize together.

240. The IL27 receptor agonist of any one of embodiments 24 to 239, which comprises a first multimerization moiety and a second multimerization moiety, wherein the first multimerization moiety and the second multimerization moiety each is or comprises an Fc domain.

241. The IL27 receptor agonist of embodiment 240, wherein the Fc domain comprises a hinge domain.

242. The IL27 receptor agonist of embodiment 240 or embodiment 241, wherein the Fc domain is an IgG1, IgG2, IgG3, or IgG4 Fc domain.

243. The IL27 receptor agonist of any one of embodiments 240 to 242, wherein the Fc domain has reduced effector function.

244. The IL27 receptor agonist of any one of embodiment 240 to 243, wherein the Fc domain is an IgG4 Fc domain.

245. The IL27 receptor agonist of any one of embodiments 240 to 244, wherein the Fc domain comprises the amino acid sequence ESKYGPPCPPCPAPPVAGPSVFLFPPKPKDTLMISRTPEVTCVVVDVSQEDPEVQFNWYVDGVEV HNAKTKPREEQFNSTYRVVSVLTVLHQDWLNGKEYKCKVSNKGLPSSIEKTISKAKGQPREPQVYT LPPSQEEMTKNQVSLTCLVKGFYPSDIAVEWESNGQPENNYKTTPPVLDSDGSFFLYSRLTVDKSR WQEGNVFSCSVMHEALHNHYTQKSLSLSLGK (SEQ ID NO: 80) or a portion thereof.

246. The IL27 receptor agonist of any one of embodiments 82 to 245, wherein the stabilization moiety is human serum albumin or a natural variant thereof, a human serum albumin binder, an XTEN, a PAS, a carbohydrate, a polysialic acid, a hydrophilic polymer, or a fatty acid.

247. The IL27 receptor agonist of embodiment 246, wherein the stabilization moiety has an amino acid sequence with at least about 90%, at least about 95%, at least about 97%, at least about 98%, or at least about 99% to human serum albumin or a natural variant thereof.

248. The IL27 receptor agonist of embodiment 247, wherein the stabilization moiety has an amino acid sequence with at least about 90% sequence identity to mature human serum albumin or a natural variant thereof.

249. The IL27 receptor agonist of embodiment 247 or embodiment 248, wherein the stabilization moiety has an amino acid sequence with at least about 95% sequence identity to mature human serum albumin or a natural variant thereof.

250. The IL27 receptor agonist of any one of embodiments 247 to 249, wherein the stabilization moiety has an amino acid sequence with at least about 97% sequence identity to mature human serum albumin or a natural variant thereof.

251. The IL27 receptor agonist of any one of embodiments 247 to 250, wherein the stabilization moiety has an amino acid sequence with at least about 98% sequence identity to mature human serum albumin or a natural variant thereof.

252. The IL27 receptor agonist of any one of embodiments 247 to 251, wherein the stabilization moiety has an amino acid sequence with at least about 99% sequence identity to mature human serum albumin or a natural variant thereof.

253. The IL27 receptor agonist of embodiment 246, wherein the stabilization moiety is a human serum albumin binder.

254. The IL27 receptor agonist of embodiment of 247, wherein the human serum albumin binder is Adnectin PKE, AlbudAb, or an albumin binding domain.

255. The IL27 receptor agonist of embodiment 247, wherein the stabilization moiety is a hydrophilic polymer.

256. The IL27 receptor agonist of embodiment 255, wherein the hydrophilic polymer is polyethylene glycol (PEG).

257. The IL27 receptor agonist of embodiment 256, wherein the PEG has a molecular weight ranging from about 7.5 kDa to about 80 kDa.

258. The IL27 receptor agonist of embodiment 257, wherein the PEG has a molecular weight ranging from about 30 kDa to about 60 kDa, optionally wherein the molecular weight is about 50 kDa.

259. The IL27 receptor agonist of any one of embodiments 24 to 258, which comprises (A) a first targeting moiety or (B) a first means for binding to a target molecule.

260. The IL27 receptor agonist of embodiment 259, which comprises:
(a) a first targeting moiety component on the first polypeptide chain and further comprises a third targeting moiety component configured to associate with the first targeting moiety component to form the first targeting moiety, wherein the third targeting moiety component is not part of the first polypeptide or the second polypeptide; or
(b) a component of a first means for binding to a target molecule on the first polypeptide chain and a counterpart component that is not part of the first polypeptide or the second polypeptide.

261. The IL27 receptor agonist of any one of embodiments 24 to 260, which comprises (A) a second targeting moiety or (B) a second means for binding to a target molecule.

262. The IL27 receptor agonist of embodiment 261, which comprises:
(a) a second targeting moiety component on the second polypeptide chain and further comprises a fourth targeting moiety component configured to associate with the second targeting moiety component to form the second targeting moiety, wherein the fourth targeting moiety component is not part of the first polypeptide or the second polypeptide; or
(b) a component of a second means for binding to a target molecule on the first polypeptide chain and a counterpart component that is not part of the first polypeptide or the second polypeptide.

263. The IL27 receptor of any one of embodiments 259 to 262, wherein
(a)
   (i) the first targeting moiety or first targeting moiety component, if present on the first polypeptide chain, is N-terminal to the first multimerization moiety; or
   (ii) the first means for binding to a target molecule or component thereof, if present on the first polypeptide chain, is N-terminal to the first multimerization moiety;
and/or
(b)
   (i) the second targeting moiety or second targeting moiety component, if present on the second polypeptide chain, is N-terminal to the second multimerization moiety; or
   (ii) the second means for binding to a target molecule or component thereof, if present on the second polypeptide chain, is N-terminal to the first multimerization moiety.

264. The IL27 receptor of embodiment 259 to 262, wherein
(a)
   (i) the first targeting moiety or first targeting moiety component, if present on the first polypeptide chain, is C-terminal to the first multimerization moiety; or
   (ii) the first means for binding to a target molecule or component thereof, if present on the first polypeptide chain, is C-terminal to the first multimerization moiety;
and/or
(b)
   (i) the second targeting moiety or second targeting moiety component, if present on the second polypeptide chain, is C-terminal to the second multimerization moiety; or
   (ii) the second means for binding to a target molecule or component thereof, if present on the second polypeptide chain, is C the first means for binding to a target molecule or component thereof, if present on the first polypeptide chain on the first polypeptide chain, is N-terminal to the first multimerization moiety-terminal to the first multimerization moiety.

265. The IL27 receptor agonist of any one of embodiments 259 to 264, which comprises (A) a first targeting moiety or first means for binding to a target molecule and/or (B) a second targeting moiety or second means for binding to a target molecule, and wherein the first targeting moiety or first means and/or second target moiety or second means:
(a) binds to a disease-associated, e.g., tumor, inflammation or autoimmune disease-associated, antigen;
(b) binds to a disease microenvironment, e.g., tumor, inflammation or autoimmune disease microenvironment antigen;
(c) binds to a cell surface molecule of disease-reactive, e.g., tumor, inflammation or autoimmune disease reactive, lymphocytes;
(d) binds to a checkpoint inhibitor;
(e) binds to a peptide-MHC complex;
(f) is a peptide-MHC complex;
(g) binds to an antigen associated with or targeted an autoimmune response; or (h) is independently selected from (a) to (g) above.

266. The IL27 receptor agonist of embodiment 265, wherein the first targeting moiety or first means and the second targeting moiety or second means are the same.

267. The IL27 receptor agonist of embodiment 265 or embodiment 266, wherein the first targeting moiety or first means and/or second targeting moiety or second means bind(s) to a disease-associated, e.g., tumor, inflammation or autoimmune disease associated, antigen.

268. The IL27 receptor agonist of any one of embodiments 265 to 267, wherein the first targeting moiety or first means and/or second targeting moiety or second means bind(s) to Fibroblast Activation Protein (FAP), the A1 domain of Tenascin-C (TNC A1), the A2 domain of Tenascin-C (TNC A2), the Extra Domain B of Fibronectin (EDB), the Melanoma-associated Chondroitin Sulfate Proteoglycan (MCSP), MART-1/Melan-A, gp100, Dipeptidyl peptidase IV (DPPIV), adenosine deaminase-binding protein (ADAbp), cyclophilin b, colorectal associated antigen (CRC)-C017-1A/GA733, Carcinoembryonic Antigen (CEA) and its immunogenic epitopes CAP-1 and CAP-2, etv6, amli, Prostate Specific Antigen (PSA) or an immunogenic epitopes thereoPSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21 ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, c-erbB-2, Her2, EGFR, IGF-1R, CD2 (T-cell surface antigen), CD3 (heteromultimer associated with the TCR), CD22 (B-cell receptor), CD23 (low binding affinity IgE receptor), CD30 (cytokine receptor), CD33 (myeloid cell surface antigen), CD40 (tumor necrosis factor receptor), IL-6R-(IL6 receptor), CD20, MCSP, PDGFPR (p-platelet-derived growth factor receptor), ErbB2 epithelial cell adhesion molecule (EpCAM), EGFR variant III (EGFRvIII), CD19, disialoganglioside GD2, ductal-epithelial mucine, gp36, TAG-72, glioma-associated antigen, p-human chorionic gonadotropin, alphafetoprotein (AFP), lectin-reactive AFP, thyroglobulin, MN-CA IX, human telomerase reverse transcriptase, RU1, RU2 (AS), intestinal carboxyl esterase, mut hsp70-2, M-CSF, prostase, prostase specific antigen (PSA), PAP, LAGA-1a, p53, prostein, PSMA, surviving and telomerase, prostate-carcinoma tumor antigen-1 (PCTA-1), ELF2M, neutrophil elastase, ephrin B2, insulin growth factor (IGF1)-I, IGF-II, IGFI receptor, 5T4, ROR1, Nkp30, NKG2D, tumor stromal antigens, the extra domain A (EDA) or extra domain B (EDB) of fibronectin, or the A1 domain of tenascin-C (TnC A1).

269. The IL27 receptor agonist any one of embodiments 265 to 268, wherein the first targeting moiety or first means and/or second targeting moiety or second means bind(s) to a viral antigen.

270. The IL27 receptor agonist of one of embodiments 265 to 269, wherein the viral antigen is Epstein-Barr virus LMP-1, hepatitis C virus E2 glycoprotein, HIV gp160, or HIV gp120, HPV E6, HPV E7, CMV early membrane antigen (EMA) or CMV late membrane antigen (LMA).

271. The IL27 receptor agonist of any one of embodiments 265 to 267, wherein the first targeting moiety or first means and/or second targeting moiety or second means bind(s) to a disease, e.g., tumor, inflammation or autoimmune disease, microenvironment antigen.

272. The IL27 receptor agonist of embodiment 271, wherein the disease, e.g., tumor, inflammation or autoimmune disease, microenvironment antigen is an extracellular matrix protein.

273. The IL27 receptor agonist of embodiment 272, wherein the extracellular matrix protein is syndecan, heparanase, an integrin, osteopontin, link, cadherins, laminin, laminin type EGF, lectin, fibronectin, notch, tenascin, collagen or matrixin.

274. The IL27 receptor agonist of any one of embodiments 265 to 267, wherein the first targeting moiety or first means and/or second targeting moiety or second means bind(s) to a cell surface molecule of disease reactive, e.g., tumor, inflammation or autoimmune disease-reactive, lymphocytes.

275. The IL27 receptor agonist of embodiment 274, wherein the cell surface molecule is CD27, CD28, 4-1 BB (CD137), OX40, CD30, CD40, PD1, ICOS, lymphocyte function-associated antigen-1 (LFA-1), CD2, CD7, LIGHT, NKG2C, LAG3, TIM3, or B7-H3.

276. The IL27 receptor agonist of embodiment 275, wherein the cell surface molecule is PD1.

277. The IL27 receptor agonist of embodiment 276, first targeting moiety or first means and/or second targeting moiety or second means is an anti-PD1 antibody or antigen binding fragment thereof.

278. The IL27 receptor agonist of embodiment 277, wherein the anti-PD1 antibody or antigen binding fragment thereof inhibits PD1 signaling.

279. The IL27 receptor agonist of embodiment 277, wherein the anti-PD1 antibody or antigen binding fragment thereof does not inhibit PD1 signaling.

280. The IL27 receptor agonist of embodiment 275, wherein the cell surface molecule is LAG3.

281. The IL27 receptor agonist of embodiment 275, wherein the cell surface molecule is MADCAM, a4b7, integrin, TSHR or Epcam.

282. The IL27 receptor agonist of embodiment 265, wherein first targeting moiety or first means and/or second targeting moiety or second means bind(s) to a checkpoint inhibitor.

283. The IL27 receptor agonist of embodiment 281, wherein the checkpoint inhibitor is CTLA-4, PD1, PDL1, PDL2, PD1, B7-H3, B7-H4, BTLA, HVEM, TIM3, GAL9, LAG3, VISTA, KIR, 2B4, CD160, CGEN-15049, CHK1, VISTA, PSGL1, or CHK2.

284. The IL27 receptor agonist of embodiment 283, wherein the checkpoint inhibitor is PD1.

285. The IL27 receptor agonist of embodiment 284, wherein the first targeting moiety or first means and/or second targeting moiety or second means is an anti-PD1 antibody or antigen binding fragment thereof.

286. The IL27 receptor agonist of embodiment 285, wherein the anti-PD1 antibody or antigen binding fragment thereof inhibits PD1 signaling.

287. The IL27 receptor agonist of embodiment 285, wherein the anti-PD1 antibody or antigen binding fragment thereof does not inhibit PD1 signaling.

288. The IL27 receptor agonist of embodiment 283, wherein the checkpoint inhibitor is LAG3.

289. The IL27 receptor agonist of embodiment 284, wherein first targeting moiety or first means and/or second targeting moiety or second means bind(s) to an MHC-peptide complex.

290. The IL27 receptor agonist of embodiment 289 wherein the peptide in the peptide-MHC complex comprises a tumor neoantigen.

291. The IL27 receptor agonist of embodiment 290, wherein the tumor neoantigen is LCMV derived peptide gp33-41, APF (126-134), BALF (276-284), CEA (571-579), CMV pp65 (495-503), FLU-M1 (58-66), gp100 (154-162), gp100 (209-217), HBV Core (18-27), Her2/neu (369-377; V2v9); HPV E7 (11-20), HPV E7 (11-19), HPV E7 (82-90), KLK4 (11-19), LMP1 (125-133), MAG-A3 (112-120), NYESO1 (157-165, C165A), NYESO1 (157-165, C165V), p54 WT (264-272), PAP-3 (136-143), PSMA (4-12), PSMA (135-145), Survivin (96-014), Tyrosinase (369-377, 371D), or WT1 (126-134).

292. The IL27 receptor agonist of any one of embodiments 289 to 291, wherein first targeting moiety or first means and/or second targeting moiety or second means comprises an antibody or antigen binding fragment thereof having complementarity determining regions (“CDRs”) comprising:
(a) a CDR-H1 having an amino acid sequence selected from any of SEQ ID NOs: 4, 20, 36, 52, 68, 84, 100, 116, 132, 148, 164, 180, 196, 212, 220, 236, 252, 268, 284, 300, 316, 332, 348, 364, 380, 396, 412, 428, 444, 460, 476, 492, 508, and 524 of International Patent Publication No. WO 2019005897 A1, which are incorporated by reference herein;
(b) a CDR-H2 having an amino acid sequence selected from any of SEQ ID NOs: 6, 22, 38, 54, 70, 86, 102, 118, 134, 150, 166, 182, 198, 214, 222, 238, 254, 270, 286, 302, 318, 334, 350, 366, 382, 414, 430, 446, 462, 478, 494, 510, and 526 of International Patent Publication No. WO 2019005897 A1, which are incorporated by reference herein;
(c) a CDR-H3 having an amino acid sequence selected from any of SEQ ID NOs: 8, 24, 40, 56, 72, 88, 104, 120, 136, 152, 168, 184, 200, 216, 224, 240, 256, 272, 288, 304, 320, 336, 352, 368, 384, 400, 416, 432, 448, 464, 480, 496, 512, and 528 of International Patent Publication No. WO 2019005897 A1, which are incorporated by reference herein;
(d) a CDR-L1 having an amino acid sequence selected from any of SEQ ID NOs: 12, 28, 44, 60, 76, 92, 108, 124, 140, 156, 172, 188, 204, 204, 228, 244, 260, 276, 292, 308, 324, 340, 356, 372, 388, 404, 420, 436, 452, 468, 484, 500, 516, and 532 of International Patent Publication No. WO 2019005897 A1, which are incorporated by reference herein;
(e) a CDR-L2 having an amino acid sequence selected from any of SEQ ID NOs: 14, 30, 46, 62, 78, 94, 110, 126, 142, 158,174, 190, 206, 230, 246, 262, 278, 294, 310, 326, 342, 358, 374, 390, 406, 422, 438, 454, 470, 486, 502, 518, and 534 of International Patent Publication No. WO 2019005897 A1, which are incorporated by reference herein; and
(f) a CDR-L3 having an amino acid sequence selected from an of SEQ ID NOs: 16. 32, 48, 64, 80, 96, 112, 128, 144, 160, 176, 192, 208, 232, 248, 264, 280, 296, 312, 328, 344, 360, 376, 392, 408, 424, 440, 456, 472, 488, 504, 520, and 536 of International Patent Publication No. WO 2019005897 A1, which are incorporated by reference herein.

293. The IL27 receptor agonist of embodiment 292, wherein the antibody or antigen binding fragment has VH-VL amino acid sequences selected from any of SEQ ID NOs: 2/10, 18/26, 34/42, 50/58, 66/74, 82/90, 98/106, 114/122, 130/138, 146/154, 162/170, 178/186, 194/202, 210/202, 218/226, 234/242, 250/258, 266/274, 282/290, 298/306, 314/322, 330/338, 346/354, 362/370, 378/386, 394/402, 410/418, 426/434, 442/450, 458/466, 474/482, 490/498, 506/514, and 522/530 of International Patent Publication No. WO 2019005897 A1, which are incorporated by reference herein.

294. The IL27 receptor agonist of embodiment 293, wherein the antibody or antigen binding fragment has VH-VL amino acid sequences selected from any of SEQ ID NOs: 2/10, 34/42, 82/90, 194/202, 282/290, and 506/514 of International Patent Publication No. WO 2019005897 A1, which are incorporated by reference herein.

295. The IL27 receptor agonist of embodiment 265 or embodiment 266, wherein the first targeting moiety or first means and/or second targeting moiety or second means bind(s) an antigen associated with or targeted by an autoimmune response.

296. The IL27 receptor agonist of embodiment 295, wherein the peptide is derived from gliadin, GAD 65, IA-2, insulin B chain, glatiramer acetate (GA), achetylcholine receptor (AChR), p205, insulin, thyroid-stimulating hormone, tyrosinase, TRP I, or a myelin antigen.

297. The IL27 receptor agonist of embodiment 296, wherein the peptide is derived from IL-4R, IL-6R, or DLL4.

298. The IL27 receptor agonist of embodiment 265 or embodiment 266, wherein the first targeting moiety or first means and/or second targeting moiety or second means bind(s) to an immune cell.

299. The IL27 receptor agonist of embodiment 298, wherein the immune cell is a T lymphocyte, a B lymphocyte, or a dendritic cell.

300. The IL27 receptor agonist of embodiment 299, wherein the immune cell is a T lymphocyte and the target is CD2, CD3, CD4, CD7, CD8, XCR1, Clec9a, or CD20.

301. The IL27 receptor agonist of any one of embodiments 265 to 300, wherein the first targeting moiety or first means and/or second targeting moiety or second means is an antibody or antigen binding fragment thereof.

302. The IL27 receptor agonist of any one of embodiments 265 to 301, wherein the first targeting moiety or first means and/or second targeting moiety or second means bind(s) is a Fab.

303. The IL27 receptor agonist of any one of embodiments 265 to 301, wherein the first targeting moiety or first means and/or second targeting moiety or second means is an scFv.

304. The IL27 receptor agonist of embodiment 265 or embodiment 266, wherein the first targeting moiety or first means and/or second targeting moiety or second means is a peptide-MHC complex.

305. The IL27 receptor agonist of embodiment 304, wherein the peptide-MHC complex binds to the T cell receptor of tumor lymphocytes.

306. The IL27 receptor agonist of embodiment 304 or embodiment 305, wherein the peptide in the peptide-MHC complex comprises a tumor neoantigen.

307. The IL27 receptor agonist of embodiment 306, wherein the tumor neoantigen is LCMV derived peptide gp33-41, APF (126-134), BALF (276-284), CEA (571-579), CMV pp65 (495-503), FLU-M1 (58-66), gp100 (154-162), gp100 (209-217), HBV Core (18-27), Her2/neu (369-377; V2v9); HPV E7 (11-20), HPV E7 (11-19), HPV E7 (82-90), KLK4 (11-19), LMP1 (125-133), MAG-A3 (112-120), NYESO1 (157-165, C165A), NYESO1 (157-165, C165V), p54 WT (264-272), PAP-3 (136-143), PSMA (4-12), PSMA (135-145), Survivin (96-014), Tyrosinase (369-377, 371D), or WT1 (126-134).

308. The IL27 receptor agonist of embodiment 304, wherein the peptide in peptide-MHC complex comprises a viral antigen.

309. The IL27 receptor agonist of embodiment 308, wherein the viral antigen is CMVpp65 or HPV16E7.

310. The IL27 receptor agonist of any one of embodiments 304 to 309, wherein the peptide-MHC complex further comprises β2 microglobulin or a fragment thereof.

311. The IL27 receptor agonist of embodiment 310, wherein the peptide MHC complex comprises a type I MHC domain.

312. The IL27 receptor agonist of embodiment 311, wherein the peptide MHC complex comprises, in an N- to C-terminal orientation a MHC peptide, a linker, a P2-microglobulin domain, a linker, and a type I MHC domain.

313. The IL27 receptor agonist of embodiment 312, wherein the linker connecting the MHC peptide and the β2-microglobulin domain comprises the amino acid sequence GCGGS (SEQ ID NO: 24).

314. The IL27 receptor agonist of any one of embodiment 304 to 309, wherein the peptide-MHC complex does not comprise P2 microglobulin or a fragment thereof.

315. The IL27 receptor agonist of embodiment 314, wherein the peptide MHC complex comprises a type II MHC domain.

316. The IL27 receptor agonist of any one of embodiments 2 to 315, further comprising an IL27 receptor subunit or an IL27 binding portion thereof.

317. The IL27 receptor agonist of embodiment 276, wherein the IL27 receptor subunit is IL27Ra (IL27Rα).

318. The IL27 receptor agonist of embodiment 276, wherein the IL27 receptor subunit is gp130.

319. A p28 protein comprising:
(a) a first polypeptide comprising:
   (i) a first targeting moiety (or component thereof) or first means for binding to a target molecule (or component thereof);
   (ii) an optional first linker; and
   (iii) a first multimerization moiety; and
(b) a second polypeptide comprising:
   (i) a p28 moiety, which is optionally a p28 moiety as defined in any one of embodiments 1 to 23;
   (ii) an optional second linker; and
   (iii) a second multimerization moiety associated with the first multimerization moiety.

320. The p28 protein of embodiment 319, wherein the first targeting moiety or first means is a Fab.

321. The p28 protein of embodiment 319, wherein the first targeting moiety or first means is an scFv.

322. A p28 protein comprising:
(a) a first polypeptide comprising:
   (i) a first targeting moiety (or component thereof) or first means for binding to a target molecule (or component thereof);
   (ii) an optional first linker; and
   (iii) a first multimerization moiety; and
(b) a second polypeptide comprising:
   (i) a second targeting moiety (or component thereof) or second means for binding to a target molecule (or component thereof);
   (ii) an optional second linker;
   (iii) a second multimerization moiety associated with the first multimerization moiety;
   (iv) an optional third linker; and
   (v) a p28 moiety, which is optionally a p28 moiety as defined in any one of embodiments 1 to 23.

323. The p28 protein of embodiment 322, wherein the first targeting moiety (or first means) and second targeting moiety (or second means) are Fabs.

324. The p28 protein of embodiment 322, wherein the first targeting moiety (or first means) and second targeting moiety (or second means) are scFvs.

325. The p28 protein of any one of embodiments 322 to 324, wherein the first and second targeting moieties (or first and second means) are the same.

326. The p28 protein of any one of embodiments 319 to 325 which lacks an EBI3 moiety.

327. An EBI3 protein comprising:
(a) a first polypeptide comprising:
   (i) an EBI3 moiety;
   (ii) an optional first linker; and
   (iii) a first multimerization moiety; and
(b) a second polypeptide comprising:
   (i) a first targeting moiety (or component thereof) or first means for binding to a target molecule (or component thereof);
   (ii) an optional second linker; and
   (iii) a second multimerization moiety associated with the first multimerization moiety.

328. The EBI3 protein of embodiment 327, wherein the first targeting moiety or first means is a Fab.

329. The EBI3 protein of embodiment 327, wherein the first targeting moiety or first means is an scFv.

330. An EBI3 protein comprising:
(a) a first polypeptide comprising:
   (i) a first targeting moiety (or component thereof) or first means for binding to a target molecule (or component thereof);
   (ii) an optional linker;
   (iii) a first multimerization moiety;
   (iv) an optional second linker; and
   (v) an EIB3 moiety; and
(b) a second polypeptide comprising:
   (i) a second targeting moiety (or component thereof) or second means for binding to a target molecule (or component thereof);
   (ii) an optional third linker; and
   (iii) a second multimerization moiety associated with the first multimerization moiety.

331. The EBI3 protein of embodiment 330, wherein the first targeting moiety (or first means) and second targeting moiety (or second means) are Fabs.

332. The EBI3 protein of embodiment 330, wherein the first targeting moiety (or first means) and second targeting moiety (or second means) are scFvs.

333. The EBI3 protein of any one of embodiments 330 to 332, wherein the first targeting moiety (or first means) and second targeting moiety (or second means) are the same.

334. The EBI3 protein of any one of embodiments 330 to 333, which lacks a p28 moiety.

335. A nucleic acid or plurality of nucleic acids encoding the IL27 agonist of any preceding embodiment, e.g., any one of embodiments 24 to 318.

336. A nucleic acid or plurality of nucleic acids encoding the p28 protein of any one of embodiments 1 to 23 and 319 to 326.

337. A nucleic acid or plurality of nucleic acids encoding the EBI3 protein of any one of embodiments 327 to 334.

338. A host cell engineered to express the IL27 agonist of any preceding embodiment, e.g., any one of embodiments 24 to 318 or the nucleic acid or plurality of nucleic acids of embodiment 335.

339. A host cell engineered to express the p28 protein of any one of embodiments 1 to 23 and 319 to 326 or the nucleic acid or plurality of nucleic acids of embodiment 336.

340. A host cell engineered to express the EBI3 protein of any one of embodiments 327 to 334 or the nucleic acid or plurality of nucleic acids of embodiment 337.

341. A method of producing the IL27 agonist of any preceding embodiment, e.g., any one of embodiments 24 to 318, comprising culturing the host cell of embodiment 338 and recovering the IL27 agonist expressed thereby.

342. A method of producing the p28 protein of any one of embodiments 1 to 23 and 319 to 326, comprising culturing the host cell of embodiment 339 and recovering the p28 protein expressed thereby.

343. A method of producing the EBI3 protein of any one of embodiments 327 to 334, comprising culturing the host cell of embodiment 340 and recovering the EBI3 protein expressed thereby.

344. A pharmaceutical composition comprising the IL27 agonist of any preceding embodiment, e.g., any one of embodiments 24 to 318, and an excipient.

345. A pharmaceutical composition comprising the p28 protein of any one of embodiments 1 to 23 and 319 to 326 and an excipient.

346. A pharmaceutical composition comprising the EBI3 protein of any one of embodiments 327 to 334 and an excipient.

347. A method of modulating the immune response or treating an autoimmune condition, comprising administering to a subject in need thereof:
(a) the IL27 agonist of any preceding embodiment, e.g., any one of embodiments 24 to to 318;
(b) the p28 protein of any one of embodiments 1 to 23 and 319 to 326;
(c) the EBI3 protein of any one of embodiments 327 to 334;
(d) the p28 protein of any one of embodiments 1 to 23 and 319 to 326 in combination with the EBI3 protein of any one of embodiments 327 to 334;
(e) the pharmaceutical composition of any one of embodiments 344 to 346; or
(f) the pharmaceutical composition of embodiment 345 in combination with the pharmaceutical composition of embodiment 346.

348. A method of targeted treatment of an inflammatory or immune condition, comprising administering to a subject in need thereof:
(a) the IL27 agonist of any preceding embodiment, e.g., any one of embodiments 24 to 318;
(b) the p28 protein of any one of embodiments 1 to 23 and 319 to 326;
(c) the EBI3 protein of any one of embodiments 327 to 334;
(d) the p28 protein of any one of embodiments 1 to 23 and 319 to 326 in combination with the EBI3 protein of any one of embodiments 327 to 334;
(e) the pharmaceutical composition of any one of embodiments 344 to 346; or
(f) the pharmaceutical composition of embodiment 345 in combination with the pharmaceutical composition of embodiment 346,

wherein the IL27 receptor agonist, the p28 protein, the EBI3 protein comprises one or two targeting moieties (or one or two means for binding to a target molecule), optionally wherein the one or two targeting moieties (or one or two means for binding to a target molecule) are as defined in any one of preceding embodiments or as described in Section 5.7.

349. A method of localized delivery of an IL27 protein, a p28 protein or EBI3 protein or combination thereof, comprising administering to a subject in need thereof:
(a) the IL27 agonist of any preceding embodiment, e.g., any one of embodiments 24 to 318;
(b) the p28 protein of any one of embodiments 1 to 23 and 319 to 326;
(c) the EBI3 protein of any one of embodiments 327 to 334;
(d) the p28 protein of any one of embodiments 1 to 23 and 319 to 326 in combination with the EBI3 protein of any one of embodiments 327 to 334;
(e) the pharmaceutical composition of any one of embodiments 344 to 346; or
(f) the pharmaceutical composition of embodiment 345 in combination with the pharmaceutical composition of embodiment 346,

wherein the IL27 receptor agonist, p28 protein or EBI3 protein or combination thereof, comprises one or two targeting moieties, optionally wherein the one or two targeting moieties are as defined in any one of preceding embodiments or as described in Section 5.7.

350. A method of administering to the subject IL27 therapy with reduced systemic exposure and/or reduced systemic toxicity, comprising administering to a subject:
(a) the IL27 agonist of any preceding embodiment, e.g., any one of embodiments 24 to 318;
(b) the p28 protein of any one of embodiments 1 to 23 and 319 to 326;
(c) the EBI3 protein of any one of embodiments 327 to 334;
(d) the p28 protein of any one of embodiments 1 to 23 and 319 to 326 in combination with the EBI3 protein of any one of embodiments 327 to 334;
(e) the pharmaceutical composition of any one of embodiments 344 to 346; or
(f) the pharmaceutical composition of embodiment 345 in combination with the pharmaceutical composition of embodiment 346,

optionally wherein the IL27 receptor agonist, p28 protein or EBI3 protein or combination thereof, (a) comprises one or two targeting moieties, optionally wherein the one or two targeting moieties are as defined in any one of preceding embodiments or as described in Section 5.7 and/or (b) an IL27 (e.g., p28 and/or EBI3) moiety that is attenuated through mutation and/or masking (e.g., by an IL27 receptor) or as described in Section 5.6.

351. A method of locally modulating an immune response in a target tissue, comprising administering to a subject in need thereof:
(a) the IL27 agonist of any preceding embodiment, e.g., any one of embodiments 24 to 318;
(b) the p28 protein of any one of embodiments 1 to 23 and 319 to 326;
(c) the EBI3 protein of any one of embodiments 327 to 334;
(d) the p28 protein of any one of embodiments 1 to 23 and 319 to 326 in combination with the EBI3 protein of any one of embodiments 327 to 334;
(e) the pharmaceutical composition of any one of embodiments 344 to 346; or
(f) the pharmaceutical composition of embodiment 345 in combination with the pharmaceutical composition of embodiment 346,

optionally wherein the IL27 receptor agonist, p28 protein or EBI3 protein or combination thereof, (a) comprises one or two targeting moieties, optionally wherein the one or two targeting moieties are as defined in any one of preceding embodiments or as described in Section 5.7 and/or (b) an IL27 (e.g., p28 and/or EBI3) moiety that is attenuated through mutation and/or masking (e.g., by an IL27 receptor) or as described in Section 5.6.

352. The method of any one of embodiments 347 to 351, wherein the method is for treating and/or wherein the subject is suffering from an autoimmune condition.

353. The method of embodiment 352, wherein the autoimmune condition is arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, scleroderma, systemic sclerosis, or allergic asthma.

354. The method of any one of embodiments 347 to 352, wherein the administration is systemic, optionally intravenous.

355. The method of any one of embodiments 347 to 352, wherein the administration is subcutaneous.

### 7. EXAMPLES

### 7.1. Materials and Methods

7.1.1. Production of IL27 Agonists

Constructs encoding the IL27 and IL27 muteins (identified with an IL27M_) listed in Table 6 below and Fc controls were generated. It is noted that the description of IL27M1 through IL27M21 and associated schematics in the present disclosure (e.g., in FIGS. 3B-3G, 4A-4G, 5A-5B and 6A-6E and in Section 5.2) are intended to describe an overall IL27 agonist architecture rather than any particular amino acid sequence. Thus, reference to a particular mutein architecture (e.g., IL27M1, IL27M2, etc.) in relation to a particular amino acid sequence (or pair of amino acid sequences) in these examples is not intended to be limiting but rather to serve as an indication of the architecture of the particular amino acid sequence (or pair of amino acid sequences). The IL27M__nomenclature is thus meant to demark generic structural configurations for a genus of IL27 agonists.

The IL27 mutein constructs included different configurations of murine or human IL27, an IgG1 or an IgG4 Fc domain, and linkers of different lengths from different repeats of G₄S (SEQ ID NO: 38) or G₃S (SEQ ID NO: 90):

Human EBI3:
(SEQ ID NO: 33)
MTPQLLLALVLWASCPPCSGRKGPPAALTLPRVQCRASRYPIAVDCSWTL
PPAPNSTSPVSFIATYRLGMAARGHSWPCLQQTPTSTSCTITDVQLFSMA
PYVLNVTAVHPWGSSSSFVPFITEHIIKPDPPEGVRLSPLAERQLQVQWE
PPGSWPFPEIFSLKYWIRYKRQGAARFHRVGPIEATSFILRAVRPRARYY
VQVAAQDLTDYGELSDWSLPATATMSLGK
Human p28:
(SEQ ID NO: 34)
MGQTAGDLGWRLSLLLLPLLLVQAGVWGFPRPPGRPQLSLQELRREFTVS
LHLARKLLSEVRGQAHRFAESHLPGVNLYLLPLGEQLPDVSLTFQAWRRL
SDPERLCFISTTLQPFHALLGGLGTQGRWTNMERMQLWAMRLDLRDLQRH
LRFQVLAAGFNLPEEEEEEEEEEEEERKGLLPGALGSALQGPAQVSWPQL
LSTYRLLHSLELVLSRAVRELLLLSKAGHSVWPLGFPTLSPQP
Murine EBI3:
(SEQ ID NO: 35)
MSKLLFLSLALWASRSPGYTETALVALSQPRVQCHASRYPVAVDCSWTPL
QAPNSTRSTSFIATYRLGVATQQQSQPCLQRSPQASRCTIPDVHLFSTVP
YMLNVTAVHPGGASSSLLAFVAERIIKPDPPEGVRLRTAGQRLQVLWHPP
ASWPFPDIFSLKYRLRYRRRGASHFRQVGPIEATTFTLRNSKPHAKYCIQ
VSAQDLTDYGKPSDWSLPGQVESAPHKP
Murine p28:
(SEQ ID NO: 36)
MGQVTGDLGWRLSLLLLPLLLVQAGSWGFPTDPLSLQELRREFTVSLYLA
RKLLSEVQGYVHSFAESRLPGVNLDLLPLGYHLPNVSLTFQAWHHLSDSE
RLCFLATTLRPFPAMLGGLGTQGTWTSSEREQLWAMRLDLRDLHRHLRFQ
VLAAGFKCSKEEEDKEEEEEEEEEEKKLPLGALGGPNQVSSQVSWPQLLY
TYQLLHSLELVLSRAVRDLLLLSLPRRPGSAWDS
mIgG1-Fc
IgG1 with N276D, N278D
(SEQ ID NO: 37)
VPRDCGCKPCICTVPEVSSVFIFPPKPKDVLTITLTPKVTCWVDISKDDP
EVQFSWFVDDVEVHTAQTQPREEQFNSTFRSVSELPIMHQDWLNGKEFKC
RVNSAAFPAPIEKTISKTKGRPKAPQVYTIPPPKEQMAKDKVSLTCMITD
FFPEDITVEWQWNGQPAENYKNTQPIMDTDGSYFVYSKLNVQKSNWEAGN
TFTCSVLHEGLHNHHTEKSLSHSPGK
G₄S:
(SEQ ID NO: 38)
GGGGS

The constructs were expressed in Expi293F™ cells (Thermo Fisher Scientific) by transient transfection. Proteins in Expi293F™ supernatant were purified using the ProteinMaker system (Protein BioSolutions, Gaithersburg, Md.) with either HiTrap™ Protein G HP or MabSelect SuRe pcc columns (Cytiva). After single step elution, the antibodies were neutralized, dialyzed into a final buffer of phosphate buffered saline (PBS) with 5% glycerol, aliquoted and stored at −80° C.

TABLE 6

| | Molecule/ | | | |
|---|---|---|---|---|
| | Generic | | | |
| | Archi- | Alternate | Description of | |
| SEQ ID | tecture | Name | Exemplary Embodiment | Sequence of Exemplary Embodiment |
| SEQ ID NO: | IL27 | mIL27 | Recombinant murine | YTETALVALSQPRVQCHASRYPVAVDCSWTPLQAPNSTRSTSFIA |
| 39 | | | IL27 (obtained from | TYRLGVATQQQSQPCLQRSPQASRCTIPDVHLFSTVPYMLNVTAV |
| | | | R&D Systems cat# | HPGGASSSLLAFVAERIIKPDPPEGVRLRTAGQRLQVLWHPPASW |
| | | | 2700-ML-010/CF) | PFPDIFSLKYRLRYRRRGASHFRQVGPIEATTFTLRNSKPHAKYC |
| | | | Mouse EBI3-(GGGS)₄- | IQVSAQDLTDYGKPSDWSLPGQVESAPHKPGGGSGGGSGGGSGGG |
| | | | Mouse p28-6-His tag | SFPTDPLSLQELRREFTVSLYLARKLLSEVQGYVHSFAESRLPGV |
| | | | | NLDLLPLGYHLPNVSLTFQAWHHLSDSERLCFLATTLRPFPAMLG |
| | | | | GLGTQGTWTSSEREQLWAMRLDLRDLHRHLRFQVLAAGFKCSKEE |
| | | | | EDKEEEEEEEEEEKKLPLGALGGPNQVSSQVSWPQLLYTYQLLHS |
| | | | | LELVLSRAVRDLLLLSLPRRPGSAWDSHHHHHH |
| SEQ ID NO: | IL27 | hIL27 | Recombinant human IL27 | RKGPPAALTLPRVQCRASRYPIAVDCSWTLPPAPNSTSPVSFIAT |
| 40 | | | (obtained from R&D | YRLGMAARGHSWPCLQQTPTSTSCTITDVQLFSMAPYVLNVTAVH |
| | | | Systems cat# | PWGSSSSFVPFITEHIIKPDPPEGVRLSPLAERQLQVQWEPPGSW |
| | | | 2526-IL-010/CF) | PFPEIFSLKYWIRYKRQGAARFHRVGPIEATSFILRAVRPRARYY |
| | | | Human EBI3- | VQVAAQDLTDYGELSDWSLPATATMSLGKGSGSSRGGSGSGGSGG |
| | | | GSGSSRGGSGSGGSGGGGSK | GGSKLAGVWGFPRPPGRPQLSLQELRREFTVSLHLARKLLSEVRG |
| | | | L-Human p28-6-His tag | QAHRFAESHLPGVNLYLLPLGEQLPDVSLTFQAWRRLSDPERLCF |
| | | | | ISTTLQPFHALLGGLGTQGRWTNMERMQLWAMRLDLRDLQRHLRF |
| | | | | QVLAAGFNLPEEEEEEEEEEEEERKGLLPGALGSALQGPAQVSWP |
| | | | | QLLSTYRLLHSLELVLSRAVRELLLLSKAGHSVWPLGFPTLSPQP |
| | | | | HHHHHH |
| SEQ ID NO: | IL27M1 | IL27 | Mouse EBI3-(GGGS)₄-p28- | YTETALVALSQPRVQCHASRYPVAVDCSWTPLQAPNSTRSTSFIA |
| 41 | | Mutein 1 | (GGGGS)₃-Fc | TYRLGVATQQQSQPCLQRSPQASRCTIPDVHLFSTVPYMLNVTAV |
| | | | Fc is SEQ ID NO: 31 of | HPGGASSSLLAFVAERIIKPDPPEGVRLRTAGQRLQVLWHPPASW |
| | | | WO2014/121087, which | PFPDIFSLKYRLRYRRRGASHFRQVGPIEATTFTLRNSKPHAKYC |
| | | | is an IgG4 with reduced | IQVSAQDLTDYGKPSDWSLPGQVESAPHKPGGGSGGGSGGGSGGG |
| | | | effector function | SFPTDPLSLQELRREFTVSLYLARKLLSEVQGYVHSFAESRLPGV |
| | | | | NLDLLPLGYHLPNVSLTFQAWHHLSDSERLCFLATTLRPFPAMLG |
| | | | | GLGTQGTWTSSEREQLWAMRLDLRDLHRHLRFQVLAAGFKCSKEE |
| | | | | EDKEEEEEEEEEEKKLPLGALGGPNQVSSQVSWPQLLYTYQLLHS |
| | | | | LELVLSRAVRDLLLLSLPRRPGSAWDSGGGGSGGGGSGGGGSESK |
| | | | | YGPPCPPCPAPPVAGPSVFLFPPKPKDTLMISRTPEVTCVVVDVS |
| | | | | QEDPEVQFNWYVDGVEVHNAKTKPREEQFNSTYRVVSVLTVLHQD |
| | | | | WLNGKEYKCKVSNKGLPSSIEKTISKAKGQPREPQVYTLPPSQEE |
| | | | | MTKNQVSLTCLVKGFYPSDIAVEWESNGQPENNYKTTPPVLDSDG |
| | | | | SFFLYSRLTVDKSRWQEGNVFSCSVMHEALHNHYTQKSLSLSLGK |
| SEQ ID NO: | IL27M2- | IL27 | Mouse p28-(GGGGS)₃-Fc | FPTDPLSLQELRREFTVSLYLARKLLSEVQGYVHSFAESRLPGVN |
| 42 | chain A | Mutein 2 | Fc hIgG4 stealth with | LDLLPLGYHLPNVSLTFQAWHHLSDSERLCFLATTLRPFPAMLGG |
| | | chain A | T245W mutation (Knob) | LGTQGTWTSSEREQLWAMRLDLRDLHRHLRFQVLAAGFKCSKEEE |
| | | | | DKEEEEEEEEEEKKLPLGALGGPNQVSSQVSWPQLLYTYQLLHSL |
| | | | | ELVLSRAVRDLLLLSLPRRPGSAWDSGGGGSGGGGSGGGGSESKY |
| | | | | GPPCPPCPAPPVAGPSVFLFPPKPKDTLMISRTPEVTCWVDVSQE |
| | | | | DPEVQFNWYVDGVEVHNAKTKPREEQFNSTYRVVSVLTVLHQDWL |
| | | | | NGKEYKCKVSNKGLPSSIEKTISKAKGQPREPQVYTLPPSQEEMT |
| | | | | KNQVSLWCLVKGFYPSDIAVEWESNGQPENNYKTTPPVLDSDGSF |
| | | | | FLYSRLTVDKSRWQEGNVFSCSVMHEALHNHYTQKSLSLSLGK |
| SEQ ID NO: | IL27M2- | IL27 | Mouse EBI3-(GGGGS)₃-Fc | YTETALVALSQPRVQCHASRYPVAVDCSWTPLQAPNSTRSTSFIA |
| 43 | Chain B | Mutein 2 | Fc hIgG4 stealth/star | TYRLGVATQQQSQPCLQRSPQASRCTIPDVHLFSTVPYMLNVTAV |
| | | chain B | with T245S, L247V, | HPGGASSSLLAFVAERIIKPDPPEGVRLRTAGQRLQVLWHPPASW |
| | | | Y286V mutations (Hole) | PFPDIFSLKYRLRYRRRGASHFRQVGPIEATTFTLRNSKPHAKYC |
| | | | | IQVSAQDLTDYGKPSDWSLPGQVESAPHKPGGGGSGGGGSGGGGS |
| | | | | ESKYGPPCPPCPAPPVAGPSVFLFPPKPKDTLMISRTPEVTCVVV |
| | | | | DVSQEDPEVQFNWYVDGVEVHNAKTKPREEQFNSTYRVVSVLTVL |
| | | | | HQDWLNGKEYKCKVSNKGLPSSIEKTISKAKGQPREPQVYTLPPS |
| | | | | QEEMTKNQVSLSCAVKGFYPSDIAVEWESNGQPENNYKTTPPVLD |
| | | | | SDGSFFLVSRLTVDKSRWQEGNVFSCSVMHEALHNRFTQKSLSLS |
| | | | | PGK |
| SEQ ID NO: | IL27M3 | | Bivalent p28-EBI3-Fc | FPTDPLSLQELRREFTVSLYLARKLLSEVQGYVHSFAESRLPGVN |
| 44 | | | Fc is human IgG4s | LDLLPLGYHLPNVSLTFQAWHHLSDSERLCFLATTLRPFPAMLGG |
| | | | | LGTQGTWTSSEREQLWAMRLDLRDLHRHLRFQVLAAGFKCSKEEE |
| | | | | DKEEEEEEEEEEKKLPLGALGGPNQVSSQVSWPQLLYTYQLLHSL |
| | | | | ELVLSRAVRDLLLLSLPRRPGSAWDSGGGGSGGGGSGGGGSGGGG |
| | | | | SGGGGSYTETALVALSQPRVQCHASRYPVAVDCSWTPLQAPNSTR |
| | | | | STSFIATYRLGVATQQQSQPCLQRSPQASRCTIPDVHLFSTVPYM |
| | | | | LNVTAVHPGGASSSLLAFVAERIIKPDPPEGVRLRTAGQRLQVLW |
| | | | | HPPASWPFPDIFSLKYRLRYRRRGASHFRQVGPIEATTFTLRNSK |
| | | | | PHAKYCIQVSAQDLTDYGKPSDWSLPGQVESAPHKPGGGGSGGGG |
| | | | | SGGGGSESKYGPPCPPCPAPPVAGPSVFLFPPKPKDTLMISRTPE |
| | | | | VTCVWDVSQEDPEVQFNWYVDGVEVHNAKTKPREEQFNSTYRVVS |
| | | | | VLTVLHQDWLNGKEYKCKVSNKGLPSSIEKTISKAKGQPREPQVY |
| | | | | TLPPSQEEMTKNQVSLTCLVKGFYPSDIAVEWESNGQPENNYKTT |
| | | | | PPVLDSDGSFFLYSRLTVDKSRWQEGNVFSCSVMHEALHNHYTQK |
| | | | | SLSLSLGK |
| SEQ ID NO: | IL27M4 | | Bivalent Fc-EBI3-p28 | ESKYGPPCPPCPAPPVAGPSVFLFPPKPKDTLMISRTPEVTCVVV |
| 45 | | | Fc is human IgG4s | DVSQEDPEVQFNWYVDGVEVHNAKTKPREEQFNSTYRVVSVLTVL |
| | | | | HQDWLNGKEYKCKVSNKGLPSSIEKTISKAKGQPREPQVYTLPPS |
| | | | | QEEMTKNQVSLTCLVKGFYPSDIAVEWESNGQPENNYKTTPPVLD |
| | | | | SDGSFFLYSRLTVDKSRWQEGNVFSCSVMHEALHNHYTQKSLSLS |
| | | | | LGKGGGGSGGGGSGGGGSYTETALVALSQPRVQCHASRYPVAVDC |
| | | | | SWTPLQAPNSTRSTSFIATYRLGVATQQQSQPCLQRSPQASRCTI |
| | | | | PDVHLFSTVPYMLNVTAVHPGGASSSLLAFVAERIIKPDPPEGVR |
| | | | | LRTAGQRLQVLWHPPASWPFPDIFSLKYRLRYRRRGASHFRQVGP |
| | | | | IEATTFTLRNSKPHAKYCIQVSAQDLTDYGKPSDWSLPGQVESAP |
| | | | | HKPGGGGSGGGGSGGGGSGGGGSFPTDPLSLQELRREFTVSLYLA |
| | | | | RKLLSEVQGYVHSFAESRLPGVNLDLLPLGYHLPNVSLTFQAWHH |
| | | | | LSDSERLCFLATTLRPFPAMLGGLGTQGTWTSSEREQLWAMRLDL |
| | | | | DRLHRHLRFQVLAAGFKCSKEEEDKEEEEEEEEEEKKLPLGALGG |
| | | | | PNQVSSQVSWPQLLYTYQLLHSLELVLSRAVRDLLLLSLPRRPGS |
| | | | | AWDS |
| SEQ ID NO: | IL27M5 | | Bivalent Fc-p28-EBI3 | ESKYGPPCPPCPAPPVAGPSVFLFPPKPKDTLMISRTPEVTCVVV |
| 46 | | | Fc is human IgG4s | DVSQEDPEVQFNWYVDGVEVHNAKTKPREEQFNSTYRVVSVLTVL |
| | | | | HQDWLNGKEYKCKVSNKGLPSSIEKTISKAKGQPREPQVYTLPPS |
| | | | | QEEMTKNQVSLTCLVKGFYPSDIAVEWESNGQPENNYKTTPPVLD |
| | | | | SDGSFFLYSRLTVDKSRWQEGNVFSCSVMHEALHNHYTQKSLSLS |
| | | | | LGKGGGGSGGGGSGGGGSFPTDPLSLQELRREFTVSLYLARKLLS |
| | | | | EVQGYVHSFAESRLPGVNLDLLPLGYHLPNVSLTFQAWHHLSDSE |
| | | | | RLCFLATTLRPFPAMLGGLGTQGTWTSSEREQLWAMRLDLRDLHR |
| | | | | HLRFQVLAAGFKCSKEEEDKEEEEEEEEEEKKLPLGALGGPNQVS |
| | | | | SQVSWPQLLYTYQLLHSLELVLSRAVRDLLLLSLPRRPGSAWDSG |
| | | | | GGGSGGGGSGGGGSGGGGSGGGGSYTETALVALSQPRVQCHASRY |
| | | | | PVAVDCSWTPLQAPNSTRSTSFIATYRLGVATQQQSQPCLQRSPQ |
| | | | | ASRCTIPDVHLFSTVPYMLNVTAVHPGGASSSLLAFVAERIIKPD |
| | | | | PPEGVRLRTAGQRLQVLWHPPASWPFPDIFSLKYRLRYRRRGASH |
| | | | | FRQVGPIEATTFTLRNSKPHAKYCIQVSAQDLTDYGKPSDWSLPG |
| | | | | QVESAPHKP |
| SEQ ID NO: | IL27M6- | Fc-p28 | Heterodimerize with | ESKYGPPCPPCPAPPVAGPSVFLFPPKPKDTLMISRTPEVTCVVV |
| 47 | Chain A | | FC-EBI3 to form | DVSQEDPEVQFNWYVDGVEVHNAKTKPREEQFNSTYRVVSVLTVL |
| | | | monovalent Fc-p28 x EBI3 | HQDWLNGKEYKCKVSNKGLPSSIEKTISKAKGQPREPQVYTLPPS |
| | | | Fc is human IgG4s(Knob) | QEEMTKNQVSLWCLVKGFYPSDIAVEWESNGQPENNYKTTPPVLD |
| | | | | SDGSFFLYSRLTVDKSRWQEGNVFSCSVMHEALHNHYTQKSLSLS |
| | | | | LGKGGGGSGGGGSGGGGSFPTDPLSLQELRREFTVSLYLARKLLS |
| | | | | EVQGYVHSFAESRLPGVNLDLLPLGYHLPNVSLTFQAWHHLSDSE |
| | | | | RLCFLATTLRPFPAMLGGLGTQGTWTSSEREQLWAMRLDLRDLHR |
| | | | | HLRFQVLAAGFKCSKEEEDKEEEEEEEEEEKKLPLGALGGPNQVS |
| | | | | SQVSWPQLLYTYQLLHSLELVLSRAVRDLLLLSLPRRPGSAWDS |
| SEQ ID NO: | IL27M6- | FC-EBI3 | Heterodimerize with | ESKYGPPCPPCPAPPVAGPSVFLFPPKPKDTLMISRTPEVTCVVV |
| 48 | Chain B | | Fc-p28 to form | DVSQEDPEVQFNWYVDGVEVHNAKTKPREEQFNSTYRVVSVLTVL |
| | | | monovalent Fc-p28 x EBI3 | HQDWLNGKEYKCKVSNKGLPSSIEKTISKAKGQPREPQVYTLPPS |
| | | | Fc is human | QEEMTKNQVSLSCAVKGFYPSDIAVEWESNGQPENNYKTTPPVLD |
| | | | IgG4s(Hole/Star) | SDGSFFLVSRLTVDKSRWQEGNVFSCSVMHEALHNRFTQKSLSLS |
| | | | | PGKGGGGSGGGGSGGGGSYTETALVALSQPRVQCHASRYPVAVDC |
| | | | | SWTPLQAPNSTRSTSFIATYRLGVATQQQSQPCLQRSPQASRCTI |
| | | | | PDVHLFSTVPYMLNVTAVHPGGASSSLLAFVAERIIKPDPPEGVR |
| | | | | LRTAGQRLQVLWHPPASWPFPDIFSLKYRLRYRRRGASHFRQVGP |
| | | | | IEATTFTLRNSKPHAKYCIQVSAQDLTDYGKPSDWSLPGQVESAP |
| | | | | HKP |
| SEQ ID NO: | IL27M12- | EBI3-p28- | Heterodimerize with | YTETALVALSQPRVQCHASRYPVAVDCSWTPLQAPNSTRSTSFIA |
| 49 | Chain A | Fc(Knob) | IgG4sFc (Hole/Star) | TYRLGVATQQQSQPCLQRSPQASRCTIPDVHLFSTVPYMLNVTAV |
| | | | to form monovalent | HPGGASSSLLAFVAERIIKPDPPEGVRLRTAGQRLQVLWHPPASW |
| | | | EBI3-p28 x Fc | PFPDIFSLKYRLRYRRRGASHFRQVGPIEATTFTLRNSKPHAKYC |
| | | | Fc is human | IQVSAQDLTDYGKPSDWSLPGQVESAPHKPGGGGSGGGGSGGGGS |
| | | | IgG4s(Knob) | GGGGSFPTDPLSLQELRREFTVSLYLARKLLSEVQGYVHSFAESR |
| | | | | LPGVNLDLLPLGYHLPNVSLTFQAWHHLSDSERLCFLATTLRPFP |
| | | | | AMLGGLGTQGTWTSSEREQLWAMRLDLRDLHRHLRFQVLAAGFKC |
| | | | | SKEEEDKEEEEEEEEEEKKLPLGALGGPNQVSSQVSWPQLLYTYQ |
| | | | | LLHSLELVLSRAVRDLLLLSLPRRPGSAWDSGGGGSGGGGSGGGG |
| | | | | SESKYGPPCPPCPAPPVAGPSVFLFPPKPKDTLMISRTPEVTCVV |
| | | | | VDVSQEDPEVQFNWYVDGVEVHNAKTKPREEQFNSTYRVVSVLTV |
| | | | | LHQDWLNGKEYKCKVSNKGLPSSIEKTISKAKGQPREPQVYTLPP |
| | | | | SQEEMTKNQVSLWCLVKGFYPSDIAVEWESNGQPENNYKTTPPVL |
| | | | | DSDGSFFLYSRLTVDKSRWQEGNVFSCSVMHEALHNHYTQKSLSL |
| | | | | SLGK |
| SEQ ID NO: | IL27M13 | p28-Fc | Bivalent p28-Fc | FPTDPLSLQELRREFTVSLYLARKLLSEVQGYVHSFAESRLPGVN |
| 50 | | | Fc is human IgG4s | LDLLPLGYHLPNVSLTFQAWHHLSDSERLCFLATTLRPFPAMLGG |
| | | | | LGTQGTWTSSEREQLWAMRLDLRDLHRHLRFQVLAAGFKCSKEEE |
| | | | | DKEEEEEEEEEEKKLPLGALGGPNQVSSQVSWPQLLYTYQLLHSL |
| | | | | ELVLSRAVRDLLLLSLPRRPGSAWDSGGGGSGGGGSGGGGSESKY |
| | | | | GPPCPPCPAPPVAGPSVFLFPPKPKDTLMISRTPEVTCWVDVSQE |
| | | | | DPEVQFNWYVDGVEVHNAKTKPREEQFNSTYRVVSVLTVLHQDWL |
| | | | | NGKEYKCKVSNKGLPSSIEKTISKAKGQPREPQVYTLPPSQEEMT |
| | | | | KNQVSLTCLVKGFYPSDIAVEWESNGQPENNYKTTPPVLDSDGSF |
| | | | | FLYSRLTVDKSRWQEGNVFSCSVMHEALHNHYTQKSLSLSLGK |
| | IL27M14 | | 2:2 complex between | |
| | | | IL27M13 (p28-Fc) and | |
| | | | EBI3-mmh | |
| SEQ ID NO: | IL27M16- | FC-EBI3- | Heterodimerize with | ESKYGPPCPPCPAPPVAGPSVFLFPPKPKDTLMISRTPEVTCVVV |
| 51 | Chain A | p28(Knob) | IgG4sFc (Hole/Star) | DVSQEDPEVQFNWYVDGVEVHNAKTKPREEQFNSTYRVVSVLTVL |
| | | | to form monovalent | HQDWLNGKEYKCKVSNKGLPSSIEKTISKAKGQPREPQVYTLPPS |
| | | | Fc x EBI3-p28 | QEEMTKNQVSLWCLVKGFYPSDIAVEWESNGQPENNYKTTPPVLD |
| | | | Fc is human | SDGSFFLYSRLTVDKSRWQEGNVFSCSVMHEALHNHYTQKSLSLS |
| | | | IgG4s(Knob) | LGKGGGGSGGGGSGGGGSYTETALVALSQPRVQCHASRYPVAVDC |
| | | | | SWTPLQAPNSTRSTSFIATYRLGVATQQQSQPCLQRSPQASRCTI |
| | | | | PDVHLFSTVPYMLNVTAVHPGGASSSLLAFVAERIIKPDPPEGVR |
| | | | | LRTAGQRLQVLWHPPASWPFPDIFSLKYRLRYRRRGASHFRQVGP |
| | | | | IEATTFTLRNSKPHAKYCIQVSAQDLTDYGKPSDWSLPGQVESAP |
| | | | | HKPGGGGSGGGGSGGGGSGGGGSFPTDPLSLQELRREFTVSLYLA |
| | | | | RKLLSEVQGYVHSFAESRLPGVNLDLLPLGYHLPNVSLTFQAWHH |
| | | | | LSDSERLCFLATTLRPFPAMLGGLGTQGTWTSSEREQLWAMRLDL |
| | | | | RDLHRHLRFQVLAAGFKCSKEEEDKEEEEEEEEEEKKLPLGALGG |
| | | | | PNQVSSQVSWPQLLYTYQLLHSLELVLSRAVRDLLLLSLPRRPGS |
| | | | | AWDS |
| SEQ ID NO: | IL27M17 | Fc-p28 | Bivalent Fc-p28 | ESKYGPPCPPCPAPPVAGPSVFLFPPKPKDTLMISRTPEVTCVVV |
| 52 | | | Fc is human IgG4s | DVSQEDPEVQFNWYVDGVEVHNAKTKPREEQFNSTYRVVSVLTVL |
| | | | | HQDWLNGKEYKCKVSNKGLPSSIEKTISKAKGQPREPQVYTLPPS |
| | | | | QEEMTKNQVSLTCLVKGFYPSDIAVEWESNGQPENNYKTTPPVLD |
| | | | | SDGSFFLYSRLTVDKSRWQEGNVFSCSVMHEALHNHYTQKSLSLS |
| | | | | LGKGGGGSGGGGSGGGGSFPTDPLSLQELRREFTVSLYLARKLLS |
| | | | | EVQGYVHSFAESRLPGVNLDLLPLGYHLPNVSLTFQAWHHLSDSE |
| | | | | RLCFLATTLRPFPAMLGGLGTQGTWTSSEREQLWAMRLDLRDLHR |
| | | | | HLRFQVLAAGFKCSKEEEDKEEEEEEEEEEKKLPLGALGGPNQVS |
| | | | | SQVSWPQLLYTYQLLHSLELVLSRAVRDLLLLSLPRRPGSAWDS |
| | IL27M18 | | 2:2 complex between | |
| | | | IL27M17 (Fc-p28) and | |
| | | | EBI3-mmh | |
| SEQ ID NO: | IL27M20 | | EBI3-p28-HSA-mmh | YTETALVALSQPRVQCHASRYPVAVDCSWTPLQAPNSTRSTSFIA |
| 53 | | | | TYRLGVATQQQSQPCLQRSPQASRCTIPDVHLFSTVPYMLNVTAV |
| | | | | HPGGASSSLLAFVAERIIKPDPPEGVRLRTAGQRLQVLWHPPASW |
| | | | | PFPDIFSLKYRLRYRRRGASHFRQVGPIEATTFTLRNSKPHAKYC |
| | | | | IQVSAQDLTDYGKPSDWSLPGQVESAPHKPGGGGSGGGGSGGGGS |
| | | | | GGGGSFPTDPLSLQELRREFTVSLYLARKLLSEVQGYVHSFAESR |
| | | | | LPGVNLDLLPLGYHLPNVSLTFQAWHHLSDSERLCFLATTLRPFP |
| | | | | AMLGGLGTQGTWTSSEREQLWAMRLDLRDLHRHLRFQVLAAGFKC |
| | | | | SKEEEDKEEEEEEEEEEKKLPLGALGGPNQVSSQVSWPQLLYTYQ |
| | | | | LLHSLELVLSRAVRDLLLLSLPRRPGSAWDSGGGGSGGGGSGGGG |
| | | | | SDAHKSEVAHRFKDLGEENFKALVLIAFAQYLQQCPFEDHVKLVN |
| | | | | EVTEFAKTCVADESAENCDKSLHTLFGDKLCTVATLRETYGEMAD |
| | | | | CCAKQEPERNECFLQHKDDNPNLPRLVRPEVDVMCTAFHDNEETF |
| | | | | LKKYLYEIARRHPYFYAPELLFFAKRYKAAFTECCQAADKAACLL |
| | | | | PKLDELRDEGKASSAKQRLKCASLQKFGERAFKAWAVARLSQRFP |
| | | | | KAEFAEVSKLVTDLTKVHTECCHGDLLECADDRADLAKYICENQD |
| | | | | SISSKLKECCEKPLLEKSHCIAEVENDEMPADLPSLAADFVESKD |
| | | | | VCKNYAEAKDVFLGMFLYEYARRHPDYSWLLLRLAKTYETTLEKC |
| | | | | CAAADPHECYAKVFDEFKPLVEEPQNLIKQNCELFEQLGEYKFQN |
| | | | | ALLVRYTKKVPQVSTPTLVEVSRNLGKVGSKCCKHPEAKRMPCAE |
| | | | | DYLSWLNQLCVLHEKTPVSDRVTKCCTESLVNRRPCFSALEVDET |
| | | | | YVPKEFNAETFTFHADICTLSEKERQIKKQTALVELVKHKPKATK |
| | | | | EQLKAVMDDFAAFVEKCCKADDKETCFAEEGKKLVAASQAALGLE |
| | | | | QKLISEEDLGGEQKLISEEDLHHHHHH |
| SEQ ID NO: | IL27M21- | p28-HSA | | FPTDPLSLQELRREFTVSLYLARKLLSEVQGYVHSFAESRLPGVN |
| 54 | Chain A | | | LDLLPLGYHLPNVSLTFQAWHHLSDSERLCFLATTLRPFPAMLGG |
| | | | | LGTQGTWTSSEREQLWAMRLDLRDLHRHLRFQVLAAGFKCSKEEE |
| | | | | DKEEEEEEEEEEKKLPLGALGGPNQVSSQVSWPQLLYTYQLLHSL |
| | | | | ELVLSRAVRDLLLLSLPRRPGSAWDSGGGGSGGGGSGGGGSDAHK |
| | | | | SEVAHRFKDLGEENFKALVLIAFAQYLQQCPFEDHVKLVNEVTEF |
| | | | | AKTCVADESAENCDKSLHTLFGDKLCTVATLRETYGEMADCCAKQ |
| | | | | EPERNECFLQHKDDNPNLPRLVRPEVDVMCTAFHDNEETFLKKYL |
| | | | | YEIARRHPYFYAPELLFFAKRYKAAFTECCQAADKAACLLPKLDE |
| | | | | LRDEGKASSAKQRLKCASLQKFGERAFKAWAVARLSQRFPKAEFA |
| | | | | EVSKLVTDLTKVHTECCHGDLLECADDRADLAKYICENQDSISSK |
| | | | | LKECCEKPLLEKSHCIAEVENDEMPADLPSLAADFVESKDVCKNY |
| | | | | AEAKDVFLGMFLYEYARRHPDYSWLLLRLAKTYETTLEKCCAAAD |
| | | | | PHECYAKVFDEFKPLVEEPQNLIKQNCELFEQLGEYKFQNALLVR |
| | | | | YTKKVPQVSTPTLVEVSRNLGKVGSKCCKHPEAKRMPCAEDYLSW |
| | | | | LNQLCVLHEKTPVSDRVTKCCTESLVNRRPCFSALEVDETYVPKE |
| | | | | FNAETFTFHADICTLSEKERQIKKQTALVELVKHKPKATKEQLKA |
| | | | | VMDDFAAFVEKCCKADDKETCFAEEGKKLVAASQAALGLEQKLIS |
| | | | | EEDLGGEQKLISEEDLHHHHHH |
| SEQ ID NO: | IL27M21- | EBI3-has | | YTETALVALSQPRVQCHASRYPVAVDCSWTPLQAPNSTRSTSFIA |
| 55 | Chain B | | | TYRLGVATQQQSQPCLQRSPQASRCTIPDVHLFSTVPYMLNVTAV |
| | | | | HPGGASSSLLAFVAERIIKPDPPEGVRLRTAGQRLQVLWHPPASW |
| | | | | PFPDIFSLKYRLRYRRRGASHFRQVGPIEATTFTLRNSKPHAKYC |
| | | | | IQVSAQDLTDYGKPSDWSLPGQVESAPHKPGGGGSGGGGSGGGGS |
| | | | | DAHKSEVAHRFKDLGEENFKALVLIAFAQYLQQCPFEDHVKLVNE |
| | | | | VTEFAKTCVADESAENCDKSLHTLFGDKLCTVATLRETYGEMADC |
| | | | | CAKQEPERNECFLQHKDDNPNLPRLVRPEVDVMCTAFHDNEETFL |
| | | | | KKYLYEIARRHPYFYAPELLFFAKRYKAAFTECCQAADKAACLLP |
| | | | | KLDELRDEGKASSAKQRLKCASLQKFGERAFKAWAVARLSQRFPK |
| | | | | AEFAEVSKLVTDLTKVHTECCHGDLLECADDRADLAKYICENQDS |
| | | | | ISSKLKECCEKPLLEKSHCIAEVENDEMPADLPSLAADFVESKDV |
| | | | | CKNYAEAKDVFLGMFLYEYARRHPDYSWLLLRLAKTYETTLEKCC |
| | | | | AAADPHECYAKVFDEFKPLVEEPQNLIKQNCELFEQLGEYKFQNA |
| | | | | LLVRYTKKVPQVSTPTLVEVSRNLGKVGSKCCKHPEAKRMPCAED |
| | | | | YLSWLNQLCVLHEKTPVSDRVTKCCTESLVNRRPCFSALEVDETY |
| | | | | VPKEFNAETFTFHADICTLSEKERQIKKQTALVELVKHKPKATKE |
| | | | | QLKAVMDDFAAFVEKCCKADDKETCFAEEGKKLVAASQAALGLEQ |
| | | | | KLISEEDLGGEQKLISEEDLHHHHHH |
| SEQ ID NO: | | | EBI3-mmh | YTETALVALSQPRVQCHASRYPVAVDCSWTPLQAPNSTRSTSFIA |
| 56 | | | | TYRLGVATQQQSQPCLQRSPQASRCTIPDVHLFSTVPYMLNVTAV |
| | | | | HPGGASSSLLAFVAERIIKPDPPEGVRLRTAGQRLQVLWHPPASW |
| | | | | PFPDIFSLKYRLRYRRRGASHFRQVGPIEATTFTLRNSKPHAKYC |
| | | | | IQVSAQDLTDYGKPSDWSLPGQVESAPHKPEQKLISEEDLGGEQK |
| | | | | LISEEDLHHHHHH |
| SEQ ID NO: | | | IgG4sFc (Hole/Star) | ESKYGPPCPPCPAPPVAGPSVFLFPPKPKDTLMISRTPEVTCVVV |
| 57 | | | | DVSQEDPEVQFNWYVDGVEVHNAKTKPREEQFNSTYRVVSVLTVL |
| | | | | HQDWLNGKEYKCKVSNKGLPSSIEKTISKAKGQPREPQVYTLPPS |
| | | | | QEEMTKNQVSLSCAVKGFYPSDIAVEWESNGQPENNYKTTPPVLD |
| | | | | SDGSFFLVSRLTVDKSRWQEGNVFSCSVMHEALHNRFTQKSLSLS |
| | | | | PGK |
| SEQ ID NO: | IL27M22 | mEB13-mp28 | | YTETALVALSQPRVQCHASRYPVAVDCSWTPLQAPNSTRSTSFIA |
| 58 | | hIgG1 Fc | | TYRLGVATQQQSQPCLQRSPQASRCTIPDVHLFSTVPYMLNVTAV |
| | | | | HPGGASSSLLAFVAERIIKPDPPEGVRLRTAGQRLQVLWHPPASW |
| | | | | PFPDIFSLKYRLRYRRRGASHFRQVGPIEATTFTLRNSKPHAKYC |
| | | | | IQVSAQDLTDYGKPSDWSLPGQVESAPHKPGGGGSGGGGSGGGGS |
| | | | | FPTDPLSLQELRREFTVSLYLARKLLSEVQGYVHSFAESRLPGVN |
| | | | | LDLLPLGYHLPNVSLTFQAWHHLSDSERLCFLATTLRPFPAMLGG |
| | | | | LGTQGTWTSSEREQLWAMRLDLRDLHRHLRFQVLAAGFKCSKEEE |
| | | | | DKEEEEEEEEEEKKLPLGALGGPNQVSSQVSWPQLLYTYQLLHSL |
| | | | | ELVLSRAVRDLLLLSLPRRPGSAWDSDKTHTCPPCPAPELLGGPS |
| | | | | VFLFPPKPKDTLMISRTPEVTCVVVDVSHEDPEVKFNWYVDGVEV |
| | | | | HNAKTKPREEQYNSTYRVVSVLTVLHQDWLNGKEYKCKVSNKALP |
| | | | | APIEKTISKAKGQPREPQVYTLPPSRDELTKNQVSLTCLVKGFYP |
| | | | | SDIAVEWESNGQPENNYKTTPPVLDSDGSFFLYSKLTVDKSRWQQ |
| | | | | GNVFSCSVMHEALHNHYTQKSLSLSPGK |
| SEQ ID NO: | IL27M23- | IL27M16 | Heterodimerize with | ESKYGPPCPPCPAPPVAGPSVFLFPPKPKDTLMISRTPEVTCVVV |
| 59 | Chain A | (Y202A, | IgG4sFc (Hole/Star) to | DVSQEDPEVQFNWYVDGVEVHNAKTKPREEQFNSTYRVVSVLTVL |
| | | Q203A) | form monovalent | HQDWLNGKEYKCKVSNKGLPSSIEKTISKAKGQPREPQVYTLPPS |
| | | | Fc x EB13-p28 | QEEMTKNQVSLWCLVKGFYPSDIAVEWESNGQPENNYKTTPPVLD |
| | | | (Y202A, Q203A) | SDGSFFLYSRLTVDKSRWQEGNVFSCSVMHEALHNHYTQKSLSLS |
| | | | Fc is human IgG4s(Knob) | LGKGGGGSGGGGSGGGGSYTETALVALSQPRVQCHASRYPVAVDC |
| | | | | SWTPLQAPNSTRSTSFIATYRLGVATQQQSQPCLQRSPQASRCTI |
| | | | | PDVHLFSTVPYMLNVTAVHPGGASSSLLAFVAERIIKPDPPEGVR |
| | | | | LRTAGQRLQVLWHPPASWPFPDIFSLKYRLRYRRRGASHFRQVGP |
| | | | | IEATTFTLRNSKPHAKYCIQVSAQDLTDYGKPSDWSLPGQVESAP |
| | | | | HKPGGGGSGGGGSGGGGSGGGGSFPTDPLSLQELRREFTVSLYLA |
| | | | | RKLLSEVQGYVHSFAESRLPGVNLDLLPLGYHLPNVSLTFQAWHH |
| | | | | LSDSERLCFLATTLRPFPAMLGGLGTQGTWTSSEREQLWAMRLDL |
| | | | | RDLHRHLRFQVLAAGFKCSKEEEDKEEEEEEEEEEKKLPLGALGG |
| | | | | PNQVSSQVSWPQLLYTAALLHSLELVLSRAVRDLLLLSLPRRPGS |
| | | | | AWDS |
| SEQ ID NO: | IL27M24- | IL27M16 | Heterodimerize with | ESKYGPPCPPCPAPPVAGPSVFLFPPKPKDTLMISRTPEVTCVVV |
| 60 | Chain A | (K52A) | IgG4sFc (Hole/Star) | DVSQEDPEVQFNWYVDGVEVHNAKTKPREEQFNSTYRVVSVLTVL |
| | | | to form monovalent | HQDWLNGKEYKCKVSNKGLPSSIEKTISKAKGQPREPQVYTLPPS |
| | | | Fc x EB13-p28 (K52A) | QEEMTKNQVSLWCLVKGFYPSDIAVEWESNGQPENNYKTTPPVLD |
| | | | Fc is human IgG4s(Knob) | SDGSFFLYSRLTVDKSRWQEGNVFSCSVMHEALHNHYTQKSLSLS |
| | | | | LGKGGGGSGGGGSGGGGSYTETALVALSQPRVQCHASRYPVAVDC |
| | | | | SWTPLQAPNSTRSTSFIATYRLGVATQQQSQPCLQRSPQASRCTI |
| | | | | PDVHLFSTVPYMLNVTAVHPGGASSSLLAFVAERIIKPDPPEGVR |
| | | | | LRTAGQRLQVLWHPPASWPFPDIFSLKYRLRYRRRGASHFRQVGP |
| | | | | IEATTFTLRNSKPHAKYCIQVSAQDLTDYGKPSDWSLPGQVESAP |
| | | | | HKPGGGGSGGGGSGGGGSGGGGSFPTDPLSLQELRREFTVSLYLA |
| | | | | RALLSEVQGYVHSFAESRLPGVNLDLLPLGYHLPNVSLTFQAWHH |
| | | | | LSDSERLCFLATTLRPFPAMLGGLGTQGTWTSSEREQLWAMRLDL |
| | | | | RDLHRHLRFQVLAAGFKCSKEEEDKEEEEEEEEEEKKLPLGALGG |
| | | | | PNQVSSQVSWPQLLYTYQLLHSLELVLSRAVRDLLLLSLPRRPGS |
| | | | | AWDS |
| SEQ ID NO: | IL27M25- | IL27M16 | Heterodimerize with | ESKYGPPCPPCPAPPVAGPSVFLFPPKPKDTLMISRTPEVTCVVV |
| 61 | Chain A | (L138A, | IgG4sFc (Hole/Star) | DVSQEDPEVQFNWYVDGVEVHNAKTKPREEQFNSTYRVVSVLTVL |
| | | R145A, | to form monovalent | HQDWLNGKEYKCKVSNKGLPSSIEKTISKAKGQPREPQVYTLPPS |
| | | H146A) | Fc x EB13-p28 | QEEMTKNQVSLWCLVKGFYPSDIAVEWESNGQPENNYKTTPPVLD |
| | | | (L138A, R145A, H146A) | SDGSFFLYSRLTVDKSRWQEGNVFSCSVMHEALHNHYTQKSLSLS |
| | | | Fc is human IgG4s(Knob) | LGKGGGGSGGGGSGGGGSYTETALVALSQPRVQCHASRYPVAVDC |
| | | | | SWTPLQAPNSTRSTSFIATYRLGVATQQQSQPCLQRSPQASRCTI |
| | | | | PDVHLFSTVPYMLNVTAVHPGGASSSLLAFVAERIIKPDPPEGVR |
| | | | | LRTAGQRLQVLWHPPASWPFPDIFSLKYRLRYRRRGASHFRQVGP |
| | | | | IEATTFTLRNSKPHAKYCIQVSAQDLTDYGKPSDWSLPGQVESAP |
| | | | | HKPGGGGSGGGGSGGGGSGGGGSFPTDPLSLQELRREFTVSLYLA |
| | | | | RKLLSEVQGYVHSFAESRLPGVNLDLLPLGYHLPNVSLTFQAWHH |
| | | | | LSDSERLCFLATTLRPFPAMLGGLGTQGTWTSSEREQLWAMRADL |
| | | | | RDLHAALRFQVLAAGFKCSKEEEDKEEEEEEEEEEKKLPLGALGG |
| | | | | PNQVSSQVSWPQLLYTYQLLHSLELVLSRAVRDLLLLSLPRRPGS |
| | | | | AWDS |
| SEQ ID NO: | IL27M26- | IL27M16 | Heterodimerize with | ESKYGPPCPPCPAPPVAGPSVFLFPPKPKDTLMISRTPEVTCVVV |
| 62 | Chain A | (L198A, | IgG4sFc (Hole/Star) | DVSQEDPEVQFNWYVDGVEVHNAKTKPREEQFNSTYRVVSVLTVL |
| | | L199A) | to form monovalent | HQDWLNGKEYKCKVSNKGLPSSIEKTISKAKGQPREPQVYTLPPS |
| | | | Fc x EB13-p28 | QEEMTKNQVSLWCLVKGFYPSDIAVEWESNGQPENNYKTTPPVLD |
| | | | (L198A, L199A) | SDGSFFLYSRLTVDKSRWQEGNVFSCSVMHEALHNHYTQKSLSLS |
| | | | Fc is human IgG4s(Knob) | LGKGGGGSGGGGSGGGGSYTETALVALSQPRVQCHASRYPVAVDC |
| | | | | SWTPLQAPNSTRSTSFIATYRLGVATQQQSQPCLQRSPQASRCTI |
| | | | | PDVHLFSTVPYMLNVTAVHPGGASSSLLAFVAERIIKPDPPEGVR |
| | | | | LRTAGQRLQVLWHPPASWPFPDIFSLKYRLRYRRRGASHFRQVGP |
| | | | | IEATTFTLRNSKPHAKYCIQVSAQDLTDYGKPSDWSLPGQVESAP |
| | | | | HKPGGGGSGGGGSGGGGSGGGGSFPTDPLSLQELRREFTVSLYLA |
| | | | | RKLLSEVQGYVHSFAESRLPGVNLDLLPLGYHLPNVSLTFQAWHH |
| | | | | LSDSERLCFLATTLRPFPAMLGGLGTQGTWTSSEREQLWAMRLDL |
| | | | | RDLHRHLRFQVLAAGFKCSKEEEDKEEEEEEEEEEKKLPLGALGG |
| | | | | PNQVSSQVSWPQAAYTYQLLHSLELVLSRAVRDLLLLSLPRRPGS |
| | | | | AWDS |
| SEQ ID NO: | IL27M27- | IL27M16 | Heterodimerize with | ESKYGPPCPPCPAPPVAGPSVFLFPPKPKDTLMISRTPEVTCVVV |
| 63 | Chain A | (W195A) | IgG4sFc (Hole/Star) | DVSQEDPEVQFNWYVDGVEVHNAKTKPREEQFNSTYRVVSVLTVL |
| | | | to form monovalent | HQDWLNGKEYKCKVSNKGLPSSIEKTISKAKGQPREPQVYTLPPS |
| | | | FcxEB13-p28 (W195A) | QEEMTKNQVSLWCLVKGFYPSDIAVEWESNGQPENNYKTTPPVLD |
| | | | Fc is human IgG4s(Knob) | SDGSFFLYSRLTVDKSRWQEGNVFSCSVMHEALHNHYTQKSLSLS |
| | | | | LGKGGGGSGGGGSGGGGSYTETALVALSQPRVQCHASRYPVAVDC |
| | | | | SWTPLQAPNSTRSTSFIATYRLGVATQQQSQPCLQRSPQASRCTI |
| | | | | PDVHLFSTVPYMLNVTAVHPGGASSSLLAFVAERIIKPDPPEGVR |
| | | | | LRTAGQRLQVLWHPPASWPFPDIFSLKYRLRYRRRGASHFRQVGP |
| | | | | IEATTFTLRNSKPHAKYCIQVSAQDLTDYGKPSDWSLPGQVESAP |
| | | | | HKPGGGGSGGGGSGGGGSGGGGSFPTDPLSLQELRREFTVSLYLA |
| | | | | RKLLSEVQGYVHSFAESRLPGVNLDLLPLGYHLPNVSLTFQAWHH |
| | | | | LSDSERLCFLATTLRPFPAMLGGLGTQGTWTSSEREQLWAMRLDL |
| | | | | RDLHRHLRFQVLAAGFKCSKEEEDKEEEEEEEEEEKKLPLGALGG |
| | | | | PNQVSSQVSAPQLLYTYQLLHSLELVLSRAVRDLLLLSLPRRPGS |
| | | | | AWDS |
| SEQ ID NOs: | | Isotype- | CH1-CH3 sequence is | Heavy Chain-A, VH + hIgG4s (Knob - SEQ ID |
| 64, 65, 66 | | IL27(L98A, | human IgG4s | NO: 64): |
| | | L99A) | (Knob x Hole/Star) | QVQLQESGPGLVKPSETLSLTCTVSGGSISSYYWSWIRQPPGKGL |
| | | | | EWIGYIYYSGRTNYNPSLKSRVTISVDTSKNQFSLKLSSVTAADT |
| | | | | AIYYCARHRVTRTADSFDYWGQGTLVTVSSASTKGPSVFPLAPCS |
| | | | | RSTSESTAALGCLVKDYFPEPVTVSWNSGALTSGVHTFPAVLQSS |
| | | | | GLYSLSSVVTVPSSSLGTKTYTCNVDHKPSNTKVDKRVESKYGPP |
| | | | | CPPCPAPPVAGPSVFLFPPKPKDTLMISRTPEVTCVVVDVSQEDP |
| | | | | EVQFNWYVDGVEVHNAKTKPREEQFNSTYRVVSVLTVLHQDWLNG |
| | | | | KEYKCKVSNKGLPSSIEKTISKAKGQPREPQVYTLPPSQEEMTKN |
| | | | | QVSLWCLVKGFYPSDIAVEWESNGQPENNYKTTPPVLDSDGSFFL |
| | | | | YSRLTVDKSRWQEGNVFSCSVMHEALHNHYTQKSLSLSLGKGGGG |
| | | | | SGGGGSGGGGSYTETALVALSQPRVQCHASRYPVAVDCSWTPLQA |
| | | | | PNSTRSTSFIATYRLGVATQQQSQPCLQRSPQASRCTIPDVHLFS |
| | | | | TVPYMLNVTAVHPGGASSSLLAFVAERIIKPDPPEGVRLRTAGQR |
| | | | | LQVLWHPPASWPFPDIFSLKYRLRYRRRGASHFRQVGPIEATTFT |
| | | | | LRNSKPHAKYCIQVSAQDLTDYGKPSDWSLPGQVESAPHKPGGGG |
| | | | | SGGGGSGGGGSGGGGSFPTDPLSLQELRREFTVSLYLARKLLSEV |
| | | | | QGYVHSFAESRLPGVNLDLLPLGYHLPNVSLTFQAWHHLSDSERL |
| | | | | CFLATTLRPFPAMLGGLGTQGTWTSSEREQLWAMRLDLRDLHRHL |
| | | | | RFQVLAAGFKCSKEEEDKEEEEEEEEEEKKLPLGALGGPNQVSSQ |
| | | | | VSWPQAAYTYQLLHSLELVLSRAVRDLLLLSLPRRPGSAWDS |
| | | | | Heavy Chain-B, VH + hIgG4s (Hole/Star - |
| | | | | SEQ ID NO: 65): |
| | | | | QVQLQESGPGLVKPSETLSLTCTVSGGSISSYYWSWIRQPPGKGL |
| | | | | EWIGYIYYSGRTNYNPSLKSRVTISVDTSKNQFSLKLSSVTAADT |
| | | | | AIYYCARHRVTRTADSFDYWGQGTLVTVSSASTKGPSVFPLAPCS |
| | | | | RSTSESTAALGCLVKDYFPEPVTVSWNSGALTSGVHTFPAVLQSS |
| | | | | GLYSLSSVVTVPSSSLGTKTYTCNVDHKPSNTKVDKRVESKYGPP |
| | | | | CPPCPAPPVAGPSVFLFPPKPKDTLMISRTPEVTCVVVDVSQEDP |
| | | | | EVQFNWYVDGVEVHNAKTKPREEQFNSTYRVVVSVLTVLHQDWLN |
| | | | | GKEYKCKVSNKGLPSSIEKTISKAKGQPREPQVYTLPPSQEEMTK |
| | | | | NQVSLSCAVKGFYPSDIAVEWESNGQPENNYKTTPPVLDSDGSFF |
| | | | | LVSRLTVDKSRWQEGNVFSCSVMHEALHNRFTQKSLSLSPGK |
| | | | | Light Chain, CH + human kappa (SEQ ID |
| | | | | NO: 66): |
| | | | | DIQMTQSPSSLSASVGDRVTITCQASQDINNYLNWYQQKTGKAPK |
| | | | | FLIYDASNLETGVSSRFSGSGSGTDFTFTISSLQPEDVGTYYCHQ |
| | | | | YGDLPYTFGQGTKLEIKRTVAAPSVFIFPPSDEQLKSGTASVVCL |
| | | | | LNNFYPREAKVQWKVDNALQSGNSQESVTEQDSKDSTYSLSSTLT |
| | | | | LSKADYEKHKVYACEVTHQGLSSPVTKSFNRGEC |
| SEQ ID NOs: | | Anti mPD1- | CH1-CH3 sequence is | Heavy Chain-A, VH + hIgG4s (Knob - SEQ ID |
| 67, 68, 69 | | IL27(L98A, | human IgG4s | NO: 67): |
| | | L99A) | (Knob x Hole/Star) | Anti-mPD1 sequence - optional linker - |
| | | | | ESKYGPPCPPCPAPPVAGPSVFLFPPKPKDTLMISRTPEVTCVVV |
| | | | | DVSQEDPEVQFNWYVDGVEVHNAKTKPREEQFNSTYRVVSVLTVL |
| | | | | HQDWLNGKEYKCKVSNKGLPSSIEKTISKAKGQPREPQVYTLPPS |
| | | | | QEEMTKNQVSLWCLVKGFYPSDIAVEWESNGQPENNYKTTPPVLD |
| | | | | SDGSFFLYSRLTVDKSRWQEGNVFSCSVMHEALHNHYTQKSLSLS |
| | | | | LGKGGGGSGGGGSGGGGSYTETALVALSQPRVQCHASRYPVAVDC |
| | | | | SWTPLQAPNSTRSTSFIATYRLGVATQQQSQPCLQRSPQASRCTI |
| | | | | PDVHLFSTVPYMLNVTAVHPGGASSSLLAFVAERIIKPDPPEGVR |
| | | | | LRTAGQRLQVLWHPPASWPFPDIFSLKYRLRYRRRGASHFRQVGP |
| | | | | IEATTFTLRNSKPHAKYCIQVSAQDLTDYGKPSDWSLPGQVESAP |
| | | | | HKPGGGGSGGGGSGGGGSGGGGSFPTDPLSLQELRREFTVSLYLA |
| | | | | RKLLSEVQGYVHSFAESRLPGVNLDLLPLGYHLPNVSLTFQAWHH |
| | | | | LSDSERLCFLATTLRPFPAMLGGLGTQGTWTSSEREQLWAMRLDL |
| | | | | RDLHRHLRFQVLAAGFKCSKEEEDKEEEEEEEEEEKKLPLGALGG |
| | | | | PNQVSSQVSWPQAAYTYQLLHSLELVLSRAVRDLLLLSLPRRPGS |
| | | | | AWDS |
| | | | | Heavy Chain-B, VH + hIgG4s |
| | | | | (Hole/Star - SEQ ID NO: 68): |
| | | | | Anti-mPD1 sequence - optional linker - |
| | | | | ESKYGPPCPPCPAPPVAGPSVFLFPPKPKDTLMISRTPEVTCVVV |
| | | | | DVSQEDPEVQFNWYVDGVEVHNAKTKPREEQFNSTYRVVSVLTVL |
| | | | | HQDWLNGKEYKCKVSNKGLPSSIEKTISKAKGQPREPQVYTLPPS |
| | | | | QEEMTKNQVSLSCAVKGFYPSDIAVEWESNGQPENNYKTTPPVLD |
| | | | | SDGSFFLVSRLTVDKSRWQEGNVFSCSVMHEALHNRFTQKSLSLS |
| | | | | PGK |
| | | | | Light Chain, CH + human kappa (SEQ ID |
| | | | | NO: 69): |
| | | | | DIVMTQGTLPNPVPSGESVSITCRSSKSLLYSDGKTYLNWYLQRP |
| | | | | GQSPQLLIYWMSTRASGVSDRFSGSGSGTDFTLKISGVEAEDVGI |
| | | | | YYCQQGLEFPTFGGGTKLELKTVAAPSVFIFPPSDEQLKSGTASV |
| | | | | VCLLNNFYPREAKVQWKVDNALQSGNSQESVTEQDSKDSTYSLSS |
| | | | | TLTLSKADYEKHKVYACEVTHQGLSSPVTKSFNRGEC |
| SEQ ID NOs: | | Anti hPD1- | CH1-CH3 sequence is | Heavy Chain-A, VH + hIgG4s |
| 67, 68, 70 | | IL27(L98A, | human IgG4s | (Knob - SEQ ID NO: 67): |
| | | L99A) | (Knob x Hole/Star) | Anti-mPD1 sequence - optional linker - |
| | | | | ESKYGPPCPPCPAPPVAGPSVFLFPPKPKDTLMISRTPEVTCVVV |
| | | | | DVSQEDPEVQFNWYVDGVEVHNAKTKPREEQFNSTYRVVSVLTVL |
| | | | | HQDWLNGKEYKCKVSNKGLPSSIEKTISKAKGQPREPQVYTLPPS |
| | | | | QEEMTKNQVSLWCLVKGFYPSDIAVEWESNGQPENNYKTTPPVLD |
| | | | | SDGSFFLYSRLTVDKSRWQEGNVFSCSVMHEALHNHYTQKSLSLS |
| | | | | LGKGGGGSGGGGSGGGGSYTETALVALSQPRVQCHASRYPVAVDC |
| | | | | SWTPLQAPNSTRSTSFIATYRLGVATQQQSQPCLQRSPQASRCTI |
| | | | | PDVHLFSTVPYMLNVTAVHPGGASSSLLAFVAERIIKPDPPEGVR |
| | | | | LRTAGQRLQVLWHPPASWPFPDIFSLKYRLRYRRRGASHFRQVGP |
| | | | | IEATTFTLRNSKPHAKYCIQVSAQDLTDYGKPSDWSLPGQVESAP |
| | | | | HKPGGGGSGGGGSGGGGSGGGGSFPTDPLSLQELRREFTVSLYLA |
| | | | | RKLLSEVQGYVHSFAESRLPGVNLDLLPLGYHLPNVSLTFQAWHH |
| | | | | LSDSERLCFLATTLRPFPAMLGGLGTQGTWTSSEREQLWAMRLDL |
| | | | | RDLHRHLRFQVLAAGFKCSKEEEDKEEEEEEEEEEKKLPLGALGG |
| | | | | PNQVSSQVSWPQAAYTYQLLHSLELVLSRAVRDLLLLSLPRRPGS |
| | | | | AWDS |
| | | | | Heavy Chain-B, VH + hIgG4s |
| | | | | (Hole/Star - SEQ ID NO: 68): |
| | | | | Anti-mPD1 sequence - optional linker - |
| | | | | ESKYGPPCPPCPAPPVAGPSVFLFPPKPKDTLMISRTPEVTCVVV |
| | | | | DVSQEDPEVQFNWYVDGVEVHNAKTKPREEQFNSTYRVVSVLTVL |
| | | | | HQDWLNGKEYKCKVSNKGLPSSIEKTISKAKGQPREPQVYTLPPS |
| | | | | QEEMTKNQVSLSCAVKGFYPSDIAVEWESNGQPENNYKTTPPVLD |
| | | | | SDGSFFLVSRLTVDKSRWQEGNVFSCSVMHEALHNRFTQKSLSLS |
| | | | | PGK |
| | | | | Light Chain, CH + human kappa (SEQ ID |
| | | | | NO: 70): |
| | | | | DIQMTQSPSSLSASVGDRVTITCRASQSISSYLNWYQQKPGKAPK |
| | | | | LLIYAASSLQSGVPSRFSGSGSGTDFTLTISSLQPEDFATYYCQQ |
| | | | | SYSTPPITFGQGTRLEIKRTVAAPSVFIFPPSDEQLKSGTASVVC |
| | | | | LLNNFYPREAKVQWKVDNALQSGNSQESVTEQDSKDSTYSLSSTL |
| | | | | TLSKADYEKHKVYACEVTHQGLSSPVTKSFNRGEC |
| SEQ ID NOs: | | Isotype- | CH1-CH3 sequence is | Heavy Chain-A, VH + hIgG4s (Knob -SEQ ID |
| 71, 65, 66 | | IL27(L138A, | human IgG4s | NO: 71): |
| | | R145A, | (Knob x Hole/Star) | QVQLQESGPGLVKPSETLSLTCTVSGGSISSYYWSWIRQPPGKGL |
| | | H146A) | | EWIGYIYYSGRTNYNPSLKSRVTISVDTSKNQFSLKLSSVTAADT |
| | | | | AIYYCARHRVTRTADSFDYWGQGTLVTVSSASTKGPSVFPLAPCS |
| | | | | RSTSESTAALGCLVKDYFPEPVTVSWNSGALTSGVHTFPAVLQSS |
| | | | | GLYSLSSVVTVPSSSLGTKTYTCNVDHKPSNTKVDKRVESKYGPP |
| | | | | CPPCPAPPVAGPSVFLFPPKPKDTLMISRTPEVTCVVVDVSQEDP |
| | | | | EVQFNWYVDGVEVHNAKTKPREEQFNSTYRVVSVLTVLHQDWLNG |
| | | | | KEYKCKVSNKGLPSSIEKTISKAKGQPREPQVYTLPPSQEEMTKN |
| | | | | QVSLWCLVKGFYPSDIAVEWESNGQPENNYKTTPPVLDSDGSFFL |
| | | | | YSRLTVDKSRWQEGNVFSCSVMHEALHNHYTQKSLSLSLGKGGGG |
| | | | | SGGGGSGGGGSYTETALVALSQPRVQCHASRYPVAVDCSWTPLQA |
| | | | | PNSTRSTSFIATYRLGVATQQQSQPCLQRSPQASRCTIPDVHLFS |
| | | | | TVPYMLNVTAVHPGGASSSLLAFVAERIIKPDPPEGVRLRTAGQR |
| | | | | LQVLWHPPASWPFPDIFSLKYRLRYRRRGASHFRQVGPIEATTFT |
| | | | | LRNSKPHAKYCIQVSAQDLTDYGKPSDWSLPGQVESAPHKPGGGG |
| | | | | SGGGGSGGGGSGGGGSFPTDPLSLQELRREFTVSLYLARKLLSEV |
| | | | | QGYVHSFAESRLPGVNLDLLPLGYHLPNVSLTFQAWHHLSDSERL |
| | | | | CFLATTLRPFPAMLGGLGTQGTWTSSEREQLWAMRADLRDLHAAL |
| | | | | RFQVLAAGFKCSKEEEDKEEEEEEEEEEKKLPLGALGGPNQVSSQ |
| | | | | VSWPQLLYTYQLLHSLELVLSRAVRDLLLLSLPRRPGSAWDS |
| | | | | Heavy Chain-B, VH + hIgG4s |
| | | | | (Hole/Star - SEQ ID NO: 65): |
| | | | | QVQLQESGPGLVKPSETLSLTCTVSGGSISSYYWSWIRQPPGKGL |
| | | | | EWIGYIYYSGRTNYNPSLKSRVTISVDTSKNQFSLKLSSVTAADT |
| | | | | AIYYCARHRVTRTADSFDYWGQGTLVTVSSASTKGPSVFPLAPCS |
| | | | | RSTSESTAALGCLVKDYFPEPVTVSWNSGALTSGVHTFPAVLQSS |
| | | | | GLYSLSSVVTVPSSSLGTKTYTCNVDHKPSNTKVDKRVESKYGPP |
| | | | | CPPCPAPPVAGPSVFLFPPKPKDTLMISRTPEVTCVVVDVSQEDP |
| | | | | EVQFNWYVDGVEVHNAKTKPREEQFNSTYRVVSVLTVLHQDWLNG |
| | | | | KEYKCKVSNKGLPSSIEKTISKAKGQPREPQVYTLPPSQEEMTKN |
| | | | | QVSLSCAVKGFYPSDIAVEWESNGQPENNYKTTPPVLDSDGSFFL |
| | | | | VSRLTVDKSRWQEGNVFSCSVMHEALHNRFTQKSLSLSPGK |
| | | | | Light Chain, CH + human kappa |
| | | | | (SEQ ID NO: 66): |
| | | | | DIQMTQSPSSLSASVGDRVTITCQASQDINNYLNWYQQKTGKAPK |
| | | | | FLIYDASNLETGVSSRFSGSGSGTDFTFTISSLQPEDVGTYYCHQ |
| | | | | YGDLPYTFGQGTKLEIKRTVAAPSVFIFPPSDEQLKSGTASVVCL |
| | | | | LNNFYPREAKVQWKVDNALQSGNSQESVTEQDSKDSTYSLSSTLT |
| | | | | LSKADYEKHKVYACEVTHQGLSSPVTKSFNRGEC |
| SEQ ID NOs: | | Anti mPD1- | CH1-CH3 sequence is | Heavy Chain-A, VH + hIgG4s (Knob - SEQ ID |
| 72, 73, 69 | | IL27(L138A, | human IgG4s | NO: 72): |
| | | R145A, | (Knob x Hole/Star) | EVQLQESGPGLVKPSQSLSLTCSVTGYSITSSYRWNWIRKFPGNR |
| | | H146A) | | LEWMGYINSAGISNYNPSLKRRISITRDTSKNQFFLQVNSVTTED |
| | | | | AATYYCARSDNMGTTPFTYWGQGTLVTVSSASTKGPSVFPLAPCS |
| | | | | RSTSESTAALGCLVKDYFPEPVTVSWNSGALTSGVHTFPAVLQSS |
| | | | | GLYSLSSVVTVPSSSLGTKTYTCNVDHKPSNTKVDKRVESKYGPP |
| | | | | CPPCPAPPVAGPSVFLFPPKPKDTLMISRTPEVTCVVVDVSQEDP |
| | | | | EVQFNWYVDGVEVHNAKTKPREEQFNSTYRVVSVLTVLHQDWLNG |
| | | | | KEYKCKVSNKGLPSSIEKTISKAKGQPREPQVYTLPPSQEEMTKN |
| | | | | QVSLWCLVKGFYPSDIAVEWESNGQPENNYKTTPPVLDSDGSFFL |
| | | | | YSRLTVDKSRWQEGNVFSCSVMHEALHNHYTQKSLSLSLGKGGGG |
| | | | | SGGGGSGGGGSYTETALVALSQPRVQCHASRYPVAVDCSWTPLQA |
| | | | | PNSTRSTSFIATYRLGVATQQQSQPCLQRSPQASRCTIPDVHLFS |
| | | | | TVPYMLNVTAVHPGGASSSLLAFVAERIIKPDPPEGVRLRTAGQR |
| | | | | LQVLWHPPASWPFPDIFSLKYRLRYRRRGASHFRQVGPIEATTFT |
| | | | | LRNSKPHAKYCIQVSAQDLTDYGKPSDWSLPGQVESAPHKPGGGG |
| | | | | SGGGGSGGGGSGGGGSFPTDPLSLQELRREFTVSLYLARKLLSEV |
| | | | | QGYVHSFAESRLPGVNLDLLPLGYHLPNVSLTFQAWHHLSDSERL |
| | | | | CFLATTLRPFPAMLGGLGTQGTWTSSEREQLWAMRADLRDLHAAL |
| | | | | RFQVLAAGFKCSKEEEDKEEEEEEEEEEKKLPLGALGGPNQVSSQ |
| | | | | VSWPQLLYTYQLLHSLELVLSRAVRDLLLLSLPRRPGSAWDS |
| | | | | Heavy Chain-B, VH + hIgG4s |
| | | | | (Hole/Star- SEQ ID NO: 73): |
| | | | | EVQLQESGPGLVKPSQSLSLTCSVTGYSITSSYRWNWIRKFPGNR |
| | | | | LEWMGYINSAGISNYNPSLKRRISITRDTSKNQFFLQVNSVTTED |
| | | | | AATYYCARSDNMGTTPFTYWGQGTLVTVSSASTKGPSVFPLAPCS |
| | | | | RSTSESTAALGCLVKDYFPEPVTVSWNSGALTSGVHTFPAVLQSS |
| | | | | GLYSLSSVVTVPSSSLGTKTYTCNVDHKPSNTKVDKRVESKYGPP |
| | | | | CPPCPAPPVAGPSVFLFPPKPKDTLMISRTPEVTCVVVDVSQEDP |
| | | | | EVQFNWYVDGVEVHNAKTKPREEQFNSTYRVVSVLTVLHQDWLNG |
| | | | | KEYKCKVSNKGLPSSIEKTISKAKGQPREPQVYTLPPSQEEMTKN |
| | | | | QVSLSCAVKGFYPSDIAVEWESNGQPENNYKTTPPVLDSDGSFFL |
| | | | | VSRLTVDKSRWQEGNVFSCSVMHEALHNRFTQKSLSLSPGK |
| | | | | Light Chain, CH + human kappa |
| | | | | (SEQ ID NO: 69): |
| | | | | DIVMTQGTLPNPVPSGESVSITCRSSKSLLYSDGKTYLNWYLQRP |
| | | | | GQSPQLLIYWMSTRASGVSDRFSGSGSGTDFTLKISGVEAEDVGI |
| | | | | YYCQQGLEFPTFGGGTKLELKTVAAPSVFIFPPSDEQLKSGTASV |
| | | | | VCLLNNFYPREAKVQWKVDNALQSGNSQESVTEQDSKDSTYSLSS |
| | | | | TLTLSKADYEKHKVYACEVTHQGLSSPVTKSFNRGEC |
| SEQ ID NOs: | | Anti hPD1- | CH1-CH3 sequence is | Heavy Chain-A, VH + hIgG4s (Knob - SEQ ID |
| 74, 75, 70) | | IL27(L138A, | human IgG4s | NO: 74): |
| | | R145A, | (Knob x Hole/Star) | QVQLVQSGAEVKRPGSSVKVSCKVSGVTFRNFAIIWVRQAPGQGL |
| | | H146A) | | EWMGGIIPFFSAANYAQSFQGRVTITPDESTSTAFMELASLRSED |
| | | | | TAVYYCAREGERGHTYGFDYWGQGTLVTVSSASTKGPSVFPLAPC |
| | | | | SRSTSESTAALGCLVKDYFPEPVTVSWNSGALTSGVHTFPAVLQS |
| | | | | SGLYSLSSVVTVPSSSLGTKTYTCNVDHKPSNTKVDKRVESKYGP |
| | | | | PCPPCPAPPVAGPSVFLFPPKPKDTLMISRTPEVTCVVVDVSQED |
| | | | | PEVQFNWYVDGVEVHNAKTKPREEQFNSTYRVVSVLTVLHQDWLN |
| | | | | GKEYKCKVSNKGLPSSIEKTISKAKGQPREPQVYTLPPSQEEMTK |
| | | | | NQVSLWCLVKGFYPSDIAVEWESNGQPENNYKTTPPVLDSDGSFF |
| | | | | LYSRLTVDKSRWQEGNVFSCSVMHEALHNHYTQKSLSLSLGKGGG |
| | | | | GSGGGGSGGGGSYTETALVALSQPRVQCHASRYPVAVDCSWTPLQ |
| | | | | APNSTRSTSFIATYRLGVATQQQSQPCLQRSPQASRCTIPDVHLF |
| | | | | STVPYMLNVTAVHPGGASSSLLAFVAERIIKPDPPEGVRLRTAGQ |
| | | | | RLQVLWHPPASWPFPDIFSLKYRLRYRRRGASHFRQVGPIEATTF |
| | | | | TLRNSKPHAKYCIQVSAQDLTDYGKPSDWSLPGQVESAPHKPGGG |
| | | | | GSGGGGSGGGGSGGGGSFPTDPLSLQELRREFTVSLYLARKLLSE |
| | | | | VQGYVHSFAESRLPGVNLDLLPLGYHLPNVSLTFQAWHHLSDSER |
| | | | | LCFLATTLRPFPAMLGGLGTQGTWTSSEREQLWAMRADLRDLHAA |
| | | | | LRFQVLAAGFKCSKEEEDKEEEEEEEEEEKKLPLGALGGPNQVSS |
| | | | | QVSWPQLLYTYQLLHSLELVLSRAVRDLLLLSLPRRPGSAWDS |
| | | | | Heavy Chain-B, VH + hIgG4s |
| | | | | (Hole/Star - SEQ ID NO: 75): |
| | | | | QVQLVQSGAEVKRPGSSVKVSCKVSGVTFRNFAIIWVRQAPGQGL |
| | | | | EWMGGIIPFFSAANYAQSFQGRVTITPDESTSTAFMELASLRSED |
| | | | | TAVYYCAREGERGHTYGFDYWGQGTLVTVSSASTKGPSVFPLAPC |
| | | | | SRSTSESTAALGCLVKDYFPEPVTVSWNSGALTSGVHTFPAVLQS |
| | | | | SGLYSLSSVVTVPSSSLGTKTYTCNVDHKPSNTKVDKRVESKYGP |
| | | | | PCPPCPAPPVAGPSVFLFPPKPKDTLMISRTPEVTCVVVDVSQED |
| | | | | PEVQFNWYVDGVEVHNAKTKPREEQFNSTYRVVSVLTVLHQDWLN |
| | | | | GKEYKCKVSNKGLPSSIEKTISKAKGQPREPQVYTLPPSQEEMTK |
| | | | | NQVSLSCAVKGFYPSDIAVEWESNGQPENNYKTTPPVLDSDGSFF |
| | | | | LVSRLTVDKSRWQEGNVFSCSVMHEALHNRFTQKSLSLSPGK |
| | | | | Light Chain, CH + human kappa: (SEQ ID |
| | | | | NO: 70) |
| | | | | DIQMTQSPSSLSASVGDRVTITCRASQSISSYLNWYQQKPGKAPK |
| | | | | LLIYAASSLQSGVPSRFSGSGSGTDFTLTISSLQPEDFATYYCQQ |
| | | | | SYSTPPITFGQGTRLEIKRTVAAPSVFIFPPSDEQLKSGTASVVC |
| | | | | LLNNFYPREAKVQWKVDNALQSGNSQESVTEQDSKDSTYSLSSTL |
| | | | | TLSKADYEKHKVYACEVTHQGLSSPVTKSFNRGEC |
| SEQ ID NOs: | mIgG1 | N/A | Murine IgG1 Fc | Heavy Chain; Variable Region (VR) + |
| 76, 77 | | (control | isotype control | mIgG1 (SEQ ID NO: 76): |
| | | antibody | | QVQLQESGPGLVAPSQSLSITCTVSGFSLTTFGVHWVRQSPGKGL |
| | | with | | EWLGVIWADETTNYNSALMSRLSISKDNSKSQVFLKMNGLRTDDT |
| | | murine | | AIYSCARSKVSYYFDYWGRGTTLTVSSAKTTPPSVYPLAPGSAAQ |
| | | IgG1 | | TNSMVTLGCLVKGYFPEPVTVTWNSGSLSSGVHTFPAVLQSDLYT |
| | | sequence) | | LSSSVTVPSSTWPSETVTCNVAHPASSTKVDKKIVPRDCGCKPCI |
| | | | | CTVPEVSSVFIFPPKPKDVLTITLTPKVTCVWDISKDDPEVQFSW |
| | | | | FVDDVEVHTAQTQPREEQFNSTFRSVSELPIMHQDWLNGKEFKCR |
| | | | | VNSAAFPAPIEKTISKTKGRPKAPQVYTIPPPKEQMAKDKVSLTC |
| | | | | MITDFFPEDITVEWQWNGQPAENYKNTQPIMDTDGSYFVYSKLNV |
| | | | | QKSNWEAGNTFTCSVLHEGLHNHHTEKSLSHSPGK |
| | | | | Light Chain; VR + mouse Kappa (SEQ ID |
| | | | | NO: 77) |
| | | | | NIMMTQSPSSLPVSPGEKVTMNCKSSRSVLYSLNQKNYLAWYQQK |
| | | | | PGQSPKLLIYWASTRESGVPDRFTGSGSGTDFSLTISSVQAEDLA |
| | | | | VYYCHQYLSSWTFGGGTKLEIKRADAAPTVSIFPPSSEQLTSGGA |
| | | | | SVVCFLNNFYPKDINVKWKIDGSERQNGVLNSWTDQDSKDSTYSM |
| | | | | SSTLTLTKDEYERHNSYTCEATHKTSTSPIVKSFNRGEC |
| SEQ ID NOs: | hIgG4s | N/A | Human IgG4 Fc | Heavy Chain VR + hIgG4s (SEQ ID NO: 78) |
| 78, 70 | | (control | isotype control. | EVQLVESGGGLVQPGGSLRLSCAASGFTLSTYAMTWVRQAPGKGL |
| | | antibody | Fc is IgG4s | EWVSAINYRAANTWYADSVKGRFTISRDNSKNTLYLQMNSLRDED |
| | | with | | TAVYYCAQDRVIIKDYYVMDVWGQGTTVTVSSASTKGPSVFPLAP |
| | | hIgG4s | | CSRSTSESTAALGCLVKDYFPEPVTVSWNSGALTSGVHTFPAVLQ |
| | | sequence) | | SSGLYSLSSVVTVPSSSLGTKTYTCNVDHKPSNTKVDKRVESKYG |
| | | | | PPCPPCPAPPVAGPSVFLFPPKPKDTLMISRTPEVTCVVVDVSQE |
| | | | | DPEVQFNWYVDGVEVHNAKTKPREEQFNSTYRVVSVLTVLHQDWL |
| | | | | NGKEYKCKVSNKGLPSSIEKTISKAKGQPREPQVYTLPPSQEEMT |
| | | | | KNQVSLTCLVKGFYPSDIAVEWESNGQPENNYKTTPPVLDSDGSF |
| | | | | FLYSRLTVDKSRWQEGNVFSCSVMHEALHNHYTQKSLSLSLGK |
| | | | | Light Chain VR + human Kappa |
| | | | | (SEQ ID NO: 70) |
| | | | | DIQMTQSPSSLSASVGDRVTITCRASQSISSYLNWYQQKPGKAPK |
| | | | | LLIYAASSLQSGVPSRFSGSGSGTDFTLTISSLQPEDFATYYCQQ |
| | | | | SYSTPPITFGQGTRLEIKRTVAAPSVFIFPPSDEQLKSGTASVVC |
| | | | | LLNNFYPREAKVQWKVDNALQSGNSQESVTEQDSKDSTYSLSSTL |
| | | | | TLSKADYEKHKVYACEVTHQGLSSPVTKSFNRGEC |
| SEQ ID NOs: | hIgG1 | N/A | Anti-FelD1 human | Heavy Chain (SEQ ID NO: 79) |
| 79, 66 | | (control | IgG1 Fc | QVQLQESGPGLVKPSETLSLTCTVSGGSISSYYWSWIRQPPGKGL |
| | | antibody | isotype control | EWIGYIYYSGRTNYNPSLKSRVTISVDTSKNQFSLKLSSVTAADT |
| | | with | | AIYYCARHRVTRTADSFDYWGQGTLVTVSSASTKGPSVFPLAPSS |
| | | hIgG1 | | KSTSGGTAALGCLVKDYFPEPVTVSWNSGALTSGVHTFPAVLQSS |
| | | sequence) | | GLYSLSSVVTVPSSSLGTQTYICNVNHKPSNTKVDKKVEPKSCDK |
| | | | | THTCPPCPAPELLGGPSVFLFPPKPKDTLMISRTPEVTCVVVDVS |
| | | | | HEDPEVKFNWYVDGVEVHNAKTKPREEQYNSTYRVVSVLTVLHQD |
| | | | | WLNGKEYKCKVSNKALPAPIEKTISKAKGQPREPQVYTLPPSRDE |
| | | | | LTKNQVSLTCLVKGFYPSDIAVEWESNGQPENNYKTTPPVLDSDG |
| | | | | SFFLYSKLTVDKSRWQQGNVFSCSVMHEALHNHYTQKSLSLSPGK |
| | | | | Light Chain (SEQ ID NO: 66) |
| | | | | DIQMTQSPSSLSASVGDRVTITCQASQDINNYLNWYQQKTGKAPK |
| | | | | FLIYDASNLETGVSSRFSGSGSGTDFTFTISSLQPEDVGTYYCHQ |
| | | | | YGDLPYTFGQGTKLEIKRTVAAPSVFIFPPSDEQLKSGTASVVCL |
| | | | | LNNFYPREAKVQWKVDNALQSGNSQESVTEQDSKDSTYSLSSTLT |
| | | | | LSKADYEKHKVYACEVTHQGLSSPVTKSFNRGEC |

7.1.2. Measurement of STAT3-Reporter Assay

The mouse mast cell line MC/9 was transduced with a lentiviral vector encoding a STAT3 driven luciferase reporter, single cell cloned and renamed MC9/STAT3-Luc. Subsequently, IL27Ra was knocked out using CRISPR/Cas9 technology, and the resulting cell line MC9/STAT3-Luc/IL27Ra KO, was validated by flow cytometry. MC9/STAT3-Luc (ACL8878) cells were incubated O/N in assay media (RPMI+10% FBS+1% P/S/G+50 μM BME+36 μg/mL L-asparagine+1% NEAA). Cells were spun down and resuspended in assay media. 5×10⁴ MC9 reporter cells were plated per well in 50 μL. Recombinant IL27 (R&D Systems cat#2700-ML-010/CF), EBI3-p28-Fc (bivalent) or EBI3-p28-Fc (monovalent) constructs or isotype control (H4sH10154P3, also referred to as REGN7540) were resuspended at 50 nM (2×), then diluted following an 11 point titration (where the 12th has no protein). 50 μL of the titration was added per well in duplicate to a final volume of 100 μL. Cells were incubated 5.5 hour at 37° C. 100 μL OneGlo was added, and incubated 3 minutes at RT. Luciferase activity was detected on an Envision plate reader.

7.1.3. Flow Cytometric Evaluation of IL27 Chimera and Muteins Binding

MC9/STAT3-Luc and MC9/STAT3-Luc/IL27Rα KO cells were pre-bound with an Fc receptor blocking antibody (Biolegend, #156604) in FACS buffer (PBS+2% FBS) prior to binding with a 500 nM 9 point 1:5 titration, with the 9^{th} point lacking the IL27-Fc chimera. After the cells were incubated 30 min on ice, they were washed once in FACS buffer and then incubated with 5 μg/mL AF647-conjugated F(ab′)2 goat anti-human Fcg-specific antibody (Jackson Immunoresearch, #109-606-170) for another 30 minutes on ice. Next, the cells were washed once, incubated 30 minutes with a viability marker (Invitrogen, #L34970) at 1:500 in PBS, washed once and fixed with Cytofix (BD, #554655). Cells were evaluated by flow cytometry using a Beckman Cytoflex™ instrument. The resulting data were plotted to represent the geometric mean fluorescence intensity (MFI) of the ahuFC signal.

7.1.4. Flow Cytometric Evaluation of pSTAT1

Spleens were collected from naïve C57Bl/6 mice and dissociated through a 70 μm strainer to produce a single cell suspension. Red blood cells were lysed using ACK buffer (Lonza cat#10-548E). Total CD4+ T cells were isolated by negative selection bead enrichment (Miltenyi cat#130-104-454) and plated in a T25 flask with a 1:1 ratio of anti-CD3/anti-CD28 T cell activation beads (Gibco by Life Technologies, Cat:11453D). Cells were incubated at 37° C. for 72 hours. Beads were removed using a magnet and cells were resuspended at 2×10⁷/mL in 0.5% BSA/RPMI and 50 uL/well plated in a 96 well plate. Cells were rested on ice for 30 minutes before stimulation with Recombinant IL27 (R&D Systems cat#2700-ML-010/CF), EBI3-p28-Fc (bivalent) or EBI3-p28-Fc (monovalent) constructs for 20 minutes at 37° C. Cells were immediately fixed by adding an equal volume of 4% PFA for 20 minutes on ice. Cells were washed twice in Biolegend Cell Staining buffer (Biolegend cat#420201) before fixation in 90% methanol. Cells were washed twice in cell staining buffer and stained overnight in cell staining buffer. Cells were evaluated by flow cytometry using an LSR Fortessa (BD Biosciences). The following monoclonal antibodies against mouse antigens from the indicated sources were used for staining: BD Biosciences: pSTAT1 (4a, cat#562985), pSTAT3 (4/P-STAT3, cat#612569), Fc Block (93, cat#101302). Biolegend: CD8a (53-6.7, cat#100730), CD45 (30-F11, cat#103140), CD4 (RM4-5, cat#100529), CD3 (17A2, cat#100236). Thermo Fisher: Live Dead Fixable Dye (cat#L34962).

7.1.5. In Vitro T Cell Polarization

Spleens were collected from naïve C57Bl/6 mice and dissociated through a 70 um strainer to produce a single cell suspension. Red blood cells were lysed using ACK buffer (Lonza cat#10-548E). Total CD4+ T cells were isolated by negative selection bead enrichment (Miltenyi cat#130-104-454) and plated in 96 well plates coated with anti-CD3/anti-CD28 antibodies (Tonbo cat#70-0031-U500, 70-0281-500U) in the presence of the indicated stimuli (Th0: 10 ug/mL anti-IFNg (BioXcell cat# BE0055)+10 μg/mL anti-IL4 (BioXcell cat# BE0045), Th2: 10 ug/mL anti-IFNg+50 μg/mL rIL4 (eBiosciences cat#BMS338), Th17: 10 μg/mL anti-IFNg+10 μg/mL anti-IL4+1 ng/mL rhTGFb (R&D Systems cat#240-B-002)+10 ng/mL rIL6 (eBiosciences cat#RMIL61))+/−50 ng/mL of the indicated IL27 construct. Cells were incubated at 37° C. for 4 days, with fresh media and reagents supplemented at 72 hours. Cells were washed with PBS and stained for surface markers in cell staining buffer. Cells were washed, fixed, and stained intracellularly with eBioscience Intracellular Fixation & Permeabilization Buffer Set (eBiosciences cat#88-8824-00) as per manufacturer instructions. Cells were evaluated by flow cytometry using an LSR Fortessa (BD Biosciences). The following monoclonal antibodies against mouse antigens from the indicated sources were used for staining: BD Biosciences: Gata3 (L50-823, cat#560405), Fc Block (2.4G2, cat#553142). Biolegend: CD8a (53-6.7, cat#100714), CD45 (30-F11, cat#103138), CD4 (RM4-5, cat#100547), TCRb (H57-597, cat#109243). Thermo Fisher: Live Dead Fixable Dye (cat#L34962), Foxp3 (FJK-16s, cat#56-5773-82).

### 7.2. Example 1: Activity of IL27 Muteins in STAT3 Reporter Assay

The ability of recombinant IL27 muteins to stimulate IL27 receptor was assessed in a STAT3-reporter cell-based bioassay as described in Section 7.1.2.

Activation curves are shown in FIG. 7A for engineered reporter cells, MC9/STAT3-Luc cells, incubated with recombinant human and murine IL27, IL27 muteins or the human IgG4s isotype control. Size exclusion traces are shown in FIGS. 7B and 7C for EBI3-p28-Fc bivalent format (e.g., a configuration of IL27M1) and EBI3×p28 Fc monovalent format (e.g., a configuration of IL27M2).

When reporter cells were treated with the control protein, no increase in luciferase activity was detected. In contrast, incubation of the reporter cells MC9/STAT3-Luc with murine IL27 induced luciferase activity with an EC50 value of approximately 6.3×10-12 M. Incubation of the MC9/STAT3-Luc with murine IL27 muteins also induced luciferase activity with EC50 values of approximately 4.8×10-10 M and 3.2×10-12 M. Thus, EBI3-p28-Fc (monovalent) signaling, but not EBI3-p28-Fc (bivalent) signaling, is similar to wild-type IL27. The bivalent format of the mutein (EBI3-p28-Fc (bivalent)) demonstrated a higher degree of aggregation compared to the monovalent format (EBI3-p28-Fc (monovalent)).

### 7.3. Example 2: Activity of IL27 Muteins in pSTAT1 Assay

The ability of recombinant IL27 muteins to stimulate IL27 receptor was assessed in a pSTAT1-flow cytometry bioassay as described in Section 7.1.3.

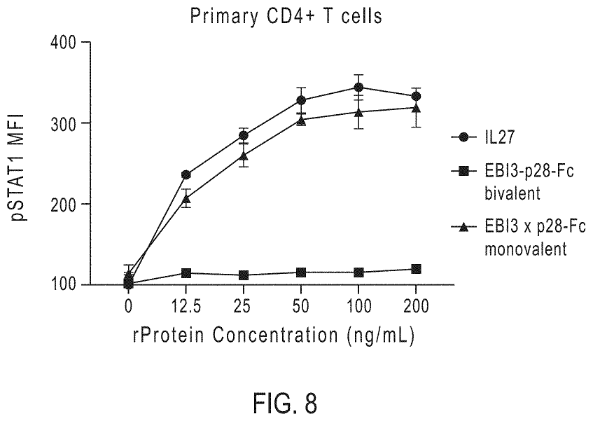

MFI curves are shown in FIG. 8 for CD4+ T-cells incubated with recombinant IL27 and IL27 muteins.

Incubation of CD4+ T-cells with murine IL27 and EBI3-P28-FC (monovalent) induced phosphorylation of STAT1 with EC50 values of approximately 24.4 ng/mL and 22.37 ng/mL, respectively. In contrast, no increase in STAT1 phosphorylation was seen in cells treated with EBI3-p28-Fc (bivalent) (with an EC₅₀ value of approximately 1999 ng/mL), again suggesting that EBI3-p28-Fc (monovalent) signaling, but not EBI3-p28-Fc (bivalent) signaling, is similar to wild-type IL27.

### 7.4. Example 3: Size Separation of IL27 Muteins

Size-exclusion ultra-performance liquid chromatography (SE-UPLC) was employed to assess the oligomeric state of different muteins. SEC analysis was conducted on a Waters Acquity UPLC H-Class system. 10 μg of each protein sample was injected onto a Acquity BEH SEC column (200 Å, 1.7 μm, 4.6×300 mm). Flow rate was set at 0.3 ml/min. Mobile phase buffer contains 10 mM sodium phosphate, 500 mM NaCl, pH 7.0. UV absorbance at 280 nm was monitored.

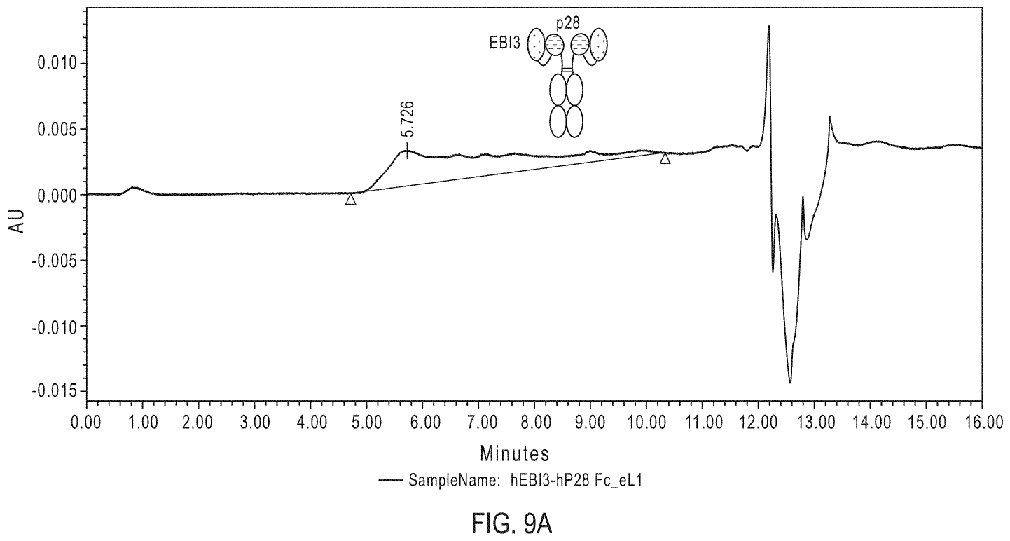

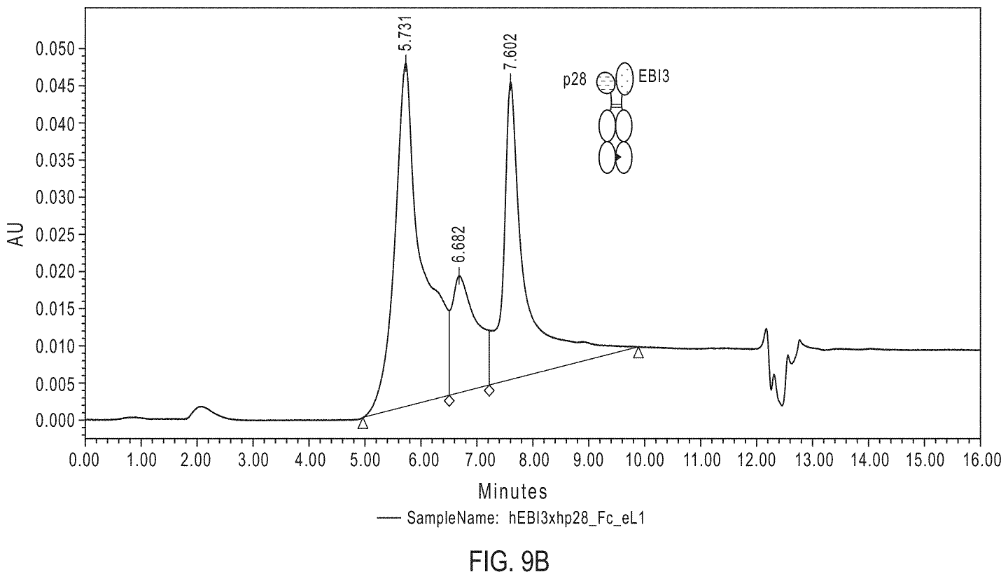

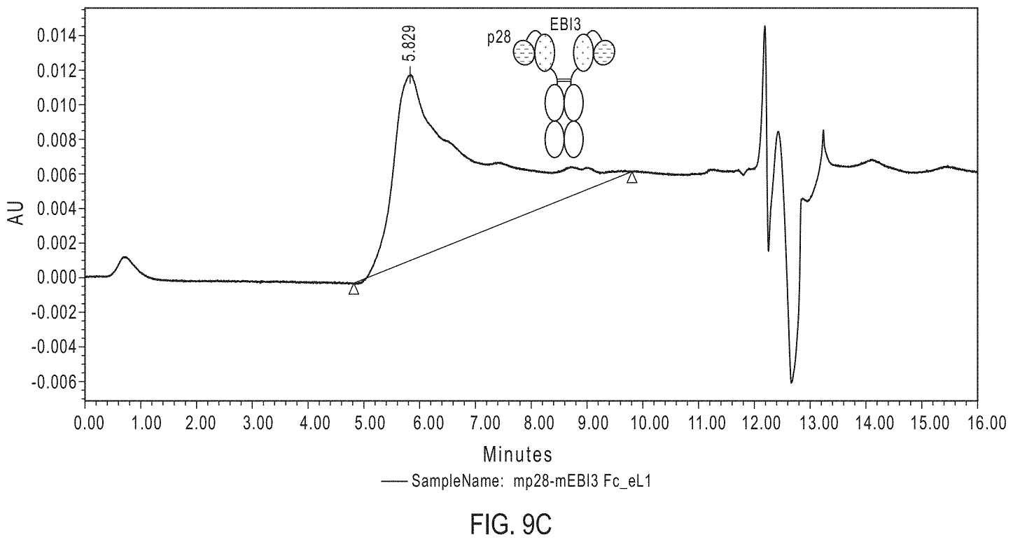

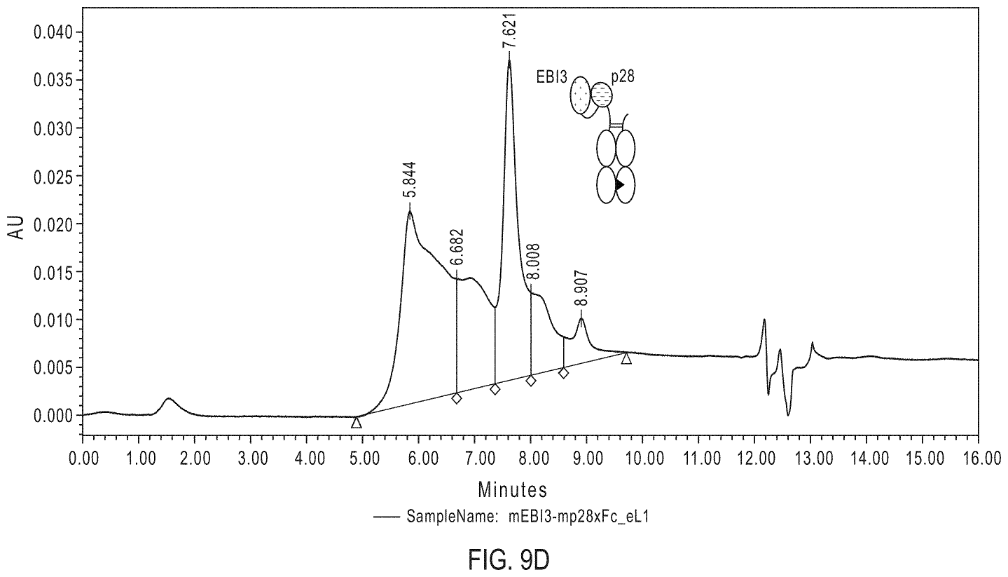

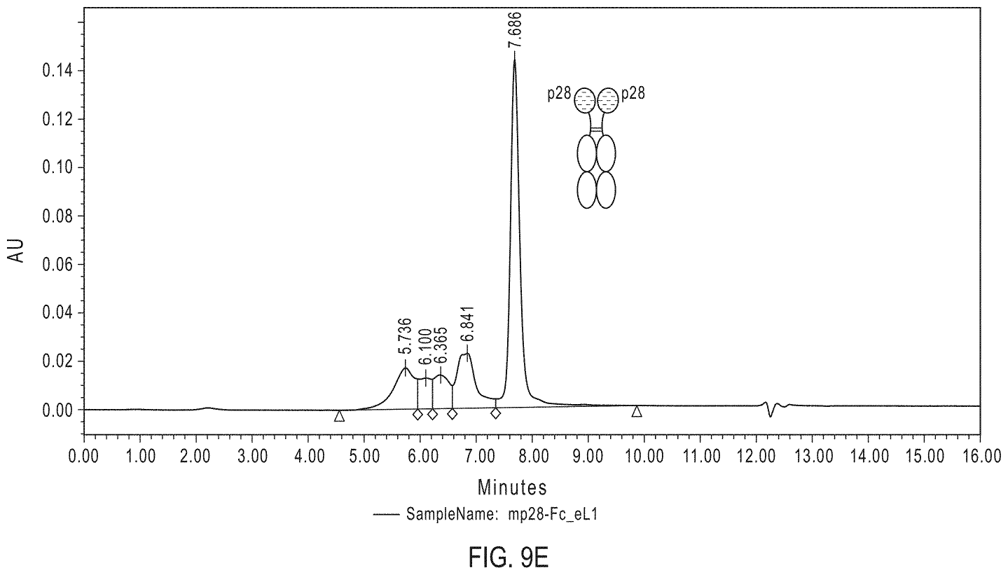

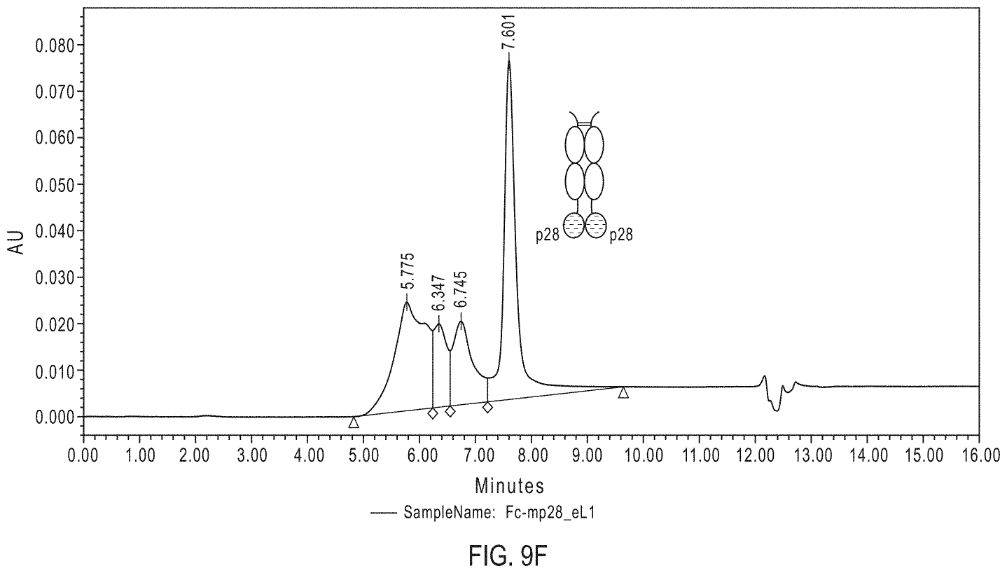

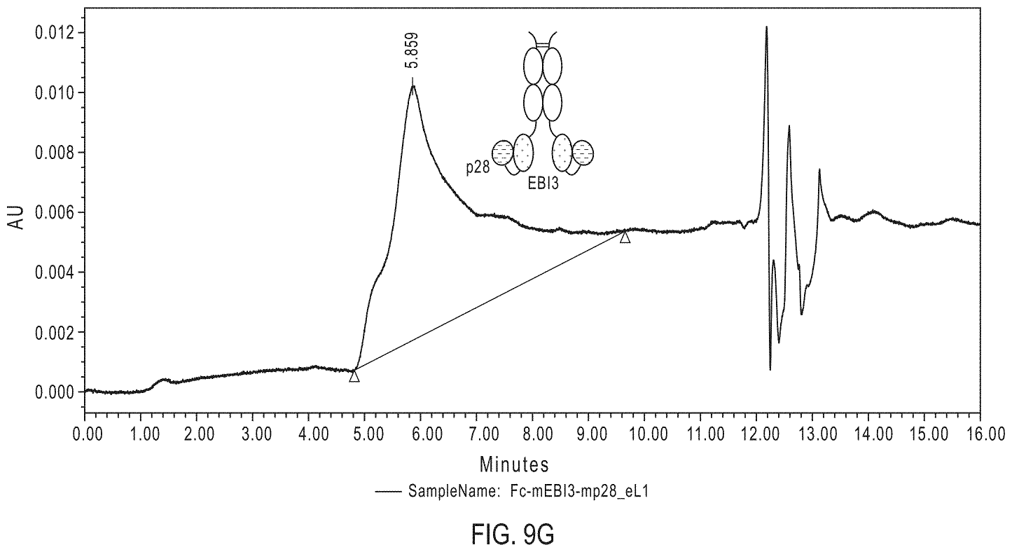

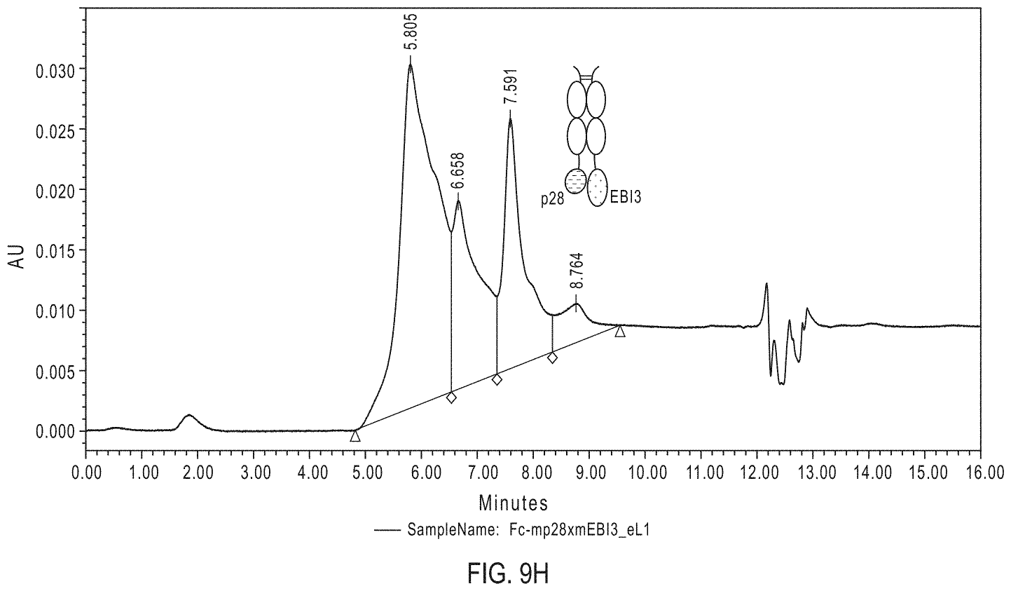

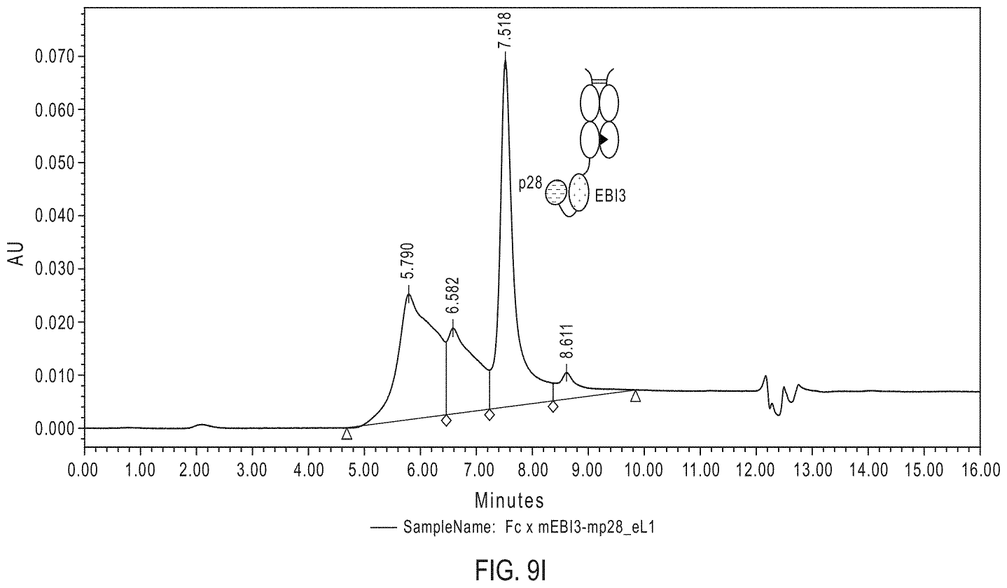

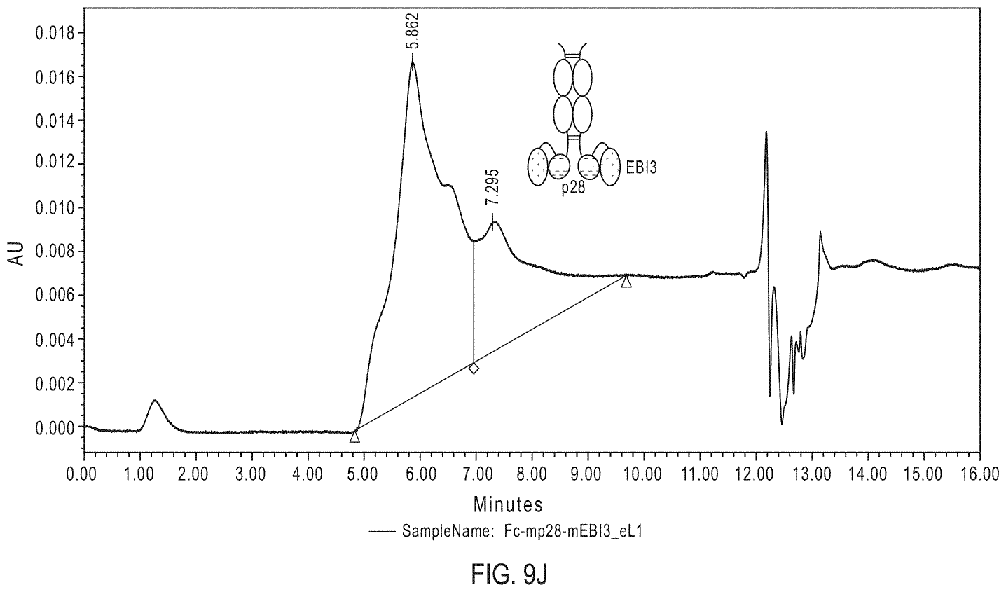

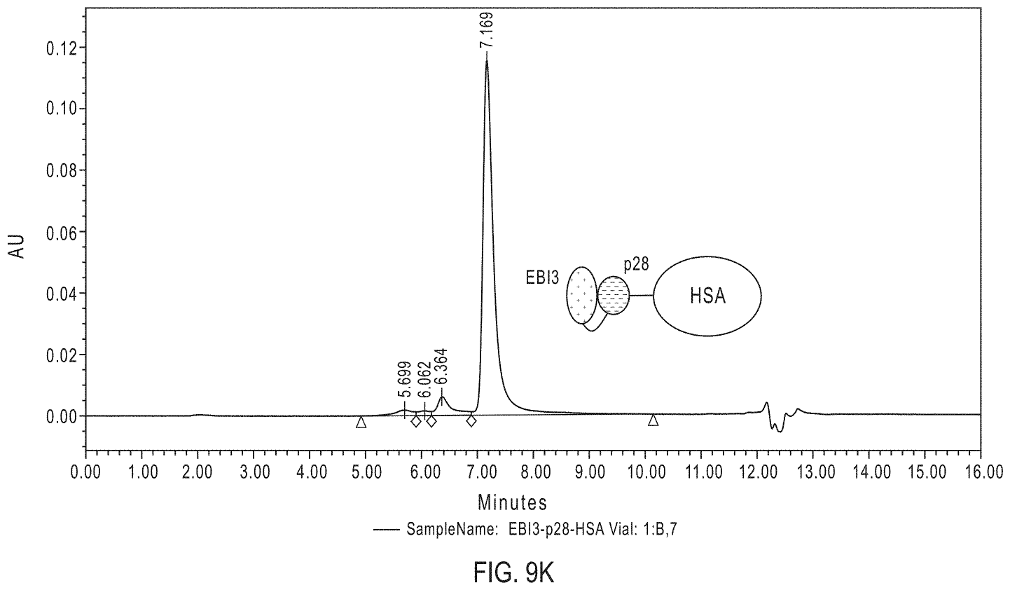

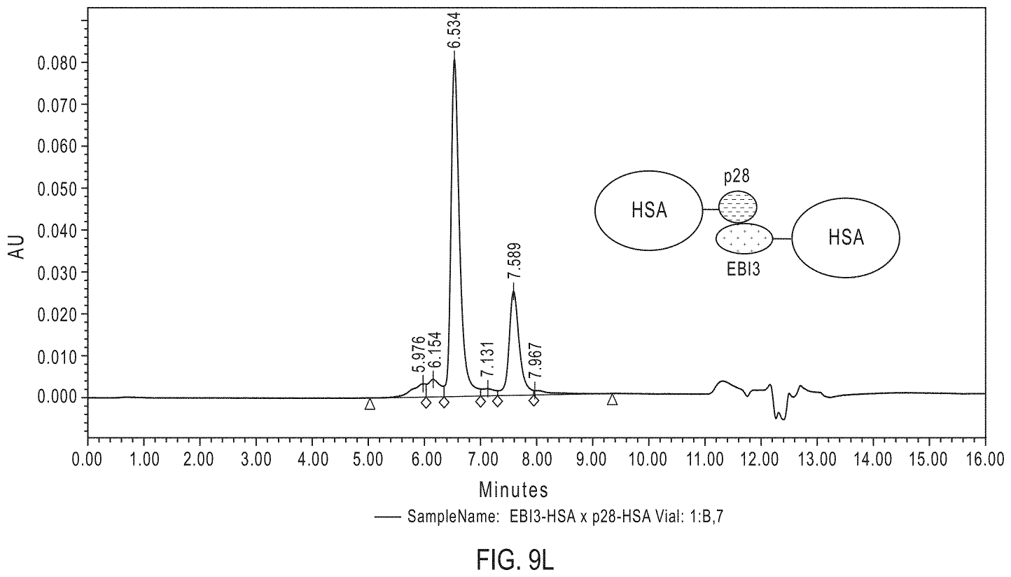

The SE-UPLC traces for IL27 muteins are shown in FIGS. 9A-9L. Most of the IL27 muteins displayed a high degree of size heterogeneity as shown by prominent HMW species on the SE-UPLC traces. Monovalent formats of the IL27 muteins show a more favorable stability profile with less oligomerization compared to the bivalent formats.

### 7.5. Example 4: Activity of IL27 Muteins in IL27 Reporter Assay

The ability of recombinant IL27 muteins to induce STAT3 activity in a reporter assay was assessed. Briefly, 5×10⁴ MC9 cells were plates and incubated with the IL27 mutein (11 point titration) for 5 h 30 min and then read (luciferase assay).

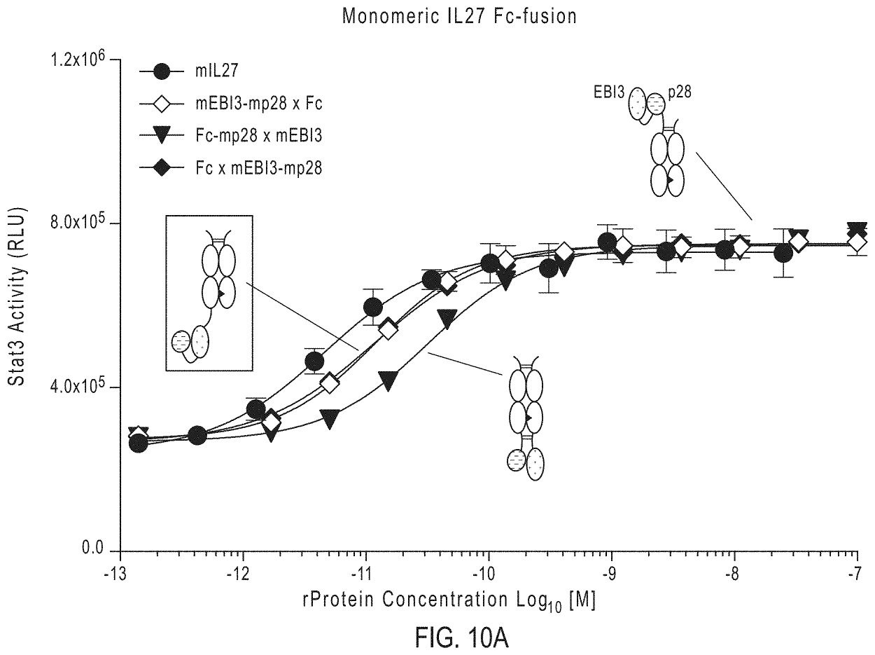

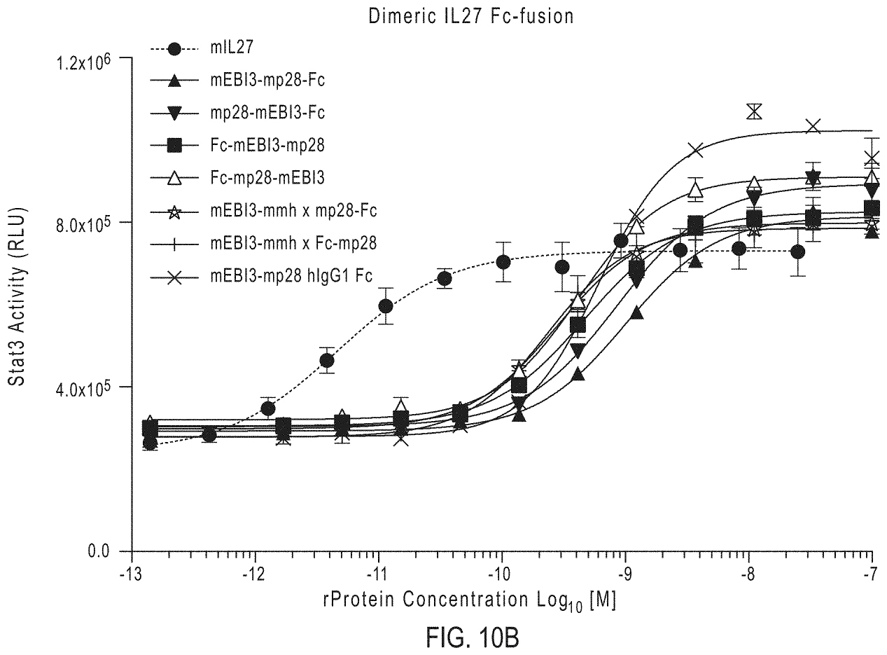

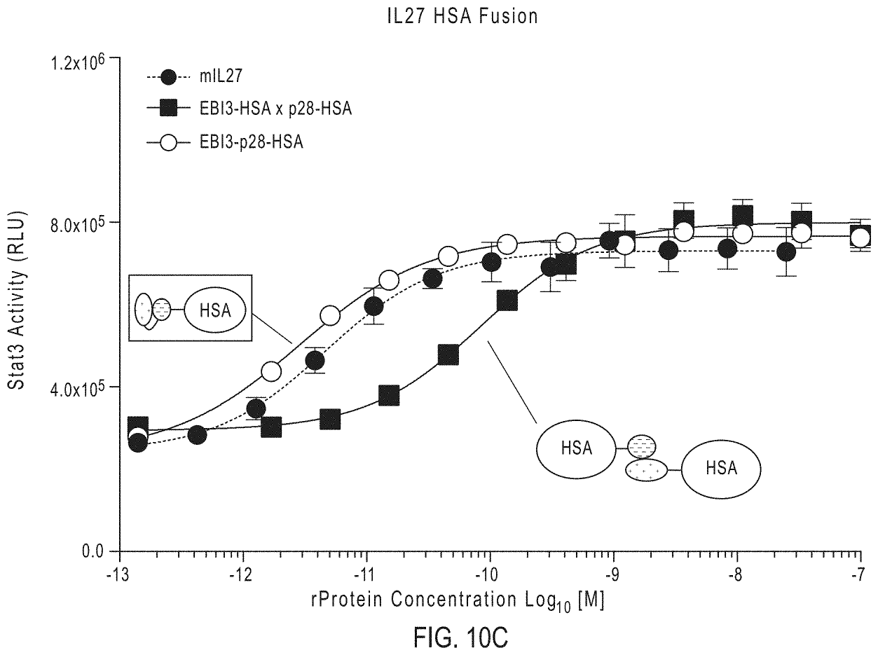

The dose response curves are shown in FIGS. 10A-10C and the calculated EC50 value and fold induction is shown below in Table 7. The monovalent IL27 formats have higher potency than the bivalent IL27 formats.

TABLE 7
IL27 reporter assay EC50 and Fold induction for mouse IL27, mouse
Fc-mIL27, IL27-Fc, HSA-IL27 and IL27-HSA tagged proteins

| Mutein | | FOLD | |
|---|---|---|---|
| Configuration | CONSTRUCT | INDUCTION* | EC50 (M) |
| | mIL27 | 2.86 | 4.62E−12 |
| IL27M1 | mEB13-mp28-Fc | 2.95 | 1.01E−09 |
| IL27M3 | mp28-mEB13-Fc | 3.02 | 8.63E−10 |
| IL27M12 | mEB13-mp28 × Fc | 2.68 | 1.22E−11 |
| IL27M13 | mp28-Fc | 1.11 | ND |
| IL27M4 | Fc-mEB13-mp28 | 2.78 | 4.71E−10 |
| IL27M5 | Fc-mp28-mEB13 | 2.91 | 4.19E−10 |
| IL27M6 | Fc-mp28 × mEB13 | 2.80 | 3.21E−11 |
| IL27M16 | Fc × mEB13-mp28 | 2.76 | 1.18E−11 |
| IL27M17 | Fc-mp28 | 1.16 | ND |
| IL27M14 | mEB13-mmh × mp28-Fc | 2.71 | 2.81E−10 |
| IL27M18 | mEB13-mmh × Fc-mp28 | 2.86 | 2.36E−10 |
| IL27M22 | mEB13-mp28 hIgG1 Fc | 3.95 | 6.08E−10 |
| IL27M20 | mEB13-mp28-HSA | 2.79 | 2.87E−12 |
| IL27M21 | mEB13-HSA × mp28-HSA | 2.70 | 8.35E−11 |

ND: not determined because a concentration-dependent response was not observed.
*Fold induction is defined as the highest mean RLU value within the tested dose-range relative to the mean RLU in absence of recombinant protein.

### 7.6. Example 5: Activity of IL27 Muteins in IL27 Reporter Assay

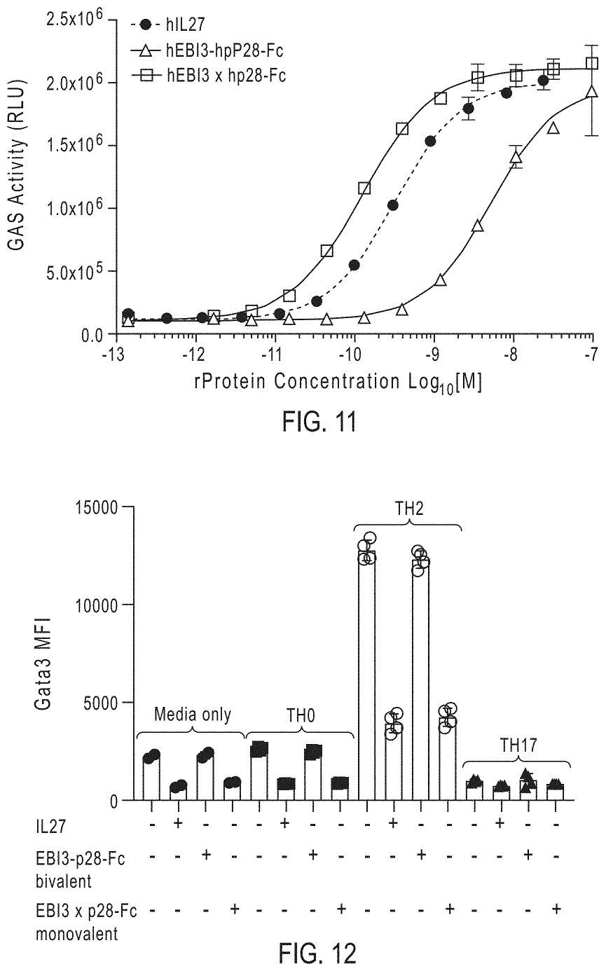

The human plasma cell line NCI-H929 was transduced with a lentiviral vector encoding an interferon Gamma-Activated Sequence (GAS) driven luciferase reporter and renamed NCI-H929/GAS-Luc. The ability of recombinant human IL27 muteins to induce STAT1-driven reporter activity was assessed by incubating NCI-H929/GAS-Luc with a titration of IL27, human EBI3-p28-Fc (bivalent) or human hEBI3×hp28-Fc (monovalent). The dose response curves are shown in FIG. 11 and the calculated EC50 value and fold induction is shown below in Table 8.

TABLE 8
IL27 reporter assay EC50 and Fold induction for human IL27,
monovalent (IL27M12) and bivalent (IL27M1) IL27-Fc

| Mutein | | FOLD | |
|---|---|---|---|
| Configuration | CONSTRUCT | INDUCTION* | EC50 (M) |
| | hIL27 | 12.34 | 3.34E−10 |
| IL27M1 | hEB13-hp28-Fc | 15.23 | 5.35E−09 |
| IL27M12 | hEB13 × hp28-Fc | 14.27 | 1.27E−10 |

*Fold induction is defined as the highest mean RLU value within the tested dose-range relative to the mean RLU in absence of recombinant protein.

### 7.7. Example 6: Activity of IL27 Muteins in In Vitro T-Cell Polarization

The ability of recombinant IL27 muteins to inhibit GATA-3 expression during TH2 polarization of murine T-cells was assessed in an in vitro T-cell polarization assay as described in Section 7.1.5.

MFI curves are shown in FIG. 12 for CD4+ T-cells incubated in the presence of the stimuli indicated, together with recombinant IL27 and IL27 muteins.

Incubation of CD4+ T-cells with murine IL27 and EBI3-p28-Fc (monovalent) inhibited expression of the Th2-specific GATA-3 transcription factor. In contrast, no inhibition of GATA-3 was seen in cells treated with the IL27 mutein EBI3-p28-Fc (bivalent), again suggesting that EBI3-p28-Fc (monovalent) signaling, but not EBI3-p28-Fc (bivalent) signaling, is similar to wild-type IL27.

### 7.8. Example 7: Activity of IL27 Muteins in In Vitro T-Cell Polarization

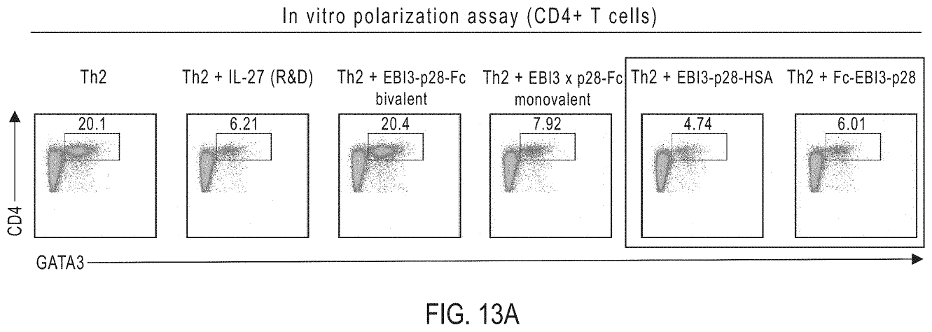

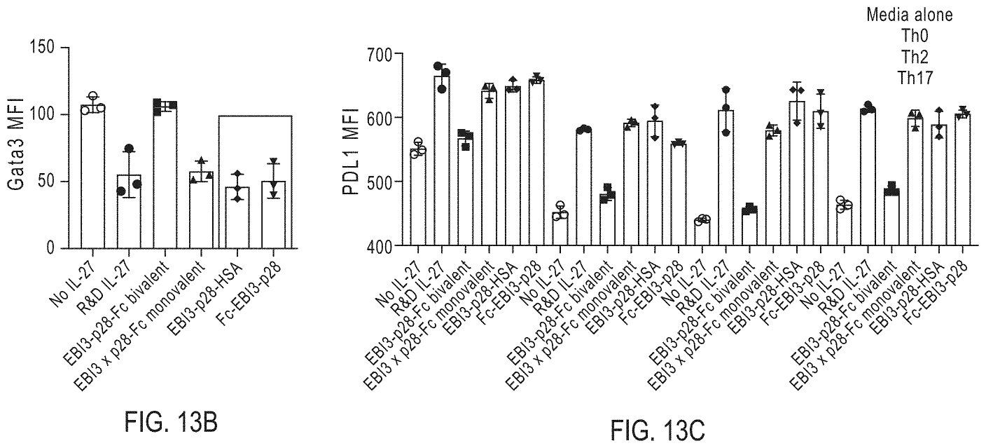

The ability of recombinant IL27 muteins to inhibit Gata3 expression during Th2 polarization (10 ug/mL anti-IFNg+50 ng/mL IL-4) was assessed in an in vitro T cell polarization assay. Referring to FIGS. 13A-13C, EBI3-p28-HSA and Fc×EBI3-p28promote PDL1 expression during in vitro Th0-, Th2-, Th17-cell polarization and inhibit Gata3 expression during in vitro Th2 polarization comparable to recombinant IL27.

### 7.9. Example 8: Evaluation of In Vitro PK of IL27 Muteins

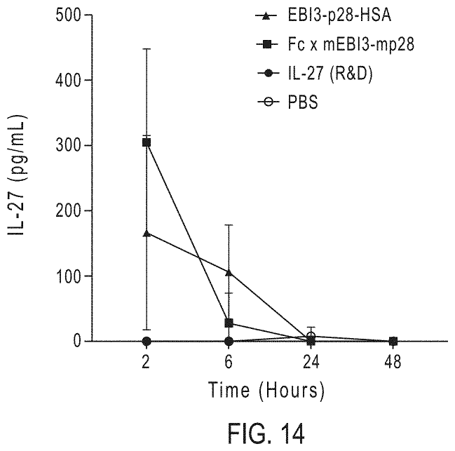

The PK of recombinant IL27 muteins was evaluated in vivo. C57BL/6 mice were injected intraperitoneally with 10 ug of Fc×EBI3-p28 or EBI3-p28-HSA. Serum was collected 2, 6, 24, and 48 hours after treatment. An ELISA was performed to quantify the respective IL-27 mutein levels in serum at each time point. FIG. 14.

### 7.10. Example 9: Identification of IL27p28 Residues Involved in Receptor Binding

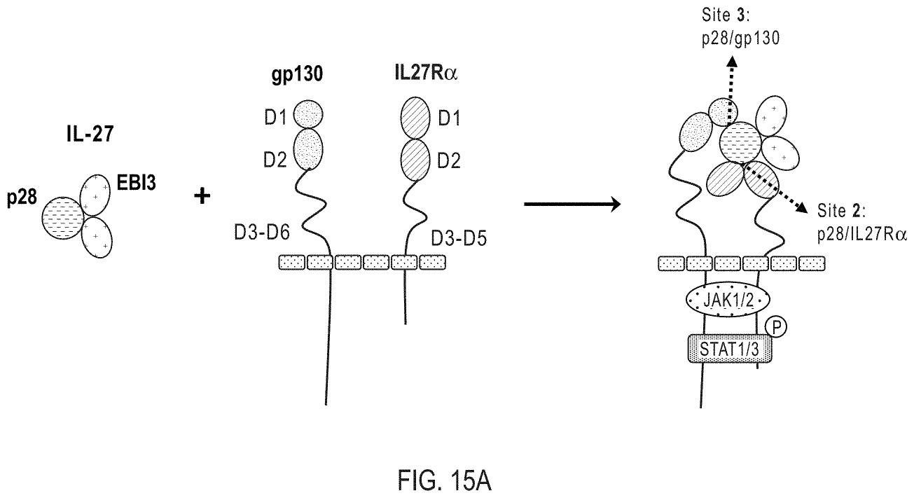

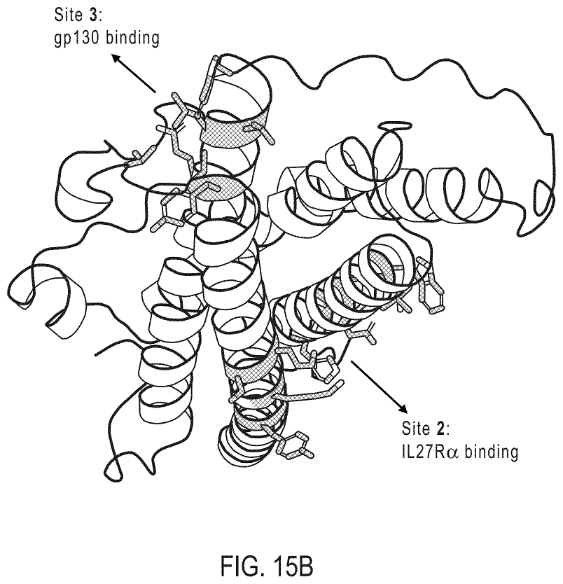

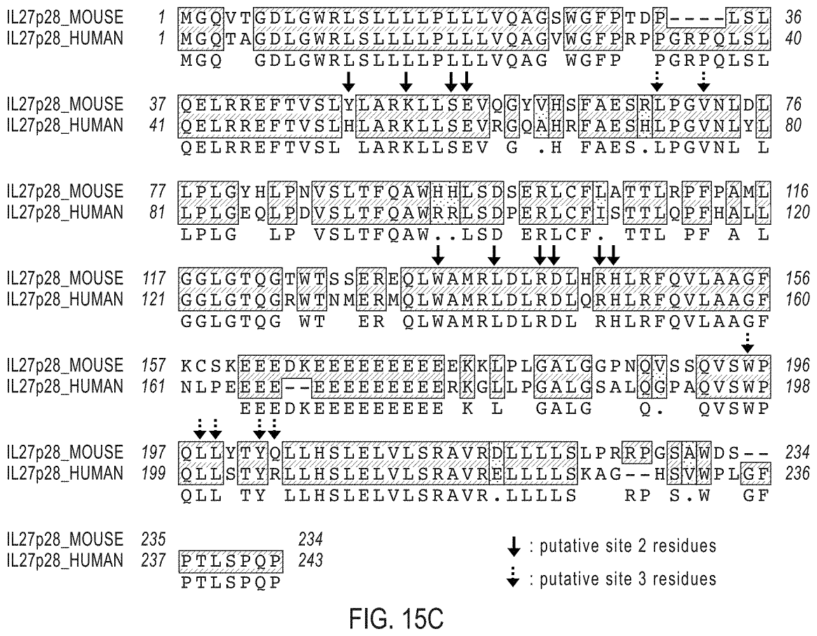

The sequence alignment of mouse IL27p28 (SEQ ID NO: 36) and human IL27p28 (SEQ ID NO:34) is shown in FIG. 15, arrows indicate sites selected for amino acid substitutions.

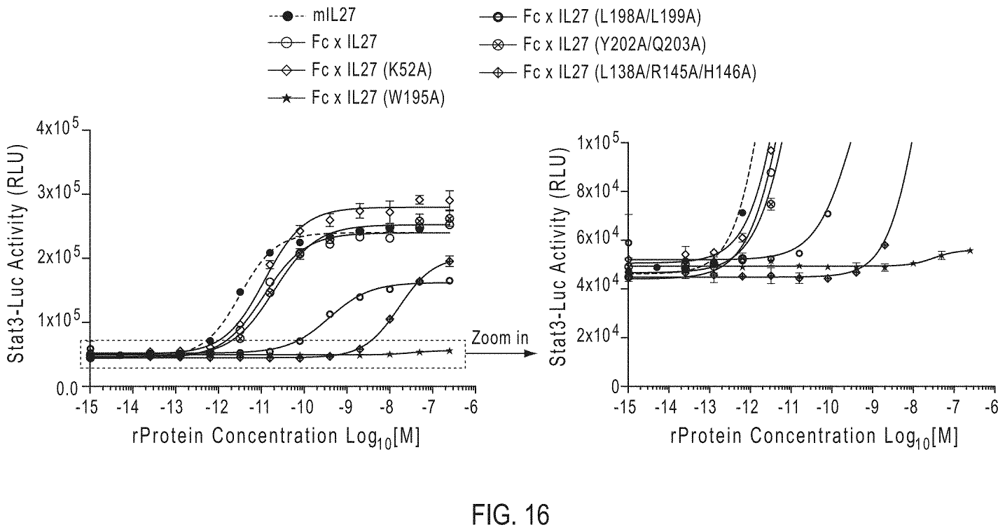

The ability of recombinant mouse IL27 muteins to induce STAT3 activity in a reporter assay was assessed. Briefly, 2.5×10⁴ cells were plated and incubated with the IL27 mutein (11 point titration) for 5 h and then read (luciferase assay). The dose response curve is shown in FIG. 16 and the calculated EC50 value and fold induction is shown below in Table 9. Mutations in putative site 2 and site 3 both impact IL27 bioactivity.

TABLE 9
IL27 reporter assay EC50 and Fold
induction for mouse IL27, monovalent
FC-IL27 and FC-IL27 muteins

| Mutein | | | |
|---|---|---|---|
| Config- | | FOLD | |
| uration | CONSTRUCT | INDUCTION* | EC50 (M) |
| | mIL27 | 5.25 | 3.22E−12 |
| IL27M16 | Fc x EB13-p28 | 5.62 | 1.16E−11 |
| IL27M27 | Fc x EB13-p28 | 1.20 | NC |
| | (W195A) | | |
| IL27M26 | FC x EB13-p28 | 2.80 | 3.85E−10 |
| | (L198A, L199A) | | |
| IL27M23 | Fe x EB13-p28 | 5.34 | 1.98E−11 |
| | (Y202A_Q203A) | | |
| IL27M24 | Fc x EB13-p28 | 5.59 | 1.18E−11 |
| | (K52A) | | |
| IL27M25 | Fc x EB13-p28 | 4.35 | NC |
| | (L138A_R145A_H146A) | | |

NC: not calculated because the data did not fit a 4-parameter logistic equation
*Fold induction is defined as the highest mean RLU value within the tested dose-range relative to the mear RLU in absence of recombinant protein.

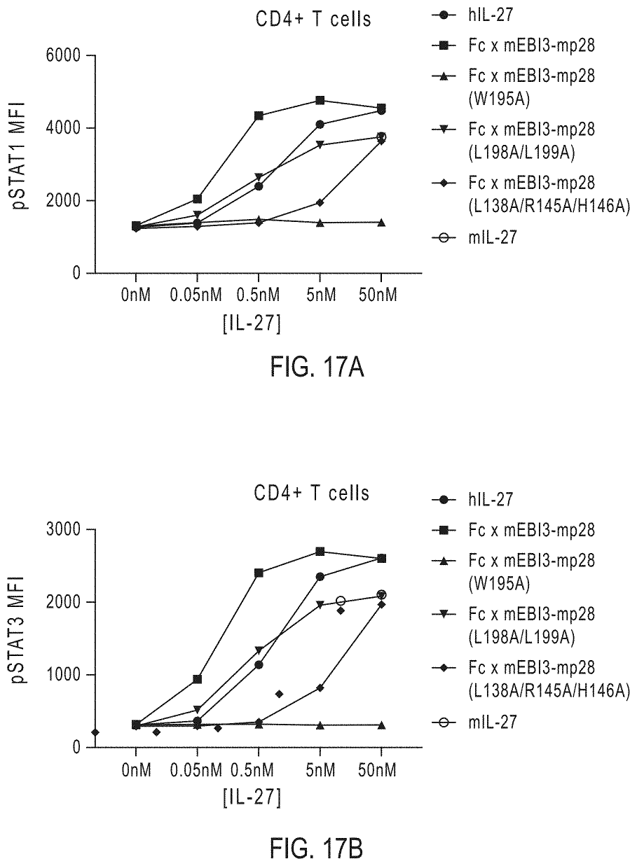

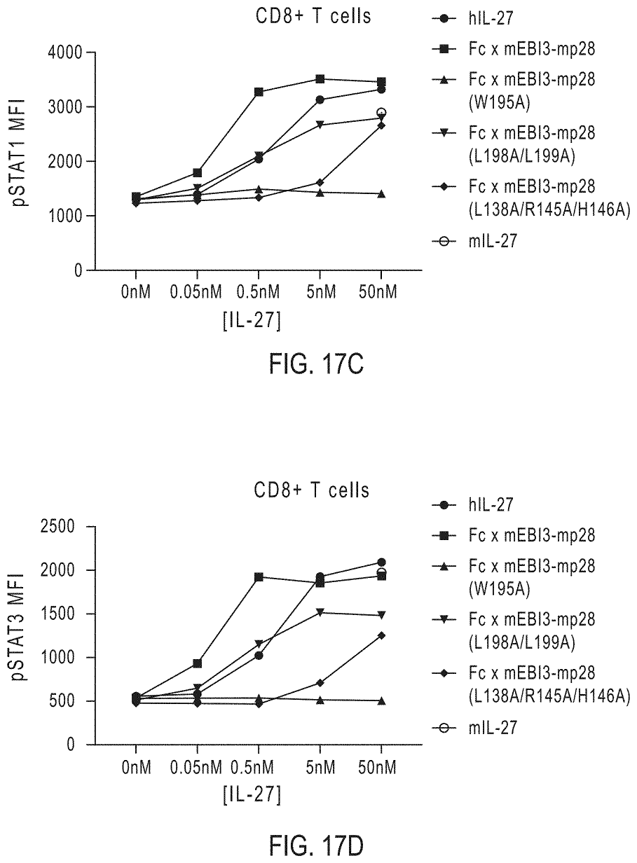

The effect of mIL27 site 2 and site 3 muteins in primary human T cells was investigated. The results are shown in FIGS. 17A-D. mIL27 was more active than hIL27 on human cells. mIL27 site 2 and site 3 muteins show reduced pSTAT1 and pSTAT3 in primary human T cells compared to WT mIL27.

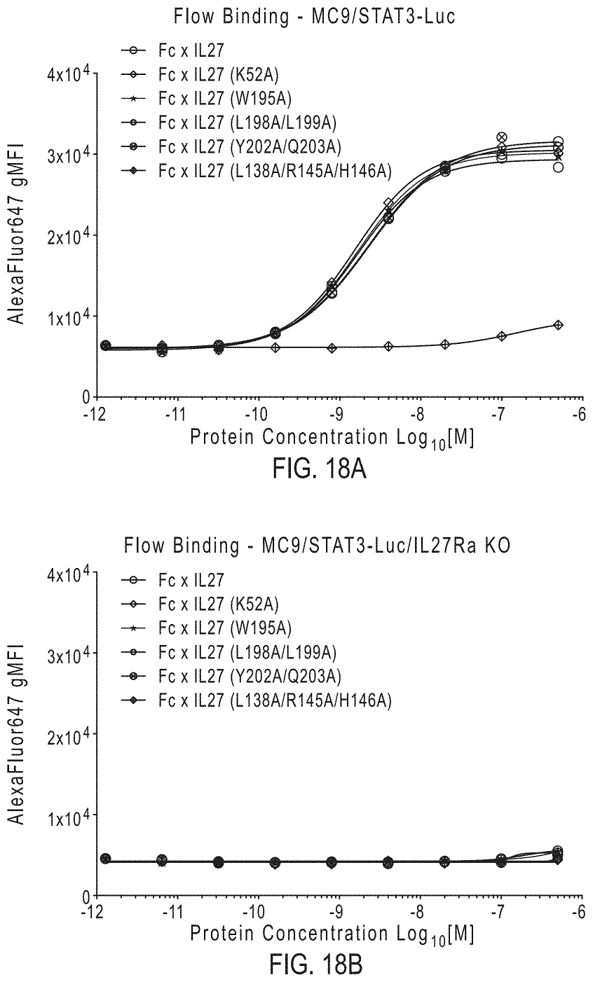

Flow binding assays were performed to investigate the binding of mIL27 muteins to wild type and mIL27Ra knockout cells. Briefly, MC9/STAT3-Luc and MC9/STAT3-Luc/IL27Ra KO cells were plated with the IL27 mutein (8 point titration), stained on ice for 30 mins followed by incubation with a secondary Fab2 anti-human IgG Fc specific AF647 then analyzed by flow cytometry. Curves are shown in FIGS. 18A-18B. While site 3 muteins had similar binding to wild type cells, the site 2 mutein had reduced binding. Furthermore, IL27Rα knockout disrupted wild type and site 3 mutein binding suggesting IL27 high affinity binding is likely mediated by site 2.

### 7.11. Example 10: Targeting of IL27 Muteins

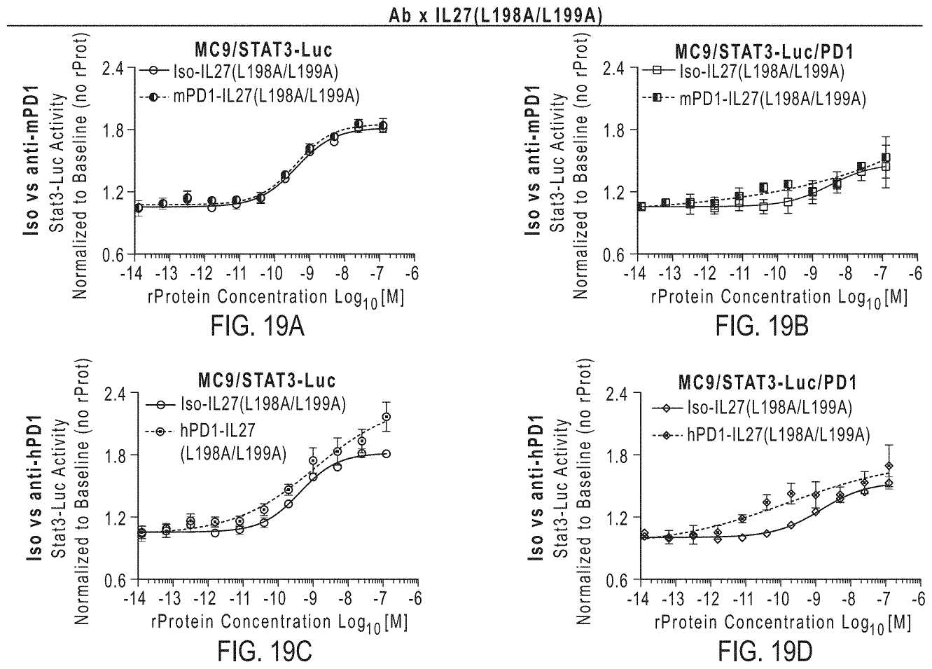

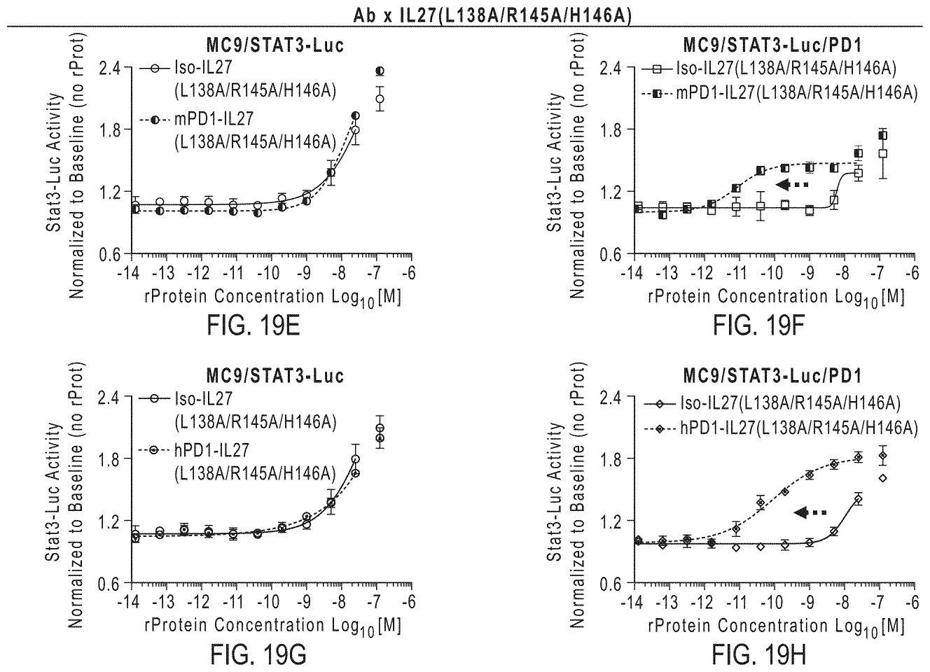

We investigated target-driven Ab-IL27 mutein capture to allow Ab targeted IL27 signaling. MC9/Stat3-Luc cells were engineered to stably express mouse PD1 (amino acids M1-L288 of accession number NP_032824.1) or human PD1 (amino acids M1-L288 of accession number NP_005009.2, with a 2Q->E mutation) and renamed MC9/STAT3-Luc/mPD1 and MC9/STAT3-Luc/hPD1 respectively. A PD1 targeting moiety was used to target IL27 site 2 and site 3 muteins in PD1 non-expressing (negative control, FIGS. 19A/C/E/G), mouse PD1 (FIGS. 19B/F) or human PD1 (FIGS. 19D/H) expressing cells, and their ability to induce STAT3 activity assessed using a reporter assays. Briefly, 2.5×10⁴ cells were plated and incubated with the IL27 mutein (12 point titration, where the 12^{th} point does not contain mutein) for 5 h and then read (luciferase assay). Curves are shown in FIGS. 19A-19H, IL27 site 2 mutein had increased potency when targeted using a PD1 targeting moiety on target (PD1) expressing cells.

### 8. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. An IL27 receptor agonist that is monovalent for IL27 and comprises:
(a) a p28 moiety comprising an IL27Ra binding domain and/or a gp130 binding domain of p28;
(b) an EBI3 moiety comprising a p28 binding domain of EBI3;
(c) a first multimerization moiety or a first stabilization moiety; and
(d) optionally, a first targeting moiety.

2. The IL27 receptor agonist of claim 1, which comprises a first stabilization moiety.

3. The IL27 receptor agonist of claim 2, comprising an IL27 monomer having the configuration of Exemplary Monomer 13, wherein Exemplary Monomer 13 comprises:
(a) the EBI3 moiety;
(b) an optional linker;
(c) the p28 moiety;
(d) an optional linker; and
(e) the first stabilization moiety

4. The IL27 receptor agonist of claim 1, which comprises a first stabilization moiety and a second stabilization moiety.

5. The IL27 receptor agonist of claim 4, comprising a first IL27 monomer having the configuration of Exemplary Monomer 9 and a second IL27 monomer having the configuration of Exemplary Monomer 10, wherein
(a) Exemplary Monomer 9 comprises:
(i) the EBI3 moiety;
(ii) an optional linker; and
(iii) the first stabilization moiety; and
(b) Exemplary Monomer 10 comprises:
(i) the p28 moiety;
(ii) an optional linker; and
(iii) the first stabilization moiety.

6. The IL27 receptor agonist of any one of claims 2 to 5, wherein the first stabilization moiety and, if present, the second stabilization moiety has an amino acid sequence with at least about 90% sequence identity to mature human serum albumin or a natural variant thereof.

7. The IL27 receptor agonist of claim 1, comprising an IL27 monomer having the configuration of Exemplary Monomer 7, wherein Exemplary Monomer 7 comprises:
(a) optionally, the first targeting moiety or first targeting moiety component associated with a counterpart first targeting moiety component on a separate polypeptide chain;
(b) an optional linker′
(c) the first multimerization moiety;
(d) the EBI3 moiety;
(e) an optional linker; and
(f) the p28 moiety.

8. The IL27 receptor agonist of claim 1 or claim 7, which comprises the first multimerization moiety and a second multimerization moiety.

9. The IL27 receptor agonist of claim 8, which further comprises a separate polypeptide chain comprising:
(a) the second multimerization moiety, which is capable of associating with the Exemplary Monomer; and
(b) optionally, a second targeting moiety or second targeting moiety component associated with a counterpart second targeting moiety component on a separate polypeptide chain.

10. The IL27 receptor agonist of claim 8 or claim 9, wherein the first multimerization moiety and the second multimerization moiety each is or comprises an Fc domain.

11. The IL27 receptor agonist of claim 10, wherein the Fc domain comprises a hinge domain.

12. The IL27 receptor agonist of claim 10 or claim 11, wherein the Fc domain is an IgG1, IgG2, IgG3, or IgG4 Fc domain.

13. The IL27 receptor agonist of any one of claims 10 to 12, wherein the Fc domain has reduced effector function.

14. The IL27 receptor agonist of any one of claim 10 to 13, wherein the Fc domain is an IgG4 Fc domain.

15. The IL27 receptor agonist of any one of claims 10 to 14, wherein the Fc domain comprises the amino acid sequence ESKYGPPCPPCPAPPVAGPSVFLFPPKPKDTLMISRTPEVTCVVVDVSQEDPEVQFNWYVDGVE VHNAKTKPREEQFNSTYRVVSVLTVLHQDWLNGKEYKCKVSNKGLPSSIEKTISKAKGQPREPQV YTLPPSQEEMTKNQVSLTCLVKGFYPSDIAVEWESNGQPENNYKTTPPVLDSDGSFFLYSRLTVD KSRWQEGNVFSCSVMHEALHNHYTQKSLSLSLGK (SEQ ID NO: 80) or a portion thereof.

16. The IL27 receptor agonist of any one of claims 1 to 15, wherein the EBI3 moiety has an amino acid sequence with at least about 90% sequence identity to a p28 binding domain of mature human EBI3.

17. The IL27 receptor agonist of claim 16, wherein the EBI3 moiety has an amino acid sequence with at least about 90% sequence identity to mature human EBI3.

18. The IL27 receptor agonist of any one of claims 1 to 17, wherein the p28 moiety has an amino acid sequence with at least about 90% sequence identity to an IL27Ra binding domain of mature human p28 and/or a gp130 binding domain of mature human p28.

19. The IL27 receptor agonist of claim 18, wherein the p28 moiety has an amino acid sequence with at least about 90% sequence identity to mature human p28.

20. The IL27 receptor agonist of any one of claims 1 to 19, wherein the p28 moiety has a variant p28 domain:
(a) comprising an amino acid sequence with at least 90% to an IL27Ra binding domain of mature human and one or more amino acid substitutions at the position corresponding to:
(i) amino acid H52 of full length human p28 or amino acid Y48 of full length murine p28, wherein the substitution is optionally alanine;
(ii) amino acid K56 of full length human p28 or amino acid K52 of full length murine p28, wherein the substitution is optionally alanine,
(iii) amino acid S59 of full length human p28 or amino acid S55 of full length murine p28, wherein the substitution is optionally alanine;
(iv) amino acid E60 of full length human p28 or amino acid E56 of full length murine p28, wherein the substitution is optionally alanine;
(v) amino acid W138 of full length human p28 or amino acid W134 of full length murine p28, wherein the substitution is optionally alanine;
(vi) amino acid L142 of full length human p28 or amino acid L138 of full length murine p28, wherein the substitution is optionally alanine;
(vii) amino acid R145 of full length human p28 or amino acid R141 of full length murine p28, wherein the substitution is optionally alanine;
(viii) amino acid D146 of full length human p28 or amino acid D142 of full length murine p28, wherein the substitution is optionally alanine;
(ix) amino acid R149 of full length human p28 or amino acid R145 of full length murine p28, wherein the substitution is optionally alanine;
(x) amino acid H150 of full length human p28 or amino acid H146 of full length murine p28, wherein the substitution is optionally alanine; or
(xi) any combination of (a)(i) to (a)(x); and/or
(b) comprising an amino acid sequence with at least 90% to a gp130 binding domain of mature human and one or more amino acid substitutions at the position corresponding to:
(i) amino acid L73 of full length human p28 or amino acid L69 of full length murine p28, wherein the substitution is optionally alanine;
(ii) amino acid V76 of full length human p28 or amino acid V72 of full length murine p28, wherein the substitution is optionally alanine;
(iii) amino acid W197 of full length human p28 or amino acid W195 of full length murine p28, wherein the substitution is optionally alanine;
(iv) amino acid L200 of full length human p28 or amino acid L198 of full length murine p28, wherein the substitution is optionally alanine;
(v) amino acid L201 of full length human p28 or amino acid L199 of full length murine p28, wherein the substitution is optionally alanine;
(vi) amino acid Y204 of full length human p28 or amino acid Y202 of full length murine p28, wherein the substitution is optionally alanine;
(vii) amino acid R205 of full length human p28 or amino acid Q203 of full length murine p28, wherein the substitution is optionally alanine; or
(viii) any combination of (b)(i) to (b)(vii).

21. A nucleic acid or plurality of nucleic acids encoding the IL27 agonist of any one of claims 1 to 20.

22. A host cell engineered to express the IL27 agonist of any one of claims 1 to 20 or the nucleic acid or plurality of nucleic acids of claim 21.

23. A method of producing the IL27 agonist of any one of claims 1 to 20, comprising culturing the host cell of claim 22 and recovering the IL27 agonist expressed thereby.

24. A pharmaceutical composition comprising the IL27 agonist of any one of claims 1 to 20 and an excipient.

25. A method of (a) modulating the immune response; (b) treating an autoimmune condition; and/or (c) administering to the subject IL27 therapy with reduced systemic exposure and/or reduced systemic toxicity, comprising administering to a subject in need thereof the IL27 agonist of any one of claims 1 to 20 or the pharmaceutical composition of claim 24.

26. The method of claim 25, which is a method of treating an autoimmune condition.

27. The method of claim 26, wherein the autoimmune condition is arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, scleroderma, systemic sclerosis, or allergic asthma.

28. An invention as described herein, e.g., as defined in any one of numbered embodiments 1 through 355.


SEQUENCE LISTING (95 sequences)

SEQ ID NO: 1 - protein, 229 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 2 - protein, 243 aa, Homo sapiens
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 3 - protein, 232 aa, synthetic construct
DKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 235 aa, synthetic construct
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 8 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 9 - protein, 609 aa, Homo sapiens [sequence omitted]

SEQ ID NO: 10 - protein, 636 aa, Homo sapiens
MRGGRGAPFWLWPLPKLALLPLLWVLFQRTRPQGSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDPLEATVHWAPPTWPSHKVLICQFHYRRCQEAAWTLLEPELKTIPLTPVEIQDLELATGYKVYGRCRMEKEEDLWGEWSPILSFQTPPSAPKDVWVSGNLCGTPGGEEPLLLWKAPGPCVQVSYKVWFWVGGRELSPEGITCCCSLIPSGAEWARVSAVNATSWEPLTNLSLVCLDSASAPRSVAVSSIAGSTELLVTWQPGPGEPLEHVVDWARDGDPLEKLNWVRLPPGNLSALLPGNFTVGVPYRITVTAVSASGLASASSVWGFREELAPLVGPTLWRLQDAPPGTPAIAWGEVPRHQLRGHLTHYTLCAQSGTSPSVCMNVSGNTQSVTLPDLPWGPCELWVTASTIAGQGPPGPILRLHLPDNTLRWKVLPGILFLWGLFLLGCGLSLATSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLA

SEQ ID NO: 11 - protein, 918 aa, Homo sapiens
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 12 - protein, 4 aa, synthetic construct
GGSG

SEQ ID NO: 13 - protein, 5 aa, synthetic construct
GGSGG

SEQ ID NO: 14 - protein, 5 aa, synthetic construct
GSGSG

SEQ ID NO: 15 - protein, 5 aa, synthetic construct
GSGGG

SEQ ID NO: 16 - protein, 5 aa, synthetic construct
GGGSG

SEQ ID NO: 17 - protein, 5 aa, synthetic construct
GSSSG

SEQ ID NO: 18 - protein, 15 aa, synthetic construct
GCGASGGGGSGGGGS

SEQ ID NO: 19 - protein, 10 aa, synthetic construct
GGGGSGGGGS

SEQ ID NO: 20 - protein, 15 aa, synthetic construct
GGGASGGGGSGGGGS

SEQ ID NO: 21 - protein, 15 aa, synthetic construct
GGGGSGGGGSGGGGS

SEQ ID NO: 22 - protein, 10 aa, synthetic construct
GGGASGGGGS

SEQ ID NO: 23 - protein, 20 aa, synthetic construct
GGGGSGGGGSGGGGSGGGGS

SEQ ID NO: 24 - protein, 5 aa, synthetic construct
GCGGS

SEQ ID NO: 25 - protein, 4 aa, synthetic construct
CPPC

SEQ ID NO: 26 - protein, 4 aa, Homo sapiens
CPSC

SEQ ID NO: 27 - protein, 20 aa, synthetic construct
EPKSCDKTHTCPPCPAPPVA

SEQ ID NO: 28 - protein, 17 aa, synthetic construct
ESKYGPPCPPCPAPPVA

SEQ ID NO: 29 - protein, 15 aa, synthetic construct
CPPCPAPGGGGPSVF

SEQ ID NO: 30 - protein, 14 aa, synthetic construct
CPPCPAPGGGPSVF

SEQ ID NO: 31 - protein, 13 aa, synthetic construct
CPPCPAPGGPSVF

SEQ ID NO: 32 - protein, 12 aa, synthetic construct
CPPCPAPGPSVF

SEQ ID NO: 33 - protein, 243 aa, Homo sapiens; identical to SEQ ID NO: 2

SEQ ID NO: 34 - skipped

SEQ ID NO: 35 - protein, 228 aa, Mus sp.
MSKLLFLSLALWASRSPGYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKP

SEQ ID NO: 36 - protein, 234 aa, Mus sp.
MGQVTGDLGWRLSLLLLPLLLVQAGSWGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 37 - protein, 227 aa, synthetic construct
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 38 - protein, 5 aa, synthetic construct
GGGGS

SEQ ID NO: 39 - protein, 438 aa, synthetic construct
YTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGSGGGSGGGSGGGSFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSHHHHHH

SEQ ID NO: 40 - protein, 456 aa, synthetic construct
RKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGSGSSRGGSGSGGSGGGGSKLAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQPHHHHHH

SEQ ID NO: 41 - protein, 675 aa, synthetic construct
YTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGSGGGSGGGSGGGSFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGSGGGGSGGGGSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 449 aa, synthetic construct
FPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGSGGGGSGGGGSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - protein, 453 aa, synthetic construct
YTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK

SEQ ID NO: 44 - protein, 684 aa, synthetic construct
FPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGSGGGGSGGGGSGGGGSGGGGSYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 679 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSGGGGSFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 46 - protein, 684 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGSGGGGSGGGGSGGGGSGGGGSYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKP

SEQ ID NO: 47 - protein, 449 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 48 - protein, 453 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGKGGGGSGGGGSGGGGSYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKP

SEQ ID NO: 49 - protein, 679 aa, synthetic construct
YTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSGGGGSFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGSGGGGSGGGGSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 449 aa, synthetic construct
FPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGSGGGGSGGGGSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 679 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSGGGGSFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 52 - protein, 449 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 53 - protein, 1064 aa, synthetic construct
YTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSGGGGSFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLEQKLISEEDLGGEQKLISEEDLHHHHHH

SEQ ID NO: 54 - protein, 834 aa, synthetic construct
FPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLEQKLISEEDLGGEQKLISEEDLHHHHHH

SEQ ID NO: 55 - protein, 838 aa, synthetic construct
YTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLEQKLISEEDLGGEQKLISEEDLHHHHHH

SEQ ID NO: 56 - protein, 238 aa, synthetic construct
YTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPEQKLISEEDLGGEQKLISEEDLHHHHHH

SEQ ID NO: 57 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK

SEQ ID NO: 58 - protein, 658 aa, synthetic construct
YTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 679 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSGGGGSFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTAALLHSLELVLSRAVRDLLLLSLPRRPGSAWDS

SEQ ID NO: 60 - protein, 679 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSGGGGSFPTDPLSLQELRREFTVSLYLARALLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 61 - protein, 679 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSGGGGSFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRADLRDLHAALRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 62 - protein, 679 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSGGGGSFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQAAYTYQLLHSLELVLSRAVRDLLLLSLPRRPGSAWDS

SEQ ID NO: 63 - protein, 679 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSGGGGSFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSAPQLLYTYQLLHSLELVLSRAVRDLLLLSLPRRPGSAWDS

SEQ ID NO: 64 - protein, 897 aa, synthetic construct
QVQLQESGPGLVKPSETLSLTCTVSGGSISSYYWSWIRQPPGKGLEWIGYIYYSGRTNYNPSLKSRVTISVDTSKNQFSLKLSSVTAADTAIYYCARHRVTRTADSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSGGGGSFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQAAYTYQLLHSLELVLSRAVRDLLLLSLPRRPGSAWDS

SEQ ID NO: 65 - protein, 446 aa, synthetic construct
QVQLQESGPGLVKPSETLSLTCTVSGGSISSYYWSWIRQPPGKGLEWIGYIYYSGRTNYNPSLKSRVTISVDTSKNQFSLKLSSVTAADTAIYYCARHRVTRTADSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK

SEQ ID NO: 66 - protein, 214 aa, synthetic construct
DIQMTQSPSSLSASVGDRVTITCQASQDINNYLNWYQQKTGKAPKFLIYDASNLETGVSSRFSGSGSGTDFTFTISSLQPEDVGTYYCHQYGDLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 679 aa, synthetic construct; identical to SEQ ID NO: 62

SEQ ID NO: 68 - protein, 228 aa, synthetic construct; identical to SEQ ID NO: 57

SEQ ID NO: 69 - protein, 217 aa, synthetic construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 897 aa, synthetic construct
QVQLQESGPGLVKPSETLSLTCTVSGGSISSYYWSWIRQPPGKGLEWIGYIYYSGRTNYNPSLKSRVTISVDTSKNQFSLKLSSVTAADTAIYYCARHRVTRTADSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSGGGGSFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRADLRDLHAALRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 72 - protein, 897 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSGGGGSFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRADLRDLHAALRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 73 - protein, 446 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK

SEQ ID NO: 74 - protein, 898 aa, synthetic construct
QVQLVQSGAEVKRPGSSVKVSCKVSGVTFRNFAIIWVRQAPGQGLEWMGGIIPFFSAANYAQSFQGRVTITPDESTSTAFMELASLRSEDTAVYYCAREGERGHTYGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSYTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKPGGGGSGGGGSGGGGSGGGGSFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRADLRDLHAALRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 75 - protein, 447 aa, synthetic construct
QVQLVQSGAEVKRPGSSVKVSCKVSGVTFRNFAIIWVRQAPGQGLEWMGGIIPFFSAANYAQSFQGRVTITPDESTSTAFMELASLRSEDTAVYYCAREGERGHTYGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSPGK

SEQ ID NO: 76 - protein, 441 aa, synthetic construct
QVQLQESGPGLVAPSQSLSITCTVSGFSLTTFGVHWVRQSPGKGLEWLGVIWADETTNYNSALMSRLSISKDNSKSQVFLKMNGLRTDDTAIYSCARSKVSYYFDYWGRGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 77 - protein, 219 aa, synthetic construct
NIMMTQSPSSLPVSPGEKVTMNCKSSRSVLYSLNQKNYLAWYQQKPGQSPKLLIYWASTRESGVPDRFTGSGSGTDFSLTISSVQAEDLAVYYCHQYLSSWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 78 - protein, 448 aa, synthetic construct
EVQLVESGGGLVQPGGSLRLSCAASGFTLSTYAMTWVRQAPGKGLEWVSAINYRAANTWYADSVKGRFTISRDNSKNTLYLQMNSLRDEDTAVYYCAQDRVIIKDYYVMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - protein, 450 aa, synthetic construct
QVQLQESGPGLVKPSETLSLTCTVSGGSISSYYWSWIRQPPGKGLEWIGYIYYSGRTNYNPSLKSRVTISVDTSKNQFSLKLSSVTAADTAIYYCARHRVTRTADSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 81 - protein, 5 aa, synthetic construct
GGGGS

SEQ ID NO: 82 - protein, 4 aa, synthetic construct
GGGG

SEQ ID NO: 83 - protein, 5 aa, synthetic construct
GGGGG

SEQ ID NO: 84 - protein, 6 aa, synthetic construct
GGGGGG

SEQ ID NO: 85 - protein, 7 aa, synthetic construct
GGGGGGG

SEQ ID NO: 86 - protein, 8 aa, synthetic construct
GGGGGGGG

SEQ ID NO: 87 - protein, 9 aa, synthetic construct
GGGGGGGGG

SEQ ID NO: 88 - protein, 5 aa, synthetic construct
GSGGS

SEQ ID NO: 89 - protein, 4 aa, synthetic construct
GGGS

SEQ ID NO: 90 - protein, 4 aa, synthetic construct
GGGS

SEQ ID NO: 91 - protein, 5 aa, synthetic construct
  VARIANT 3: note = Any amino acid
WSXWS

SEQ ID NO: 92 - protein, 11 aa, synthetic construct
  VARIANT 1..10: note = This region may encompass 1-10 repeating residues
GGGGGGGGGGS

SEQ ID NO: 93 - protein, 11 aa, synthetic construct
  VARIANT 2..11: note = This region may encompass 1-10 repeating residues
SGGGGGGGGGG

SEQ ID NO: 94 - protein, 234 aa, Mus sp.; identical to SEQ ID NO: 36

SEQ ID NO: 95 - protein, 4 aa, Homo sapiens
CPPC